(19) United States
(12) Patent Application Publication
(10) Pub. No.: US 2022/0227765 A1
(43) Pub. Date: Jul. 21, 2022

(54) PYRIDO-INDOLE ANALOGUES AS GPX4 INHIBITORS

(71) Applicant: Ferro Therapeutics, Inc., Palo Alto, CA (US)

(72) Inventors: Anjali Pandey, Fremont, CA (US); Chun Jiang, Hillsborough, CA (US); Ruihong Chen, Burlingame, CA (US); Biswajit Kalita, Bangalore (IN); Athisayamani Jeyaraj Duraiswamy, Bangalore (IN)

(21) Appl. No.: 17/638,744

(22) PCT Filed: Aug. 26, 2020
(86) PCT No.: PCT/US2020/047993

§ 371 Date: Feb. 25, 2022

Related U.S. Application Data
(60) Provisional application No. 62/893,137, filed on Aug. 28, 2019.
(60) Provisional application No. 62/893,128, filed on Aug. 28, 2019.

(51) Int. Cl. C07D 471/04; C07D 519/00; A61K 45/06; A61K 31/437; A61K 31/496; A61K 31/4375; A61P 35/00

(52) U.S. Cl. CPC C07D 471/04; C07D 519/00; A61K 45/06; A61P 35/00; A61K 31/496; A61K 31/4375; A61K 31/437

(57) ABSTRACT

This present disclosure relates to compounds with ferroptosis inducing activity, a method of treating a subject with cancer with the compounds, and combination treatments with a second therapeutic agent.

### CROSS-REFERENCE TO RELATED APPLICATIONS

This application claims priority under 35 U.S.C. 119(e) to U.S. Provisional Application Ser. No. 62/893,128, filed on Aug. 28, 2019, and 62/893,137, filed on Aug. 28, 2019, which are incorporated herein by reference in their entireties.

### BACKGROUND

Glutathione peroxidase 4 (GPX4) can directly reduce phospholipid hydroperoxide. Depletion of GPX4 induces lipid peroxidation-dependent cell death. Cancer cells in a drug-induced, therapy-resistant state have an enhanced dependence on the lipid peroxidase activity of GPX4 to prevent undergoing ferroptotic cell death. Studies have shown that lipophilic antioxidants, such as ferrostatin, can rescue cells from GPX4 inhibition-induced ferroptosis. For instance, mesenchymal state GPX4-knockout cells can survive in the presence of ferrostatin, however, when the supply of ferrostatin is terminated, these cells undergo ferroptosis (see, e.g., Viswanathan et al., Nature 547:453-7, 2017). It has also been experimentally determined that that GPX4i can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, a GPX4 inhibitor can be useful to induce ferroptotic cancer cell death and thus treat cancer.

### SUMMARY

The present disclosure relates to compounds having ferroptosis inducing activity, and methods of using the compounds for the treatment of cancer. In certain embodiments, provided herein is a compound of Formula A-I:

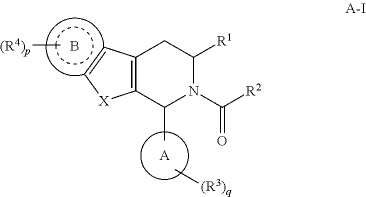

or a tautomer, stereoisomer, mixture of stereoisomers, isotopically enriched analog, or pharmaceutically acceptable salt thereof, wherein:

ring A is C₄-C₁₀cycloalkyl, heterocyclyl, aryl, or heteroaryl;

ring B is a 6-membered heteroaryl containing one or two N-atoms;

X is NR⁵, O or S;

p is 0, 1, 2 or 3;

q is 0, 1, 2 or 3;

R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OH, —C₁-C₆alkyl-OR⁸, or —Si(R¹⁵)₃;

R² is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;

each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two 127 together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, or heteroarylC₂-C₆alkenyl-.

In certain embodiments, provided herein is a compound of Formula C-I:

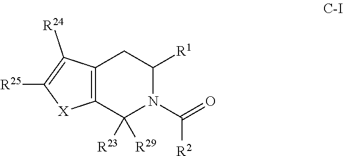

or a tautomer, stereoisomer, mixture of stereoisomers, isotopically enriched analog, or pharmaceutically acceptable salt thereof, wherein:

X is —NH—, —N(C₁-C₆alkyl)-, —O—, —S—, —N═CR¹⁶—, —CR¹⁶═CR¹⁶—, or —CR¹⁶═N—;

R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OR⁸, or —Si(R¹⁵)₃;

R² is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;

R²³ is C₁-C₉alkyl, C₂-C₉alkenyl, C₂-C₉alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl, wherein each C₁-C₉alkyl, C₂-C₉alkenyl, C₂-C₉alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl of R³ is independently optionally substituted with one to three R¹⁷;

R²⁹ is hydrogen or C₁-C₆alkyl; provided that when R²³ is C₃-C₁₀cycloalkyl, heterocyclyl, aryl, heteroaryl, then R²⁹ is C₁-C₆alkyl;

R²⁴ and R²⁵ are each independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ and R⁵ is independently optionally substituted with one to three R¹⁰; or

when X is —NH—, —N(C₁-C₆alkyl)-, —O—, or —S—; then R⁴ and R⁵ together with the atoms to which they are attached, can form a 6-membered aryl or 6-membered heteroaryl, wherein each aryl or heteroaryl is optionally substituted with one to three R¹⁴;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two 127 together with the nitrogen atom to which they are attached, form a 4 to 7 membered heterocyclyl; wherein each 127 or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl;

each R¹⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁴ is independently optionally substituted with one to three R¹⁰;

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, or heteroarylC₂-C₆alkenyl-;

each R¹⁶ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁶ is independently optionally substituted with one to three R¹⁰; and

each R¹⁷ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁷ is independently optionally substituted with one to three R¹⁰.

In certain embodiments, provided herein is a compound of Table A-1, or a tautomer, stereoisomer, mixture of stereoisomers, isotopically enriched analog, or pharmaceutically acceptable salt thereof. In certain embodiments, provided herein is a compound of Table B-1, or a tautomer, stereoisomer, mixture of stereoisomers, isotopically enriched analog, or pharmaceutically acceptable salt thereof. In certain embodiments, provided herein is a compound of Table C-1, or a tautomer, stereoisomer, mixture of stereoisomers, isotopically enriched analog, or pharmaceutically acceptable salt thereof.

In certain embodiments, the compounds exhibit GPX4 inhibiting activity, and in certain embodiments, exhibit altered or enhanced stability (e.g., metabolic stability) and/or enhanced activity or other characteristics as compared to other GPX4 inhibitors. In certain embodiments, the compounds described herein are selective for GPX4 over other GPXs. In certain embodiments, the compounds are used in a method of inhibiting GPX4 in a cell, comprising contacting a cell with an effective amount of the compound described herein to inhibit GPX4 in the cell. In certain embodiments, the cell is a cancer cell.

In certain embodiments, provided is a method of inducing ferroptosis in a cell comprising contacting the cell with an effective amount of a compound or composition provided herein.

In certain embodiments, provided is a method for treating a cancer in a patient in need thereof, comprising administering an effective amount of a compound or composition provided herein. In certain embodiments, provided is a method for treating a malignant solid tumor in a patient in need thereof, comprising administering an effective amount of a compound or composition provided herein to the patient. In certain embodiments, the malignant solid tumor is a sarcoma, carcinoma, or lymphoma.

### DETAILED DESCRIPTION

As used in this specification and the appended claims, the singular forms “a,” “an” and “the” include plural referents unless the context clearly indicates otherwise. Thus, for example, reference to “a protein” includes more than one protein, and reference to “a compound” refers to more than one compound.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

It is to be understood that both the foregoing general description, including the drawings, and the following detailed description are exemplary and explanatory only and are not restrictive of this disclosure. The section headings used herein are for organizational purposes only and not to be construed as limiting the subject matter described.

In reference to the present disclosure, the technical and scientific terms used in the descriptions herein will have the meanings commonly understood by one of ordinary skill in the art, unless specifically defined otherwise. Accordingly, the following terms are intended to have the meanings as described below.

“Ferroptosis” refers to a form of cell death understood in the art as involving generation of reactive oxygen species mediated by iron, and characterized by, in part, lipid peroxidation.

“Ferroptosis inducer” or “ferroptosis activator” refers to an agent which induces, promotes or activates ferroptosis.

“GPX4 inhibitor” refers to any agent that inhibits the activity of the enzyme glutathione peroxidase 4 (GPX4). A GPX4 inhibitor can be either a direct or indirect inhibitor. GPX4 is a phospholipid hydroperoxidase that in catalyzing the reduction of hydrogen peroxide and organic peroxides, thereby protects cells against membrane lipid peroxidation, or oxidative stress. GPX4 has a selenocysteine in the active site that is oxidized to a selenenic acid by the peroxide to afford a lipid-alcohol. The glutathione acts to reduce the selenenic acid (—SeOH) back to the selenol (—SeH). Should this catalytic cycle be disrupted, cell death occurs through an intracellular iron-mediated process known as ferroptosis.

“Subject” as used herein refers to a mammal, for example a dog, a cat, a horse, or a rabbit. In certain embodiments, the subject is a non-human primate, for example a monkey, chimpanzee, or gorilla. In certain embodiments, the subject is a human, sometimes referred to herein as a patient.

“Treating” or “treatment” of a disease, disorder, or syndrome, as used herein, includes (i) preventing the disease, disorder, or syndrome from occurring in a subject, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art, particularly in view of the guidance provided in the present disclosure.

“Therapeutically effective amount” refers to that amount which, when administered to an animal (e.g., human) for treating a disease, is sufficient to effect such treatment for the disease, disorder, or condition. In certain embodiments, the treatment provides a therapeutic benefit such as amelioration of symptoms or slowing of disease progression. For example, a therapeutically effective amount may be an amount sufficient to decrease a symptom of a disease or condition of as described herein.

The use of a dash, in certain embodiments, refers to a point of attachment. By way of example only, cycloalkylalkenyl- means that the point of attachment for a cycloalkylalkenyl substituent is the alkylene moiety.

“Alkyl” refers to a straight or branched chain hydrocarbon group of 1 to 20 carbon atoms (C₁-C₂₀ or C₁₋₂₀), e.g., 1 to 12 carbon atoms (C₁-C₁₂ or C₁₋₁₂), or 1 to 8 carbon atoms (C₁-C₈ or C₁₋₈). Exemplary “alkyl” includes, but are not limited to, methyl, ethyl, n-propyl, i-propyl, n-butyl, s-butyl, t-butyl, n-pentyl, and s-pentyl, and the like.

“Alkenyl” refers to a straight or branched chain hydrocarbon group of 2 to 20 carbon atoms (C₂-C₂₀ or C₂₋₂₀), e.g., 2 to 12 carbon atoms (C₂-C₁₂ or C₂₋₁₂), or 2 to 8 carbon atoms (C₂-C₈ or C₂₋₈), having at least one double bond. Exemplary “alkenyl” includes, but are not limited to, vinyl ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl, and the like.

“Alkynyl” refers to a straight or branched chain hydrocarbon group of 2 to 12 carbon atoms (C₂-C₁₂ or C₂₋₁₂), e.g., 2 to 8 carbon atoms (C₂-C₈ or C₂₋₈), containing at least one triple bond. Exemplary “alkynyl” includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl, and the like.

“Alkylene,” “alkenylene” and “alkynylene” refers to a straight or branched chain divalent hydrocarbon radical of the corresponding alkyl, alkenyl, and alkynyl, respectively. In certain embodiments, “alkyl,” “alkenyl,” and “alkynyl” can represent the corresponding “alkylene,” “alkenylene” and “alkynylene,” such as, by way of example and not limitation, cycloalkylalkyl-, heterocycloalkylalkyl-, arylalkyl-, heteroarylalkyl-, cycloalkylalkenyl-, heterocycloalkylalkenyl-, arylalkenyl-, heteroarylalkenyl-, cycloalkylalkynyl-, heterocycloalkylalkynyl-, arylalkynyl-, heteroarylalkynyl-, and the like, wherein the cycloalkyl, heterocycloalkyl, aryl, and heteroaryl group is connected, as a substituent via the corresponding alkylene, alkenylene, or alkynylene group.

“Lower” in reference to substituents refers to a group having between one and six carbon atoms.

“Alkylhalo” or “haloalkyl” refers to a straight or branched chain hydrocarbon group of 1 to 20 carbon atoms (C₁-C₂₀ or C₁₋₂₀), e.g., 1 to 12 carbon atoms (C₁-C₁₂ or C₁₋₁₂), or 1 to 8 carbon atoms (C₁-C₈ or C₁₋₈) wherein one or more (e.g., one to three, or one) hydrogen atom is replaced by a halogen (e.g., Cl, F, etc.). In certain embodiments, the term “alkylhalo” refers to an alkyl group as defined herein, wherein one hydrogen atom is replaced by a halogen (e.g., Cl, F, etc.). In certain embodiments, the term “alkylhalo” refers to an alkylchloride.

“Alkenylhalo” or “haloalkenyl” refers to a straight or branched chain hydrocarbon group of 2 to 20 carbon atoms (C₂-C₂₀ or C₂₋₂₀), e.g., 2 to 12 carbon atoms (C₂-C₁₂ or C₂₋₁₂), or 2 to 8 carbon atoms (C₂-C₈ or C₂₋₈), having at least one double bond, wherein one or more (e.g., one to three, or one) hydrogen atom is replaced by a halogen (e.g., Cl, F, etc.). In certain embodiments, the term “alkenylhalo” refers to an alkenyl group as defined herein, wherein one hydrogen atom is replaced by a halogen (e.g., Cl, F, etc.). In certain embodiments, the term “alkenylhalo” refers to an alkenylchloride.

“Cycloalkyl” refers to any stable monocyclic or polycyclic system which consists of carbon atoms, any ring of which being saturated. “Cycloalkenyl” refers to any stable monocyclic or polycyclic system which consists of carbon atoms, with at least one ring thereof being partially unsaturated. Examples of cycloalkyls include, but are not limited to, cyclopropyl, cyclobutyl, cyclopentyl, cyclohexyl, cycloheptyl, cyclooctyl, bicycloalkyls and tricycloalkyls (e.g., adamantyl).

“Heterocycloalkyl” or “heterocyclyl” refers to a 4 to 14 membered, mono- or polycyclic (e.g., bicyclic), non-aromatic hydrocarbon ring, wherein 1 to 3 carbon atoms are replaced by a heteroatom. Heteroatoms and/or heteroatomic groups which can replace the carbon atoms include, but are not limited to, —O—, —S—, —S—O—, —NR⁴⁰—, —PH—, —C(O)—, —S(O)—, —S(O)₂—, —S(O)NR⁴⁰—, —S(O)₂NR⁴⁰—, and the like, including combinations thereof, where each R⁴⁰ is independently hydrogen or lower alkyl. Examples include thiazolidinyl, thiadiazolyl, triazinyl, morpholinyl, pyrrolidinonyl, pyrrolidinyl, piperidinyl, piperazinyl, 2,3-dihydropyranyl, dihydropyranyl, hydantoinyl, valerolactamyl, oxiranyl, oxetanyl, tetrahydropyranyl, tetrahydropyranyl, dihydropyridinyl, tetrahydropyridinyl, tetrahydropyrimidinyl, tetrahydrothiophenyl, tetrahydrothiopyranyl, and the like. In certain embodiments, the “heterocycloalkyl” or “heterocyclyl” is a substituted or unsubstituted 4 to 7 membered monocyclic ring, wherein 1 to 3 carbon atoms are replaced by a heteroatom as described above.

In certain embodiments, the “heterocycloalkyl” or “heterocyclyl” is a 4 to 10, or 4 to 9, or 5 to 9, or 5 to 7, or 5 to 6 membered mono- or polycyclic (e.g., bicyclic) ring, wherein 1 to 3 carbon atoms are replaced by a heteroatom as described above. In certain embodiments, when the “heterocycloalkyl” or “heterocyclyl” is a substituted or unsubstituted bicyclic ring, one ring may be aromatic, provided at least one ring is non-aromatic, regardless of the point of attachment to the remainder of the molecule (e.g., indolinyl, isoindolinyl, and the like).

“Aryl” refers to a 6 to 14-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Examples of “aryl” groups include phenyl, naphthyl, indenyl, biphenyl, phenanthrenyl, naphthacenyl, and the like.

“Heteroaryl” means an aromatic heterocyclic ring, including monocyclic and polycyclic (e.g., bicyclic) ring systems, where at least one carbon atom of one or both of the rings is replaced with a heteroatom independently selected from nitrogen, oxygen, and sulfur, or at least two carbon atoms of one or both of the rings are replaced with a heteroatom independently selected from nitrogen, oxygen, and sulfur. In certain embodiments, the heteroaryl can be a 5 to 6 membered monocyclic, or 7 to 11 membered bicyclic ring systems. Examples of “heteroaryl” groups include pyrrolyl, pyrazolyl, imidazolyl, pyrazinyl, oxazolyl, isoxazolyl, thiazolyl, furyl, thienyl, pyridyl, pyrimidyl, benzothiazolyl, purinyl, benzimidazolyl, indolyl, isoquinolyl, quinoxalinyl, quinolyl, and the like.

“Bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 5-12 ring members and 0-4 heteroatoms independently selected from nitrogen, oxygen, and sulfur. Such bridged bicyclic groups include those groups set forth below where each group is attached to the rest of the molecule at any substitutable carbon or nitrogen atom. Exemplary bridged bicyclics include, but are not limited to:

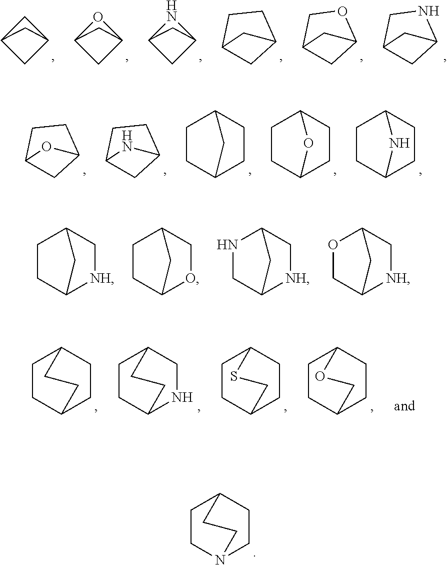

“Fused ring” refers a ring system with two or more rings having at least one bond and two atoms in common. A “fused aryl” and a “fused heteroaryl” refer to ring systems having at least one aryl and heteroaryl, respectively, that share at least one bond and two atoms in common with another ring.

“Halogen” or “halo” refers to fluorine, chlorine, bromine and iodine.

“Acyl” refers to —C(O)R⁴³, where R⁴³ is hydrogen, or an optionally substituted alkyl, heteroalkyl, cycloalkyl, cycloalkylalkyl, heterocycloalkyl, heterocycloalkylalkyl, aryl, arylalkyl, heteroaryl, or heteroarylalkyl as defined herein. Exemplary acyl groups include, but are not limited to, formyl, acetyl, cyclohexylcarbonyl, cyclohexylmethylcarbonyl, benzoyl, benzylcarbonyl, and the like.

“Alkyloxy” or “alkoxy” refers to OR⁴⁴, wherein R⁴⁴ is an optionally substituted alkyl.

“Aryloxy” refers to OR⁴⁵, wherein R⁴⁵ is an optionally substituted aryl.

“Carboxy” refers to COO⁻ or COOM, wherein M is H or a counterion (e.g., a cation, such as Na⁺, Ca²⁺, Mg²⁺, etc.).

“Carbamoyl” refers to —C(O)NR⁴⁶R⁴⁶, wherein each R⁴⁶ is independently selected from H or an optionally substituted alkyl, cycloalkyl, cycloalkylalkyl, heterocycloalkyl, heterocylcoalkylalkyl, aryl, arylalkyl, heteroaryl, or heteroarylalkyl.

“Ester” refers to a group such as —C(═O)OR⁴⁷, alternatively illustrated as C(O)OR⁴⁷, wherein R⁴⁷ is selected from an optionally substituted alkyl, cycloalkyl, cycloalkylalkyl, heterocycloalkyl, heterocyclolalkylalkyl, aryl, arylalkyl, heteroaryl, and heteroarylalkyl.

“Ether” refers to the group -alkyl-O-alkyl, where the term alkyl is as defined herein.

“Sulfanyl” refers to —SR⁴⁸, wherein R⁴⁸ is selected from an optionally substituted alkyl, cycloalkyl, cycloalkylalkyl, heterocycloalkyl, heterocycloalkylalkyl, aryl, arylalkyl, heteroaryl, and heteroarylalkyl. For example, —SR⁴⁸, wherein R⁴⁸ is an alkyl is an alkylsulfanyl.

“Sulfonyl” refers to —S(O)₂—, which may have various substituents to form different sulfonyl groups including sulfonic acids, sulfonamides, sulfonate esters, and sulfones. For example, —S(O)₂R⁴⁹, wherein R⁴⁹ is an alkyl refers to an alkylsulfonyl. In certain embodiments of —S(O)₂R⁴⁹, R⁴⁹ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Sulfinyl” refers to —S(O)—, which may have various substituents to form different sulfinyl groups including sulfinic acids, sulfinamides, and sulfinyl esters. For example, —S(O)R⁵⁰, wherein R⁵⁰ is an alkyl refers to an alkylsulfinyl. In certain embodiments of —S(O)R⁵⁰, R⁵⁰ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Silyl” refers to Si, which may have various substituents, for example —SiR⁵¹R⁵¹R⁵¹, where each R⁵¹ is independently selected from alkyl, cycloalkyl, cycloalkylalkyl, heterocycloalkyl, heterocycloalkylalkyl, aryl, arylalkyl, heteroaryl, and heteroarylalkyl. As defined herein, any heterocycloalkyl or heteroaryl group present in a silyl group has from 1 to 3 heteroatoms selected independently from O, N, and S.

“Amino” or “amine” refers to the group NR⁵²R⁵² or N⁺R⁵²R⁵²R⁵², wherein each R⁵² is independently selected from hydrogen and an optionally substituted alkyl, cycloalkyl, heterocycloalkyl, alkyloxy, aryl, heteroaryl, heteroarylalkyl, acyl, —C(O)—O-alkyl, sulfanyl, sulfinyl, sulfonyl, and the like. Exemplary amino groups include, but are not limited to, dimethylamino, diethylamino, trimethylammonium, triethylammonium, methylysulfonylamino, furanyl-oxy-sulfamino, and the like.

“Amide” refers to a group such as —C(═O)NR⁵³R⁵³, wherein each R⁵³ is independently selected from H and an optionally substituted alkyl, cycloalkyl, cycloalkylalkyl, heterocycloalkyl, heterocycloalkylalkyl, aryl, arylalkyl, heteroaryl, and heteroarylalkyl.

“Carbamate” referes to a group such as —O—C(═O)NR⁵³R⁵³ or —NR⁵³—C(═O)OR⁵³, wherein each R⁵³ is independently selected from H and an optionally substituted alkyl, cycloalkyl, cycloalkylalkyl, heterocycloalkyl, heterocycloalkylalkyl, aryl, arylalkyl, heteroaryl, and heteroarylalkyl.

“Sulfonamide” refers to S(O)₂NR⁵⁴R⁵⁴, wherein each R⁵⁴ is independently selected from H and an optionally substituted alkyl, heteroalkyl, heteroaryl, heterocycle, alkenyl, alkynyl, arylalkyl, heteroarylalkyl, heterocyclylalkyl, alkylene-C(O)—OR⁵⁵, or alkylene-O—C(O)—OR⁵⁵, where R⁵⁵ is selected from H, alkyl, heteroalkyl, cycloalkyl, heterocyclyl, aryl, heteroaryl, alkenyl, alkynyl, arylalkyl, heterocycloalkyl, heteroarylalkyl, amino, and sulfinyl.

“Adamantyl” refers to a compound of structural formula:

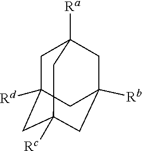

where optional substitutions can be present on one or more of R^{a}, R^{b}, R^{c}, and R^{d}. Adamantyl includes substituted adamantyl, e.g., 1- or 2-adamantyl, substituted by one or more substituents, including alkyl, halo, —OH, —NH₂, and alkoxy. Exemplary derivatives include methyladamatane, haloadamantane, hydroxyadamantane, and aminoadamantane (e.g., amantadine).

“N-protecting group” as used herein refers to those groups intended to protect a nitrogen atom against undesirable reactions during synthetic procedures. Exemplary N-protecting groups include, but is not limited to, acyl groups such acetyl and t-butylacetyl, pivaloyl, alkoxycarbonyl groups such as methyloxycarbonyl and t-butyloxycarbonyl (Boc), aryloxycarbonyl groups such as benzyloxycarbonyl (Cbz) and fluorenylmethoxycarbonyl (Fmoc and aroyl groups such as benzoyl. N-protecting groups are described in Greene's Protective Groups in Organic Synthesis, 5th Edition, P. G. M. Wuts, ed., Wiley (2014).

“Optional” or “optionally” refers to a described event or circumstance may or may not occur, and that the description includes instances where the event or circumstance occurs and instances where the event or circumstance does not. For example, “optionally substituted alkyl” refers to an alkyl group that may or may not be substituted and that the description encompasses both substituted alkyl group and unsubstituted alkyl group.

“Substituted” as used herein means one or more hydrogen atoms of the group is replaced with a substituent atom or group commonly used in pharmaceutical chemistry. Each substituent can be the same or different. Examples of suitable substituents include, but are not limited to, alkyl, alkenyl, alkynyl, cycloalkyl, aryl, arylalkyl, heterocycloalkyl, heteroaryl, —OR⁵⁶ (e.g., hydroxyl, alkyloxy (e.g., methoxy, ethoxy, and propoxy), ether, ester, carbamate, etc.), hydroxyalkyl, —C(O)O-alkyl, —O-alkyl-O-alkyl, haloalkyl, alkyl-O-alkyl, SR⁵⁶ (e.g., —SH, —S-alkyl, —S-aryl, —S-heteroaryl, arylalkyl-S—, etc.), S⁺R⁵⁶₂, S(O)R⁵⁶, SO₂R⁵⁶, NR⁵⁶R⁵⁷ (e.g., primary amine (i.e., NH₂), secondary amine, tertiary amine, amide, carbamate, urea, etc.), hydrazide, halo, nitrile, nitro, sulfide, sulfoxide, sulfone, sulfonamide, —SH, carboxy, aldehyde, keto, carboxylic acid, ester, amide, imine, and imide, including seleno and thio derivatives thereof, wherein each R⁵⁶ and R⁵⁷ are independently alkyl, alkenyl, alkynyl, heteroalkyl, cycloalkyl, cycloalkylalkyl, heterocycloalkyl, heterocycloalkylalkyl, aryl, arylalkyl, heteroaryl, or heteroarylalkyl, and wherein each of the substituents can be optionally further substituted. In embodiments in which a functional group with an aromatic carbon ring is substituted, such substitutions will typically number less than about 10 substitutions, or about 1 to 5, with about 1 or 2 substitutions in certain embodiments.

“Pharmaceutically acceptable salt” is meant to include salts of the active compounds which are prepared with relatively nontoxic acids or bases, depending on the particular substituents found on the compounds described herein. When compounds as disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds as disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure may contain both basic and acidic functionalities that allow the compounds to be converted into either base or acid addition salts. Lists of suitable salts are found in Remington's Pharmaceutical Sciences, 17th Ed., Mack Publishing Company, Easton, Pa., (1985) and Journal of Pharmaceutical Science, 66:2 (1977), each of which is incorporated herein by reference in its entirety.

“Pharmaceutically acceptable carrier” or “pharmaceutically acceptable excipient” refers to an excipient, carrier or adjuvant that can be administered to a subject, together with at least one compound, and which does not destroy the pharmacological activity thereof and is generally safe, nontoxic and neither biologically nor otherwise undesirable when administered in doses sufficient to deliver a therapeutic amount of the agent.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹F, ³²F, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, e.g., a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds as disclosed herein, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

### 2. Compounds

In certain embodiments, provided herein is a compound of Formula A-I or a tautomer, stereoisomer, mixture of stereoisomers, isotopically enriched analog, or pharmaceutically acceptable salt thereof:

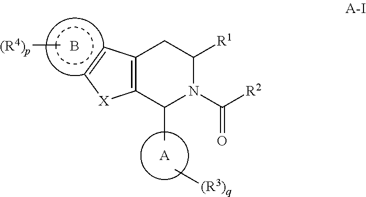

wherein:

ring A is C₄-C₁₀cycloalkyl, heterocyclyl, aryl, or heteroaryl;

ring B is a 6-membered heteroaryl containing one or two N-atoms;

X is NR⁵, O or S;

p is 0, 1, 2 or 3;

q is 0, 1, 2 or 3;

R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OR⁸, or —Si(R¹⁵)₃;

R² is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;

each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;

each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl; and

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, or heteroarylC₂-C₆alkenyl-.

In certain embodiments, provided herein is a compound of Formula A-IA, or a tautomer, stereoisomer, mixture of stereoisomers, isotopically enriched analog, or pharmaceutically acceptable salt thereof:

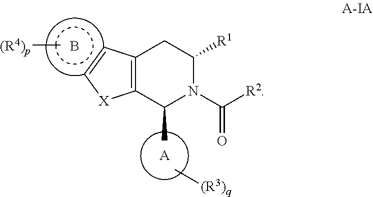

In certain embodiments, X is NR⁵ or S.

In certain embodiments, when X is NR⁵, then R² is C₂alkynyl.

In certain embodiments, when X is NR⁵, and R² is —C₁-C₂haloalkyl, —C₂-C₃alkenyl, —C₂-C₃haloalkenyl, or —CH₂OS(O)₂-phenyl, wherein the C₁-C₂alkylhalo and —C₂-C₃alkenylhalo are optionally substituted with one or two —CH₃, and the phenyl is optionally substituted with —CH₃, then R¹ is other than —C(O)OR⁶ and —C(O)N(R⁷)₂.

In certain embodiments, when X is NR⁵, then (i) R² is C₂alkynyl; or (ii) R² is —C₁-C₂haloalkyl, —C₂-C₃alkenyl, —C₂-C₃haloalkenyl, or —CH₂OS(O)₂-phenyl, wherein the C₁-C₂alkylhalo and —C₂-C₃alkenylhalo are optionally substituted with one or two —CH₃, and the phenyl is optionally substituted with —CH₃, and R¹ is other than —C(O)OR⁶ and —C(O)N(R⁷)₂.

In certain embodiments, provided herein is a compound of Formula A-II, or a tautomer, stereoisomer, mixture of stereoisomers, isotopically enriched analog, or pharmaceutically acceptable salt thereof:

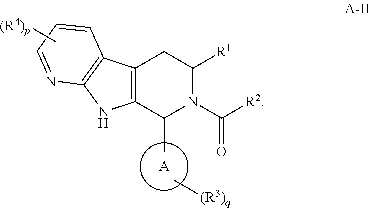

In certain embodiments, provided herein is a compound of Formula A-III, or a tautomer, stereoisomer, mixture of stereoisomers, isotopically enriched analog, or pharmaceutically acceptable salt thereof:

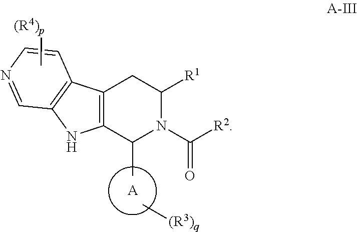

In certain embodiments, provided herein is a compound of Formula A-IV, or a tautomer, stereoisomer, mixture of stereoisomers, isotopically enriched analog, or pharmaceutically acceptable salt thereof:

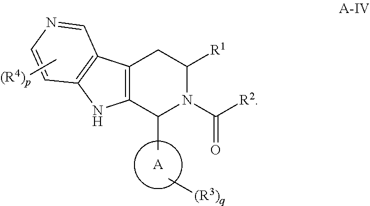

In certain embodiments, provided herein is a compound of Formula A-V, or a tautomer, stereoisomer, mixture of stereoisomers, isotopically enriched analog, or pharmaceutically acceptable salt thereof:

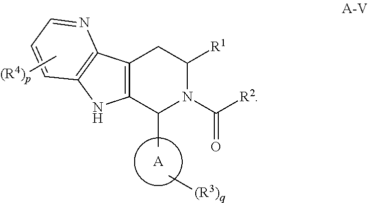

In certain embodiments, provided herein is a compound of Formula A-VI, or a tautomer, stereoisomer, mixture of stereoisomers, isotopically enriched analog, or pharmaceutically acceptable salt thereof:

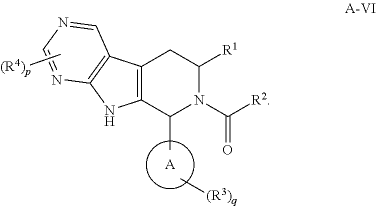

In certain embodiments, provided herein is a compound of Formula A-VII, or a tautomer, stereoisomer, mixture of stereoisomers, isotopically enriched analog, or pharmaceutically acceptable salt thereof:

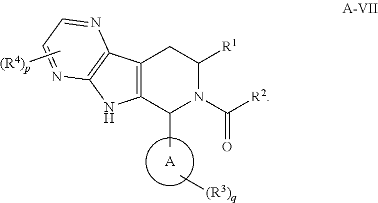

In certain embodiments, provided herein is a compound of Formula A-VIII, or a tautomer, stereoisomer, mixture of stereoisomers, isotopically enriched analog, or pharmaceutically acceptable salt

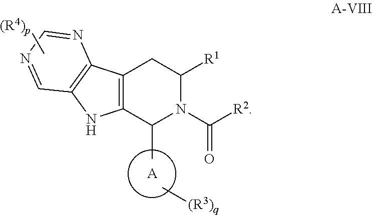

In certain embodiments, R² is C₁-C₂alkylhalo.

In certain embodiments, R² is-CH₂Cl or -CD₂Cl.

In certain embodiments, R² is —C≡CH.

In certain embodiments, provided herein is a compound of Formula A-IX, or a tautomer, stereoisomer, mixture of stereoisomers, isotopically enriched analog, or pharmaceutically acceptable salt thereof:

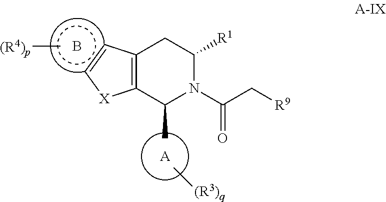

wherein R⁹ is halo.

In certain embodiments, provided herein is a compound of Formula A-X, or a tautomer, stereoisomer, mixture of stereoisomers, isotopically enriched analog, or pharmaceutically acceptable salt thereof:

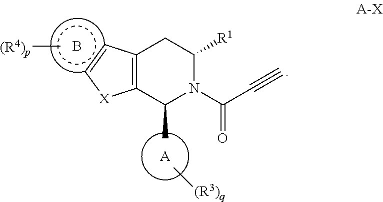

In certain embodiments, IV is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸.

In certain embodiments, R¹ is C₁-C₆alkyl, C₂-C₆alkenyl, C₂-C₆alkynyl, C₁-C₆haloalkyl, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸.

In certain embodiments, R¹ is C₁-C₆alkyl, C₂-C₆alkenyl, C₂-C₆alkynyl, C₁-C₆haloalkyl, —CN, C₃-C₁₀cycloalkyl, —NH₂, —NHR⁸, —N(R⁸)₂, —OH, —OR⁸, —C₁-C₆alkyl-OH or —C₁-C₆alkyl-OR⁸.

In certain embodiments, R¹ is C₁-C₆alkyl, C₁-C₆haloalkyl, C₃-C₁₀cycloalkyl, —CN, —C(O)OR⁶, —C(O)N(R⁷)₂, —C₁-C₆alkyl-OH or —C₁-C₆alkyl-OR⁸.

In certain embodiments, R¹ is C₁-C₆alkyl, C₂-C₆alkenyl, C₂-C₆alkynyl, C₁-C₆haloalkyl, —NH₂, —NHR⁸, —N(R⁸)₂, —OH, —OR⁸, —C₁-C₆alkyl-OH or —C₁-C₆alkyl-OR⁸.

In certain embodiments, R¹ is —C(O)OR⁶ or —C(O)N(R⁷)₂. In certain embodiments, R¹ is C₁-C₆alkyl. In certain embodiments, R¹ is C₃-C₁₀cycloalkyl.

In certain embodiments, R¹ is C₁-C₆alkyl. In certain embodiments, In certain embodiments, R¹ is C₂-C₆alkyl. In certain embodiments, R¹ is C₃-C₆alkyl. In certain embodiments, R¹ is C₅-C₆alkyl. In certain embodiments, R¹ is C₂-C₃alkyl. In certain embodiments, R¹ is C₄-C₆alkyl. In certain embodiments, R¹ is methyl. In certain embodiments, R¹ is n-butyl.

In certain embodiments, R¹ is —CH₂—R²⁶, wherein R²⁶ is C₁-C₅alkyl, C₂-C₅alkenyl, C₂-C₅alkynyl, C₁-C₅haloalkyl, —C₁-C₆alkyl-OH or —C₁-C₆alkyl-OR⁸.

In certain embodiments, R¹ is C₂-C₆alkenyl, C₂-C₆alkynyl, C₁-C₆haloalkyl, C₃-C₁₀cycloalkyl, —CN, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R¹⁵)₃.

In certain embodiments, R¹ is other than —C(O)OR⁶. In certain embodiments, R¹ is other than —C(O)OCH₃.

In certain embodiments, provided herein is a compound of Formula A-XII, or a tautomer, stereoisomer, mixture of stereoisomers, isotopically enriched analog, or pharmaceutically acceptable salt thereof:

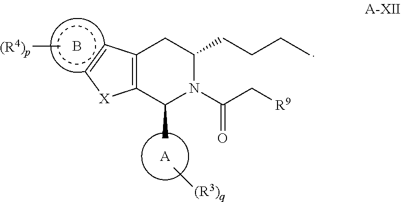

In certain embodiments, provided herein is a compound of Formula A-XIII, or a tautomer, stereoisomer, mixture of stereoisomers, isotopically enriched analog, or pharmaceutically acceptable salt thereof:

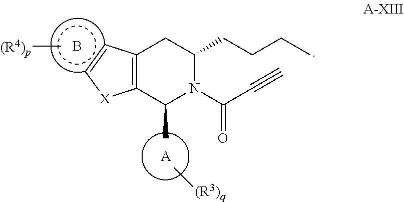

In certain embodiments, ring A is:

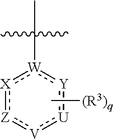

wherein 0 to 3 of U, V, W, X, Y, and Z is independently N, S, or O, and the remaining variables are CH or CR³ and each [symbol] independently represents a single or double bond, which comply with valency requirements based on U, V, W, X, Y and Z.

In certain embodiments, ring A is:

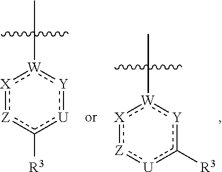

wherein 1 to 3 of U, W, X, Y, and Z is N, S, or O, and the remaining variables are CH or CR³ and [symbol] represents a single or double bond, which comply with valency requirements based on U, W, X, Y and Z.

In certain embodiments, ring A is aryl or heteroaryl. In certain embodiments, ring A is a monocyclic aryl or monocyclic heteroaryl. In certain embodiments, ring A is heterocyclyl. In certain embodiments, ring A is a 4 to 7 membered heterocyclyl. In certain embodiments, ring A is aryl. In certain embodiments, ring A is phenyl. In certain embodiments, ring A is heteroaryl. In certain embodiments, ring A is pyridyl. In certain embodiments, ring A is pyrazolyl. In certain embodiments, ring A is phenyl, pyridyl, piperidynyl, piperazinyl, or morpholinyl.

In certain embodiments, ring A is aryl or heteroaryl, each of which is substituted by one to three R³. In certain embodiments, ring A is aryl or heteroaryl, each of which is substituted by one to three R³, where at least one R³ is C₃-C₁₀cycloalkyl, heterocyclyl, aryl, or heteroaryl; wherein each C₃-C₁₀cycloalkyl, heterocyclyl, aryl, and heteroaryl of R³ is optionally substituted with one to three R¹⁰.

In certain embodiments, ring A is aryl or heteroaryl, each of which is substituted by two or three R³. In certain embodiments, ring A is aryl or heteroaryl, each of which is substituted by two or three R³; wherein at least one R³ is halo.

In certain embodiments, ring A is cyclohexyl. In certain embodiments, ring A is C₄-C₁₀cycloalkyl. In certain embodiments, ring A is a C₄-C₇cycloalkyl. In certain embodiments, ring A is bicyclo[1.1.1]pentanyl. In certain embodiments, ring A is selected from cyclobutyl, cyclopentyl, cyclohexyl, and cycloheptyl.

In certain embodiments, ring A is:

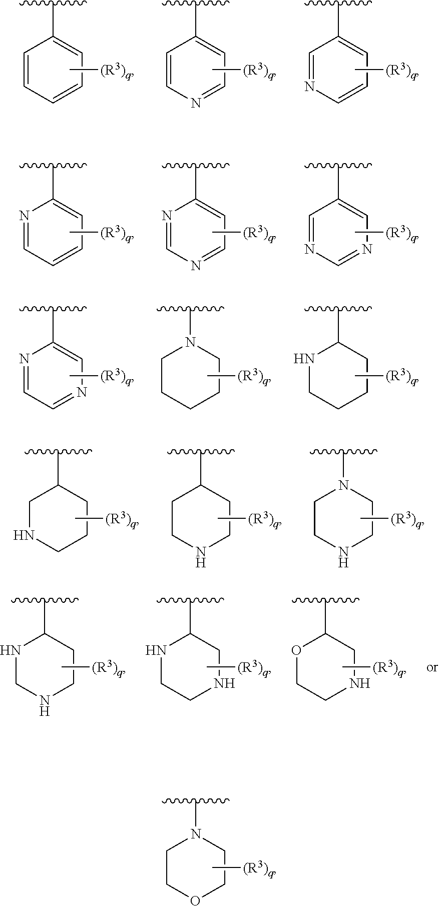

where q and each R³ is independently as defined herein.

In certain embodiments, ring A is:

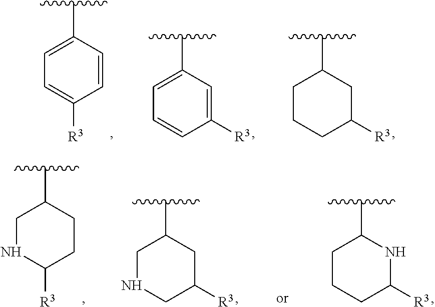

where R³ is independently as defined herein.

In certain embodiments, ring A is a bridged bicyclic ring selected from:

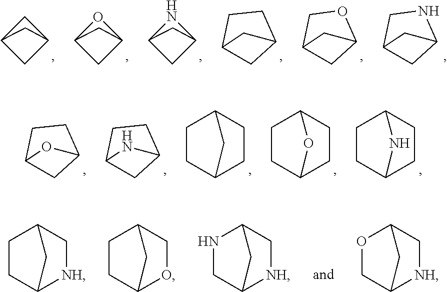

wherein each is substituted with one to three R³. In certain embodiments, ring A is a bridged bicyclic ring selected from:

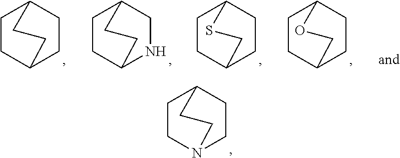

wherein each R³ is attached to a carbon atom on the bridged bicyclic ring.

In certain embodiments, ring A is:

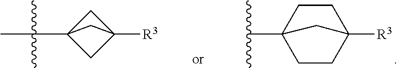

In certain embodiments, at least one R³ is —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

In certain embodiments, at least one R³ is —NHR⁸ or —N(R⁸)₂.

In certain embodiments, at least one R³ is —C(O)OR⁶ or —C(O)R⁶.

In certain embodiments, at least one R³ is —S(O)₂N(R⁷)₂, —S(O)N(R⁷)₂, or —C(O)N(R⁷)₂.

In certain embodiments, at least one R³ is —S(O)₂R⁸, —S(O)R⁸, —NR¹²C(O)R⁸, —NR¹²C(O)OR⁸, —OC(O)R⁸, or —OC(O)CHR⁸N(R¹²)₂.

In certain embodiments, at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

In certain embodiments, at least one R³ is halo.

In certain embodiments, at least one R³ is —NHR⁸. In certain embodiments, at least one R³ is —N(R⁸)₂. In certain embodiments, q is 2, and one R³ is halo and the other R³ is —N(R⁸)₂. In certain embodiments, q is 3, and two R³ are independently halo and one R³ is —N(R⁸)₂.

In certain embodiments, R³ is —C(O)OR⁶ or —C(O)R⁶.

In certain embodiments, R³ is —S(O)₂N(R⁷)₂, —S(O)N(R⁷)₂, or —C(O)N(R⁷)₂.

In certain embodiments, R³ is —S(O)₂R⁸, —S(O)R⁸, —NR¹²C(O)R⁸, —NR¹²C(O)OR⁸, —OC(O)R⁸, or —OC(O)CHR⁸N(R¹²)₂.

In certain embodiments, each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently optionally substituted with one to three R¹⁰.

In certain embodiments, each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl is independently optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰. In certain embodiments, each R⁴ is independently halo, —CN, —OH, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

In certain embodiments, each R⁴ is independently halo, —CN, —OH, —OR⁸, C₁-C₆alkyl, or C₂-C₆alkynyl; wherein the C₁-C₆alkyl of R⁴ is optionally substituted with one to three R¹⁰.

In certain embodiments, each R⁴ is independently halo, —CN, —OH, —OR⁸, C₁-C₆alkyl, C₂-C₆alkynyl; wherein the C₁-C₆alkyl of R⁴ is optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three R¹¹.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R¹⁰ is independently —OR¹², —N(R¹²)₂, S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, ring A is C₄-C₁₀cycloalkyl, heterocyclyl, aryl, or heteroaryl;

X is NR⁵ or S;

p is 0, 1, 2 or 3;

q is 0, 1, 2 or 3;

R¹ is C₁-C₆alkyl, C₁-C₆haloalkyl, C₃-C₁₀cycloalkyl, —CN, —C(O)OR⁶, —C(O)N(R⁷)₂, —C₁-C₆alkyl-OH or —C₁-C₆alkyl-OR⁸;

R² is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;
each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently optionally substituted with one to three R¹⁰;

each R⁴ is independently halo, —CN, —OH, —OR⁸, C₁-C₆alkyl, or C₂-C₆alkynyl; wherein the C₁-C₆alkyl of R⁴ is optionally independently optionally substituted with one to three R¹⁰;

R⁵ is hydrogen or C₁-C₆alkyl;

each R⁶ is independently hydrogen, C₁-C₆alkyl, C₂-C₆alkenyl, or —C₁-C₆alkylC₃-C₁₀cycloalkyl;

wherein each R⁶ is independently further substituted with one to three R¹¹;

each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹;

each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹;

each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R¹⁵ is independently C₁-C₆alkyl.

In certain embodiments, p is 1, 2 or 3. In certain embodiments, p is 1. In certain embodiments, p is 2. In certain embodiments, p is 3. In certain embodiments, p is 0. In certain embodiments, p is 0 or 1. In certain embodiments, p is 1 or 2.

In certain embodiments, q is 1, 2 or 3. In certain embodiments, q is 1. In certain embodiments, q is 2. In certain embodiments, q is 3. In certain embodiments, q is 0. In certain embodiments, q is 0 or 1. In certain embodiments, q is 1 or 2.

In certain embodiments, provided is a compound, or a tautomer, stereoisomer, mixture of stereoisomers, isotopically enriched analog, or pharmaceutically acceptable salt thereof, selected from Table A-1:

TABLE A-1

| No. | Structure |
|---|---|
| A-1 | 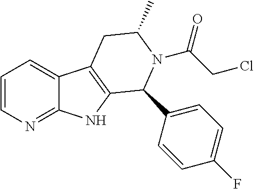 [SMILES: C[C@H]1Cc2c([nH]c3ncccc23)[C@H](c2ccc(F)cc2)N1C(=O)CCl] |
| A-2 | 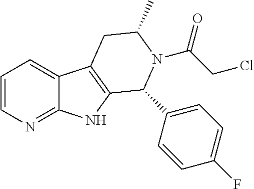 [SMILES: C[C@H]1Cc2c([nH]c3ncccc23)[C@@H](c2ccc(F)cc2)N1C(=O)CCl] |
| A-3 | 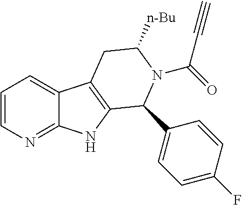 |
| A-4 | 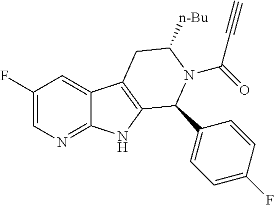 |
| A-5 | 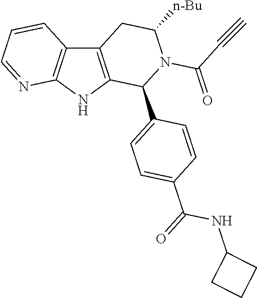 |
| A-6 | 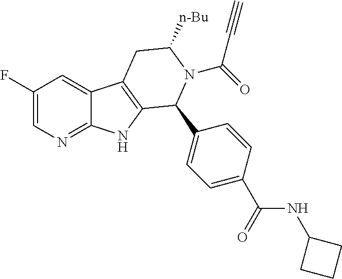 |
| A-7 | 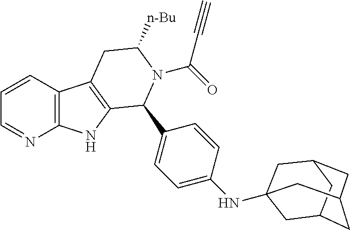 |
| A-8 | 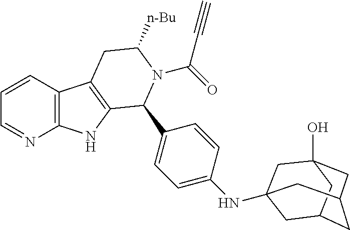 |
| A-9 | 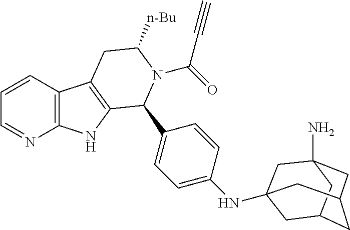 |
| A-10 | 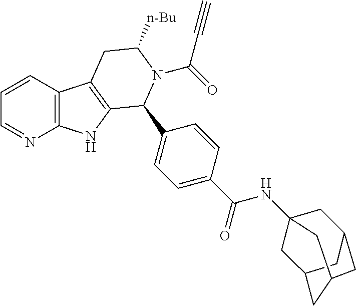 |
| A-11 | 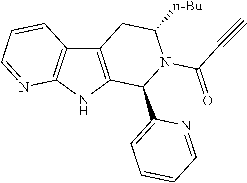 |
| A-12 | 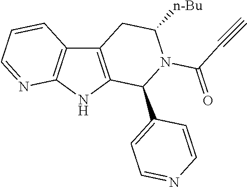 |
| A-13 | 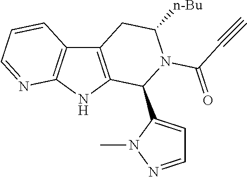 |
| A-14 | 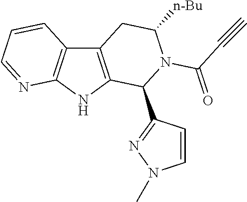 |
| A-15 | 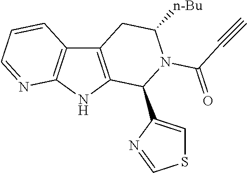 |
| A-16 | 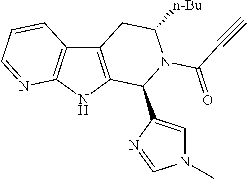 |
| A-17 | 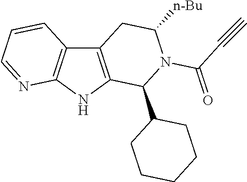 |
| A-18 | 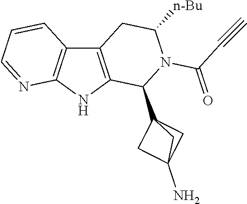 |

In certain embodiments, provided is a compound, or a tautomer, stereoisomer, mixture of stereoisomers, isotopically enriched analog, or pharmaceutically acceptable salt thereof, selected from Table B-1:

TABLE B-1

| No. | Structure |
|---|---|
| B-1 | 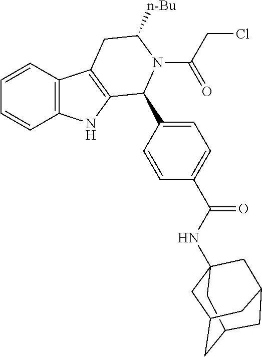 |
| B-2 | 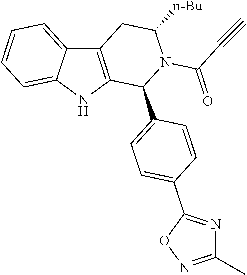 |
| B-3 | 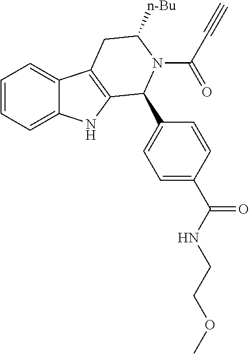 |
| B-4 | 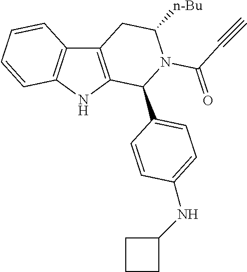 |
| B-5 | 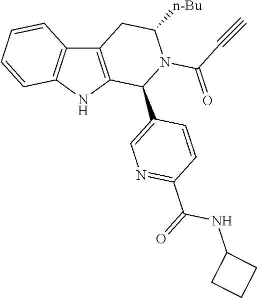 |
| B-6 | 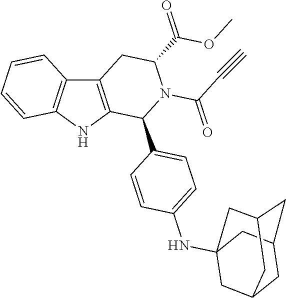 [SMILES: C#CC(=O)N1[C@@H](c2ccc(NC34CC5CC(CC(C5)C3)C4)cc2)c2[nH]c3ccccc3c2C[C@@H]1C(=O)OC] |
| B-7 | 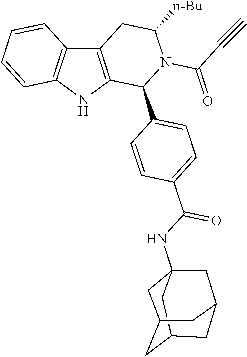 |
| B-8 | 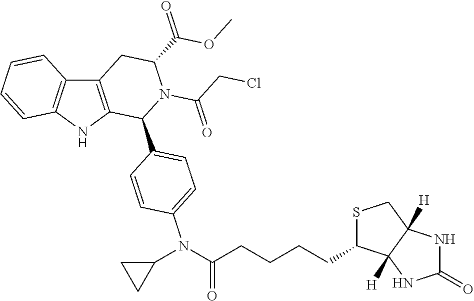 [SMILES: COC(=O)[C@H]1Cc2c([nH]c3ccccc23)[C@H](c2ccc(N(C(=O)CCCC[C@@H]3SC[C@@H]4NC(=O)N[C@@H]43)C3CC3)cc2)N1C(=O)CCl] |
| B-9 | 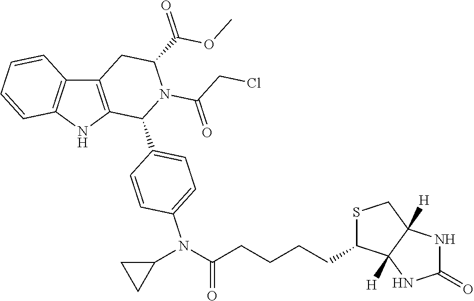 [SMILES: COC(=O)[C@H]1Cc2c([nH]c3ccccc23)[C@@H](c2ccc(N(C(=O)CCCC[C@@H]3SC[C@@H]4NC(=O)N[C@@H]43)C3CC3)cc2)N1C(=O)CCl] |
| B-10 | 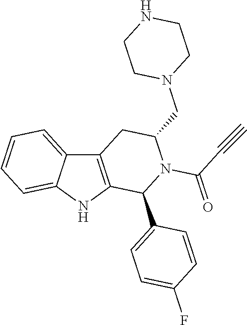 [SMILES: C#CC(=O)N1[C@@H](CN2CCNCC2)Cc2c([nH]c3ccccc23)[C@@H]1c1ccc(F)cc1] |
| B-11 | 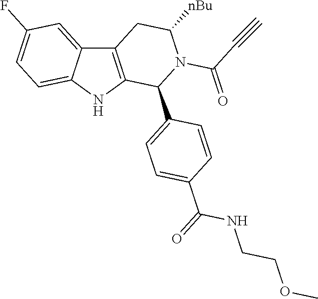 |
| B-12 | 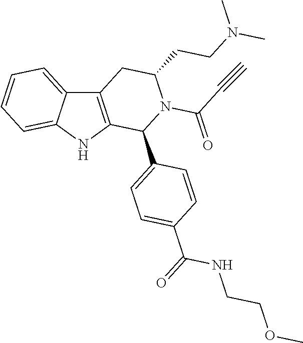 [SMILES: C#CC(=O)N1[C@@H](CCN(C)C)Cc2c([nH]c3ccccc23)[C@@H]1c1ccc(C(=O)NCCOC)cc1] |
| B-13 | 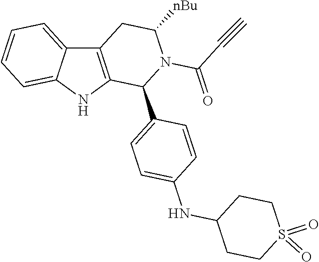 |
| B-14 | 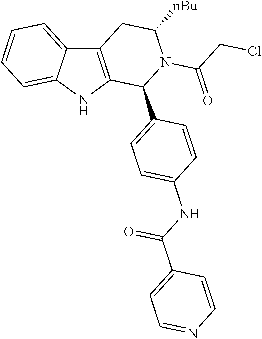 |
| B-15 | 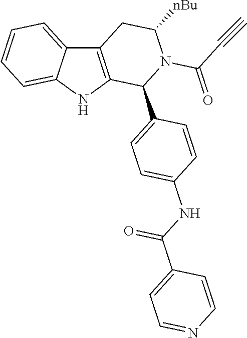 |
| B-16 | 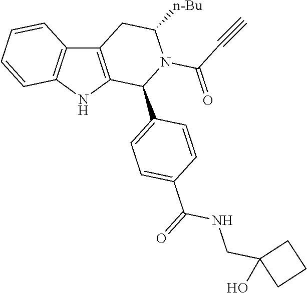 |
| B-17 | 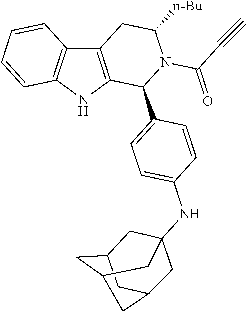 |
| B-18 | 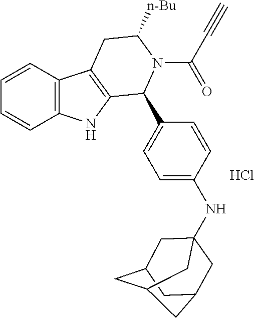 |
| 19 | 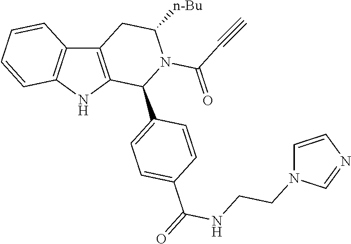 |
| B-20 | 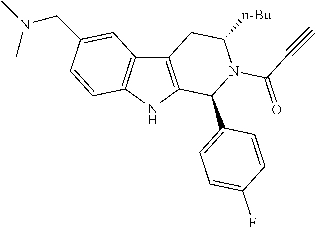 |
| B-21 | 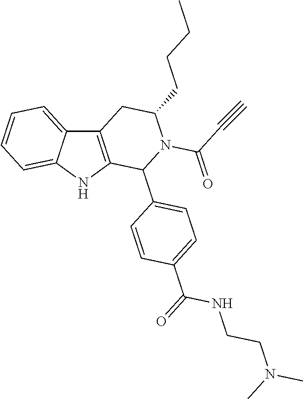 [SMILES: C#CC(=O)N1C(c2ccc(C(=O)NCCN(C)C)cc2)c2[nH]c3ccccc3c2C[C@@H]1CCCC] |
| B-22 | 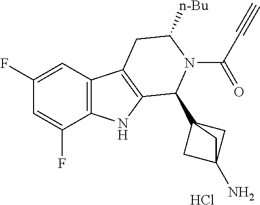 |
| B-23 | 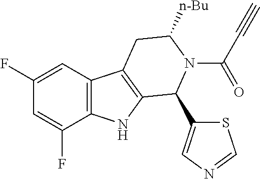 |
| B-24 | 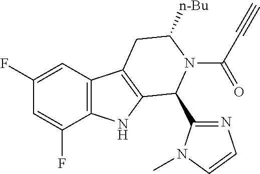 |
| B-25 | 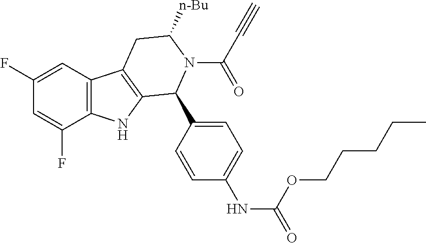 |
| B-26 | 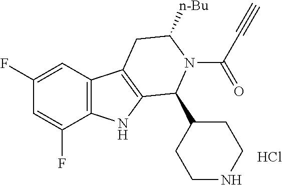 |
| B-27 | 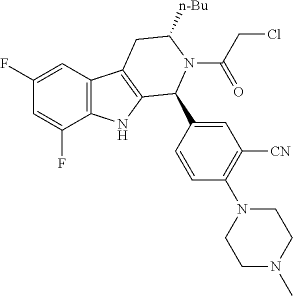 |
| B-28 | 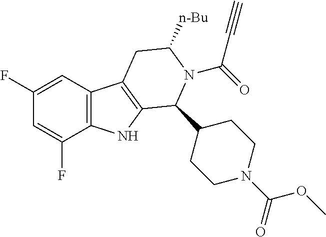 |
| B-29 | 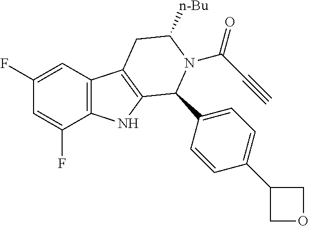 |
| B-30 | 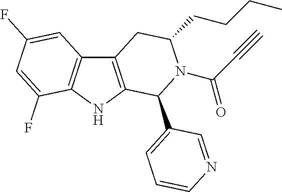 [SMILES: C#CC(=O)N1[C@@H](CCCC)Cc2c([nH]c3c(F)cc(F)cc23)[C@@H]1c1cccnc1] |
| B-31 | 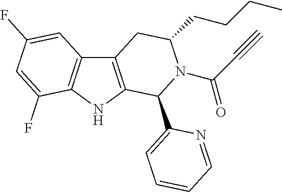 [SMILES: C#CC(=O)N1[C@@H](CCCC)Cc2c([nH]c3c(F)cc(F)cc23)[C@@H]1c1ccccn1] |
| B-32 | 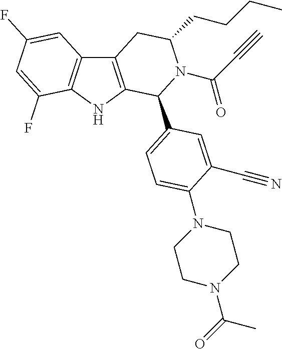 [SMILES: C#CC(=O)N1[C@@H](CCCC)Cc2c([nH]c3c(F)cc(F)cc23)[C@@H]1c1ccc(N2CCN(C(C)=O)CC2)c(C#N)c1] |
| B-33 | 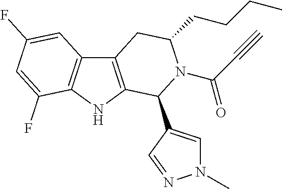 [SMILES: C#CC(=O)N1[C@@H](CCCC)Cc2c([nH]c3c(F)cc(F)cc23)[C@@H]1c1cnn(C)c1] |
| B-34 | 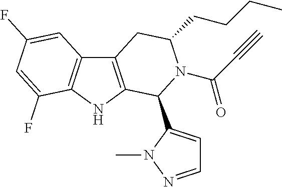 [SMILES: C#CC(=O)N1[C@@H](CCCC)Cc2c([nH]c3c(F)cc(F)cc23)[C@@H]1c1ccnn1C] |
| B-35 | 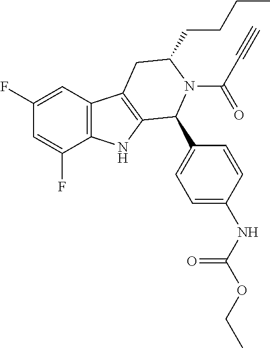 [SMILES: C#CC(=O)N1[C@@H](CCCC)Cc2c([nH]c3c(F)cc(F)cc23)[C@@H]1c1ccc(NC(=O)OCC)cc1] |
| B-36 | 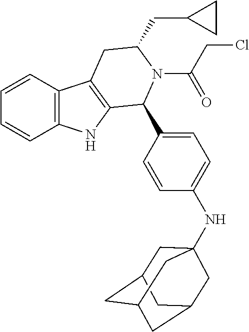 [SMILES: O=C(CCl)N1[C@@H](CC2CC2)Cc2c([nH]c3ccccc23)[C@@H]1c1ccc(NC23CC4CC(CC(C4)C2)C3)cc1] |
| B-37 | 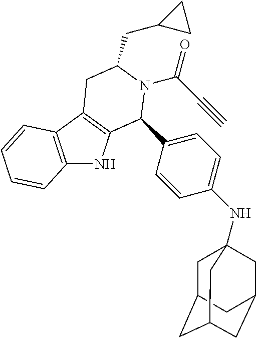 [SMILES: C#CC(=O)N1[C@@H](CC2CC2)Cc2c([nH]c3ccccc23)[C@@H]1c1ccc(NC23CC4CC(CC(C4)C2)C3)cc1] |
| B-38 | 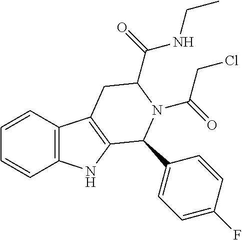 [SMILES: CCNC(=O)C1Cc2c([nH]c3ccccc23)[C@H](c2ccc(F)cc2)N1C(=O)CCl] |
| B-39 | 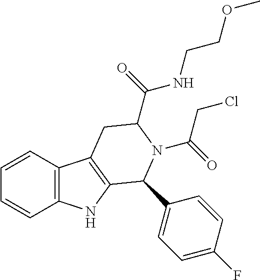 [SMILES: COCCNC(=O)C1Cc2c([nH]c3ccccc23)[C@H](c2ccc(F)cc2)N1C(=O)CCl] |
| B-40 | 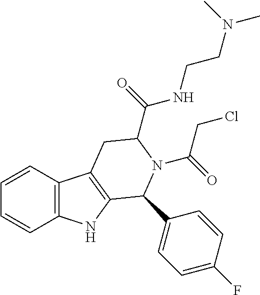 [SMILES: CN(C)CCNC(=O)C1Cc2c([nH]c3ccccc23)[C@H](c2ccc(F)cc2)N1C(=O)CCl] |
| B-41 | 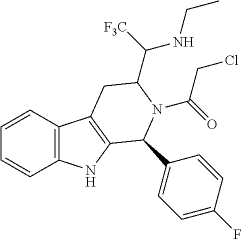 [SMILES: CCNC(C1Cc2c([nH]c3ccccc23)[C@H](c2ccc(F)cc2)N1C(=O)CCl)C(F)(F)F] |
| B-42 | 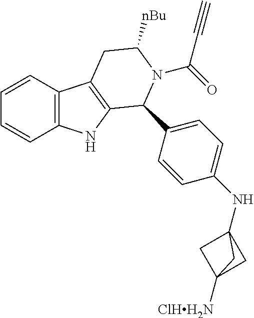 |
| B-43 | 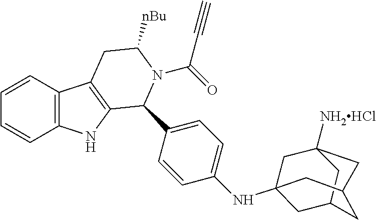 |
| B-44 | 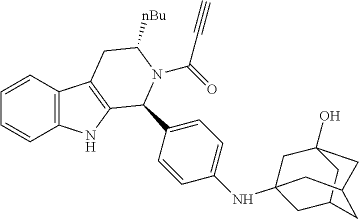 |
| B-45A | 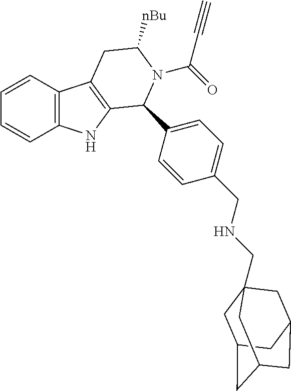 |
| B-45B | 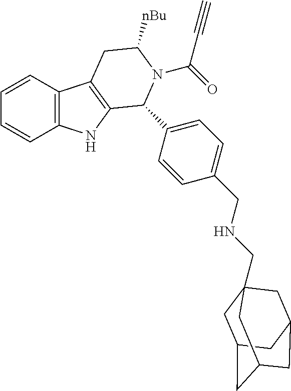 |
| B-46 | 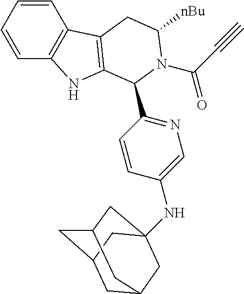 |
| B-47 | 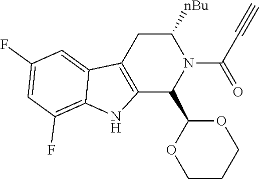 |
| B-48 | 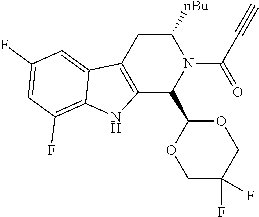 |
| B-49 | 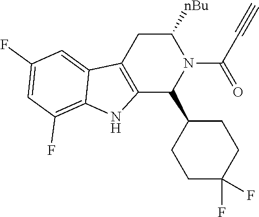 |
| B-50 | 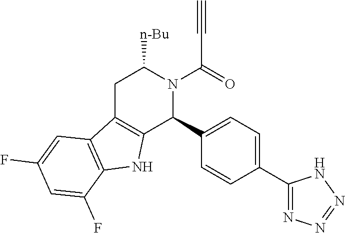 |
| B-51 | 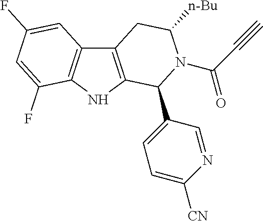 |
| B-52 | 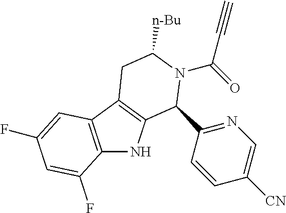 |
| B-53 | 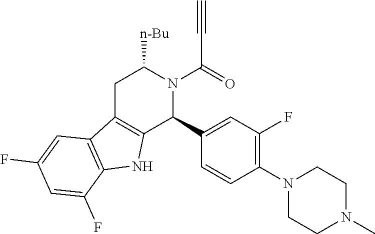 |
| B-54 | 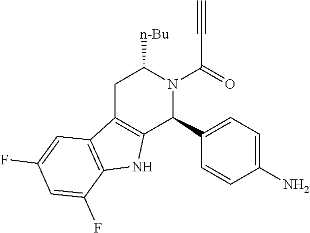 |
| B-55 | 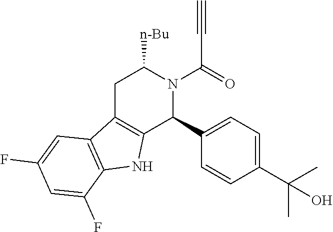 |
| B-56 | 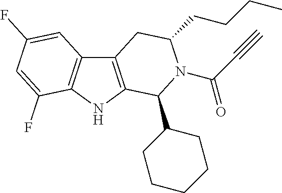 [SMILES: C#CC(=O)N1[C@@H](CCCC)Cc2c([nH]c3c(F)cc(F)cc23)[C@@H]1C1CCCCC1] |
| B-57 | 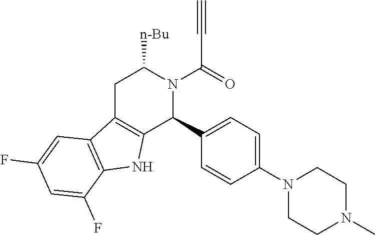 |
| B-58 | 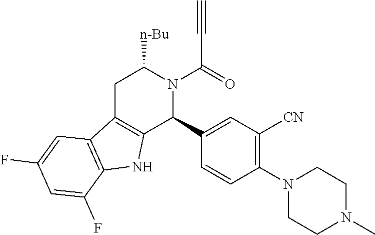 |
| B-59 | 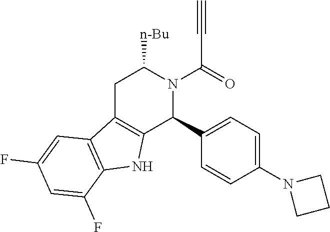 |
| B-60 | 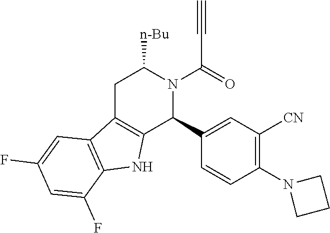 |
| B-61 | 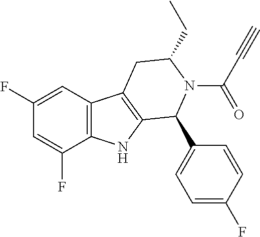 [SMILES: C#CC(=O)N1[C@@H](CC)Cc2c([nH]c3c(F)cc(F)cc23)[C@@H]1c1ccc(F)cc1] |
| B-62 | 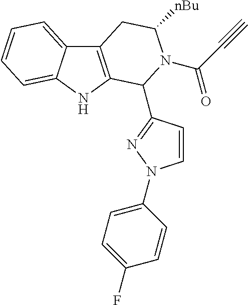 |
| B-63 | 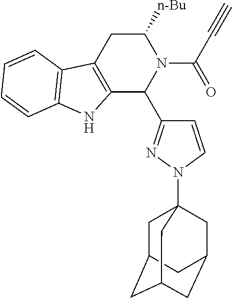 |
| B-64 | 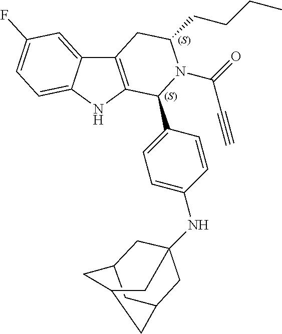 |
| B-65 | 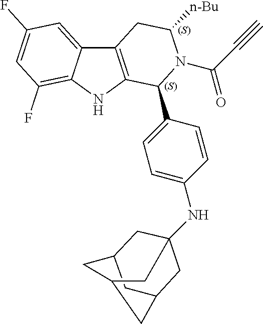 |
| B-66 | 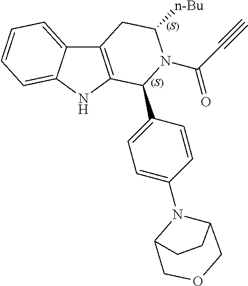 |
| B-67 | 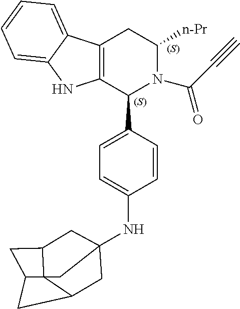 |
| B-68 | 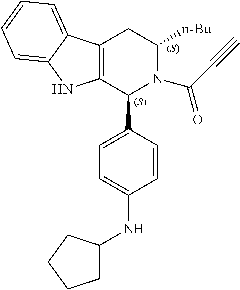 |
| B-69 | 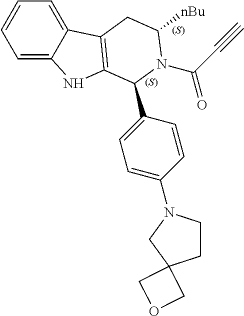 |
| B-70 | 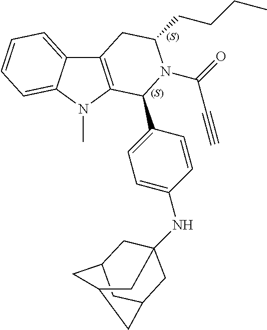 |
| B-71 | 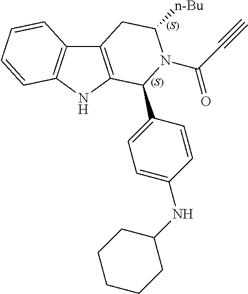 |
| B-72 | 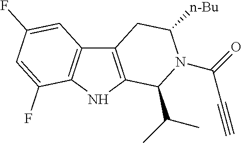 |
| B-73 | 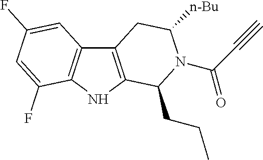 |
| B-74 | 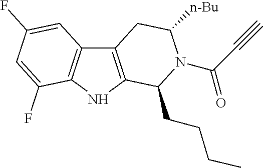 |
| B-75 | 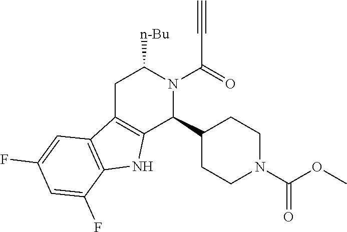 |
| B-76 | 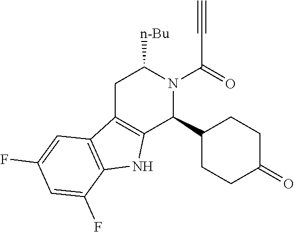 |
| B-77 | 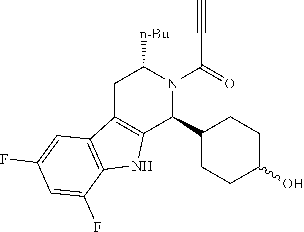 |
| B-78 | 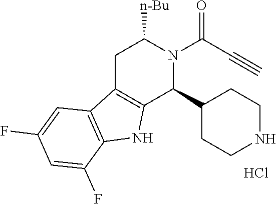 |
| B-79 | 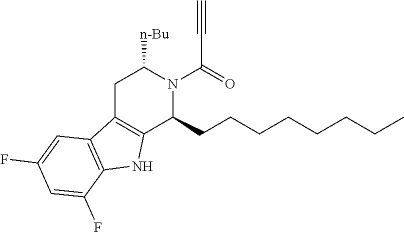 |
| B-80 | 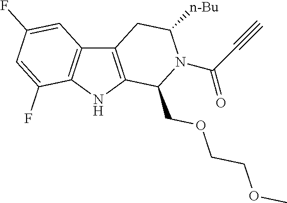 |
| B-81 | 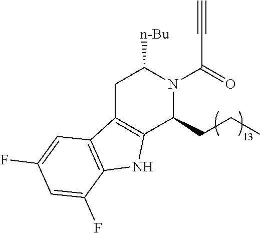 |
| B-82 | 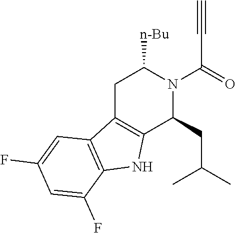 |
| B-83 | 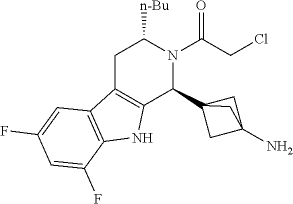 |
| B-84 | 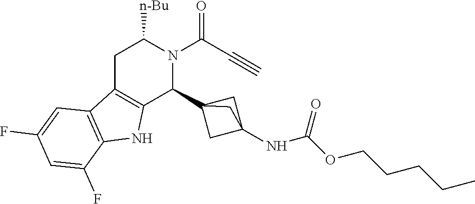 |
| B-85 | 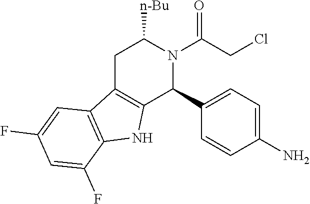 |
| B-86 | 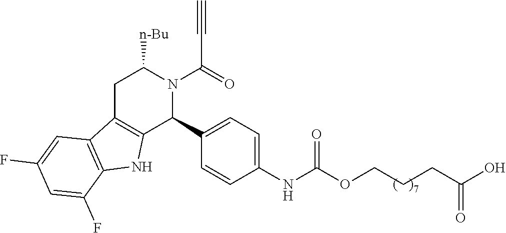 |
| B-87 | 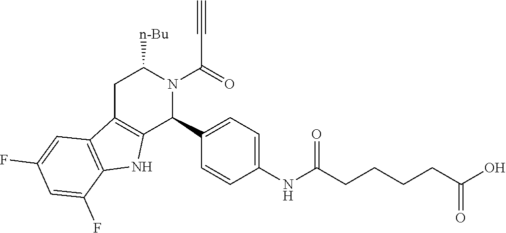 |
| B-88 | 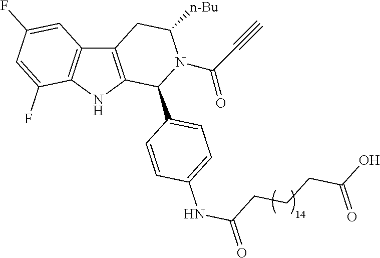 |
| B-89 | 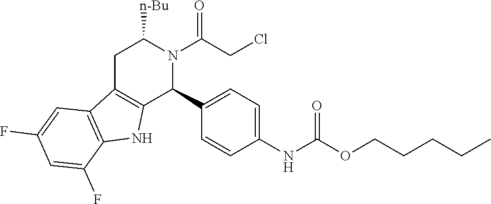 |
| B-90 | 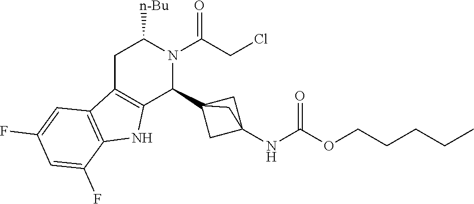 |
| B-91 | 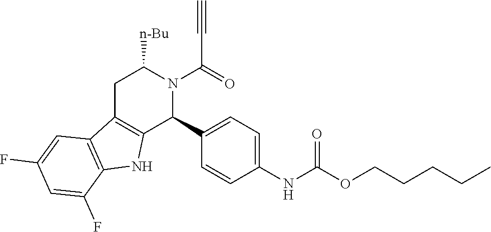 |
| B-92 | 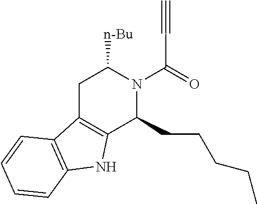 |
| B-93 | 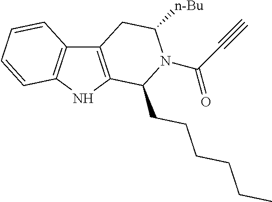 |
| B-94 | 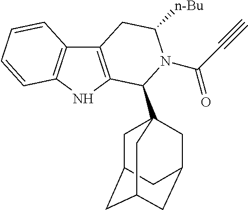 |
| B-95 | 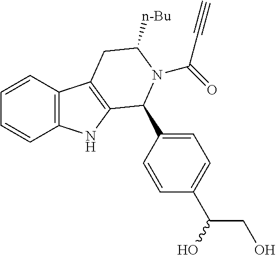 |
| B-96 | 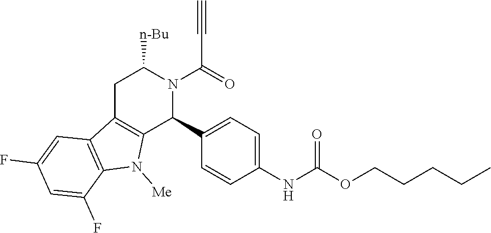 |
| B-97 | 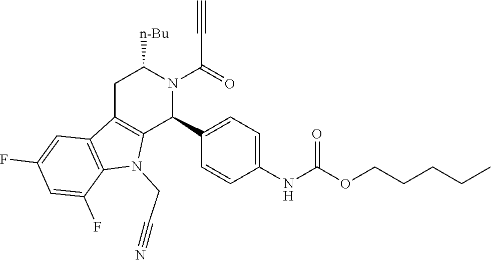 |
| B-98 | 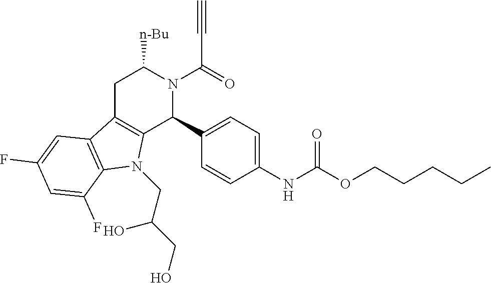 |
| B-99 | 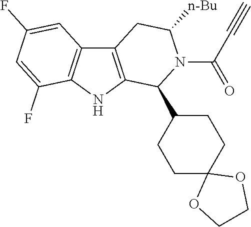 |
| B-100 | 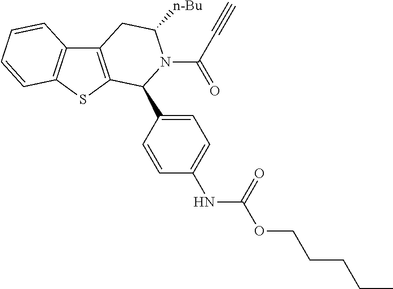 |
| B-101 | 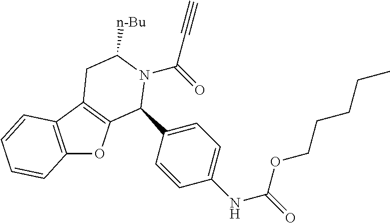 |

In certain embodiments, provided herein is a compound of Formula

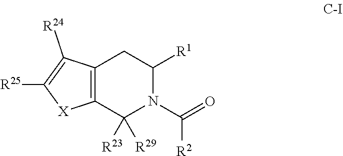

or a tautomer, stereoisomer, mixture of stereoisomers, isotopically enriched analog, or pharmaceutically acceptable salt thereof, wherein:

X is —NH—, —N(C₁-C₆alkyl)-, —O—, —S—, —N═CR¹⁶—, —CR¹⁶═CR¹⁶—, or —CR¹⁶═N—;

R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OR⁸, or —Si(R¹⁵)₃;

R² is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;

R²³ is C₁-C₉alkyl, C₂-C₉alkenyl, C₂-C₉alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl, wherein each C₁-C₉alkyl, C₂-C₉alkenyl, C₂-C₉alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl of R²³ is independently optionally substituted with one to three R¹⁷;

R²⁹ is hydrogen or C₁-C₆alkyl; provided that when R²³ is C₃-C₁₀cycloalkyl, heterocyclyl, aryl, heteroaryl, then R²⁹ is C₁-C₆alkyl;

R²⁴ and R²⁵ are each independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R²⁴ and R²⁵ is independently optionally substituted with one to three R¹⁰; or

when X is —NH—, —N(C₁-C₆alkyl)-, —O—, or —S—; then R²⁴ and R²⁵ together with the atoms to which they are attached, can form a 6-membered aryl or 6-membered heteroaryl, wherein each aryl or heteroaryl is optionally substituted with one to three R¹⁴;

each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;

each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each 127 or ring formed thereby is independently further substituted with one to three R¹¹;

each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;

each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;

each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;

each R¹² is independently hydrogen, C₁-C₆alkyl or C₃-C₁₀cycloalkyl;

each R¹³ is independently C₁-C₆alkyl or C₃-C₁₀cycloalkyl;

each R¹⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁴ is independently optionally substituted with one to three R¹¹);

each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, or heteroarylC₂-C₆alkenyl-;

each R¹⁶ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SFS, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁶ is independently optionally substituted with one to three R¹¹; and

each R¹⁷ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁷ is independently optionally substituted with one to three R¹⁰.

In certain embodiments, provided herein is a compound of Formula C-II, or a tautomer, stereoisomer, mixture of stereoisomers, isotopically enriched analog, or pharmaceutically acceptable salt thereof:

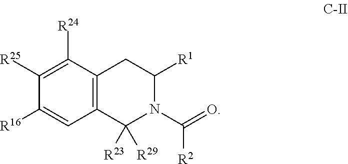

In certain embodiments, provided herein is a compound of Formula C-III, or a tautomer, stereoisomer, mixture of stereoisomers, isotopically enriched analog, or pharmaceutically acceptable salt thereof:

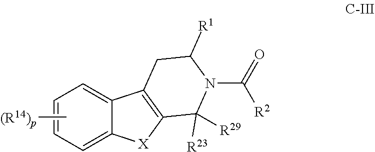

wherein p is 0, 1, 2 or 3.

In certain embodiments, provided herein is a compound of Formula C-IV, or a tautomer, stereoisomer, mixture of stereoisomers, isotopically enriched analog, or pharmaceutically acceptable salt thereof:

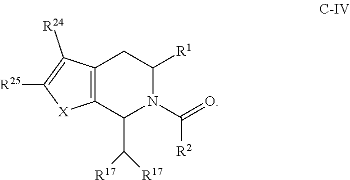

In certain embodiments, provided herein is a compound of Formula C-VI, or a tautomer, stereoisomer, mixture of stereoisomers, isotopically enriched analog, or pharmaceutically acceptable salt thereof:

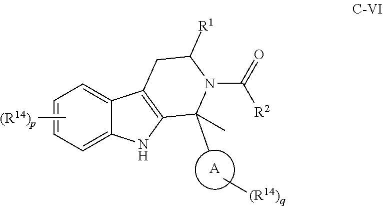

wherein

p is 0, 1, 2 or 3; and

q is 0, 1, 2 or 3.

In certain embodiments, each R¹ is C₁-C₆alkyl.

In certain embodiments, R²³ or ring A is aryl or heteroaryl. In certain embodiments, R²³ or ring A is a monocyclic aryl or monocyclic heteroaryl. In certain embodiments, R²³ or ring A is heterocyclyl. In certain embodiments, R²³ or ring A is a 4 to 7 membered heterocyclyl. In certain embodiments, R²³ or ring A is aryl. In certain embodiments, R²³ or ring A is phenyl. In certain embodiments, R²³ or ring A is heteroaryl. In certain embodiments, R²³ or ring A is pyridyl. In certain embodiments, R²³ or ring A is phenyl, pyridyl, piperidinyl, piperazinyl, or morpholinyl.

In certain embodiments, R²³ or ring A is aryl or heteroaryl, each of which is substituted by one to three R¹⁷. In certain embodiments, R²³ or ring A is aryl or heteroaryl, each of which is substituted by one to three R¹⁷, where at least one R¹⁷ is C₃-C₁₀cycloalkyl, heterocyclyl, aryl, or heteroaryl; wherein each C₃-C₁₀cycloalkyl, heterocyclyl, aryl, and heteroaryl of R¹⁷ is optionally substituted with one to three R¹⁰.

In certain embodiments, R²³ or ring A is aryl or heteroaryl, each of which is substituted by two or three R¹⁷. In certain embodiments, ring A is aryl or heteroaryl, each of which is substituted by two or three R¹⁷; wherein at least one R¹⁷ is halo.

In certain embodiments, R²³ or ring A is cyclohexyl. In certain embodiments, R²³ or ring A is C₄-C₁₀cycloalkyl, substituted with one to three R¹⁷. In certain embodiments, R²³ or ring A is a C₄-C₇cycloalkyl, substituted with one to three R¹⁷. In certain embodiments, R²³ or ring A is bicyclo[1.1.1]pentanyl, substituted with one to three R¹⁷. In certain embodiments, R²³ or ring A is selected from cyclobutyl, cyclopentyl, cyclohexyl, and cycloheptyl, wherein each is substituted with one to three R¹⁷.

In certain embodiments, R²³ or ring A is bicyclo[1.1.1]pentanyl. In certain embodiments, R²³ or ring A is selected from cyclobutyl, cyclopentyl, cyclohexyl, and cycloheptyl.

In certain embodiments, R²³ or ring A is a bridged bicyclic ring selected from:

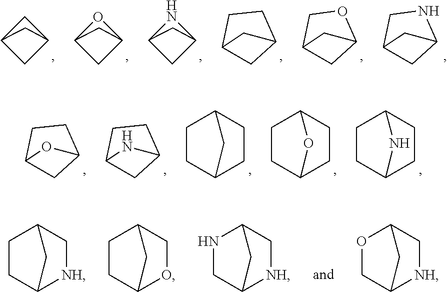

wherein each is substituted with one to three R¹⁷. In certain embodiments, R²³ or ring A is a bridged bicyclic ring selected from:

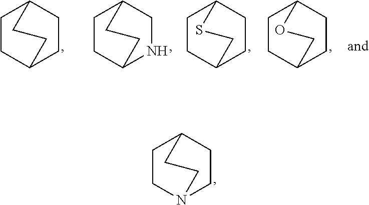

wherein each R¹⁷ is attached to a carbon atom on the bridged bicyclic ring.

In certain embodiments, X is —NH—, —N(C₁-C₆alkyl)-, —O—, or —S—. In certain embodiments, X is —NH—.

In certain embodiments, X is —N═CR¹⁶═CR¹⁶═CR¹⁶—, —CR¹⁶═N—. In certain embodiments, X is —CR¹⁶═CR¹⁶—.

In certain embodiments, X is —CH═CH—.

In certain embodiments, IV is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —N(R⁷)₂, —OR⁷, or —C₁-C₆alkyl-OR⁷.

In certain embodiments, R¹ is —C(O)OR⁶ or —C(O)N(R⁷)₂.

In certain embodiments, R¹ is C₁-C₆alkyl. In certain embodiments, In certain embodiments, R¹ is C₂-C₆alkyl. In certain embodiments, R¹ is C₃-C₆alkyl. In certain embodiments, R¹ is C₅-C₆alkyl. In certain embodiments, R¹ is C₂-C₃alkyl. In certain embodiments, R¹ is C₄-C₆alkyl. In certain embodiments, R¹ is methyl. In certain embodiments, R¹ is n-butyl.

In certain embodiments, R¹ is —CH₂—R²⁶, wherein R²⁶ is C₁-C₅alkyl, C₂-C₅alkenyl, C₂-C₅alkynyl, C₁-C₅haloalkyl, or —C₁-C₅alkyl-OR⁷.

In certain embodiments, R¹ is C₂-C₆alkenyl, C₂-C₆alkynyl, C₁-C₆haloalkyl, C₃-C₁₀cycloalkyl, —CN, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R¹⁵)₃.

In certain embodiments, R¹ is other than methyl. In certain embodiments, R¹ is other than n-butyl. In certain embodiments, R¹ is other than —C(O)OR⁶. In certain embodiments, R¹ is other than —C(O)OCH₃.

In certain embodiments, at least one R¹⁷ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SFS, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

In certain embodiments, at least one R¹⁷ is halo.

In certain embodiments, at least one R¹⁷ is —NHR⁸. In certain embodiments, at least one R²³ is —N(R⁸)₂. In certain embodiments, q is 2, and one R¹⁷ is halo and the other R¹⁷ is —N(R⁸)₂. In certain embodiments, q is 3, and two R¹⁷ are independently halo and one R¹⁷ is —N(R⁸)₂.

In certain embodiments, at least one R¹⁷ is —C(O)OR⁶ or —C(O)R⁶.

In certain embodiments, at least one R¹⁷ is —S(O)₂N(R⁷)₂, —S(O)N(R⁷)₂, or —C(O)N(R⁷)₂.

In certain embodiments, at least one R¹⁷ is —S(O)₂R⁸, —S(O)R⁸, —NR¹²C(O)OR⁸, —NR¹²C(O)OR⁸, —OC(O)R⁸, or —OC(O)CHR⁸N(R¹²)₂.

In certain embodiments, each R¹⁷ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R¹⁷ is independently optionally substituted with one to three R¹⁰.

In certain embodiments, each R¹⁷ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl is independently optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R²⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₂-C₆alkenyl, C₂-C₆alkynyl, or C₃-C₁₀cycloalkyl; wherein each C₂-C₆alkenyl, C₂-C₆alkynyl, or C₃-C₁₀cycloalkyl of R²⁴ is independently optionally substituted with one to three R¹⁰.

In certain embodiments, each R²⁴ is independently halo, —CN, —OR⁷, C₂-C₆alkynyl, or C₃-C₁₀cycloalkyl; wherein each C₁-C₆alkyl, C₂-C₆alkynyl, or C₃-C₁₀cycloalkyl of R²⁴ is independently optionally substituted with one to three R¹⁰.

In certain embodiments, each R²⁴ is independently halo, —CN, —OH, C₂-C₆alkynyl, or C₃-C₁₀cycloalkyl.

In certain embodiments, each R²⁴ is independently halo, —CN, —OH, —OR⁸, C₁-C₆alkyl, or C₂-C₆alkynyl; wherein the C₁-C₆alkyl of R²⁴ is optionally substituted with one to three R¹⁰.

In certain embodiments, each R²⁴ is independently halo, —CN, —OH, —OR⁸, C₂-C₆alkynyl; wherein the C₁-C₆alkyl of R²⁴ is optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R¹⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R¹⁴ is independently optionally substituted with one to three R¹⁰.

In certain embodiments, each R¹⁴ is independently halo, —CN, —OR⁷, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R¹⁴ is independently optionally substituted with one to three R¹⁰.

In certain embodiments, each R¹⁴ is independently halo, —CN, —OH, C₁-C₆alkyl, C₂-C₆alkynyl, or C₃-C₁₀cycloalkyl.

In certain embodiments, each R¹⁴ is independently halo, —CN, —OH, —OR⁸, C₁-C₆alkyl, or C₂-C₆alkynyl; wherein the C₁-C₆alkyl of R¹⁴ is optionally substituted with one to three R¹⁰.

In certain embodiments, each R¹⁴ is independently halo, —CN, —OH, —OR⁸, C₁-C₆alkyl, C₂-C₆alkynyl; wherein the C₁-C₆alkyl of R¹⁴ is optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl; and

each R¹³ is independently C₁-C₆alkyl or C₃-C₁₀cycloalkyl.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three R¹¹.

In certain embodiments, each R⁶ is independently hydrogen, C₁-C₆alkyl, C₂-C₆alkenyl, or —C₁-C₆alkylC₃-C₁₀cycloalkyl; wherein each R⁶ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹.

In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹.

In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein

each R¹² is independently hydrogen, C₁-C₆alkyl or C₃-C₁₀cycloalkyl.

In certain embodiments, each R¹⁰ is independently —OR¹², —N(R¹²)₂, S(O)₂R¹³, —OC(O)CHR¹²N(R¹²)₂, or C₁-C₆alkyl, wherein the C₁-C₆alkyl, of R¹⁰ is optionally independently substituted with one to three R¹¹;

each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;

each R¹² is independently hydrogen, C₁-C₆alkyl or C₃-C₁₀cycloalkyl; and

each R¹¹ is independently C₁-C₆alkyl or C₃-C₁₀cycloalkyl.

In certain embodiments, each R¹⁵ is independently C₁-C₆alkyl.

In certain embodiments, p is 0. In certain embodiments, p is 0 or 1. In certain embodiments, p is 1 or 2. In certain embodiments, p is 1. In certain embodiments, p is 2.

In certain embodiments, q is 0. In certain embodiments, q is 0 or 1. In certain embodiments, q is 1 or 2. In certain embodiments, q is 1. In certain embodiments, q is 2. In certain embodiments, q is 3.

Also provided is a compound, or a tautomer, stereoisomer, mixture of stereoisomers, isotopically enriched analog, or pharmaceutically acceptable salt thereof, selected from Table C-1:

TABLE C-1

| No. | Structure |
|---|---|
| C-1 | 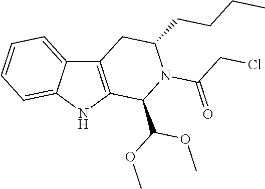 [SMILES: CCCC[C@H]1Cc2c([nH]c3ccccc23)[C@H](C(OC)OC)N1C(=O)CCl] |
| C-2 | 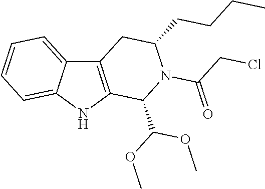 [SMILES: CCCC[C@H]1Cc2c([nH]c3ccccc23)[C@@H](C(OC)OC)N1C(=O)CCl] |
| C-3 | 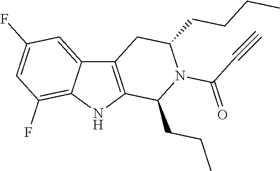 [SMILES: C#CC(=O)N1[C@@H](CCCC)Cc2c([nH]c3c(F)cc(F)cc23)[C@@H]1CCC] |
| C-4 | 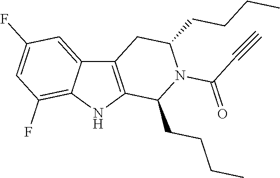 [SMILES: C#CC(=O)N1[C@@H](CCCC)Cc2c([nH]c3c(F)cc(F)cc23)[C@@H]1CCCC] |
| C-5 | 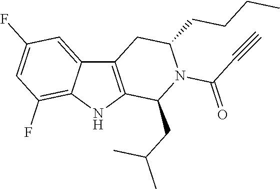 [SMILES: C#CC(=O)N1[C@@H](CCCC)Cc2c([nH]c3c(F)cc(F)cc23)[C@@H]1CC(C)C] |
| C-6 | 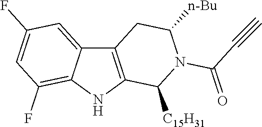 |
| C-7 | 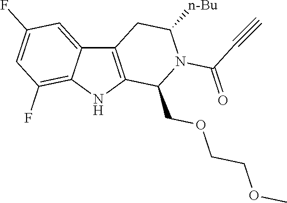 |
| C-8 | 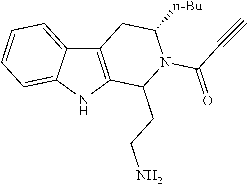 |
| C-9 | 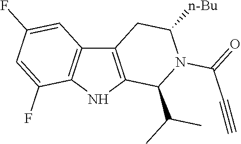 |
| C-10 | 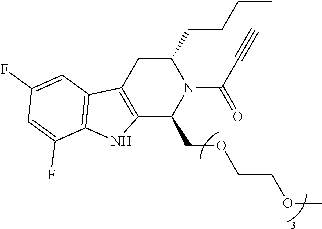 |
| C-11 | 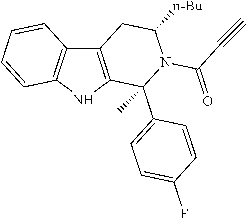 |
| C-14 | 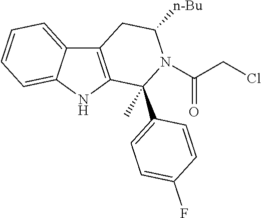 |
| C-16 | 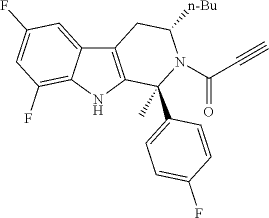 |
| C-17 | 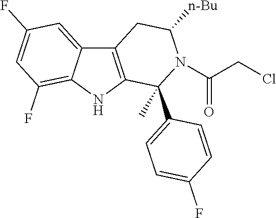 |
| C-18 | 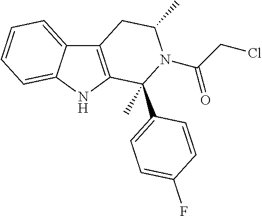 [SMILES: C[C@H]1Cc2c([nH]c3ccccc23)[C@](C)(c2ccc(F)cc2)N1C(=O)CCl] |
| C-19 | 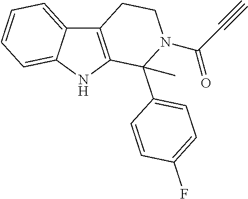 [SMILES: C#CC(=O)N1CCc2c([nH]c3ccccc23)C1(C)c1ccc(F)cc1] |
| C-20 | 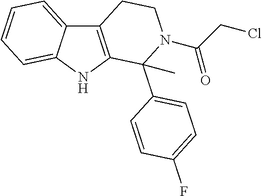 [SMILES: CC1(c2ccc(F)cc2)c2[nH]c3ccccc3c2CCN1C(=O)CCl] |
| C-21 | 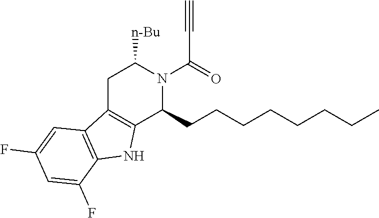 |
| C-22 | 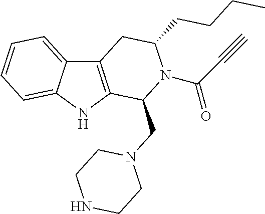 [SMILES: C#CC(=O)N1[C@@H](CCCC)Cc2c([nH]c3ccccc23)[C@@H]1CN1CCNCC1] |
| C-23 | 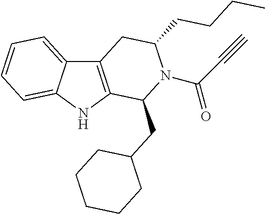 [SMILES: C#CC(=O)N1[C@@H](CCCC)Cc2c([nH]c3ccccc23)[C@@H]1CC1CCCCC1] |
| C-24 | 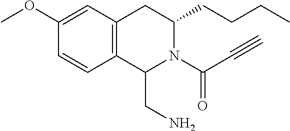 [SMILES: C#CC(=O)N1C(CN)c2ccc(OC)cc2C[C@@H]1CCCC] |
| C-25 | 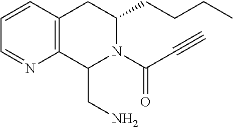 [SMILES: C#CC(=O)N1C(CN)c2ncccc2C[C@@H]1CCCC] |
| C-26 | 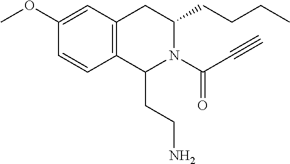 [SMILES: C#CC(=O)N1C(CCN)c2ccc(OC)cc2C[C@@H]1CCCC] |
| C-27 | 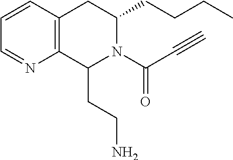 [SMILES: C#CC(=O)N1C(CCN)c2ncccc2C[C@@H]1CCCC] |
| C-28 | 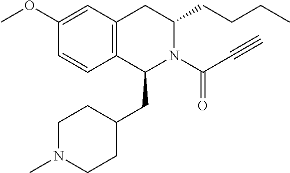 [SMILES: C#CC(=O)N1[C@@H](CCCC)Cc2cc(OC)ccc2[C@@H]1CC1CCN(C)CC1] |
| C-29 | 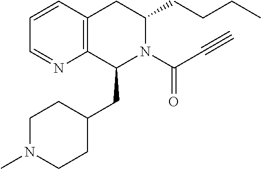 [SMILES: C#CC(=O)N1[C@@H](CCCC)Cc2cccnc2[C@@H]1CC1CCN(C)CC1] |
| C-30 | 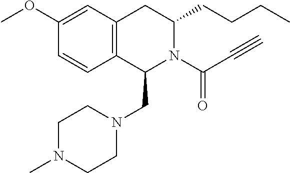 [SMILES: C#CC(=O)N1[C@@H](CCCC)Cc2cc(OC)ccc2[C@@H]1CN1CCN(C)CC1] |
| C-31 | 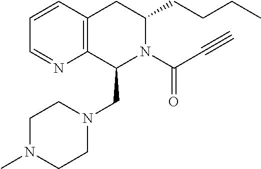 [SMILES: C#CC(=O)N1[C@@H](CCCC)Cc2cccnc2[C@@H]1CN1CCN(C)CC1] |
| C-32 | 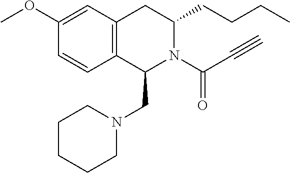 [SMILES: C#CC(=O)N1[C@@H](CCCC)Cc2cc(OC)ccc2[C@@H]1CN1CCCCC1] |
| C-33 | 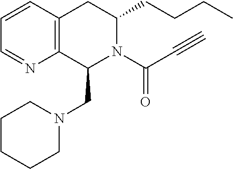 [SMILES: C#CC(=O)N1[C@@H](CCCC)Cc2cccnc2[C@@H]1CN1CCCCC1] |
| C-35 | 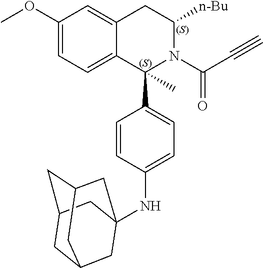 |
| C-36 | 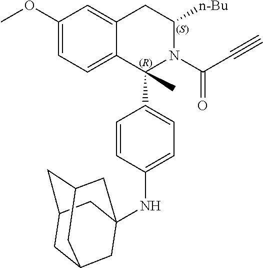 |
| C-37 | 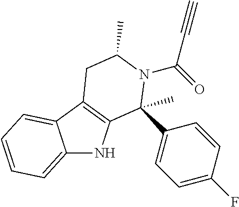 [SMILES: C#CC(=O)N1[C@@H](C)Cc2c([nH]c3ccccc23)[C@]1(C)c1ccc(F)cc1] |
| C-38 | 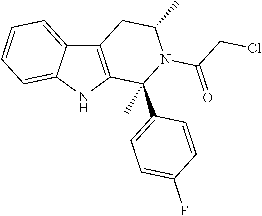 [SMILES: C[C@H]1Cc2c([nH]c3ccccc23)[C@](C)(c2ccc(F)cc2)N1C(=O)CCl] |

### 3. Methods of Use

In certain embodiments, the compounds described herein are used in a method of treating cancer. In certain embodiments, the method of treating cancer comprises administering to a subject in need thereof a therapeutically effective amount any of the compounds described herein.

In certain embodiments, the compounds are used in a method of inhibiting GPX4 in a cell, comprising contacting a cell with an effective amount of a compound or composition described herein to inhibit GPX4 in the cell. In certain embodiments, the cell is a cancer cell. In certain embodiments, the method comprises administering an effective amount of a compound or composition described herein to a patient in need thereof.

In certain embodiments, the compounds are used in a method of inducing ferroptosis in a cell comprising contacting the cell with an effective amount of a compound or composition provided herein. In certain embodiments, the method comprises administering an effective amount of a compound or composition described herein to a patient in need thereof.

In certain embodiments, provided is a method for treating a cancer in a patient in need thereof, comprising administering an effective amount of a compound or composition provided herein.

In certain embodiments, the compounds are used in a method of treating cancer in a subject in need thereof, comprising administering to a subject having cancer a therapeutically effective amount of a ferroptosis inducing compound disclosed herein. Various cancers for treatment with the compounds include, but are not limited to, adrenocortical cancer, anal cancer, biliary cancer, bladder cancer, bone cancer, gliomas, astrocytoma, neuroblastoma, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, and soft tissue carcinomas. In certain embodiments, the compound is used to treat pancreatic cancer.

In certain embodiments, the cancer is renal cell carcinoma (RCC), pancreatic cancer, lung cancer, breast cancer, or prostate cancer. In certain embodiments, provided is a method for treating renal cell carcinoma (RCC) in a patient in need thereof, comprising administering an effective amount of a compound or composition provided herein. In certain embodiments, provided is a method for treating pancreatic cancer in a patient in need thereof, comprising administering an effective amount of a compound or composition provided herein. In certain embodiments, provided is a method for treating lung cancer in a patient in need thereof, comprising administering an effective amount of a compound or composition provided herein. In certain embodiments, provided is a method for treating breast cancer in a patient in need thereof, comprising administering an effective amount of a compound or composition provided herein. In certain embodiments, provided is a method for treating prostate cancer in a patient in need thereof, comprising administering an effective amount of a compound or composition provided herein.

In certain embodiments, provided is a method for treating a malignant solid tumor in a patient in need thereof, comprising administering an effective amount of a compound or composition provided herein to the patient. In certain embodiments, the malignant solid tumor is a carcinoma. In certain embodiments, the malignant solid tumor is a lymphoma. In certain embodiments, the malignant solid tumor is a sarcoma.

In certain embodiments, the cancer for treatment with the compound can be selected from, among others, adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemia and lymphoma), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, vaginal cancer, sarcoma, and soft tissue carcinomas. In certain embodiments, the cancer is renal cell carcinoma (RCC). In certain embodiments, the cancer is pancreatic cancer. In certain embodiments, the cancer is lung cancer. In certain embodiments, the cancer is breast cancer. In certain embodiments, the cancer is prostate cancer.

In certain embodiments, the cancer for treatment with the compound is pancreatic cancer. In certain embodiments, the pancreatic cancer for treatment with the compounds is pancreatic adenocarcinoma or metastatic pancreatic cancer. In certain embodiments, the cancer for treatment with the compounds is stage I, stage II, stage III, or stage IV pancreatic adenocarcinoma.

In certain embodiments, the cancer for treatment with the compounds is lung cancer. In certain embodiments, the lung cancer for treatment with the compounds is small cell lung cancer or non-small cell lung cancer. In certain embodiments, the non-small cell lung cancer for treatment with the compounds is an adenocarcinoma, squamous cell carcinoma, or large cell carcinoma. In certain embodiments, the lung cancer for treatment with the compounds is metastatic lung cancer.

In certain embodiments, the cancer for treatment with the compounds is a hematologic cancer. In certain embodiments, the hematologic cancer is selected from acute lymphoblastic leukemia (ALL), acute myeloid leukemia (AML), lymphoma (e.g., Hodgkin's lymphoma, Non-Hodgkin's lymphoma, Burkitt's lymphoma), chronic lymphocytic leukemia (CLL), chronic myelogenous leukemia (CML), Hairy Cell chronic myelogenous leukemia (CML), and multiple myeloma.

In certain embodiments, the cancer for treatment with the compounds is a leukemia selected from acute lymphoblastic leukemia (ALL), acute myeloid leukemia (AML), chronic lymphocytic leukemia (CLL), chronic myelogenous leukemia (CML), Hairy Cell chronic myelogenous leukemia (CML), and multiple myeloma.

In certain embodiments, the cancer for treatment with the compound is a lymphoma selected from Hodgkin's lymphoma, Non-Hodgkin's lymphoma, and Burkitt's lymphoma.

In certain embodiments, the cancer for treatment with the compound is a cancer characterized by mesenchymal features or mesenchymal phenotype. In some cancers, gain of mesenchymal features is associated with migratory (e.g., intravasation) and invasiveness of cancers. Mesenchymal features can include, among others, enhanced migratory capacity, invasiveness, elevated resistance to apoptosis, and increased production of extracellular matrix (ECM) components. In addition to these physiological characteristics, the mesenchymal features can include expression of certain biomarkers, including among others, E-cadherin, N-cadherin, integrins, FSP-1, α-SMA, vimentin, β-catenin, collagen I, collagen II, collagen III, collagen IV, fibronectin, laminin 5, SNAIL-1, SNAIL-2, Twist-1, Twist-2, and Lef-1. In certain embodiments, the cancer selected for treatment with the compounds herein include, among others, breast cancer, lung cancer, head and neck cancer, prostate cancer, and colon cancer. In certain embodiments, the mesenchymal features can be inherent to the cancer type or induced by or selected for by treatment of cancers with chemotherapy and/or radiation therapy.

In certain embodiments, the cancer for treatment with the compound is identified as having or determined to have an activating or oncogenic RAS activity. In certain embodiments, the RAS is K-RAS, H-RAS or N-RAS. In certain embodiments, the activating or oncogenic RAS is an activating or oncogenic RAS mutation.

In certain embodiments, the cancer selected for treatment with the compounds are determined to have or identified as having an activating or oncogenic RAS activity. In certain embodiments, the activating or oncogenic RAS activity is an activating or oncogenic RAS mutations. In certain embodiments, the activating or oncogenic RAS activity is an activating or activating K-RAS activity, particularly an activating or oncogenic K-RAS mutation. In certain embodiments, the activating or oncogenic RAS activity is an activating or activating N-RAS activity, particularly an activating or oncogenic N-RAS mutation. In certain embodiments, the activating or oncogenic RAS activity is an activating or activating H-RAS activity, particularly an activating or oncogenic H-RAS mutation.

In certain embodiments, the cancer for treatment with the compounds can be a cancer having prevalence (e.g., at least about 10% or more, or about 15% or more of the cancers), of an activating or oncogenic RAS mutation, such as biliary tract cancer, cervical cancer, endometrial cancer, pancreatic cancer, lung cancer, colorectal cancer, head and neck cancer, stomach (gastric) cancer, hematologic cancer (e.g., leukemia, lymphomas, etc.), ovarian cancer, prostate cancer, salivary gland cancer, skin cancer, small intestinal cancer, thyroid cancer, aerodigestive tract, urinary tract cancer, and bladder cancer.

In certain embodiments, the compounds can be used to treat a cancer that is refractory to one or more other chemotherapeutic agents, particularly cytotoxic chemotherapeutic agents; or treat a cancer resistant to radiation treatment. In certain embodiments, the compounds are used to treat cancers that have developed tolerance to chemotherapeutic agents activating other cell death pathways, such as apoptosis, mitotic catastrophe, necrosis, senescence and/or autophagy.

In certain embodiments, the cancer for treatment with the compounds is identified as being refractory or resistant to chemotherapy. In certain embodiments, the cancer is refractory or resistant to one or more of alkylating agents, anti-cancer antibiotic agents, antimetabolic agents (e.g., folate antagonists, purine analogs, pyrimidine analogs, etc.), topoisomerase inhibiting agents, anti-microtubule agents (e.g., taxanes, vinca alkaloids), hormonal agents (e.g., aromatase inhibitors), plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors.

In certain embodiments, the cancer for treatment with the compounds is a cancer identified as being refractory or resistant to one or more of afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelborae), vincristine, vinblastine, vinorelbine, and vindesine.

In certain embodiments, the cancer for treatment with the compound is identified as being refractory or resistant to one or more chemotherapeutics agents selected from cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and erlotinib.

In certain embodiments, the cancer for treatment with the compounds is a cancer resistant to ionizing radiation therapy. The radioresistance of the cancer can be inherent or as a result of radiation therapy. In certain embodiments, the cancers for treatment with the compounds is, among others, a radioresistant adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemia and lymphoma), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, or vaginal cancer. In certain embodiments, the cancer is pancreatic cancer, breast cancer, glioblastoma, advanced non-small-cell lung cancer, bladder cancer, sarcoma, or soft tissue carcinoma.

### 4. Combination Treatments

In certain embodiments, the compounds described herein are used in combination with one or more of other (e.g., second therapeutic agent) therapeutic treatments for cancer. In certain embodiments, the compounds can be used as monotherapy, or as further provided below, in a combination therapy with one or more therapeutic treatments, particularly in combination with one or more chemotherapeutic agents. In certain embodiments, the compounds are used in combination with a second therapeutic agent, where the compounds are used at levels that sensitizes the cancer or cancer cell to the second therapeutic agent, for example at levels of the compound that do not cause significant cell death. In certain embodiments, the compounds can be used in combination with radiation therapy, either to sensitize the cells to radiation therapy or as an adjunct to radiation therapy (e.g., at doses sufficient to activate cell death pathway).

In certain embodiments, a subject with cancer is treated with a combination of a compound described herein and radiation therapy. In certain embodiments, the method comprises administering to a subject with cancer a therapeutically effective amount of a compound of the disclosure, and adjunctively treating the subject with an effective amount of radiation therapy. In certain embodiments, the compound is administered to the subject in need thereof prior to, concurrently with, or subsequent to the treatment with radiation.

In certain embodiments, the method comprises administering an effective amount of a compound described herein to a subject with cancer to sensitize the cancer to radiation treatment, and administering a therapeutically effective amount of radiation therapy to treat the cancer. In certain embodiments, an effective amount of X-ray and gamma ray is administered to the subject. In certain embodiments, an effective amount of particle radiation is administered to the subject, where the particle radiation is selected from electron beam, proton beam, and neutron beam radiation. In certain embodiments, the radiation therapy is fractionated.

In certain embodiments, a subject with cancer is administered a therapeutically effective amount of a compound described herein, or a first pharmaceutical composition thereof, and adjunctively administered a therapeutically effective amount of a second chemotherapeutic agent, or a second pharmaceutical composition thereof.

In certain embodiments, the second chemotherapeutic agent is selected from an platinating agent, alkylating agent, anti-cancer antibiotic agent, antimetabolic agent (e.g., folate antagonists, purine analogs, pyrimidine analogs, etc.), topoisomerase I inhibiting agent, topoisomerase II inhibiting agent antimicrotubule agent (e.g., taxanes, vinca alkaloids), hormonal agent (e.g., aromatase inhibitors), plant-derived agent and synthetic derivatives thereof, anti-angiogenic agent, differentiation inducing agent, cell growth arrest inducing agent, apoptosis inducing agent, cytotoxic agent, agent affecting cell bioenergetics, i.e., affecting cellular ATP levels and molecules/activities regulating these levels, anti-cancer biologic agent (e.g., monoclonal antibodies), kinase inhibitors and inhibitors of growth factors and their receptors.

In certain embodiments, the second chemotherapeutic agent is an angiogenesis inhibitor, such as but not limited to, an inhibitor of soluble VEGFR-1, NRP-1, angiopoietin 2, TSP-1, TSP-2, angiostatin and related molecules, endostatin, vasostatin, calreticulin, platelet factor-4, TIMP, CDAI, Meth-1, Meth-2, IFN-α, IFN-β, IFN-γ, CXCL10, IL-4, IL-12, IL-18, prothrombin (kringle domain-2), antithrombin III fragment, prolactin, VEGI, SPARC, osteopontin, maspin, canstatin (a fragment of COL4A2), or proliferin-related protein. In certain embodiments, the angiogenesis inhibitor is bevacizumab (Avastin), itraconazole, carboxyamidotriazole, TNP-470 (an analog of fumagillin), CM101, IFN-α, IL-12, platelet factor-4, suramin, SU5416, thrombospondin, a VEGFR antagonist, an angiostatic steroid plus heparin, cartilage-derived angiogenesis inhibitory factor (CDAI), a matrix metalloproteinase inhibitor, angiostatin, endostatin, 2-methoxyestradiol, tecogalan, tetrathiomolybdate, thalidomide, thrombospondin, prolactin, a αVβ3 inhibitor, linomide, ramucirumab, tasquinimod, ranibizumab, sorafenib (Nexavar), sunitinib (Sutent), pazopanib (Votrient), or everolimus (Afinitor).

In certain embodiments, the second chemotherapeutic agent is a cyclin-dependent kinase (CDK) inhibitor (e.g., a CDK4/CDK6 inhibitor). Examples include, but are not limited to, palbociclib (Ibrance), Ribociclib (optionally further in combination with letrozole), abemaciclib (LY2835219; Verzenio), P1446A-05, and Trilaciclib (G1T28).

In certain embodiments, the second chemotherapeutic agent is a Bruton's tyrosine kinase (BTK) inhibitor, such as but not limited to, Ibrutinib (PCI-32765), acalabrutinib, ONO-4059 (GS-4059), spebrutinib (AVL-292, CC-292), BGB-3111, and HM71224.

In certain embodiments, the second chemotherapeutic agent is a BRAF inhibitor. Examples include, but are not limited to, BAY43-9006 (Sorafenib, Nexavar), PLX-4032 (Vemurafenib), GDC-0879, PLX-4720, dabrafenib and LGX818.

In certain embodiments, the second chemotherapeutic agent is a EGFR inhibitor. Examples include, but are not limited to, gefitinib, erlotinib, afatinib, brigatinib, icotinib, cetuximab, osimertinib, panitumumab, brigatinib, lapatinib, cimaVax-EGF, and veristrat.

In certain embodiments, the second chemotherapeutic agent is a human epidermal growth factor receptor 2 (HER2) inhibitor. Examples include, but are not limited to, trastuzumab, pertuzumab (optionally further in combination with trastuzumab), margetuximab, and NeuVax.

In certain embodiments, disclosed herein is a method of increasing a subject's responsiveness to an immunotherapeutic or immunogenic chemotherapeutic agent, the method comprising administering to the subject in need thereof an effective amount of a compound described herein and an effective amount of an immunotherapeutic agent and/or an immunogenic chemotherapeutic agent. In certain embodiments, the method further includes administering to the subject a lipoxygenase inhibitor. In certain embodiments, the subject has a tumor whose cellular microenvironment is stromal cell rich. In certain embodiments, the administration of compound described herein results in killing one or more stromal cells in the tumor cells' microenvironment. In certain embodiments, the administration of an effective amount of an immunotherapeutic agent and/or an immunogenic chemotherapeutic agent results in killing one or more tumor cells. Also provided herein is a combination comprising a compound described herein and an immunotherapeutic agent, lipoxygenase inhibitor, or immunogenic chemotherapeutic agent. In certain embodiments, the immunotherapeutic agent is selected from a CTLA4, PDL1 or PD1 inhibitor. In certain embodiments, the immunotherapeutic agent can be selected from CTLA4 inhibitor such as ipilimumab, a PD1 inhibitor such as pembrolizumab or nivolumab or a PDL1 inhibitor such as atezolizumab or durvalumab. In certain embodiments, the immunotherapeutic agent is pembrolizumab. In other embodiments, the immunogenic chemotherapeutic agent is a compound selected from anthracycline, doxorubicin, cyclophosphamide, paclitaxel, docetaxel, cisplatin, oxaliplatin or carboplatin. In certain embodiments, provided herein is a combination comprising a compound described herein and a lipoxygenase inhibitor. In certain embodiments, the lipoxygenase inhibitor is selected from PD147176 and/or ML351. In certain embodiments, the lipoxygenase inhibitor may be a 15-lipoxygenase inhibitor (see, e.g., Sadeghian et al., Expert Opinion on Therapeutic Patents, 2015, 26:1, 65-88).

In certain embodiments, the second chemotherapeutic agent is selected from an alkylating agent, including, but not limiting to, adozelesin, altretamine, bendamustine, bizelesin, busulfan, carboplatin, carboquone, carmofur, carmustine, chlorambucil, cisplatin, cyclophosphamide, dacarbazine, estramustine, etoglucid, fotemustine, hepsulfam, ifosfamide, improsulfan, irofulven, lomustine, mannosulfan, mechlorethamine, melphalan, mitobronitol, nedaplatin, nimustine, oxaliplatin, piposulfan, prednimustine, procarbazine, ranimustine, satraplatin, semustine, streptozocin, temozolomide, thiotepa, treosulfan, triaziquone, triethylenemelamine, triplatin tetranitrate, trofosphamide, and uramustine; an antibiotic, including, but not limiting to, aclarubicin, amrubicin, bleomycin, dactinomycin, daunorubicin, doxorubicin, elsamitrucin, epirubicin, idarubicin, menogaril, mitomycin, neocarzinostatin, pentostatin, pirarubicin, plicamycin, valrubicin, and zorubicin; an antimetabolite, including, but not limiting to, aminopterin, azacitidine, azathioprine, capecitabine, cladribine, clofarabine, cytarabine, decitabine, floxuridine, fludarabine, 5-fluorouracil, gemcitabine, hydroxyurea, mercaptopurine, methotrexate, nelarabine, pemetrexed, raltitrexed, tegafur-uracil, thioguanine, trimethoprim, trimetrexate, and vidarabine; an immunotherapy, an antibody therapy, including, but not limiting to, alemtuzumab, bevacizumab, cetuximab, galiximab, gemtuzumab, panitumumab, pertuzumab, rituximab, brentuximab, tositumomab, trastuzumab, 90 Y ibritumomab tiuxetan, ipilimumab, tremelimumab and anti-CTLA-4 antibodies; a hormone or hormone antagonist, including, but not limiting to, anastrozole, androgens, buserelin, diethylstilbestrol, exemestane, flutamide, fulvestrant, goserelin, idoxifene, letrozole, leuprolide, magestrol, raloxifene, tamoxifen, and toremifene; a taxane, including, but not limiting to, DJ-927, docetaxel, TPI 287, larotaxel, ortataxel, paclitaxel, DHA-paclitaxel, and tesetaxel; a retinoid, including, but not limiting to, alitretinoin, bexarotene, fenretinide, isotretinoin, and tretinoin; an alkaloid, including, but not limiting to, demecolcine, homoharringtonine, vinblastine, vincristine, vindesine, vinflunine, and vinorelbine; an antiangiogenic agent, including, but not limiting to, AE-941 (GW786034, Neovastat), ABT-510, 2-methoxyestradiol, lenalidomide, and thalidomide; a topoisomerase inhibitor, including, but not limiting to, amsacrine, belotecan, edotecarin, etoposide, etoposide phosphate, exatecan, irinotecan (also active metabolite SN-38 (7-ethyl-10-hydroxy-camptothecin)), lucanthone, mitoxantrone, pixantrone, rubitecan, teniposide, topotecan, and 9-aminocamptothecin; a kinase inhibitor, including, but not liming to, axitinib (AG 013736), dasatinib (BMS 354825), erlotinib, gefitinib, flavopiridol, imatinib mesylate, lapatinib, motesanib diphosphate (AMG 706), nilotinib (AMN107), seliciclib, sorafenib, sunitinib malate, AEE-788, BMS-599626, UCN-01 (7-hydroxystaurosporine), vemurafenib, dabrafenib, selumetinib, paradox breakers (such as PLX8394 or PLX7904), LGX818, BGB-283, pexidartinib (PLX3397) and vatalanib; a targeted signal transduction inhibitor including, but not limiting to bortezomib, geldanamycin, and rapamycin; a biological response modifier, including, but not limiting to, imiquimod, interferon-α, and interleukin-2; and other chemotherapeutics, including, but not limiting to 3-AP (3-amino-2-carboxyaldehyde thiosemicarbazone), altrasentan, aminoglutethimide, anagrelide, asparaginase, bryostatin-1, cilengitide, elesclomol, eribulin mesylate (E7389), ixabepilone, lonidamine, masoprocol, mitoguanazone, oblimersen, sulindac, testolactone, tiazofurin, mTOR inhibitors (e.g. sirolimus, temsirolimus, everolimus, deforolimus, INK28, AZD8055, PI3K inhibitors (e.g. BEZ235, GDC-0941, XL147, XL765, BMK120), cyclin dependent kinase (CDK) inhibitors (e.g., a CDK4 inhibitor or a CDK6 inhibitor, such as Palbociclib (PD-0332991), Ribocyclib (LEE011), Abemaciclib (LY2835219), P1446A-05, Abemaciclib (LY2835219), Trilaciclib (G1T28), etc.), AKT inhibitors, Hsp90 inhibitors (e.g. geldanamycin, radicicol, tanespimycin), farnesyltransferase inhibitors (e.g. tipifarnib), Aromatase inhibitors (anastrozole letrozole exemestane); an MEK inhibitor including, but are not limited to, AS703026, AZD6244 (Selumetinib), AZD8330, BIX 02188, CI-1040 (PD184352), GSK1120212 (also known as trametinib or JTP-74057), cobimetinib, PD0325901, PD318088, PD98059, RDEA119 (BAY 869766), TAK-733 and U0126-EtOH; tyrosine kinase inhibitors, including, but are not limited to, AEE788, AG-1478 (Tyrphostin AG-1478), AG-490, Apatinib (YN968D1), AV-412, AV-951 (Tivozanib), Axitinib, AZD8931, BIBF1120 (Vargatef), BIBW2992 (Afatinib), BMS794833, BMS-599626, Brivanib (BMS-540215), Brivanib alaninate (BMS-582664), Cediranib (AZD2171), Chrysophanic acid (Chrysophanol), Crenolanib (CP-868569), CUDC-101, CYC116, Dovitinib Dilactic acid (TKI258 Dilactic acid), E7080, Erlotinib Hydrochloride (Tarceva, CP-358774, OSI-774, NSC-718781), Foretinib (GSK1363089, XL880), Gefitinib (ZD-1839 or Iressa), Imatinib (Gleevec), Imatinib Mesylate, Ki8751, KRN 633, Lapatinib (Tykerb), Linifanib (ABT-869), Masitinib (Masivet, AB1010), MGCD-265, Motesanib (AMG-706), MP-470, Mubritinib (TAK 165), Neratinib (HKI-272), NVP-BHG712, OSI-420 (Desmethyl Erlotinib, CP-473420), OSI-930, Pazopanib HCl, PD-153035 HCl, PD173074, Pelitinib (EKB-569), PF299804, Ponatinib (AP24534), PP121, RAF265 (CHIR-265), Raf265 derivative, Regorafenib (BAY 73-4506), Sorafenib Tosylate (Nexavar), Sunitinib Malate (Sutent), Telatinib (BAY 57-9352), TSU-68 (SU6668), Vandetanib (Zactima), Vatalanib dihydrochloride (PTK787), WZ3146, WZ4002, WZ8040, quizartinib, Cabozantinib, XL647, EGFR siRNA, FLT4 siRNA, KDR siRNA, Antidiabetic agents such as metformin, PPAR agonists (rosiglitazone, pioglitazone, bezafibrate, ciprofibrate, clofibrate, gemfibrozil, fenofibrate, indeglitazar), DPP4 inhibitors (sitagliptin, vildagliptin, saxagliptin, dutogliptin, gemigliptin, alogliptin) or an EGFR inhibitor, including, but not limited to, AEE-788, AP-26113, BIBW-2992 (Tovok), CI-1033, GW-572016, Iressa, LY2874455, RO-5323441, Tarceva (Erlotinib, OSI-774), CUDC-101 and WZ4002.

In certain embodiments, the second chemotherapeutic agent is selected from afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, idelalisib, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, olaparib, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelboraf), vincristine, vinblastine, vinorelbine, vindesine, and the like. In certain embodiments, the compounds herein are administered prior to, concurrently with, or subsequent to the treatment with the chemotherapeutic agent.

In certain embodiments, the method of treating a cancer comprises administering a therapeutically effective amount of a compound described herein and a therapeutically effective amount a biologic agent used to treat cancer. In certain embodiments, the biologic agent is selected from anti-BAFF (e.g., belimumab); anti-CCR4 (e.g., mogamulizumab); anti-CD19/CD3 (e.g., blinatumomab); anti-CD20 (e.g., obinutuzumab, rituximab, ibritumomab tiuxetan, ofatumumab, tositumomab); anti-CD22 (e.g., moxetumomab pasudotox); anti-CD30 (e.g., brentuximab vedotin); anti-CD33 (e.g., gemtuzumab); anti-CD37 (e.g., otlertuzumab); anti-CD38 (e.g., daratumumab); anti-CD52 (e.g., alemtuzumab); anti-CD56 (e.g., lorvotuzumab mertansine); anti-CD74 (e.g., milatuzumab); anti-CD105; anti-CD248 (TEM1) (e.g., ontuxizumab); anti-CTLA4 (e.g., tremelimumab, ipilimumab); anti-EGFL7 (e.g., parsatuzumab); anti-EGFR (HER1/ERBB1) (e.g., panitumumab, nimotuzumab, necitumumab, cetuximab, imgatuzumab, futuximab); anti-FZD7 (e.g., vantictumab); anti-HER2 (ERBB2/neu) (e.g., margetuximab, pertuzumab, ado-trastuzumab emtansine, trastuzumab); anti-HER3 (ERBB3); anti-HGF (e.g., rilotumumab, ficlatuzumab); anti-IGF-1R (e.g., ganitumab, figitumumab, cixutumumab, dalotuzumab); anti-IGF-2R; anti-KIR (e.g., lirilumab, onartuzumab); anti-MMP9; anti-PD-1 (e.g., nivolumab, pidilizumab, lambrolizumab); anti-PD-L1 (e.g. Atezolizumab); anti-PDGFRa (e.g., ramucirumab, tovetumab); anti-PD-L2; anti-PIGF (e.g., ziv-aflibercept); anti-RANKL (e.g., denosumab); anti-TNFRSF 9 (CD 137/4-1 BB) (e.g., urelumab); anti-TRAIL-RI/DR4,R2/D5 (e.g., dulanermin); anti-TRAIL-R1/D4 (e.g., mapatumumab); anti-TRAIL-R2/D5 (e.g., conatumumab, lexatumumab, apomab); anti-VEGFA (e.g., bevacizumab, ziv-aflibercept); anti-VEGFB (e.g., ziv-aflibercept); and anti-VEGFR2 (e.g., ramucirumab).

Biological sample for the method herein include any samples are amenable to analysis herein, such as tissue or biopsy samples containing cancer cells, or any biological fluids that contain the material of interests (e.g., DNA), such as blood, plasma, saliva, tissue swabs, and intestinal fluids. In certain embodiments, exosomes extruded by cancer cells and obtained from blood or other body fluids can be used to detect nucleic acids and proteins produced by the cancer cells. In general, a “sample” can refer to a biomolecule, such as a protein, a peptide, a nucleic acid, a lipid, a carbohydrate or a combination thereof, that is obtained from an organism, particularly a mammal. Examples of mammals include humans; veterinary animals like cats, dogs, horses, cattle, and swine; and laboratory animals like mice, rats and primates. In certain embodiments, a human subject in the clinical setting is referred to as a patient. Biological samples include tissue samples (such as tissue sections and needle biopsies of tissue), cell samples (for example, cytological smears such as Pap or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, milk, vaginal fluid, saliva, swabs (such as buccal swabs), or any material containing biomolecules that is derived from a first biological sample. In certain embodiments, the biological sample is a “cell free sample,” such as cell free or extracellular polynucleotides, and cell free or extracellular proteins. In certain embodiments, cell free DNA or cfDNA refers to extracellular DNA obtained from blood, particularly the serum.

General biological, biochemical, immunological and molecular biological methods applicable to the present disclosure are described in Sambrook et al., Molecular Cloning: A Laboratory Manual 2^{nd} Ed. (1989) Cold Spring Harbor Laboratory Press, Cold Spring Harbor, N.Y.; Current Protocols in Molecular Biology, Ausubel et al., ed., John Wiley & Sons (2015); Current Protocols in Immunology, Coligan, JE ed., John Wiley & Sons (2015); and Methods in Enzymology, Vol. 200, Abelson et al., ed., Academic Press (1991). All publications are incorporated herein by reference.

### 5. Formulations and Administration

In certain embodiments, the pharmaceutical compositions of the compounds can be formulated by standard techniques using one or more physiologically acceptable carriers or excipients. Suitable pharmaceutical carriers are described herein and in Remington: The Science and Practice of Pharmacy, 21^{st} Ed. (2005). The therapeutic compounds and their physiologically acceptable salts, hydrates and solvates can be formulated for administration by any suitable route, including, among others, topically, nasally, orally, parenterally, rectally or by inhalation. In certain embodiments, the administration of the pharmaceutical composition may be made by intradermal, subdermal, intravenous, intramuscular, intranasal, intracerebral, intratracheal, intraarterial, intraperitoneal, intravesical, intrapleural, intracoronary or intratumoral injection, with a syringe or other devices. Transdermal administration is also contemplated, as are inhalation or aerosol administration. Tablets, capsules, and solutions can be administered orally, rectally or vaginally.

For oral administration, a pharmaceutical composition can take the form of, for example, a tablet or a capsule prepared by conventional means with a pharmaceutically acceptable excipient. Tablets and capsules comprising the active ingredient can be prepared together with excipients such as: (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate; (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; (c) binders, e.g., magnesium aluminum silicate, starch paste, gelatin, tragacanth, methylcellulose, sodium carboxymethylcellulose, polyvinylpyrrolidone and/or hydroxypropyl methylcellulose; (d) disintegrants, e.g., starches (including potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners. The compositions are prepared according to conventional mixing, granulating or coating methods.

In certain embodiments, the carrier is a cyclodextrin, such as to enhance solubility and/or bioavailability of the compounds herein. In certain embodiments, the cyclodextrin for use in the pharmaceutical compositions can be selected from α-cyclodextrin, β-cyclodextrin, γ-cyclodextrin, derivatives thereof, and combinations thereof. In certain embodiments, the cyclodextrin is selected from β-cyclodextrin, γ-cyclodextrin, derivatives thereof, and combinations thereof.

In certain embodiments, the compounds can be formulated with a cyclodextrin or derivative thereof selected from carboxyalkyl cyclodextrin, hydroxyalkyl cyclodextrin, sulfoalkylether cyclodextrin, and an alkyl cyclodextrin. In various embodiments, the alkyl group in the cyclodextrin is methyl, ethyl, propyl, butyl, or pentyl.

In certain embodiments, the cyclodextrin is α-cyclodextrin or a derivative thereof. In certain embodiments, the α-cyclodextrin or derivative thereof is selected from carboxyalkyl-α-cyclodextrin, hydroxyalkyl-α-cyclodextrin, sulfoalkylether-α-cyclodextrin, alkyl-α-cyclodextrin, and combinations thereof.

In certain embodiments, the alkyl group in the α-cyclodextrin derivative is methyl, ethyl, propyl, butyl, or pentyl.

In certain embodiments, the cyclodextrin is β-cyclodextrin or a derivative thereof. In certain embodiments, the β-cyclodextrin or derivative thereof is selected from carboxyalkyl-β-cyclodextrin, hydroxyalkyl-β-cyclodextrin, sulfoalkylether-β-cyclodextrin, alkyl-β-cyclodextrin, and combinations thereof. In certain embodiments, the alkyl group in the β-cyclodextrin derivative is methyl, ethyl, propyl, butyl, or pentyl.

In certain embodiments, the β-cyclodextrin or a derivative thereof is hydroxyalkyl-β-cyclodextrin or sulfoalkylether-β-cyclodextrin. In certain embodiments, the hydroxyalkyl-β-cyclodextrin is hydroxypropyl-β-cyclodextrin. In certain embodiments, the sulfoalkylether-β-cyclodextrin is sulfobutylether-β-cyclodextrin.

In certain embodiments, β-cyclodextrin or a derivative thereof is alkyl-β-cyclodextrin, or methyl-β-cyclodextrin. In certain embodiments using methyl-β-cyclodextrin, the β-cyclodextrin is randomly methylated β-cyclodextrin.

In certain embodiments, the cyclodextrin is γ-cyclodextrin or a derivative thereof. In certain embodiments, the γ-cyclodextrin or derivative thereof is selected from carboxyalkyl-γ-cyclodextrin, hydroxyalkyl-γ-cyclodextrin, sulfoalkylether-γ-cyclodextrin, and alkyl-γ-cyclodextrin. In certain embodiments, the alkyl group in the γ-cyclodextrin derivative is methyl, ethyl, propyl, butyl, or pentyl. In certain embodiments, the γ-cyclodextrin or derivative thereof is hydroxyalkyly-cyclodextrin or sulfoalkylether-γ-cyclodextrin. In certain embodiments, the hydroxyalkyly-cyclodextrin is hydroxypropyl-cyclodextrin.

When used in a formulation with the compound of the present disclosure, the cyclodextrin can be present at about 0.1 w/v to about 30% w/v, about 0.1 w/v to about 20% w/v, about 0.5% w/v to about 10% w/v, or about 1% w/v to about 5% w/v. In certain embodiments, the cyclodextrin is present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 14% w/v, about 16% w/v, about 18% w/v, about 20% w/v, about 25% w/v, or about 30% w/v or more.

Tablets may be either film coated or enteric coated according to methods known in the art. Liquid preparations for oral administration can take the form of, for example, solutions, syrups, or suspensions, or they can be presented as a dry product for reconstitution with water or other suitable vehicle before use. Such liquid preparations can be prepared by conventional means with pharmaceutically acceptable carriers and additives, for example, suspending agents, e.g., sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

The compounds can be formulated for parenteral administration, for example by bolus injection or continuous infusion. Formulations for injection can be presented in unit dosage form, for example, in ampoules or in multi-dose containers, with an optionally added preservative. Injectable compositions can be aqueous isotonic solutions or suspensions. In certain embodiments for parenteral administration, the compounds can be prepared with a surfactant, such as Cremaphor, or lipophilic solvents, such as triglycerides or liposomes. The compositions may be sterilized and/or contain adjuvants, such as preserving, stabilizing, wetting or emulsifying agents, solution promoters, salts for regulating the osmotic pressure and/or buffers. Alternatively, the compound can be in powder form for reconstitution with a suitable vehicle, for example, sterile pyrogen-free water, before use. In addition, they may also contain other therapeutically effective substances.

For administration by inhalation, the compound may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

Suitable formulations for transdermal application include an effective amount of a compound with a carrier. Preferred carriers include absorbable pharmacologically acceptable solvents to assist passage through the skin of the subject. For example, transdermal devices are in the form of a bandage or patch comprising a backing member, a reservoir containing the compound optionally with carriers, optionally a rate controlling barrier to deliver the compound to the skin of the host at a controlled and predetermined rate over a prolonged period of time, and a means to secure the device to the skin. Matrix transdermal formulations may also be used.

Suitable formulations for topical application, e.g., to the skin and eyes, are preferably aqueous solutions, ointments, creams or gels well-known in the art. The formulations may contain solubilizers, stabilizers, tonicity enhancing agents, buffers and preservatives.

In certain embodiments, the compound can also be formulated as a rectal composition, for example, suppositories or retention enemas, for example, containing conventional suppository bases, for example, cocoa butter or other glycerides, or gel forming agents, such as carbomers.

In certain embodiments, the compound can be formulated as a depot preparation. Such long-acting formulations can be administered by implantation (for example, subcutaneously or intramuscularly) or by intramuscular injection. The compound can be formulated with suitable polymeric or hydrophobic materials (for example as an emulsion in an acceptable oil), ion exchange resins, biodegradable polymers, or as sparingly soluble derivatives, for example, as a sparingly soluble salt.

The pharmaceutical compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

### 6. Effective Amount and Dosing

In certain embodiments, a pharmaceutical composition of the compound is administered to a subject, preferably a human, at a therapeutically effective dose to prevent, treat, or control a condition or disease as described herein. The pharmaceutical composition is administered to a subject in an amount sufficient to elicit an effective therapeutic response in the subject. An effective therapeutic response is a response that at least partially arrests or slows the symptoms or complications of the condition or disease. An amount adequate to accomplish this is defined as “therapeutically effective dose” or “therapeutically effective amount.” The dosage of compounds can take into consideration, among others, the species of warm-blooded animal (mammal), the body weight, age, condition being treated, the severity of the condition being treated, the form of administration, route of administration. The size of the dose also will be determined by the existence, nature, and extent of any adverse effects that accompany the administration of a particular therapeutic compound in a particular subject.

In certain embodiments, a suitable dosage of the compounds of the disclosure or a composition thereof is from about 1 ng/kg to about 1000 mg/kg, from 0.01 mg/kg to 900 mg/kg, 0.1 mg/kg to 800 mg/kg, from about 1 mg/kg to about 700 mg/kg, from about 2 mg/kg to about 500 mg/kg, from about 3 mg/kg to about 400 mg/kg, 4 mg/kg to about 300 mg/kg, or from about 5 mg/kg to about 200 mg/kg. In certain embodiments, the suitable dosages of the compound can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. In certain embodiments, the dose of the compound can be administered once per day or divided into subdoses and administered in multiple doses, e.g., twice, three times, or four times per day.

In certain embodiments, the compounds can be administered with one or more of a second compound, sequentially or concurrently, either by the same route or by different routes of administration. When administered sequentially, the time between administrations is selected to benefit, among others, the therapeutic efficacy and/or safety of the combination treatment. In certain embodiments, the compounds herein can be administered first followed by a second compound, or alternatively, the second compound administered first followed by the compounds of the present disclosure. By way of example and not limitation, the time between administrations is about 1 hr, about 2 hr, about 4 hr, about 6 hr, about 12 hr, about 16 hr or about 20 hr. In certain embodiments, the time between administrations is about 1, about 2, about 3, about 4, about 5, about 6, or about 7 more days. In certain embodiments, the time between administrations is about 1 week, 2 weeks, 3 weeks, or 4 weeks or more. In certain embodiments, the time between administrations is about 1 month or 2 months or more.

When administered concurrently, the compound can be administered separately at the same time as the second compound, by the same or different routes, or administered in a single composition by the same route. In certain embodiments, the amount and frequency of administration of the second compound can used standard dosages and standard administration frequencies used for the particular compound. See, e.g., Physicians' Desk Reference, 70th Ed., PDR Network, 2015; incorporated herein by reference.

In certain embodiments where the compounds of the present disclosure is administered in combination with a second compound, the dose of the second compound is administered at a therapeutically effective dose. In certain embodiments, a suitable dose can be from about 1 ng/kg to about 1000 mg/kg, from about 0.01 mg/kg to about 900 mg/kg, from about 0.1 mg/kg to about 800 mg/kg, from about 1 mg/kg to about 700 mg/kg, from about 2 mg/kg to about 500 mg/kg, from about 3 mg/kg to about 400 mg/kg, from about 4 mg/kg to about 300 mg/kg, or from about 5 mg/kg to about 200 mg/kg. In certain embodiments, the suitable dosages of the second compound can be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. In certain embodiments, guidance for dosages of the second compound is provided in Physicians' Desk Reference, 70^{th} Ed, PDR Network (2015), incorporated herein by reference.

It to be understood that optimum dosages, toxicity, and therapeutic efficacy of such compounds may vary depending on the relative potency of individual compound and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. compounds or combinations thereof that exhibit large therapeutic indices are preferred. While certain agents that exhibit toxic side effects can be used, care should be used to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods disclosed herein, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

### 7. Methods of Preparation

The following examples are provided to further illustrate the methods of the present disclosure, and the compounds and compositions for use in the methods. The examples described are illustrative only and are not intended to limit the scope of the invention(s) in any way. The disclosures of all articles and references mentioned in this application, including patents, are incorporated herein by reference in their entirety.

The compounds of the present disclosure can be synthesized in view of the guidance provided herein, incorporating known chemical reactions and related procedures such as separation and purification. Representative methods and procedures for preparation of the compounds in this disclosure are described below and in the Examples. Acronyms are abbreviations are used per convention which can be found in literature and scientific journals.

It is understood that the starting materials and reaction conditions may be varied, the sequence of the reactions altered, and additional steps employed to produce compounds encompassed by the present disclosure, as demonstrated by the following examples. General references for known chemical reactions useful for synthesizing the disclosed compounds are available (see, e.g., Smith and March, March's Advanced Organic Chemistry: Reactions, Mechanisms, and Structure, Fifth Edition, Wiley Interscience, 2001; or Carey and Sundberg, Advanced Organic Chemistry, Part B. Reaction and Synthesis; Fifth Edition, Springer, 2007; or Li, J. J. Name Reactions, A Collection of Detailed Mechanisms and Synthetic Applications; Fifth Edition, Springer, 2014).

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

General Synthesis

In certain embodiments, compounds disclosed herein can be according to the general schemes shown below. For example, compounds of Formula A-I can be prepared according to the general syntheses outlined below in Scheme A-1, where suitable reagents can be purchased form commercial sources or synthesized via known methods or methods adapted from the examples provided herein. In Scheme 1, each of ring A, ring B, X, R¹, R², R³, R⁴, p and q are independently as defined herein.

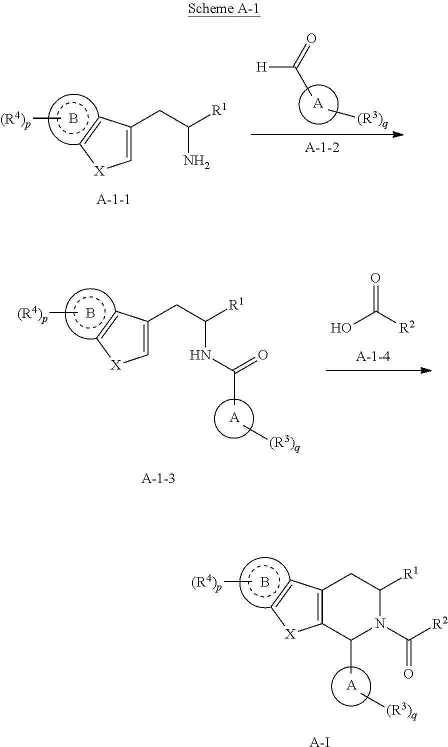

In Scheme A-1, compound A-1-3 can be provided by coupling amine A-1-1 with A-1-2. Cyclization provides compound A-1-3, which can be achieved under standard cyclization reaction conditions. Exemplary cyclization reaction conditions include, but are not limited to, reducing agents, such as a hydride (e.g., NaBH₄, LiAlH₄, etc.), or an aprotic solvent in the presence of an acid catalyst. Compounds of Formula I can then be provided by coupling compound A-1-3 with compound A-1-4 under reaction conditions suitable to provide compounds of Formula A-I. Upon each reaction completion, each of the intermediate or final compounds can be recovered, and optionally purified, by conventional techniques such as neutralization, extraction, precipitation, chromatography, filtration and the like.

Appropriate starting materials and reagents for use in Scheme A-1 can be purchased or prepared by methods known to one of skill in the art. As shown in Scheme A-2, chiral or enantiomerically enriched starting materials can be provided for use in the method of Scheme A-1 by converting a chiral or enantiomerically enriched amino alcohol to a oxathiazolidine dioxide A-2-2. In Scheme A-2, ring B, X, R¹, R⁴, and p are independently as defined herein, M is a metal halide (e.g., MgBr) and PG is a protecting group (e.g., Boc).

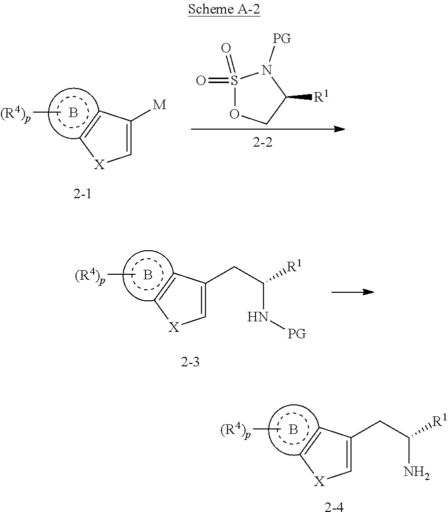

Referring to Scheme A-2, compound A-2-1 is coupled to compound A-2-2 under standard coupling conditions to produce compound A-2-3. The reaction is typically conducted in the presence of suitable catalyst (e.g., Cup using suitable solvents/solvent mixtures. Deprotection of compound A-2-3 provides compound A-2-4. Upon reaction completion, each intermediate can be recovered by conventional techniques such as neutralization, extraction, precipitation, chromatography, filtration and the like. In some embodiments of the methods of Scheme A-1, and Scheme A-2, the various substituents on the starting compound (e.g., compound I-1 and compound 1-2, (e.g., R¹, R², R³, R⁴, etc.) are as defined for Formula A-I. However, it should also be appreciated that chemical derivatization and/or functional group interconversion, can be used to further modify of any of the compounds of Scheme A-1 or Scheme A-2 in order to provide the various compounds of Formula A-I.

In certain embodiments, compounds of Formula (B-I) can be prepared according to the general syntheses outlined below in Scheme B-1, where suitable reagents can be purchased form commercial sources or synthesized via known methods or methods adapted from the examples provided herein. In Scheme B-1, each of ring A, X, R¹, R², R³, R⁴, p and q are defined by the compounds disclosed in Table B-1 and the following procedures, further in view of chemical definitions of functional groups as defined above. LG is a leaving group (e.g., halo).

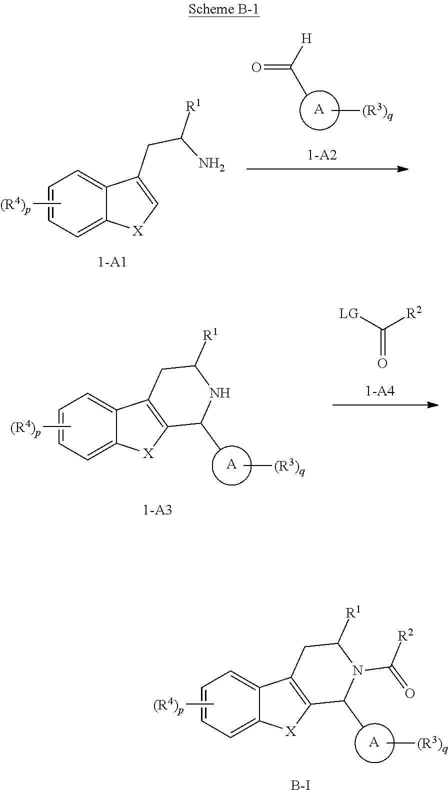

In Scheme B-1, compound 1-A3 can be provided by cyclizing an amine 1-A1 with aldehyde 1-A2. Exemplary cyclization reaction conditions include, but are not limited to, reducing agents, such as a hydride (e.g., NaBH₄, LiAlH₄, etc.), or an aprotic solvent in the presence of an acid catalyst. Compounds disclosed herein can then be provided by coupling 1-A3 with 1-A4 under reaction conditions suitable to provide compounds of Formula (A-I). Suitable reagents and starting materials can be purchased form commercial sources or synthesized via known methods or methods adapted from the Synthetic Examples provided herein. Upon each reaction completion, each of the intermediate or final compounds can be recovered, and optionally purified, by conventional techniques such as neutralization, extraction, precipitation, chromatography, filtration and the like.

In some embodiments of the methods of Scheme B-1, the various substituents on a starting compound of for Formula B-I are as defined for Formula A-I. However, it should also be appreciated that chemical derivatization and/or functional group interconversion, can be used to further modify of any of the compounds of Scheme B-1 in order to provide the various compounds of Formula B-I.

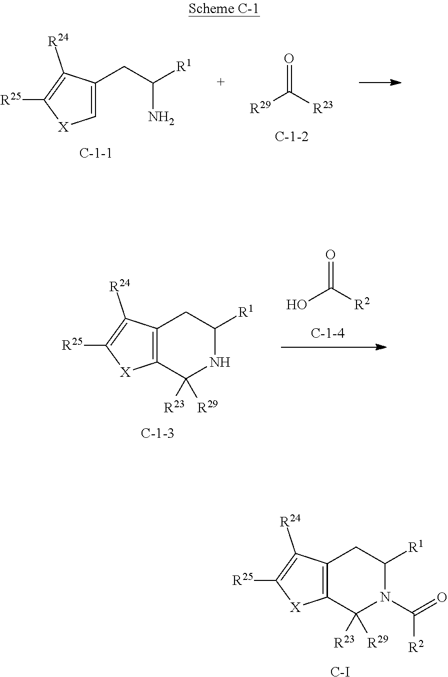

In Scheme C-1, compound C-1-3 can be provided by coupling amine C-1-1 with C-1-2. Cyclization provides compound C-1-3, which can be achieved under standard cyclization reaction conditions. Exemplary cyclization reaction conditions include, but are not limited to, reducing agents, such as a hydride (e.g., NaBH₄, LiAlH₄, etc.), or an aprotic solvent in the presence of an acid catalyst. Compounds of Formula I can then be provided by coupling compound C-1-3 with compound C-1-4 under reaction conditions suitable to provide compounds of Formula C-I. Upon each reaction completion, each of the intermediate or final compounds can be recovered, and optionally purified, by conventional techniques such as neutralization, extraction, precipitation, chromatography, filtration and the like.

Appropriate starting materials and reagents for use in Scheme C-1 can be purchased or prepared by methods known to one of skill in the art. As shown in Scheme C-2, chiral or enantiomerically enriched starting materials can be provided for use in the method of Scheme C-1 by converting a chiral or enantiomerically enriched amino alcohol to a oxathiazolidine dioxide C-2-2. In Scheme C-2, X, R¹, R²⁴, and R²⁵ are independently as defined herein, M is a metal halide (e.g., MgBr) and PG is a protecting group (e.g., Boc).

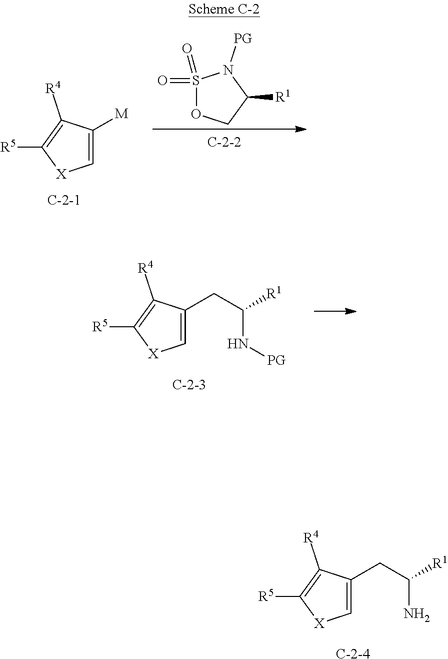

Referring to Scheme C-2, compound C-2-1 is coupled to compound C-2-2 under standard coupling conditions to produce compound C-2-3. The reaction is typically conducted in the presence of suitable catalyst (e.g., Cup using suitable solvents/solvent mixtures. Deprotection of compound C-2-3 provides compound C-2-4. Upon reaction completion, each intermediate can be recovered by conventional techniques such as neutralization, extraction, precipitation, chromatography, filtration and the like.

In some embodiments of the methods of Scheme C-1 and Scheme C-2, the various substituents on the starting compound (e.g., compound C-1-1 and compound C-1-2, (e.g., X, R¹, R², R²³, R²⁴, R²⁵, and R²⁹) are as defined for Formula C-I. However, it should also be appreciated that chemical derivatization and/or functional group interconversion, can be used to further modify of any of the compounds of Scheme C-1 or Scheme C-2 in order to provide the various compounds of Formula C-I.

Other compounds of the disclosure can be synthesized using the synthetic routes above and adapting chemical synthetic procedures available to the skilled artisan. Exemplary methods of synthesis are provided in the Examples. It is to be understood that each of the procedures describing synthesis of exemplary compounds are part of the specification, and thus incorporated herein into the Detailed Description of this disclosure.

### SYNTHETIC EXAMPLES

### Procedure 1: Synthesis of Compounds A-1 and A-2

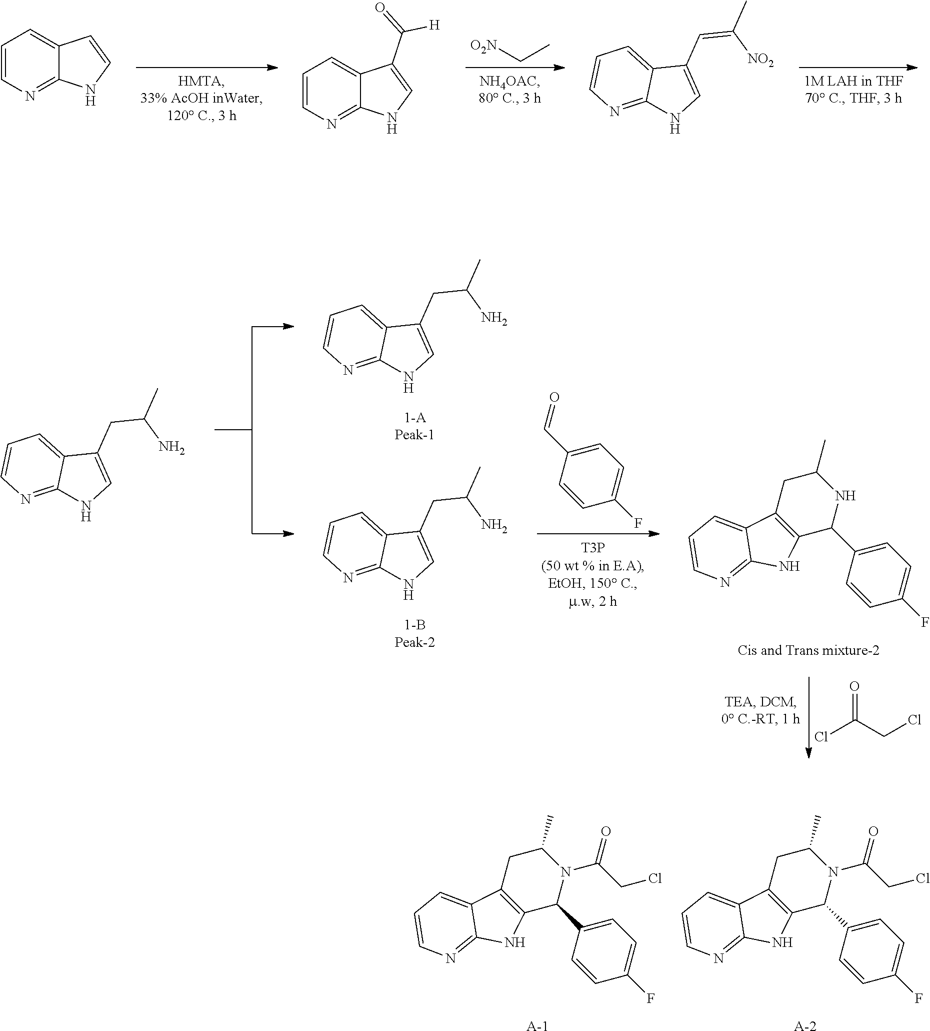

1H-pyrrolo[2,3-b]pyridine-3-carbaldehyde: To a suspension of 1H-pyrrol[2,3-b]pyridine (10 g, 84.74 mmol, 1 eq) in water containing 33% acetic acid (100 mL) was added hexamethylenetetramine (13 g, 93.22 mmol, 1.1 eq) at room temperature. This solution was heated to 120° C. and stirred for 3 h. Then the reaction mixture was cooled to room temperature and during this period lot of solid was formed. This suspension was poured into a beaker (1 L) containing an ice and the flask has rinsed with water (50 mL). This was then neutralized with saturated bicarbonate solution slowly. After neutralization, the solid was collected by filtration and washed with water. After drying under vacuum to give 1H-pyrrol[2,3-b]pyridine-3-carbaldehyde. LC-MS (m/z): 146 [M+H]⁺. ¹H NMR (400 MHz, DMSO-d6) δ 7.24-7.27 (m, 1H), 8.34-8.54 (m, 3H), 9.94 (s, 1H), 112.67 (bs, 1H).

(Z)-3-(2-nitroprop-1-en-1-yl)-1H-pyrrolo[2,3-b]pyridine: To a suspension of 1H-pyrrol[2,3-b]pyridine-3-carbaldehyde (10 g, 68.49 mmol, 1 eq) in nitro ethane (40 mL) and ammonium acetate (2.63 g, 34.24 mmol, 0.5 eq) was added under cooling condition, it was stirred at 0° C. for 5 mins, then reaction mixture was stirred at 80° C. for 3 hours, after completion of the reaction, reaction mixture was quenched with 15 mL of water, precipitate was formed, obtained solid was filtered through sintered funnel and washed with n-pentane (25 mL), dried under high vacuum to get the product. (Z)-3-(2-nitroprop-1-en-1-yl)-1H-pyrrolo[2,3-b]pyridine. LC-MS (m/z): 204 [M+H]⁺. ¹H NMR (400 MHz, DMSO-d6) δ 2.55 (s, 3H), 7.44 (s, 1H), 9.94 (s, 1H), 7.78 (s, 1H), 8.34 (s, 1H), 8.42 (s, 2H).

1-(1H-pyrrolo[2,3-b]pyridin-3-yl)propan-2-amine: To a solution of (Z)-3-(2-nitroprop-1-en-1-yl)-1H-pyrrolo[2,3-b]pyridine (11 g, 54.13 mmol, 1 eq) in THF (200.0 mL) at −0° C. was added 1 M LAH solution in THF (270 mL, 270.66 mmol, 5 eq). Reaction mixture was warmed to room temperature, then the mixture was stirred at 70° C. for 7 h under N₂ atmosphere. TLC (10% MeOH in DCM) showed the reaction was completed. Reaction mixture was cooled to room temperature, The reaction was diluted with Diethyl ether (50 mL), after fisher—workup, reaction mixture was filtered through sintered funnel, using di ethyl ether, filtrate was concentrated under reduced pressure to get the product, without further purification crude product was forward to next step. LC-MS (m/z): 176 [M+H]⁺.

Chiral purification of 1-(1H-pyrrolo[2,3-b]pyridin-3-yl)propan-2-amine: 5 g crude material was separated by chiral prep HPLC by using the following analytical condition:

Column: CHIRALPAK IC (100 mm×4.6 mm×3 μm).

Mobile phase: n-Hexane: IPA with 0.1% DEA (80:20) Flow rate: 1 mL/min

Peak—1 (compound 1-A, 1.5 g, HPLC RT=8.00 min) and peak-2 (compound 1-B, 1.5 g, HPLC RT=13.25 min).

8-(4-fluorophenyl)-6-methyl-6,7,8,9-tetrahydro-5H-pyrrolo[2,3-b:5,4-c′]dipyridine: To a solution of 1-(1H-pyrrolo[2,3-b]pyridin-3-yl)propan-2-amine (peak-2, RT=13.25 min (0.5 g, 2.85 mmol, 1 eq) in EtOH (10.0 mL) was added 4-fluorobenzaldehyde (0.53 g, 4.28 mmol, 1.5 eq) and T3P (50 wt % in EtOAc was added to the reaction mixture. The mixture was irradiate with microwave at 150° C. for 2 h. TLC (5% MeOH in DCM) showed the reaction was completed. The reaction mixture was cooled to room temperature, reaction mixture was quenched with saturated bicarbonate solution (5 mL), extracted with 2×20 mL of ethyl acetate, combined organic layers were concentrated under reduced pressure to get the crude product. The crude product was triturated with diethyl ether (10 mL) and n-pentane (10 mL), and dried under high vacuum to give the product 8-(4-fluorophenyl)-6-methyl-6,7,8,9-tetrahydro-5H-pyrrolo[2,3-b:5,4-e]dipyridine (Cis and trans mixture-2). LC-MS (m/z): 282 [M+H]⁺.

To a solution of 8-(4-fluorophenyl)-6-methyl-6,7,8,9-tetrahydro-5H-pyrrolo[2,3-b:5,4-c′]dipyridine (isomer pair-2) (0.2 g, 0.71 mmol, 1 eq) in CH₂Cl₂ (15.0 mL) was added TEA (0.3 mL, 2.13 mmol, 3.0 eq) at 0° C., stirred for 5 min and then and 2-chloroacetyl chloride (0.08 mL, 1.06 mmol, 1.5 eq) was added at 0° C. The reaction mixture was stirred at room temperature for 1 hour. Reaction progress checked by TLC monitoring, after completion of the reaction, The reaction mixture was diluted with saturated NaHCO₃ solution (10 mL) and was extracted with DCM (2×50 mL). The organic layers were dried over Na₂SO₄ and concentrated to give the crude product. The crude product was purified by Prep TLC followed by prep HPLC purification using following analytical method.

Analytical Condition:

Column: X-Bridge C—18 (250 mm×4.6 mm×5 μm)

Mobile phase (A): 0.1% Ammonia in water

Mobile phase (B): Acetonitrile, Flow rate: 1.0 mL/min

Compound A-1: LC-MS (m/z): 358.2 [M+H]⁺

¹H NMR (400 MHz, DMSO-d6) δ ppm 0.90-1.18 (m, 3H), 2.65-2.73 (m, 1H), 3.10-3.15 (m, 1H), 4.48-4.61 (m, 3H), 6.86 (bs, 1H), 7.06-7.20 (m, 3H), 7.33-7.40 (m, 2H), 7.89 (bs, 1H), 8.19 (bs, 1H), 11.61 (bs, 1H).

Compound A-2: LC-MS (m/z): 358.2 [M+H]⁺

¹H NMR (400 MHz, DMSO-d6) δ ppm 0.90-1.21 (m, 3H), 2.89-2.92 (m, 1H), 3.16-3.28 (m, 1H), 4.32 (bs, 1H), 4.68-4.77 (m, 2H), 5.95 (bs, 1H), 7.20-7.00 (m, 3H), 7.42-7.61 (m, 2H), 7.85 (d, J=6.8 Hz, 1H), 8.11 (bs, 1H), 11.57 (bs, 1H).

Compounds as shown in Table A-1, can be or were, synthesized according to the procedures described above using the appropriate reagents and starting materials. Select data are shown in Table A-2.

TABLE A-2

| No. | MS [M + H]⁺ |
|---|---|
| 1 | 358 |
| 2 | 358 |
| 3 | 375.4 |

### Procedure B-1: Compound B-1

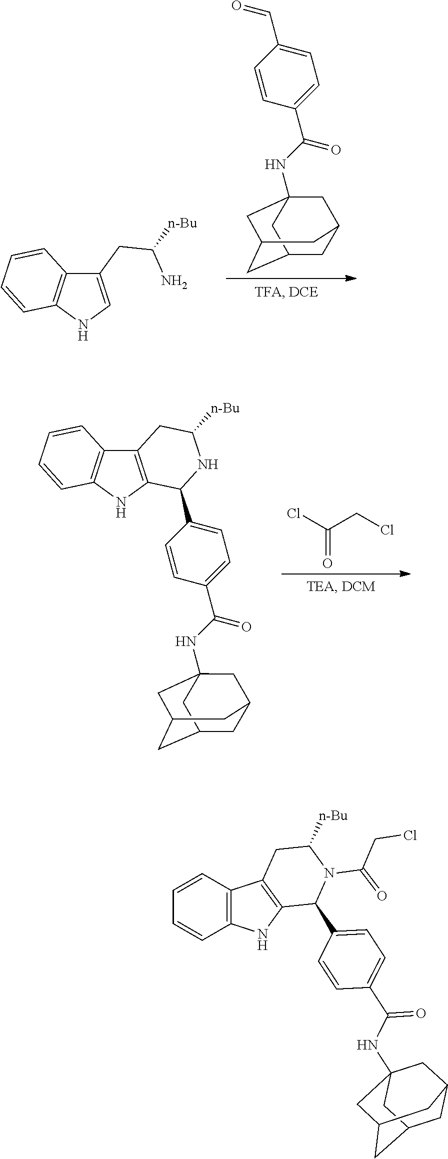

N-((1R,3R)-adamantan-1-yl)-4-((1S,3S)-3-butyl-2,3,4,9-tetrahydro-1H-pyrido[3,4-b]indol-1-yl)benzamide: To a solution of (S)-1-(1H-indol-3-yl)hexan-2-amine (1.0 g, 4.62 mmol, 1 eq) in DCE (7 mL) was added N-((3s,5s,7s)-adamantan-1-yl)-4-formylbenzamide (1.11 g, 3.92 mmol, 0.85 eq). To this TFA (0.7 mL, 9.24 mmol, 2 eq) was added at 0° C. The reaction mixture was heated at 80° C. for 8 h. TLC (5% MeOH in DCM) showed the reaction was completed. The reaction was cooled to room temperature and was concentrated under reduced pressure to get the crude. The crude was purified by flash chromatography using 2-3% MeOH in DCM as an eluent to give N-((1R,3R)-adamantan-1-yl)-4-((1S,3S)-3-butyl-2,3,4,9-tetrahydro-1H-pyrido[3,4-b]indol-1-yl)benzamide. LCMS (ES) m/z=482 [M+H]⁺.

N-((1R,3R)-adamantan-1-yl)-4-((1S,3S)-3-butyl-2-(2-chloroacetyl)-2,3,4,9-tetrahydro-1H-pyrido[3,4-b]indol-1-yl)benzamide: To a solution of N-((1R,3R)-adamantan-1-yl)-4-((1S,3S)-3-butyl-2,3,4,9-tetrahydro-1H-pyrido[3,4-b]indol-1-yl)benzamide (0.1 g, 0.20 mmol, 1 eq) in DCM (5.0 mL) was added triethyl amine (0.087 mL, 0.62 mmol, 3.0 eq) at RT, stirred for 15 mins and then 2-chloroacetyl chloride (0.024 mL, 0.31 mmol, 1.5 eq) was added at 0° C. The mixture was stirred at room temperature for 2 h under N₂ atmosphere. TLC (40% EtOAc in hexane) showed the reaction was completed. The reaction was cooled to room temperature and was diluted with ice cold water (5 mL) and was extracted with ethyl acetate (25 mL). The organic layer was dried over anhydrous Na₂SO₄, concentrated under reduced pressure to get the crude. The crude product was purified by preparative TLC using 2-4% MeOH in DCM as an eluent to get the product. Compound was further purified by Prep HPLC (Analytical condition: Column: Kinetex C18 (100 mm×4.6 mm×2.6 μm), mobile phase (A): 0.1% TFA in water, mobile phase (B): ACN, Flow rate: 0.75 mL/min, to give N-((1R,3R)-adamantan-1-yl)-4-((1S,3S)-3-butyl-2-(2-chloroacetyl)-2,3,4,9-tetrahydro-1H-pyrido[3,4-b]indol-1-yl)benzamide. ¹H NMR (400 MHz, DMSO-d6): δ 0.77-0.84 (m, 2H), 1.22 (s, 5H), 1.41 (bs, 2H), 1.61 (s, 6H), 2.00 (s, 9H), 3.01-3.04 (m, 2H), 4.31 (bs, 1H), 4.54 (s, 1H), 4.66 (s, 1H), 5.93 (s, 1H), 6.93-7.00 (m, 2H), 7.23 (d, J=7.6 Hz, 1H), 7.43 (s, 4H), 7.60 (s, 2H), 10.86 (s, 1H). LCMS (ES) m/z=558.4[M+H]⁺.

### Procedure B-2: Compound B-2

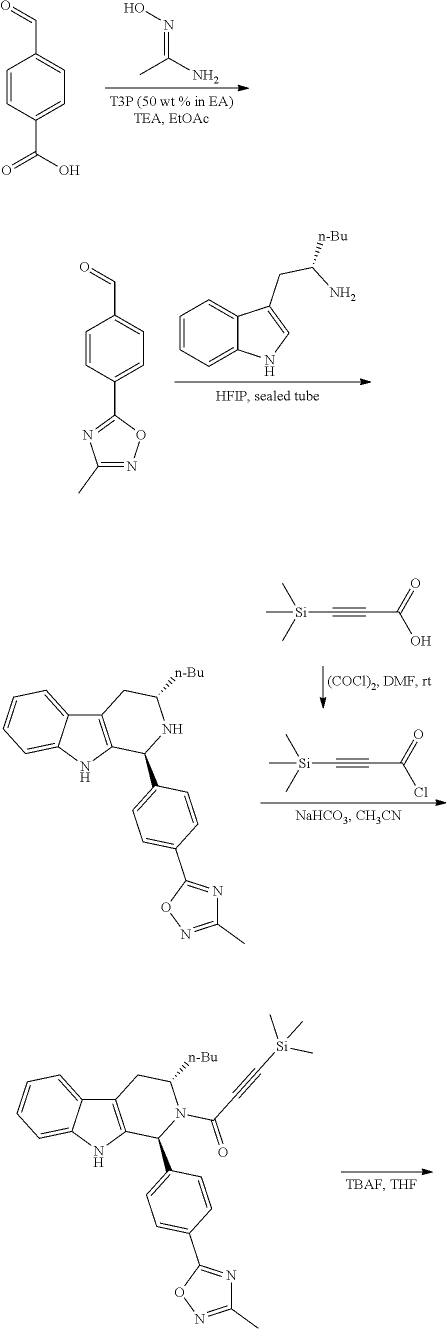

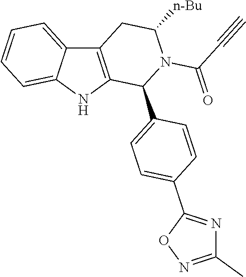

4-(3-methyl-1,2,4-oxadiazol-5-yl)benzaldehyde: To a stirred mixture of 4-formylbenzoic acid (2.0 g, 13.321 mmol, 1 eq), (E)-N′-hydroxyacetimidamide (1.1 g, 14.653 mmol, 1 eq), and trimethylamine (7.4 mL, 53.287 mmol, 4 eq) in ethyl acetate was added T3P (50 wt. % in ethyl acetate) (21 mL, 33.30 mmol, 2.5 eq) at room temperature. The mixture was heated to 80° C. and stirred for 4 h. The progress of the reaction was monitored by TLC (30% ethyl acetate in hexane). After completion of reaction, the reaction was allowed to cool to room temperature, quenched with water (100 mL), extracted with ethyl acetate (3×200 mL). The combined organic layers were washed with water (50 mL), brine (50 mL), dried over anhydrous sodium sulfate, filtered and concentrated under reduced pressure to obtain crude, which was purified flash column chromatography 20% ethyl acetate in hexane as an eluent to obtain 4-(3-methyl-1,2,4-oxadiazol-5-yl)benzaldehyde. LC-MS (m/z)=189 [M+H]⁺. ¹H NMR (400 MHz, DMSO-d6) δ ppm 2.44 (s, 3H), 1.24 (s, 5H), 8.11 (d, J=8.0 Hz, 2H), 8.29 (d, J=7.2 Hz, 2H), 10.11 (s, 1H).

5-(4-((1S,3S)-3-butyl-2,3,4,9-tetrahydro-1H-pyrido[3,4-b]indol-1-yl)phenyl)-3-methyl-1,2,4-oxadiazole: In a seal tube, (S)-1-(1H-indol-3-yl)hexan-2-amine (0.5 g, 2.311 mmol, 1.0 eq), 4-(3-methyl-1,2,4-oxadiazol-5-yl)benzaldehyde (0.43 g, 2.311 mmol, 1.0 eq) and hexafluoro-2-propanol (HFIP) (2.0 mL) were taken. The seal tube was closed and the mixture was heated to 110° C. and stirred for 16 h. The progress of the reaction was monitored by TLC (5% methanol in dichloromethane), the reaction was cooled to room temperature, concentrated under reduced pressure to obtain crude, which was purified by flash column chromatography using 3% methanol in dichloromethane as an eluent to obtain 5-(4-((1S,3S)-3-butyl-2,3,4,9-tetrahydro-1H-pyrido[3,4-b]indol-1-yl)phenyl)-3-methyl-1,2,4-oxadiazole. LCMS (ES) m/z=387 [M+H]⁺. ¹H NMR (400 MHz, DMSO-d6) δ ppm 0.81 (t, J=7.2 Hz, 3H), 1.16-1.21 (m, 2H), 1.29-1.42 (m, 4H), 2.32-2.33 (m, 1H), 2.39 (s, 3H), 2.74-2.77 (m, 2H), 5.19 (s, 1H), 6.95 (t, J=7.6 Hz, 1H), 7.03 (t, J=6.8 Hz, 1H), 7.26 (d, J=8.0 Hz, 1H), 7.41-7.47 (m, 3H), 8.01 (d, J=8.0 Hz, 2H), 10.76 (s, 1H).

3-(trimethylsilyl)propioloyl chloride: To a stirred solution of 3-(trimethylsilyl)propiolic acid (0.30 g, 7.030 mmol, 1 eq) in DMF (0.002 mL, 0.028 mmol, 0.04 eq) was added oxalyl chloride (0.20 mL, 15.468 mmol, 2.2 eq) at 0° C. The mixture was allowed to warm room temperature and stirred 30 minutes. Then, the reaction mixture was concentrated under reduced pressure to obtain 3-(trimethylsilyl)propioloyl chloride. The crude was taken as such to next step.

14(1S,3S)-3-butyl-1-(4-(3-methyl-1,2,4-oxadiazol-5-yl)phenyl)-1,3,4,9-tetrahydro-2H-pyrido[3,4-b]indol-2-yl)-3-(trimethylsilyl)prop-2-yn-1-one: To a stirred solution of 5-(4-((1S,3S)-3-butyl-2,3,4,9-tetrahydro-1H-pyrido[3,4-b]indol-1-yl)phenyl)-3-methyl-1,2,4-oxadiazole (0.150 g, 7.308 mmol, 1.0 eq) in acetonitrile (10 mL) was added sodium bicarbonate (0.245 g, 52.731 mmol, 7.5 eq) at 0° C. After stirring for 5 minutes, a solution of 3-(trimethylsilyl)propioloyl chloride (0.33 g, 49.216 mmol, 7.0 eq) in acetonitrile was added. The resulting mixture was gradually allowed to warm to room temperature and stirred for 15 minutes. The progress of the reaction was monitored by TLC (50% ethyl acetate in hexane). After completion of reaction, the reaction mixture was filtered through celite pad, washed the celite pad with acetonitrile. The filtrate was concentrated under reduced pressure to obtain 1-((1S,3S)-3-butyl-1-(4-(3-methyl-1,2,4-oxadiazol-5-yl)phenyl)-1,3,4,9-tetrahydro-2H-pyrido[3,4-b]indol-2-yl)-3-(trimethylsilyl)prop-2-yn-1-one. The isolated crude product was taken as such to next step without further purification. LC-MS (m/z)=510.9 ([1\4+H]⁺. 1-((1S,3S)-3-butyl-1-(4-(3-methyl-1,2,4-oxadiazol-5-yl)phenyl)-1,3,4,9-tetrahydro-2H-pyrido[3,4-b]indol-2-yl)prop-2-yn-1-one: To a stirred solution of 1-((1S,3S)-3-butyl-1-(4-(3-methyl-1,2,4-oxadiazol-5-yl)phenyl)-1,3,4,9-tetrahydro-2H-pyrido[3,4-b]indol-2-yl)-3-(trimethylsilyl)prop-2-yn-1-one (0.198 g, 0.387 mmol, 1 eq) in THF (8.0 mL) was added tetra butyl ammonium fluoride (1M in THF) (0.426 mL, 0.426 mmol, 1.1 eq) at −78° C. The mixture was stirred at −78° C. for 10 minutes. The progress of the reaction was monitored by TLC (50% ethyl acetate in hexane). After completion of reaction, the reaction mixture was concentrated under reduced pressure, the obtained crude was diluted with water (5 mL) and extracted with ethyl acetate (2×5 mL). The organic layers were dried over anhydrous sodium sulfate, filtered and concentrated under reduced pressure. The crude product was purified by preparative TLC using 50% ethyl acetate in hexane as an eluent to obtain 1-((1S,3S)-3-butyl-1-(4-(3-methyl-1,2,4-oxadiazol-5-yl)phenyl)-1,3,4,9-tetrahydro-2H-pyrido[3,4-b]indol-2-yl)prop-2-yn-1-one. LC-MS (m/z)=439.1 [M+H]⁺. ¹H NMR (400 MHz, DMSO-d6) δ ppm 0.90 (s, 3H), 1.24 (s, 5H), 2.31-2.48 (m, 3H), 3.23 (s, 2H) 4.47 (s, 1H), 4.96 (s, 1H), 5.98 (s, 1H), 6.97 (d, J=7.6 Hz, 2H), 7.03 (s, 1H), 7.46 (d, J=8.0 Hz, 1H), 7.57 (d, J=8.0 Hz, 2H), 7.94 (s, 2H), 10.76 (s, 1H).

### Procedure B-3: Compound B-3

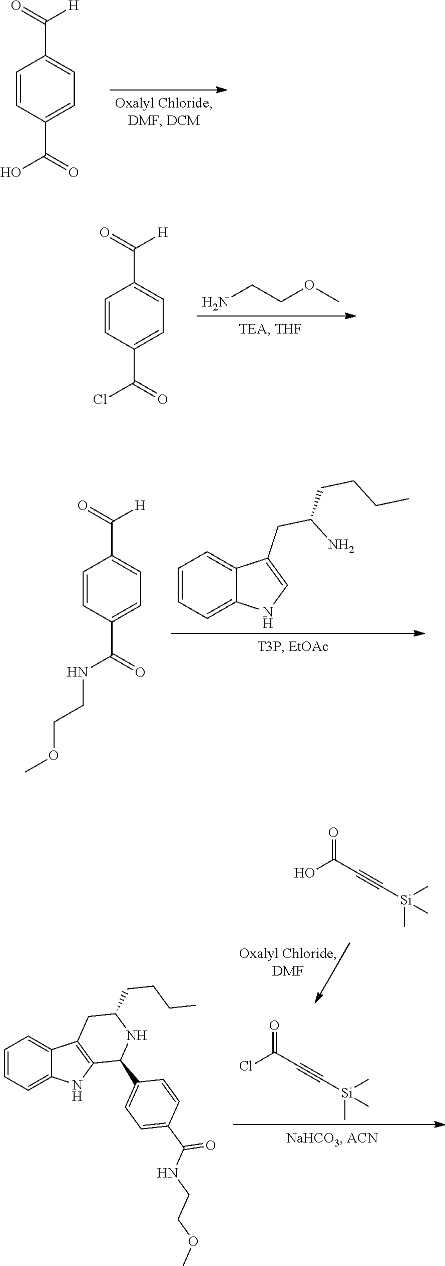

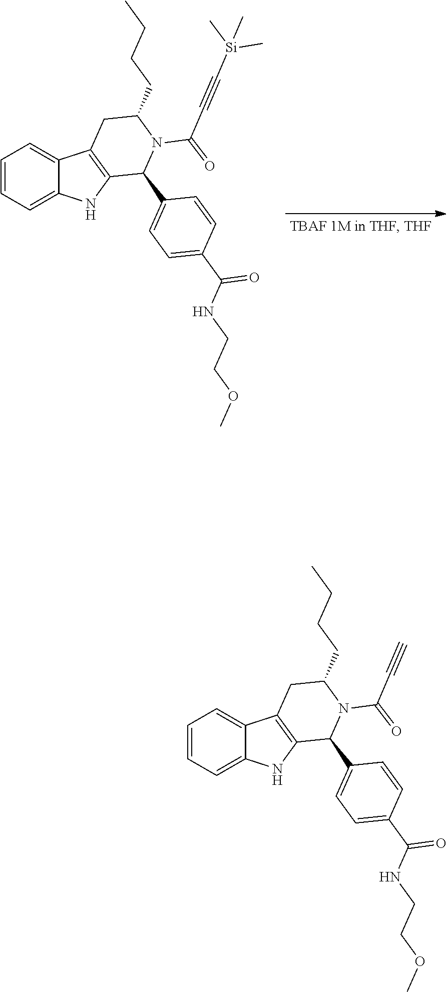 [SMILES: CCCC[C@H]1Cc2c([nH]c3ccccc23)[C@H](c2ccc(C(=O)NCCOC)cc2)N1C(=O)C#C[Si](C)(C)C ; C#CC(=O)N1[C@@H](CCCC)Cc2c([nH]c3ccccc23)[C@@H]1c1ccc(C(=O)NCCOC)cc1]

4-formylbenzoyl chloride: To a stirred solution of 4-formylbenzoic acid (1.0 g, 6.660 mmol, 1 eq) in DCM (10 mL) was added DMF (1 drop), and oxalyl chloride (1.14 mL, 13.32 mmol, 2.0 eq) at 0° C. The mixture was allowed to warm to room temperature and stirred for 2 hours. Then, the reaction mixture was concentrated under reduced pressure to 4-formylbenzoyl chloride. The crude was taken as such to next step.

4-formyl-N-(2-methoxyethyl)benzamide: To a stirred solution of 2-methoxyethan-1-amine (1.047 mL, 12.04 mmol, 1.8 eq) in THF (15 mL) was added TEA (1.68 mL, 12.04 mmol, 10.0 eq) and the reaction mixture was cooled to 0° C. than 4-formylbenzoyl chloride (1.128 g, 6.691 mmol, 1.0 eq) diluted in THF was added drop wise to the reaction mixture at 0° C. and the resulting mixture was gradually allowed to warm to room temperature and stirred for 1 h. The progress of the reaction was monitored by TLC (70% ethyl acetate in hexane). After completion of reaction, the reaction mixture was diluted with water (5 mL) and extracted with ethyl acetate (2×5 mL). The organic layers were dried over anhydrous sodium sulfate, filtered and concentrated under reduced pressure to obtain crude product, which was purified by flash column chromatography using 50% ethyl acetate in hexane as an eluent to obtain 4-formyl-N-(2-methoxyethyl)benzamide. LC-MS (m/z)=208.0 [M+H]⁺. Based on LCMS data confirmation, proceeded to the next step.

4-((1S,3S)-3-butyl-2,3,4,9-tetrahydro-1H-pyrido[3,4-b]indol-1-yl)-N-(2-methoxyethyl)benzamide: To a stirred solution of (S)-1-(1H-indol-3-yl)hexan-2-amine (0.7 g, 3.235 mmol, 1 eq) and 4-formyl-N-(2-methoxyethyl)benzamide (0.669 g, 3.235 mmol, 1 eq) in EtOAc (20 mL) was added T3P (5.14 mL, 8.087 mmol, 2.5 eq) at 0° C. Then the reaction mixture was refluxed at 110° C. in microwave for 1 hour. The progress of the reaction was monitored by TLC (40% ethyl acetate in hexane). After completion of reaction, the reaction mixture was diluted with water (5 mL) and extracted with ethyl acetate (2×5 mL). The combined organic layers were dried over anhydrous sodium sulfate, filtered and concentrated under reduced pressure. The crude product was purified by flash column chromatography using 50% ethyl acetate in hexane as an eluent to obtain 4-((1S,3S)-3-butyl-2,3,4,9-tetrahydro-1H-pyrido[3,4-b]indol-1-yl)-N-(2-methoxyethyl)benzamide. LC-MS (m/z)=406.2 [M+H]⁺.

3-(trimethylsilyl)propioloyl chloride: To a stirred solution of 3-(trimethylsilyl)propiolic acid (0.087 g, 0.616 mmol, 1 eq) in DMF (0.002 mL, 0.024 mmol, 0.04 eq) was added oxalyl chloride (0.058 mL, 0.677 mmol, 1.1 eq) at 0° C. The mixture was allowed to warm room temperature and stirred 30 minutes. Then, the reaction mixture was concentrated under reduced pressure to obtain 3-(trimethylsilyl)propioloyl chloride. The crude was taken as such to the next step.

4-((1S,3S)-3-butyl-2-(3-(trimethylsilyl)propioloyl)-2,3,4,9-tetrahydro-1H-pyrido[3,4-b]indol-1-yl)-N-(2-methoxyethyl)benzamide: To a stirred solution of 4-((1S,3S)-3-butyl-2,3,4,9-tetrahydro-1H-pyrido[3,4-b]indol-1-yl)-N-(2-methoxyethyl)benzamide (0.1 g, 0.246 mmol, 1.0 eq) in acetonitrile (8 mL) was added sodium bicarbonate (0.155 g, 1.84 mmol, 7.5 eq) at 0° C. After stirring for 5 minutes, a solution of 3-(trimethylsilyl)propioloyl chloride (0.087 g, 0.616 mmol, 2.5 eq) in acetonitrile was added. The resulting mixture was gradually allowed to warm to room temperature and stirred for 15 minutes. The progress of the reaction was monitored by TLC (50% ethyl acetate in hexane). After completion of reaction, the reaction mixture was filtered through celite pad, washed the celite pad with acetonitrile. The filtrate was concentrated under reduced pressure to obtain 4-((1S,3S)-3-butyl-2-(3-(trimethylsilyl)propioloyl)-2,3,4,9-tetrahydro-1H-pyrido[3,4-b]indol-1-yl)-N-(2-methoxyethyl)benzamide. The isolated crude product was taken as such to next step without further purification. LC-MS (m/z)=530.0 ([1\4+H]⁺. 4-((1S,3S)-3-butyl-2-propioloyl-2,3,4,9-tetrahydro-1H-pyrido[3,4-b]indol-1-yl)-N-(2-methoxyethyl)benzamide: To a stirred solution of 4-((1S,3S)-3-butyl-2-(3-(trimethylsilyl)propioloyl)-2,3,4,9-tetrahydro-1H-pyrido[3,4-b]indol-1-yl)-N-(2-methoxyethyl)benzamide (0.169 g, 0.320 mmol, 1 eq) in THF (5.0 mL) was added tetra butyl ammonium fluoride (1M in THF) (0.35 mL, 0.350 mmol, 2 eq) at −78° C. The mixture was allowed to stir at −78° C. for 10 minutes. The progress of the reaction was monitored by TLC (5% methanol in DCM). After completion of reaction, the reaction mixture was quenched with NaHCO₃ (5 mL) and extracted with ethyl acetate (2×5 mL). The organic layers were dried over anhydrous sodium sulfate, filtered and concentrated under reduced pressure. The crude product was purified by preparative TLC using 2% methanol in DCM as an eluent to obtain 4-((1S,3S)-3-butyl-2-propioloyl-2,3,4,9-tetrahydro-1H-pyrido[3,4-b]indol-1-yl)-N-(2-methoxyethyl)benzamide. LC-MS (m/z)=458.2 [M+H]⁺. ¹H NMR (400 MHz, DMSO-d₆) δ ppm 0.85 (s, 3H), 1.24-1.29 (m, 4H), 1.49 (s, 2H), 3.24 (s, 2H), 3.38-3.41 (m, 5H), 4.44 (s, 1H), 4.94 (s, 1H), 5.93 (s, 1H), 6.96-7.02 (m, 2H), 7.24 (s, 1H), 7.38-7.46 (m, 3H), 7.96 (s, 2H), 8.15 (s, 1H), 10.71 (s, 1H).

### Procedure B-4: Compound B-4

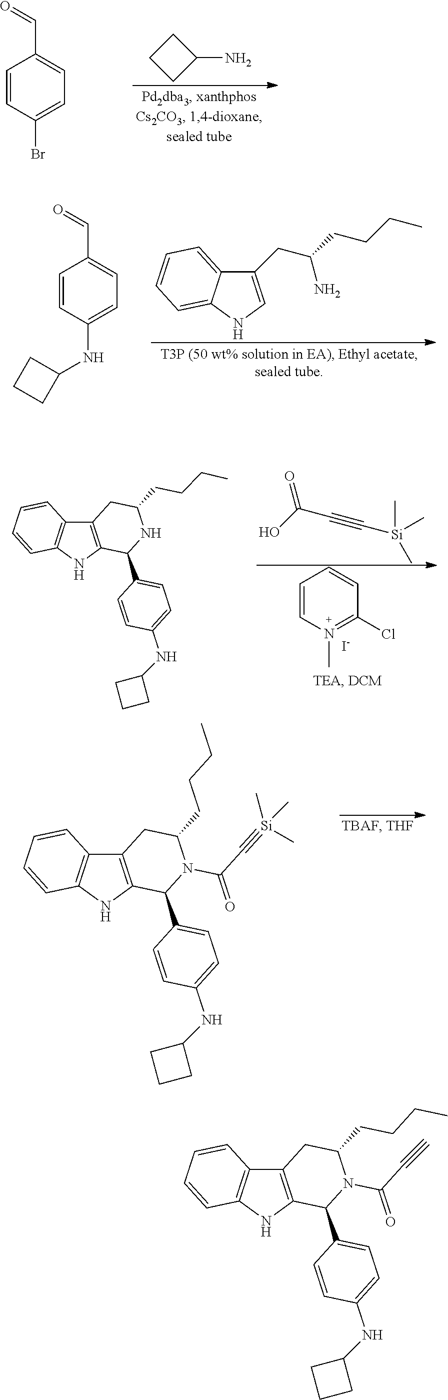

4-(cyclobutylamino)benzaldehyde: Ina seal tube, 4-bromobenzaldehyde (2.0 g, 10.809 mmol, 1.0 eq), cyclobutanamine (3.7 mL, 43.238 mmol, 1.3 eq) and 1,4-dioxane were taken, the resulting mixture was degasified by argon for 5 minutes. Then xanthphos (1.25 g, 2.161 mmol, 0.2 eq), Pd₂dba₃ (0.98 g, 1.080 mmol, 0.1 eq) were added and followed by the addition of cesium carbonate (14.08 g, 43.238 mmol, 4.0 eq) under argon. The seal tube was closed and the mixture was heated to 80° C. for 16 h. The progress of the reaction was monitored by TLC (20 EA/Hexane). After completion of reaction, the reaction was cooled to room temperature, filtered through celite pad, washed with celite pad with ethyl acetate. The filtrate was concentrated to obtain crude product, which was purified by flash column chromatography using 12% ethyl acetate in hexane as an eluent to obtain 4-(cyclobutylamino)benzaldehyde. LC-MS (m/z)=176.0 [M−H]⁺. NMR (400 MHz, DMSO-d6) δ 1.66-1.90 (m, 4H), 2.30-2.34 (m, 2H), 3.87-3.94 (m, 1H), 6.56 (d, J=8.8 Hz, 2H), 7.08 (d, J=5.6 Hz, 1H), 7.57 (d, J=8.8 Hz, 2H), 9.56 (s, 1H).

4-((1S,3S)-3-butyl-2,3,4,9-tetrahydro-1H-pyrido[3,4-b]indol-1-yl)-N-cyclobutylaniline: In a seal tube, to stirred mixture of (S)-1-(1H-indol-3-yl)hexan-2-amine (1.0 g, 4.622 mmol, 1.0 eq), and 4-(cyclobutylamino)benzaldehyde (0.81 g, 4.622 mmol, 1.0 eq) in ethyl acetate (5 mL) was added T3P (50 wt. % in ethyl acetate) (7.30 g, 11.556 mmol, 2.5 eq) at room temperature and the seal tube was closed and the mixture was heated to 130° C. for 16 h. The progress of the reaction was monitored by TLC (10% methanol in dichloromethane), the reaction mixture was cooled to room temperature. The mixture was dissolved with dichloromethane (100 mL) and basified with saturated sodium bicarbonate solution. The organic layer was separated and the aqueous layer was extracted with dichloromethane (2×25 mL). The combined organics were washed with brine (50 mL), dried over anhydrous sodium sulfate, filtered and concentrated to obtain crude, which was purified by flash column chromatography using 7% methanol in dichloromethane as an eluent. The isolated product was treated with metal scavenger quadrasil TA to remove residual catalyst (the compound was dissolved with THF (30 mL) and 7 g of quadrasil TA was added, the mixture was stirred for 1 h at room temperature, The mixture was filtered and the filtrate was concentrated under reduced pressure to obtain 4-((1S,3S)-3-butyl-2,3,4,9-tetrahydro-1H-pyrido[3,4-b]indol-1-yl)-N-cyclobutylaniline. LC-MS (m/z)=374.0 [M+H]⁺. 1-((1S,3S)-3-butyl-1-(4-(cyclobutylamino)phenyl)-1,3,4,9-tetrahydro-2H-pyrido[3,4-b]indol-2-yl)-3-(trimethylsilyl)prop-2-yn-1-one: To a stirred mixture of 4-((1S,3S)-3-butyl-2,3,4,9-tetrahydro-1H-pyrido[3,4-b]indol-1-yl)-N-cyclobutylaniline (0.25 g, 0.669 mmol, 1.0 eq), in dichloromethane (5 mL) was added triethylamine (0.23 mL, 1.673 mmol, 2.5 eq), 3-(trimethylsilyl)propiolic acid (0.095 g, 0.669 mmol, 1.0 eq) and followed by the addition of 2-Chloro-1-methylpyridinium iodide (0.2 g, 0.803 mmol, 1.2 eq) at room temperature. The mixture was stirred for 1 h. The progress of the reaction was monitored by TLC (40% ethyl acetate in dichloromethane). After completion of reaction, the mixture was diluted with dichloromethane (50 mL), washed with water (20 mL), brine (10 mL), dried over anhydrous sodium sulfate, filtered and concentrated under reduced pressure to obtain crude 1-((1S,3S)-3-butyl-1-(4-(cyclobutylamino)phenyl)-1,3,4,9-tetrahydro-2H-pyrido[3,4-b]indol-2-yl)-3-(trimethylsilyl)prop-2-yn-1-one. which was taken as such without purification. LC-MS (m/z)=498.3 [M+H]⁺.

¹H-NMR (400 MHz, DMSO-d6) 0.22 (s, 9H), 0.6-0.9 (m, 3H), 1.00-1.4 (m, 4H), 1.45-1.51 (m, 2H), 1.52-1.71 (m, 4H), 2.02 (bs, 1H), 2.30-2.4 (m, 3H), 3.77 (bs, 1H), 4.9 (bs, 1H), 5.49 (bs, 1H), 5.58 (bs, 1H), 6.42 (bs, 2H), 6.96-7.02 (m, 4H), 7.25-7.3 (m, 1H), 7.41-7.43 (m, 1H), 10.9 (bs, 1H).

1-((1S,3S)-3-butyl-1-(4-(cyclobutylamino)phenyl)-1,3,4,9-tetrahydro-2H-pyrido[3,4-b]indol-2-yl)prop-2-yn-1-one: To a stirred solution of 1-((1S,3S)-3-butyl-1-(4-(cyclobutylamino)phenyl)-1,3,4,9-tetrahydro-2H-pyrido[3,4-b]indol-2-yl)-3-(trimethylsilyl)prop-2-yn-1-one (0.4 g crude, 0.803 mmol, 1.0 eq) in THF (5.0 mL) was added tetra butyl ammonium fluoride (TBAF) (1M in THF) (0.8 mL, 1.607 mmol, 2.0 eq) at 0° C. The mixture was allowed to warm room temperature and stirred for 30 minutes. The progress of the reaction was monitored by TLC (5% methanol in DCM). After completion of reaction, the reaction mixture was diluted with ethyl acetate, washed with water (2×20 mL), brine (20 mL) and dried over anhydrous sodium sulfate, filtered and concentrated under reduced pressure to obtain crude, which was purified by flash column chromatography using 40% ethyl acetate in hexane as eluent, the isolated product was re-purified by preparative TLC using 40% ethyl acetate in hexane as an eluent to obtain 1-4-((1S,3S)-3-butyl-1-(4-(cyclobutylamino)phenyl)-1,3,4,9-tetrahydro-2H-pyrido[3,4-b]indol-2-yl)prop-2-yn-1-one. LC-MS (m/z)=426.2 [M+H]⁺. ¹H NMR (400 MHz, DMSO-d6, at 70° C.) δ ppm 0.76 (bs, 3H), 1.08 (bs, 4H), 1.52 (bs, 1H), 1.67-1.74 (m, 4H), 2.27 (bs, 1H), 2.77 (m, 1H), 2.98 (m, 2H), 3.77 (bs, 1H), 4.36 (s, 1 H), 4.76 (bs, 1H), 5.50 (bs, 1H), 5.70-5.80 (m, 1H), 6.37-6.46 (m, 3H), 6.95-7.02 (m, 4H), 7.25 (bs, 1H), 7.42 (d, J=8.0 Hz, 1H), 10.80-10.90 (m, 1H). HPLC purity: 96.64% at 220 nm.

### Procedure B-5: Compound B-5

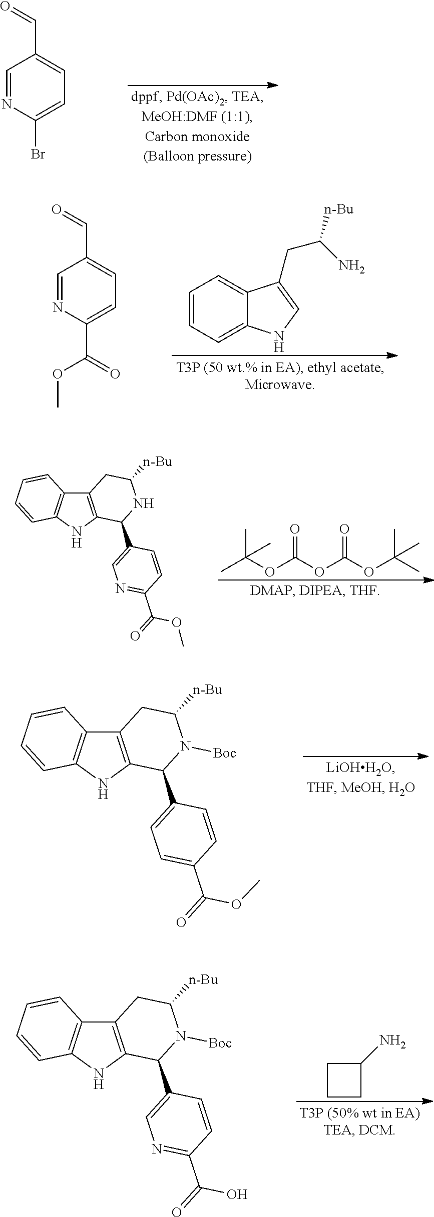

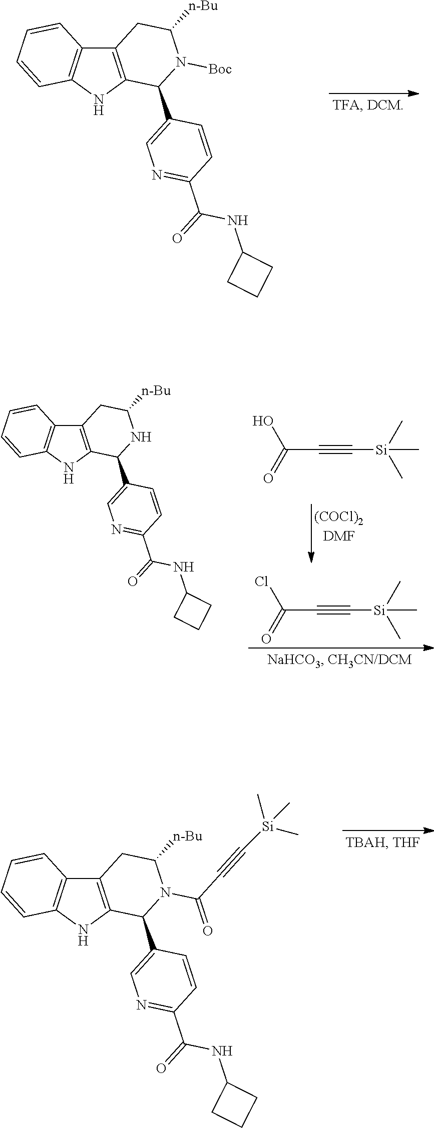

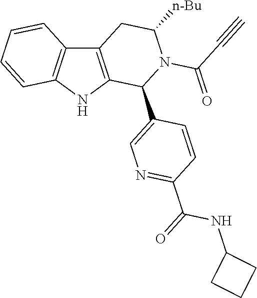

methyl 5-formylpicolinate: To a stirred solution of 6-bromonicotinaldehyde (4.4 g, 23.654 mmol, 1.0 eq) in a mixture of methanol (40 mL) and DMF (25 mL) was added triethylamine (9.9 mL, 70.963 mmol, 3.0 eq) at room temperature. The mixture was degasified by purging with argon for 5 min, then Pd(OAc)₂ (0.26 g, 1.182 mmol, 0.05 eq) and dppf (1.3 g, 2.365 mmol, 0.1 eq) was added under argon atmosphere. The mixture was degasified by purging with carbon monoxide (balloon pressure) 3 times. The mixture was heated to 55° C. for 56 h under carbon monoxide atmosphere. The progress of the reaction was monitored by TLC (50% ethyl acetate in hexane). After completion of reaction, the reaction mixture was cooled to room temperature, filtered through celite pad, washed the celite pad with ethyl acetate. The filtrate was concentrated under reduced pressure, the obtained residue was dissolved with ethyl acetate (300 mL), washed with water (2×100 mL), brine (50 mL), dried over anhydrous sodium sulfate, filtered and concentrated under reduced pressure to obtain crude, which was purified by flash column chromatography using 5% methanol in dichloromethane as eluent obtain methyl 5-formylpicolinate. LC-MS (m/z)=166.1 [M+H]⁺. ¹H NMR (400 MHz, DMSO-d6) δ ppm 4.06 (s, 3H), 8.29-8.34 (m, 2H), 9.19 (s, 1H), 10.21 (s, 1H).

methyl 5-((1S,3S)-3-butyl-2,3,4,9-tetrahydro-1H-pyrido[3,4-b]indol-1-yl)picolinate: In a CEM microwave vial, to a stirred mixture of (S)-1-(1H-indol-3-yl)hexan-2-amine (0.5 g, 2.311 mmol, 1.0 eq), and methyl 5-formylpicolinate (0.4 g, 2.426 mmol, 1.05 eq) in ethyl acetate (5 mL) was added T3P (?50 wt. % in ethyl acetate) (3.67 mL, 5.778 mmol, 2.5 eq), at room temperature and the vial was closed. The mixture was subjected to microwave irradiation at 110° C. for 1 h. The progress of the reaction was monitored by TLC (10% methanol in dichloromethane). After completion of reaction, the mixture was cooled to room temperature. The mixture was dissolved with ethyl acetate (100 mL) and basified with saturated sodium bicarbonate solution. The organic layer was separated and the aqueous layer was extracted with ethyl acetate (2×25 mL). The combined organics were washed with brine (50 mL), dried over anhydrous sodium sulfate, filtered and concentrated to obtain crude, which was purified by flash column chromatography using 4% methanol in dichloromethane as an eluent to obtain methyl 5-((1S,3S)-3-butyl-2,3,4,9-tetrahydro-1H-pyrido[3,4-b]indol-1-yl)picolinate. LC-MS (m/z)=364.0 [M+H]⁺.

tert-butyl (1S,3S)-3-butyl-1-(6-(methoxycarbonyl)pyridin-3-yl)-1,3,4,9-tetrahydro-2H-pyrido[3,4-b]indole-2-carboxylate: To a stirred solution of methyl 5-((1S,3S)-3-butyl-2,3,4,9-tetrahydro-1H-pyrido[3,4-b]indol-1-yl)picolinate (0.4 g, 1.1 mmol, 1.0 eq) and N,N-Diisopropylethylamine (0.28 mL, 1.65 mmol, 1.5 eq), in THF (10 mL) was added di-tert-butyl dicarbonate (0.35 mL, 1.54 mmol, 1.4 eq) at 0° C. The mixture was allowed to warm room temperature and stirred for 72 h. The progress of the reaction was monitored by TLC (5% methanol in DCM). After completion of reaction, the reaction mixture was diluted with ethyl acetate (100 mL), washed with water (2×50 mL), dried over anhydrous sodium sulfate, filtered and concentrated under reduced pressure to obtain crude, which was purified by flash column chromatography using 5% methanol in dichloromethane as an eluent to obtain tert-butyl (1S,3S)-3-butyl-1-(6-(methoxycarbonyl)pyridin-3-yl)-1,3,4,9-tetrahydro-2H-pyrido[3,4-b]indole-2-carboxylate. LC-MS (m/z)=464.0 [M+H]⁺.

5-((1S,3S)-2-(tert-butoxycarbonyl)-3-butyl-2,3,4,9-tetrahydro-1H-pyrido[3,4-b]indol-1-yl)picolinic acid: To a solution of tert-butyl (1S,3S)-3-butyl-1-(6-(methoxycarbonyl)pyridin-3-yl)-1,3,4,9-tetrahydro-2H-pyrido[3,4-b]indole-2-carboxylate (0.5 g, 1.078 mmol, 1.0 eq) in a mixture of THF:MeOH:H₂O (9 mL:1 mL:1 mL), was added LiOH.H₂O (0.22 g, 5.392 mmol, 5.0 eq) at 0° C., the mixture was allowed to warm to room temperature and stirred for 2 h. The progress of the reaction was monitored by TLC (5% methanol in DCM). After completion of reaction, the reaction mixture was concentrated under reduced pressure, the obtained crude was dissolved with water (10 mL), acidified with 10% citric acid solution (pH=3). The aqueous layer was extracted with ethyl acetate (2×100 mL). The combined organics were washed with water (20 mL), brine (20 mL), dried over anhydrous sodium sulfate, filtered and concentrated under reduced pressure to obtain 5-((1S,3S)-2-(tert-butoxycarbonyl)-3-butyl-2,3,4,9-tetrahydro-1H-pyrido[3,4-b]indol-1-yl)picolinic acid. LC-MS (m/z): 450.0 [M+H]⁺.

tert-butyl (1S,3S)-3-butyl-1-(6-(cyclobutylcarbamoyl)pyridin-3-yl)-1,3,4,9-tetrahydro-2H-pyrido[3,4-b]indole-2-carboxylate: To a solution of 5-((1S,3S)-2-(tert-butoxycarbonyl)-3-butyl-2,3,4,9-tetrahydro-1H-pyrido[3,4-b]indol-1-yl)picolinic acid (0.5 g crude, 1.112 mmol, 1.0 eq) in dichloromethane (10 mL) was added triethylamine (0.62 mL, 4.448 mmol, 4.0 eq) and cyclobutanamine (0.1 mL, 1.223 mmol, 1.1 eq), the mixture was cooled to 0° C. Then, T3P (>50 wt. % in ethyl acetate) (1.0 mL, 1.668 mmol, 1.5 eq), was added at 0° C., the mixture was allowed to warm to room temperature and stirred for 6 h.

The progress of the reaction was monitored by TLC (5% methanol in DCM). After completion of reaction, the reaction mixture was concentrated under reduced pressure, the obtained residue was quenched with saturated sodium bicarbonate solution, extracted with dichloromethane (2×100 mL). The combined organics were washed with water (2×25 mL), brine (25 mL), dried over anhydrous sodium sulfate, filtered and concentrated under reduced pressure to obtain crude, which was purified by flash column chromatography using 5% methanol in dichloromethane as an eluent to obtain tert-butyl (1S,3S)-3-butyl-1-(6-(cyclobutylcarbamoyl)pyridin-3-yl)-1,3,4,9-tetrahydro-2H-pyrido[3,4-b]indole-2-carboxylate. LC-MS (m/z): 503 [M+H]⁺.

5-((1S,3S)-3-butyl-2,3,4,9-tetrahydro-1H-pyrido[3,4-b]indol-1-yl)-N-cyclobutylpicolinamide: To a solution of compound tert-butyl (1S,3S)-3-butyl-1-(6-(cyclobutylcarbamoyl)pyridin-3-yl)-1,3,4,9-tetrahydro-2H-pyrido[3,4-b]indole-2-carboxylate (0.5 g, 0.994 mmol, 1.0 eq) in DCM (2 mL) was added trifluoroacetic acid (2 mL) at 0° C., the mixture was allowed to warm to room temperature and stirred for 16 h. The progress of the reaction was monitored by TLC (5% methanol in DCM). After completion of reaction, the reaction mixture was concentrated under reduced pressure. The obtained residue was cooled to 0° C. and basified with 10% sodium hydroxide solution to ˜pH 12. The product was extracted with ethyl acetate (3×100 mL). The combined organics were washed with water (50 mL), brine (25 mL), dried over anhydrous sodium sulfate, filtered and concentrated under reduced pressure to obtain crude, which was purified by flash column chromatography using 5% methanol in dichloromethane as an eluent to obtain 5-((1S,3S)-3-butyl-2,3,4,9-tetrahydro-1H-pyrido[3,4-b]indol-1-yl)-N-cyclobutylpicolinamide. LC-MS (m/z): 403.3 [M+H]⁺.

3-(trimethylsilyl)propioloyl chloride: To a mixture of 3-(trimethylsilyl)propiolic acid (0.2 g, 1.406 mmol, 1.0 eq) in DMF (0.004 mL, 0.056 mmol, 0.04 eq) was added oxalyl chloride (0.14 mL, 1.546 mmol, 1.1 eq) at room temperature and stirred for 30 min. Then, the reaction mixture was concentrated under reduced pressure to obtain 3-(trimethylsilyl)propioloyl chloride. The crude was taken as such to next step without work up.

5-((1S,3S)-3-butyl-2-(3-(trimethylsilyl)propioloyl)-2,3,4,9-tetrahydro-1H-pyrido[3,4-b]indol-1-yl)-N-cyclobutylpicolinamide: To a stirred solution of 5-((1S,3S)-3-butyl-2,3,4,9-tetrahydro-1H-pyrido[3,4-b]indol-1-yl)-N-cyclobutylpicolinamide (0.18 g, 0.447 mmol, 1.0 eq) in acetonitrile (10 mL) was added sodium bicarbonate (0.37 g, 4.47 mmol, 10.0 eq). The mixture was cooled to 0° C., then, a solution of 3-(trimethylsilyl)propioloyl chloride (0.22 g, 1.341 mmol, 3.0 eq) in acetonitrile was added and stirred for 2 h. The progress of the reaction was monitored by TLC (5% methanol in DCM). After completion of reaction, the reaction mixture was filtered through celite pad, washed the celite pad with acetonitrile. The filtrate was concentrated under reduced pressure to obtain 5-((1S,3S)-3-butyl-2-(3-(trimethylsilyl)propioloyl)-2,3,4,9-tetrahydro-1H-pyrido[3,4-b]indol-1-yl)-N-cyclobutylpicolinamide. The isolated crude product was taken as such to next step without further purification. LC-MS (m/z)=526.9 ([1\4+H]⁺.

5-((1S,3S)-3-butyl-2-propioloyl-2,3,4,9-tetrahydro-1H-pyrido[3,4-b]indol-1-yl)-N-cyclobutylpicolinamide: To a stirred solution of 5-((1S,3S)-3-butyl-2-(3-(trimethylsilyl)propioloyl)-2,3,4,9-tetrahydro-1H-pyrido[3,4-b]indol-1-yl)-N-cyclobutylpicolinamide (0.3 g crude, 0.569 mmol, 1.0 eq) in THF (5.0 mL) was added tetra butyl ammonium fluoride (1M in THF) (0.62 mL, 0.626 mmol, 1.1 eq) at−78° C. and stirred for 15 min. The progress of the reaction was monitored by TLC (50% ethyl acetate in hexane). After completion of reaction, the reaction mixture was quenched with saturated sodium bicarbonate solution and the mixture was allowed to warm to room temperature, extracted with ethyl acetate (3×100 mL), washed with water (50 mL), brine (50 mL), dried over anhydrous sodium sulfate, filtered and concentrated under reduced pressure to obtain crude. The crude product was purified by flash column chromatography using 2% methanol in dichloromethane as eluent. The isolated product was purified by preparative TLC using 3% methanol in dichloromethane as an eluent to obtain 5-((1S,3S)-3-butyl-2-propioloyl-2,3,4,9-tetrahydro-1H-pyrido[3,4-b]indol-1-yl)-N-cyclobutylpicolinamide. LC-MS (m/z)=455.2 [M+H]⁺. ¹H NMR (400 MHz, DMSO-d6 at 70° C.) δ ppm 0.80 (bs, 3H), 1.25 (m, 4H), 1.51-1.61 (m, 2H), 1.63-1.70 (m, 2H), 2.07-2.11 (m, 2H), 2.19-2.21 (m, 2H), 3.10 (m, 1H), 3.26 (bs, 1H), 4.35-4.41 (m, 1H), 4.48 (s, 1H), 4.97 (bs, 1H), 5.99 (s, 1H), 6.96-7.03 (m, 2H), 7.25 (bs, 1H), 7.48 (d, J=7.6 Hz, 1H), 7.79-7.83 (m, 2H), 8.53 (bs, 1H), 8.61 (s, 1H), 10.74 (s, 1H). HPLC purity 98.82% at 220 nm.

### Procedure B-6: Compound B-6

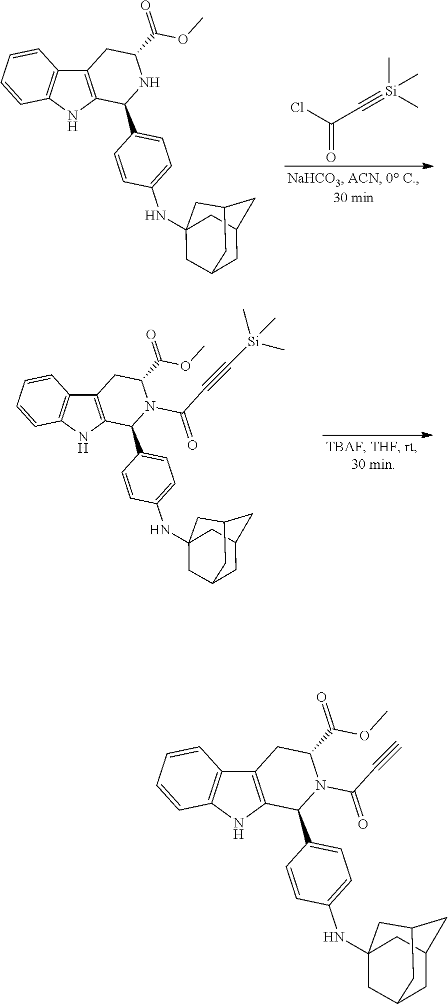

methyl (1S,3R)-1-(4-(((3R,5R,7R)-adamantan-1-yl)amino)phenyl)-2-(3-(trimethylsilyl)propioloyl)-2,3,4,9-tetrahydro-1H-pyrido[3,4-b]indole-3-carboxylate: To 3-(trimethylsilyl)propiolic acid (0.084 g, 0.59 mmol, 1 eq), DMF (0.001 g, 0.023 mmol, 0.04 eq) and oxalyl chloride (0.055 mL, 0.64 mmol, 1.1 eq) was added and stirred for 30 mins. After this time reaction mixture was concentrated under reduced pressure to get the crude 3-(trimethylsilyl)propioloyl chloride and this crude was diluted with ACN (1 mL) and added to a reaction mixture containing a stirred solution of methyl (1S,3R)-1-(4-(((3R,5R,7R)-adamantan-1-yl)amino)phenyl)-2,3,4,9-tetrahydro-1H-pyrido[3,4-b]indole-3-carboxylate (0.18 g, 0.395 mmol, 1 eq) and NaHCO₃ (0.248 g, 2.96 mmol, 7.5 eq) in ACN (5 mL) at 0° C. and stirred for 15 mins. LCMS and TLC (40% EtOAc in hexane) showed the reaction was completed. The reaction was filtered and concentrated under reduced pressure to give the crude product methyl (1S,3R)-1-(4-(((3R,5R,7R)-adamantan-1-yl)amino)phenyl)-2-(3-(trimethylsilyl)propioloyl)-2,3,4,9-tetrahydro-1H-pyrido[3,4-b]indole-3-carboxylate which was taken to next step without further purification. LC-MS (m/z): 578.0 [M+H]⁺.

Methyl(1S,3R)-1-(4-(((3R,5R,7R)-adamantan-1-yl)amino)phenyl)-2-propioloyl-2,3,4,9-tetrahydro-1H-pyrido[3,4-b]indole-3-carboxylate: To methyl (1S,3R)-1-(4-(((3R,5R,7R)-adamantan-1-yl)amino)phenyl)-2-(3-(trimethylsilyl)propioloyl)-2,3,4,9-tetrahydro-1H-pyrido[3,4-b]indole-3-carboxylate (0.140 g, 0.241 mmol, 1 eq) in THF (10.0 mL) TBAF (1M solution in THF) (0.48 mL, 0.48 mmol, 2 eq) was added and stirred for 30 mins. After this time reaction mixture was concentrated under reduced pressure, diluted with Ethylacetate (100 mL) and was washed with water (2×10 mL). The organic layers were dried over Na₂SO₄ and concentrated to give to get the crude which was further purified by preparative TLC chromatography using 30% EtOAc in Hexane as an eluent to methyl (1S,3R)-1-(4-(((3R,5R,7R)-adamantan-1-yl)amino)phenyl)-2-propioloyl-2,3,4,9-tetrahydro-1H-pyrido[3,4-b]indole-3-carboxylate. LC-MS (m/z): 508.3 [M+H]⁺. ¹H NMR (400 MHz, DMSO-d₆): δ 1.59-1.61 (m, 6H), 1.79-1.84 (m, 6H), 2.02 (s, 3H), 3.03-3.08 (m, 2H), 3.54 (s, 3H), 4.37-4.41 (m, 1H), 4.75 (S, 1H), 6.50 (s, 1H), 6.57-6.59 (m, 1H), 6.71-6.73 (m, 1H), 6.83-7.05 (m, 4H), 7.22-7.29 (m, 1H), 7.49 (s, 1H), 11.12 (bs, 1).

### Procedure B-7: Compound B-7

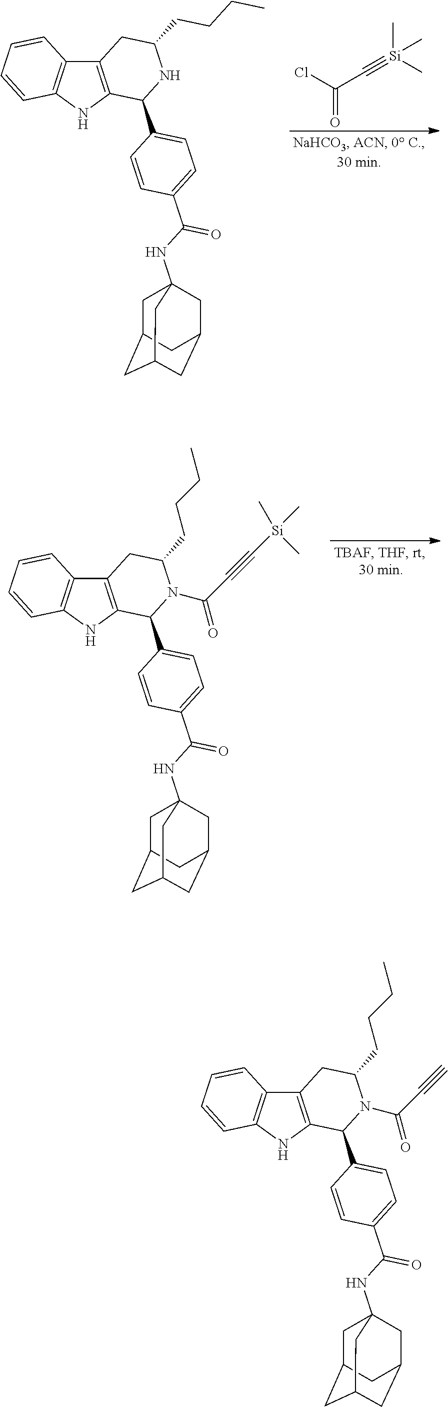

N-((3R,5R,7R)-adamantan-1-yl)-4-((1S,3S)-3-butyl-2-(3-(trimethylsilyl)propioloyl)-2,3,4,9-tetrahydro-1H-pyrido[3,4-b]indol-1-yl)benzamide: To 3-(trimethylsilyl)propiolic acid (0.044 g, 0.31 mmol, 1 eq), DMF (0.0008 g, 0.01 mmol, 0.04 eq) and oxalyl chloride (0.028 mL, 0.64 mmol, 1.1 eq) was added and stirred for 30 mins. After this time reaction mixture was concentrated under reduced pressure to get the crude 3-(trimethylsilyl)propioloyl chloride and this crude was diluted with ACN (1 mL) and added to a reaction mixture containing a stirred solution of N-((3R,5R,7R)-adamantan-1-yl)-4-((1S,3S)-3-butyl-2,3,4,9-tetrahydro-1H-pyrido[3,4-b]indol-1-yl)benzamide (0.10 g, 0.207 mmol, 1 eq) and NaHCO₃ (0.130 g, 1.55 mmol, 7.5 eq) in ACN (5 mL) at 0° C. and stirred for 15 mins. LCMS and TLC (50% EtOAc in hexane) showed the reaction was completed. The reaction was filtered and concentrated under reduced pressure to give the crude product N-((3R,5R,7R)-adamantan-1-yl)-4-((1S,3S)-3-butyl-2-(3-(trimethylsilyl)propioloyl)-2,3,4,9-tetrahydro-1H-pyrido[3,4-b]indol-1-yl)benzamide which was taken to next step without further purification. LC-MS (m/z): 605.0 [M+H]⁺.

N-((3R,5R,7R)-adamantan-1-yl)-4-((1S,3S)-3-butyl-2-propioloyl-2,3,4,9-tetrahydro-1H-pyrido[3,4-b]indol-1-yl)benzamide: To N-((3R,5R,7R)-adamantan-1-yl)-4-((1S,3S)-3-butyl-2-(3-(trimethylsilyl)propioloyl)-2,3,4,9-tetrahydro-1H-pyrido[3,4-b]indol-1-yl)benzamide (0.130 g, 0.214 mmol, 1 eq) in THF (10.0 mL) TBAF (1M solution in THF) (0.42 mL, 0.42 mmol, 2 eq) was added and stirred for 30 mins. After this time reaction mixture was concentrated under reduced pressure, diluted with Ethylacetate (100 mL) and was washed with water (2×10 mL). The organic layers were dried over Na₂SO₄ and concentrated to give to get the crude which was further purified by preparative TLC chromatography using 30% EtOAc in Hexane as an eluent to N-((3R,5R,7R)-adamantan-1-yl)-4-((1S,3S)-3-butyl-2-propioloyl-2,3,4,9-tetrahydro-1H-pyrido[3,4-b]indol-1-yl)benzamide. LC-MS (m/z): 534.5 [M+H]⁺; Chiral HPLC: 95.4% ee. ¹H NMR (400 MHz, DMSO-d₆): δ 0.78-0.84 (m, 2H), 1.22-1.28 (m, 9H), 2.01 (s, 4H), 2.04 (s, 7H), 2.65-3.16 (m, 2H), 4.57 (bs, 1H), 5.89 (S, 1H), 6.51 (s, 1H), 6.94-7.00 (m, 2H), 7.21-7.34 (m, 2H), 7.35-7.46 (m, 3H), 7.56-7.71 (m, 2H), 10.84 (s, 1H).

### Procedure B-8: Compounds B-8 and B-9

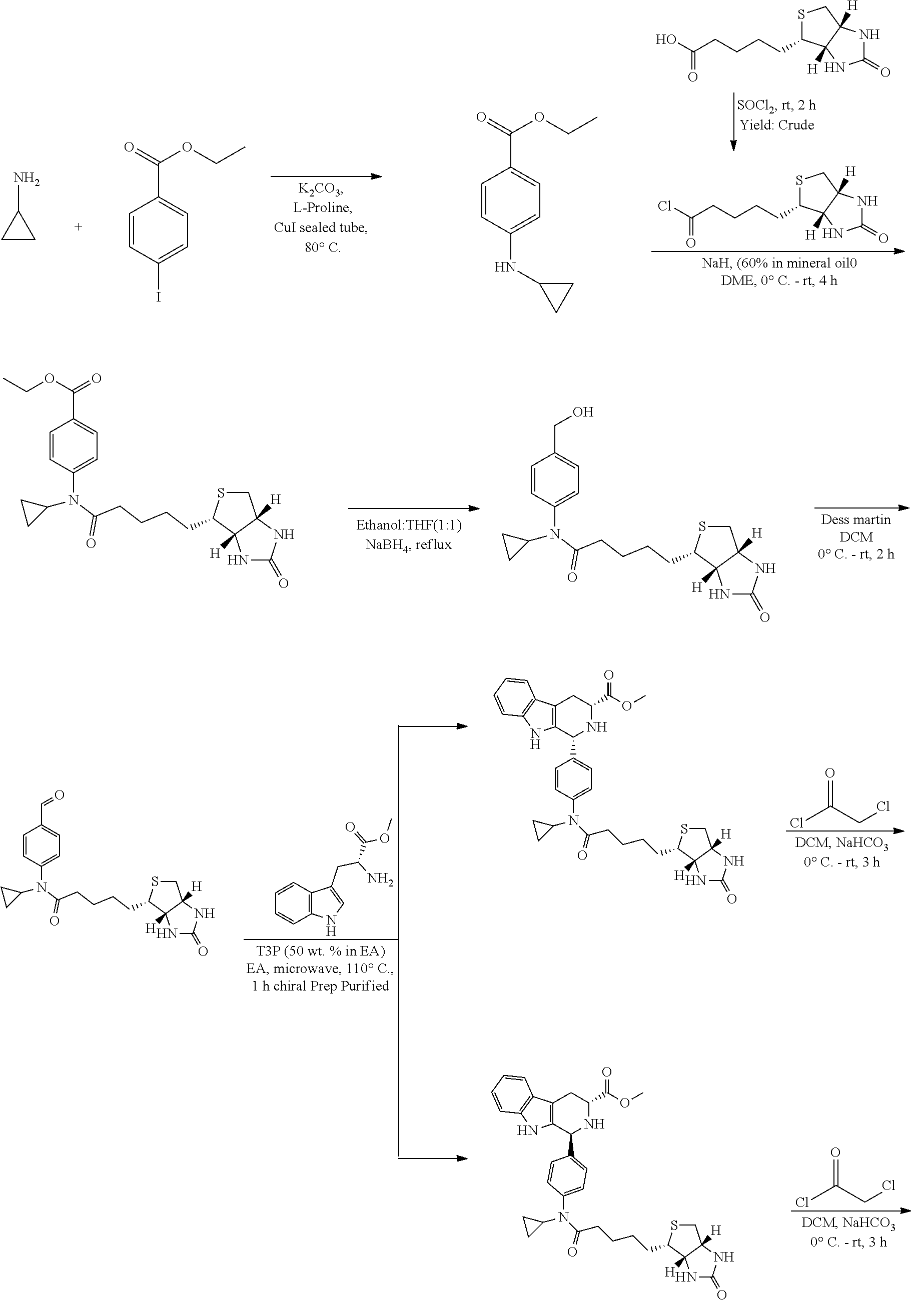

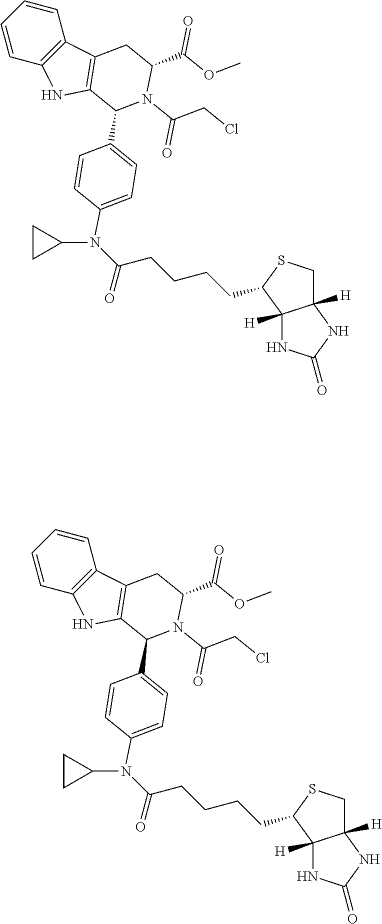 [SMILES: COC(=O)[C@H]1Cc2c([nH]c3ccccc23)[C@@H](c2ccc(N(C(=O)CCCC[C@@H]3SC[C@@H]4NC(=O)N[C@@H]43)C3CC3)cc2)N1C(=O)CCl ; COC(=O)[C@H]1Cc2c([nH]c3ccccc23)[C@H](c2ccc(N(C(=O)CCCC[C@@H]3SC[C@@H]4NC(=O)N[C@@H]43)C3CC3)cc2)N1C(=O)CCl]

ethyl 4-(cyclopropylamino)benzoate: To a solution of ethyl 4-iodobenzoate (25.0 g, 90.59 mmol, 1 eq.) in DMSO (135 mL) at 0° C. cyclopropanamine (19.36 mL, 271.77 mmol, 3 eq) and potassium carbonate (25.0 g, 181.18 mmol, 2 equiv) followed by L-Proline (2.08 g, 18.11 mmol, 0.2 equiv) was added. Then reaction mixture was sealed and heated at 80° C. for 5 h. Reaction mixture cool to room temperature and diluted with water and extracted with diethyl ether (2×200 mL). Combined organic layer washed with water (50 mL), brine (25 mL), dried over anhydrous Na₂SO₄, filtered and concentrated under reduced pressure to get the crude product. The crude was purified by flash chromatography using 15-25% EtOAc in hexane as an eluent to give ethyl 4-(cyclopropylamino)benzoate. LCMS (ES) m/z=206.0[M+H]⁺. ¹H NMR (400 MHz, CDCl₃) δ ppm: 0.52-0.55 (m, 2H), 0.77-0.80 (m, 2H), 1.35 (t, J=7.6 Hz, 3H), 2.47-2.49 (m, 1H), 4.28-4.38 (m, 2H), 4.53 (s, 1H), 6.53 (d, J=8.8 Hz, 2H), 7.88 (d, J=8.4 Hz, 2H).

5-((3aS,4S,6aR)-2-oxohexahydro-1H-thieno[3,4-d]imidazol-4-yl)pentanoyl chloride: To 5-((3aS,4S,6aR)-2-oxohexahydro-1H-thieno[3,4-d]imidazol-4-yl)pentanoic acid (4.0 g, 16.38 mmol, 1 eq) at 0° C. thionyl chloride (25 mL) added. Then reaction mixture stirred at room temperature for 2 h. Then the reaction was evaporated under reduced pressure obtained crude (5-((3aS,4S,6aR)-2-oxohexahydro-1H-thieno[3,4-d]imidazol-4-yl)pentanoyl chloride and taken for analysis. LCMS (ES) m/z=Desired mass not ionized.

ethyl4-(N-cyclopropyl-5-((3aS,4S,6aR)-2-oxohexahydro-1H-thieno[3,4-d]imidazol-4-yl)pentanamido)benzoate: To a solution of ethyl 4-(cyclopropylamino)benzoate (3.0 g, 14.62 mmol, 1 eq) at 0° C. sodium hydride (60% in mineral oil) (1.46 g, 35.56 mmol, 2.5 equiv) was added over a period of 15 minutes. Then reaction mixture was stirred at rt for 15 minutes and cool to 0° C. and 5-((3aS,4S,6aR)-2-oxohexahydro-1H-thieno[3,4-d]imidazol-4-yl)pentanoyl chloride (4.22 g, 16.08 mmol, 1.1 eq) in DMF (20.0 mL) added. The mixture was poured on crushed ice and extracted with EtOAc (2×50 mL). Combined organic layer washed with ice water and brine. The organic layer was dried over anhydrous Na₂SO₄, concentrated under reduced pressure to get the crude. The crude was purified by flash chromatography using 5-7% methanol in DCM as an eluent to give ethyl 4-(N-cyclopropyl-5-((3aS,4S,6aR)-2-oxohexahydro-1H-thieno[3,4-d]imidazol-4-yl)pentanamido)benzoate. ¹H NMR (400 MHz, DMSO-d₆) δ: ppm 0.43 (s, 2H), 0.84-0.86 (m, 2H), 1.28-1.39 (m, 5H), 1.41-1.63 (m, 4H), 2.43 (s, 2H), 2.54-2.57 (m, 1H), 2.79-2.83 (m, 1H), 3.05-3.09 (m, 1H), 3.14-3.17 (m, 1H), 4.05-4.11 (m, 1H), 4.27-4.32 (m, 3H), 6.33 (s 1H), 6.40 (s, 1H), 7.34 (d, J=8.8 Hz, 2H), 7.93 (d, J=8.0 Hz, 2H).

N-cyclopropyl-N-(4-(hydroxymethyl)phenyl)-5-((3aS,4S,6aR)-2-oxohexahydro-1H-thieno[3,4-d]imidazol-4-yl)pentanamide: To a solution of ethyl 4-(N-cyclopropyl-5-((3aS,4S,6aR)-2-oxohexahydro-1H-thieno[3,4-cl]imidazol-4-yl)pentanamido)benzoate (0.730 g, 1.69 mmol, 1 eq) in THF (10 mL) and Ethanol (10.0 mL) was added sodium borohydride (0.640 g, 16.91 mmol, 10.0 eq) at 0° C. and the reaction was stirred at refluxed for 14 h. The reaction mixture was concentrated under reduced pressure to get the crude which was dissolved in EtOAc (100 mL) and was washed with water (2×10 mL). The organic layers were dried over Na₂SO₄ and concentrated to give the crude product. The crude product was purified by flash column chromatography using 7-8% methanol in DCM as an eluent to give N-cyclopropyl-N-(4-(hydroxymethyl)phenyl)-5-((3aS,4S,6aR)-2-oxohexahydro-1H-thieno[3,4-d]imidazol-4-yl)pentanamide. LCMS (ES) m/z=390.2 [M+H]⁺.

N-cyclopropyl-N-(4-formylphenyl)-5-((3aS,4S,6aR)-2-oxohexahydro-1H-thieno[3,4-d]imidazol-4-yl)pentanamide: To a solution of N-cyclopropyl-N-(4-(hydroxymethyl)phenyl)-5-((3aS,4S,6aR)-2-oxohexahydro-1H-thieno[3,4-d]imidazol-4-yl)pentanamide (0.310 g, 0.795 mmol, 1 eq) in DCM (8.0 mL) was added Desmartin periodinane (0.405 g, 0.955 mmol, 1.2 eq) at 0° C. The mixture was allowed to stir at room temperature for 2 h. The reaction mixture was quenched with saturated NaHCO₃ solution at 0° C. and extracted with DCM (100 mL). The organic layers were dried over Na₂SO₄ and concentrated to get the crude. The crude product was purified by flash column chromatography using 8-9% methanol in DCM as an eluent to give N-cyclopropyl-N-(4-formylphenyl)-5-((3aS,4S,6aR)-2-oxohexahydro-1H-thieno[3,4-d]imidazol-4-yl)pentanamide. LCMS (ES) m/z=387.9 [M+H]⁺.

methyl (1S,3R)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate and methyl (1R,3R)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate: In a microwave vial to a solution of methyl D-tryptophan (0.101 g, 0.464 mmol, 1 eq) in ethyl acetate (6.0 mL) was added N-cyclopropyl-N-(4-formylphenyl)-5-((3aS,4S,6aR)-2-oxohexahydro-1H-thieno[3,4-d]imidazol-4-yl)pentanamide (0.180 g, 0.464 mmol, 1.0 eq) followed by T3P (50 wt. % in EA) was added. Then mixture was irradiated at 110° C. for 1 h. TLC The reaction mixture was cooled to room temperature and quenched with saturated sodium bi carbonate and extracted with Ethyl acetate (2×15 mL). Combined organic layer washed with water (15 mL), brine (5 mL) dried over anhydrous sodium sulphate. Then concentrated under reduced pressure to give the crude product. The crude product was purified by followed chiral preparative HPLC. Column: chiralpak IA (100 mm×4.6 mm×3 μm); Mobile phase (A): Ethanol with 0.1% DEA (100%). Product fractions collected and concentrated under reduce pressure to give methyl (1S,3R)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate and methyl (1R,3R)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate. In TLC non-polar (cis): LC-MS (m/z): 587.9 [M+H]⁺. In TLC Polar (trans): LC-MS (m/z): 587.9 [M+H]⁺.

methyl (1S,3R)-2-(2-chloroacetyl)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate: To a solution of methyl (1S,3R)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate (0.035 g, 0.059 mmol, 1 eq) in DCM (3.0 mL) was added NaHCO₃ (0.007 g, 0.089 mmol, 1.5 eq) at 0° C., stirred for 5 mins and then and 2-chloroacetyl chloride (0.004 mL, 0.089 mmol, 0.9 eq) was added at 0° C. The mixture was allowed to stir at room temperature for 3 hr. The reaction mixture was concentrated under reduced pressure to give the crude product. This reaction mixture taken for Preparative HPLC Purification by using following method: Column: X-BridgeC-18 (150 mm×4.6 mm×5 μm); Mobile phase A: 0.1% Ammonia in water; Mobile phase B: Acetonitrile. Product fractions collected and concentrated under reduced pressure to give methyl (1S,3R)-2-(2-chloroacetyl)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate. LC-MS (m/z): 664.2 [M+H]⁺. ¹H NMR (400 MHz, DMSO-d₆, VT at 70° C.) δ: ppm 0.37 (s, 2H), 0.71-0.73 (m, 2H), 1.27 (s, 2H), 1.39-1.59 (m, 4H), 2.24 (s, 2H), 2.54-2.59 (m, 1H), 2.78-2.79 (m, 1H), 3.01-3.10 (m, 2H), 3.20-3.30 (m, 1H), 3.40-3.49 (m, 1H), 3.52 (s, 3H), 4.11 (s, 1H), 4.20-4.28 (m, 2H), 4.52-4.59 (m, 1H), 5.15 (bs, 1H), 6.13-6.18 (m, 3H), 6.96-6.98 (m, 1H), 7.03-7.10 (m, 3H), 7.27 (d, J=8.0 Hz, 1H), 7.44-7.46 (m, 3H), 10.85 (s, 1H).

methyl (1R,3R)-2-(2-chloroacetyl)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate: To a solution of methyl (1R,3R)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate (0.034 g, 0.057 mmol, 1 eq) in DCM (3.0 mL) was added NaHCO₃ (0.007 g, 0.089 mmol, 1.5 eq) at 0° C., stirred for 5 mins and then and 2-chloroacetyl chloride (0.004 mL, 0.086 mmol, 0.9 eq) was added at 0° C. The mixture was allowed to stir at room temperature for 3 hr. The reaction mixture was concentrated under reduced pressure to give the crude product. This reaction mixture taken for Preparative HPLC Purification by using following method: Column: X-BridgeC-18 (150 mm×4.6 mm×5 μm); Mobile phase A: 0.1% Ammonia in water; Mobile phase B: Acetonitrile. Product fractions collected and concentrated under reduced pressure to give methyl (1R,3R)-2-(2-chloroacetyl)-1-(4-(N-cyclopropyl-5-((3aS,4S,6aR)-2-oxohexahydro-1H-thieno[3,4-d]imidazol-4-yl)pentanamido)phenyl)-2,3,4,9-tetrahydro-1H-pyrido[3,4-b]indole-3-carboxylate. LC-MS (m/z): 664.3 [M+H]⁺. ¹H NMR (400 MHz, DMSO-d6), δ: ppm 0.43 (s, 2H), 0.74 (s, 2H), 1.40-1.52 (m, 4H), 2.26-2.31 (m, 1H), 2.57 (s, 1H), 2.77-2.81 (m, 1H), 2.95 (s, 3H), 3.06-3.12 (m, 3H), 3.44-3.48 (m, 1H), 3.61 (s, 1H), 3.88 (s, 1H), 4.10 (s, 1H), 4.28 (s, 1H), 4.43-4.47 (m, 1H), 4.56 (1H), 4.82-4.86 (m, 1H), 5.21 (s, 1H), 6.30-6.34 (m, 2H), 6.89 (s, 1H), 7.00-7.09 (m, 6H), 7.28 (d, J=8.0 Hz, 1H), 7.54 (d, J=7.6 Hz, 1H), 10.92 (s, 1H).

### Procedure B-9: Compound B-24

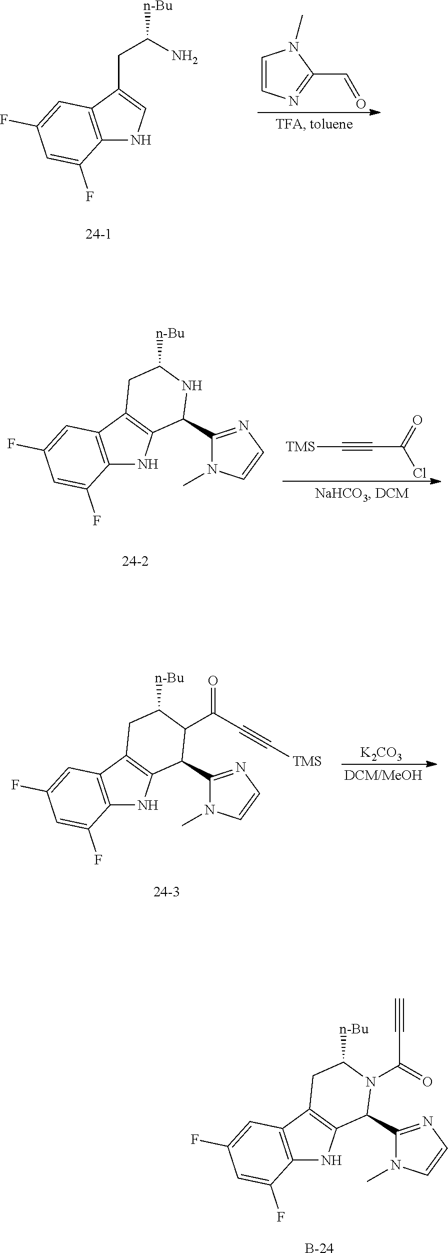

To a solution of 24-1 (200 mg, 792.70 μmol, 1 eq) in toluene (20 mL) were added 1-methylimidazole-2-carbaldehyde (87.29 mg, 792.70 μmol, 1 eq) and TFA (135.58 mg, 1.19 mmol, 88.04 uL, 1.5 eq). The mixture was stirred at 120° C. for 2 hr to give a yellow solution. TLC (PE: EtOAc=0:1) showed the reaction was completed. The mixture was concentrated under vacuum and added TEA (2 mL). The residue was purified by column chromatography (SiO₂, Petroleum ether/Ethyl acetate=100/0 to 0/100) to give the impure product (142 mg). The impure product (142 mg) was purified two times by prep-TLC (PE/EtOAc=2:1 (0.5 mL NH₃.H₂O)) to give 24-2. ¹H NMR (400 MHz, CHLOROFORM-d) δ ppm 8.66 (brs, 1H), 6.97-6.85 (m, 3H), 6.68-6.57 (m, 1H), 5.33-5.30 (m, 1H), 3.75 (s, 3H), 3.11-3.02 (m, 1H), 2.95-2.86 (m, 1H), 2.46-2.40 (m, 1H), 1.59-1.30 (m, 6H), 0.90 (t, J=7.2 Hz, 3H).

To a solution of 24-2 (25 mg, 72.59 μmol, 1 eq) in DCM (3 mL) were added NaHCO₃ (48.78 mg, 580.72 μmol, 22.59 μL, 8 eq) and 3-trimethylsilylprop-2-ynoyl chloride (0.1 M, 2.18 mL, 3 eq) at 0° C. The mixture was stirred at 0° C. for 1 hr to give a yellow suspension. TLC (PE:EtOAc=0:1) showed the mixture was completed. The reaction mixture was quenched with H₂O (10 mL) and extracted with DCM (10 mL*3).

The organic layers were dried over Na₂SO₄ and concentrated to give the crude product. The residue was purified by column chromatography (SiO₂, Petroleum ether/Ethyl acetate=100/0 to 49/51) to give 24-3.

To a solution of 24-3 (10 mg, 21.34 μmol, 1 eq) in DCM (5 mL)/MeOH (0.5 mL) were added K₂CO₃ (17.70 mg, 128.04 μmol, 6 eq) at 0° C. The mixture was stirred at 0° C. for 2 hr to give a white solution. TLC (PE/EtOAc=2/1) showed the mixture was completed. The mixture was diluted with DCM (5 mL) and added H₂O (5 mL). The organic layer was extracted with DCM (10 mL*3) and dried over Na₂SO₄ and concentrated to give the crude product. The residue was purified by prep-TLC (SiO₂, PE:EtOAc=1:5) to give B-24. LC-MS (m/z): 397.0 [M+H]⁺

¹H NMR (400 MHz, CDCl₃) δ ppm 7.55-7.32 (m, 1H), 7.08-6.82 (m, 2H), 6.80-6.65 (m, 2H), 5.40-4.93 (m, 1H), 3.59-3.31 (m, 3H), 3.11-2.94 (m, 3H), 2.00-1.95 (m, 1H), 0.88 (t, J=4.8 Hz, 3H).

### Procedure B-8: Compound B-25

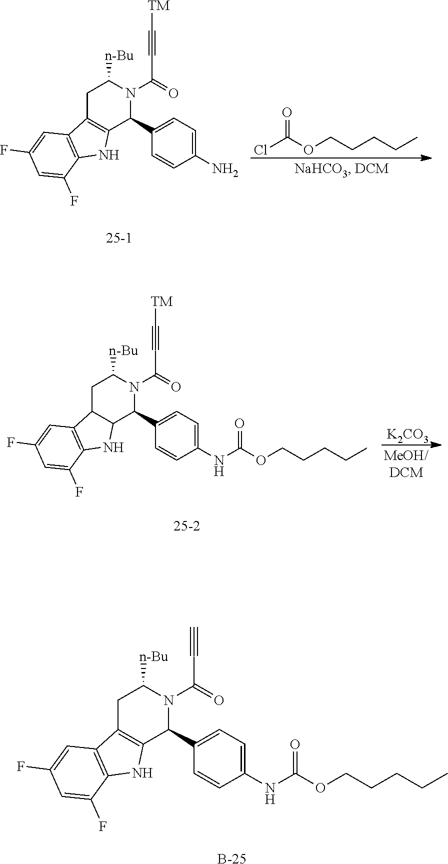

To a solution of 25-1 (40 mg, 83.40 μmol, 1 eq) in DCM (5 mL)/H₂O (1 mL) were added NaHCO₃ (105.09 mg, 1.25 mmol, 48.65 uL, 15 eq) at 0° C. The mixture was stirred at 0° C. for 0.5 hr to give a yellow solution. Then the mixture were added pentyl carbonochloridate (37.68 mg, 250.19 μmol, 3 eq) at 0° C. for 5 hr to give a yellow solution. TLC (eluting with: PE/EtOAc=2/1) showed have new spot was found. The reaction mixture was diluted with H₂O (10 ml) and extracted with DCM (15 mL*2). The organic layers were dried over Na₂SO₄ and concentrated to give the crude product. The residue was purified by prep-TLC (SiO₂, PE:EtOAc=2:1) to give 25-2. ¹H NMR (400 MHz, CDCl₃) δ ppm 8.04-7.79 (m, 1H), 7.45-7.19 (m, 3H), 6.98 (dd, J=2.0 Hz, J=8.8 Hz, 1H), 6.97 (m, 1H), 6.7-6.50 (m, 2H), 5.93-5.83 (m, 1H), 4.91 (brs, 1H), 4.20-4.10 (m, 2H), 3.28-2.79 (m, 2H), 1.71-1.60 (m, 4H), 1.39-1.30 (m, 7H), 0.93-0.77 (m, 7H), 0.26 (s, 9H).

To a solution of 25-2 (38 mg, 64.00 μmol, 1 eq) in DCM (5 mL)/MeOH (0.5 mL) were added K₂CO₃ (35.38 mg, 255.99 μmol, 4 eq) at 0° C. The mixture was stirred at 0° C. for 2 hr to give a yellow solution. TLC (PE/EtOAc=2/1) showed the reaction was completed. The mixture was diluted with DCM (5 mL) and added H₂O (5 mL). The organic layer was extracted with DCM (10 mL*3) and dried over Na₂SO₄ and concentrated to give the crude product. The residue was purified by column chromatography (SiO₂, Petroleum ether/Ethyl acetate=100/0 to 70/30) to give B-25. LC-MS (m/z): 522.1 [M+H]⁺

¹H NMR (400 MHz, CDCl₃) δ ppm 8.01-7.74 (m, 1H), 7.40-7.28 (m, 2H), 7.25-7.20 (m, 2H), 7.00-6.94 (m, 1H), 6.74-6.52 (m, 1H), 5.87 (s, 1H), 4.96-4.10 (m, 3H), 3.27 (dd, J=1.2 Hz, J=16.4 Hz, 1H), 3.12 (s, 1H), 3.03-2.78 (m, 1H), 1.69-1.65 (m, 3H), 1.48-1.12 (m, 9H), 0.94-0.78 (m, 6H).

### Procedure B-8: Compound B-26

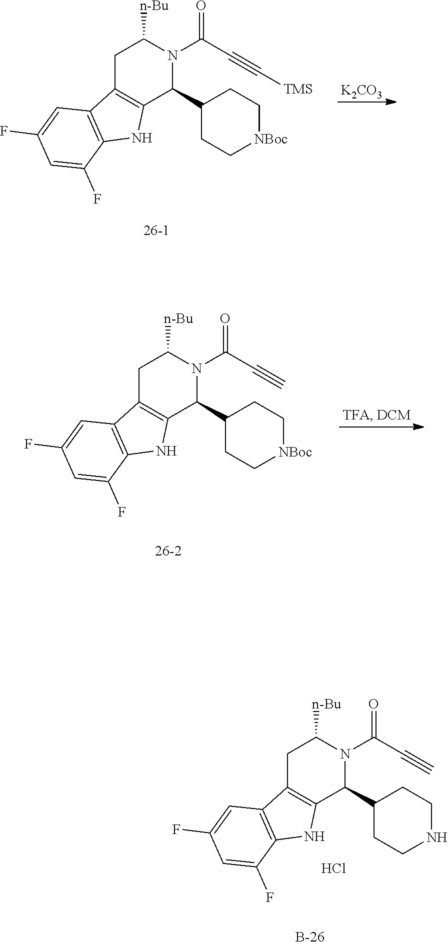

To a solution 26-1 (21 mg, 36.73 μmol, 1 eq) in DCM (5 mL)/MeOH (0.5 mL) were added K₂CO₃ (20.30 mg, 146.91 μmol, 4 eq) at 0° C. The mixture was stirred at 0° C. for 1 hr to give a white solution. TLC (PE:EtOAc=2:1) showed the reaction was completed. The mixture was diluted with DCM (5 mL) and added H₂O (5 mL). The organic layer was extracted with DCM (10 mL*3) and dried over Na₂SO₄ and concentrated to give the crude product (22 mg). The residue was purified by column chromatography (SiO₂, Petroleum ether/Ethyl acetate=100/0 to 70/30) to give 26-2 (16 mg, 32.03 μmol, 87.20% yield) as white solid. ¹H NMR (400 MHz, CDCl₃) δ ppm 8.35-8.00 (m, 1H), 6.99-6.93 (m, 1H), 6.76 (t, J=5.2 Hz, 1H), 5.32-5.10 (m, 1H), 4.69 (brs, 1H), 3.22-3.10 (m, 1H), 3.06-2.11 (m, 5H), 1.98-1.55 (m, 4H), 1.38 (s, 9H), 1.35-1.15 (m, 8H), 0.93-0.80 (m, 3H).

To a solution of 26-2 (16 mg, 32.03 μmol, 1 eq) in DCM (10 mL) were added TFA (1.54 g, 13.51 mmol, 1 mL, 421.72 eq) at 0° C. The mixture was stirred at 0° C. for 2 hr to give a white solution. LCMS showed the mixture was completed. The mixture was concentrated under vacuum. The solid was added H₂O (25 mL) and HCl (0.5 mL, 12 M) for lyophilize to give B-26. LC-MS (m/z): 400.1 [M+H]⁺

¹H NMR (400 MHz, CDCl₃) δ ppm 11.79-11.60 (m, 1H), 9.15-8.00 (m, 2H), 7.30-7.10 (m, 1H), 7.00-6.90 (m, 1H), 5.20-5.00 (m, 1H), 4.70-4.62 (m, 1H), 4.58-3.88 (m, 1H), 3.43-2.72 (m, 6H), 2.25-0.98 (m, 10H), 0.88-0.74 (m, 3H)

### Procedure B-8: Compound B-27

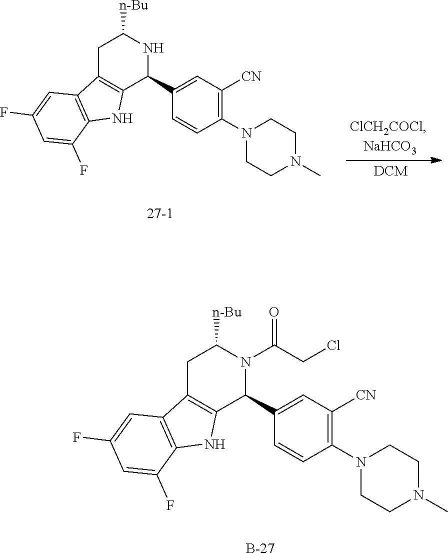

To a solution of 27-1 (50.00 mg, 107.86 μmol, 1 eq) in DCM (10 mL) were added NaHCO₃ (90.61 mg, 1.08 mmol, 41.95 uL, 10 eq) and 2-chloroacetyl chloride (36.55 mg, 323.58 μmol, 25.74 μL, 3 eq). The mixture was stirred at 15° C. for 1 hr to give a yellow solution. LCMS and TLC (eluting with: EtOAc/MeOH/NH₃.H₂O=10/1/0.1) showed the reaction was completed. The reaction mixture was diluted with H₂O (15 mL) and extracted with DCM (30 mL*3). The organic layers were dried over Na₂SO₄ and concentrated to give the crude product. The crude product was purified by flash column (eluting with: PE/EtOAc=100% PE to 100% EtOAc) to give B-27. LC-MS (m/z): 540.1[M+1-1]⁺

¹H NMR (400 MHz, CDCl₃) δ=7.87 (s, 1H), 7.34 (d, J=2.0 Hz, 1H), 7.30-7.27 (m, 1H), 6.85-6.70 (m, 2H), 6.58-6.48 (m, 1H), 5.66 (s, 1H), 4.35-4.27 (m, 1H), 4.09 (brs, 1H), 3.98-3.80 (m, 1H), 3.13-3.06 (m, 4H), 2.89-2.80 (m, 1H), 2.52-2.48 (m, 4H), 2.24 (s, 3H), 1.48-1.38 (m, 2H), 1.10-0.90 (m, 6H), 0.71 (t, J=7.2 Hz, 3H).

### Procedure B-8: Compound B-28

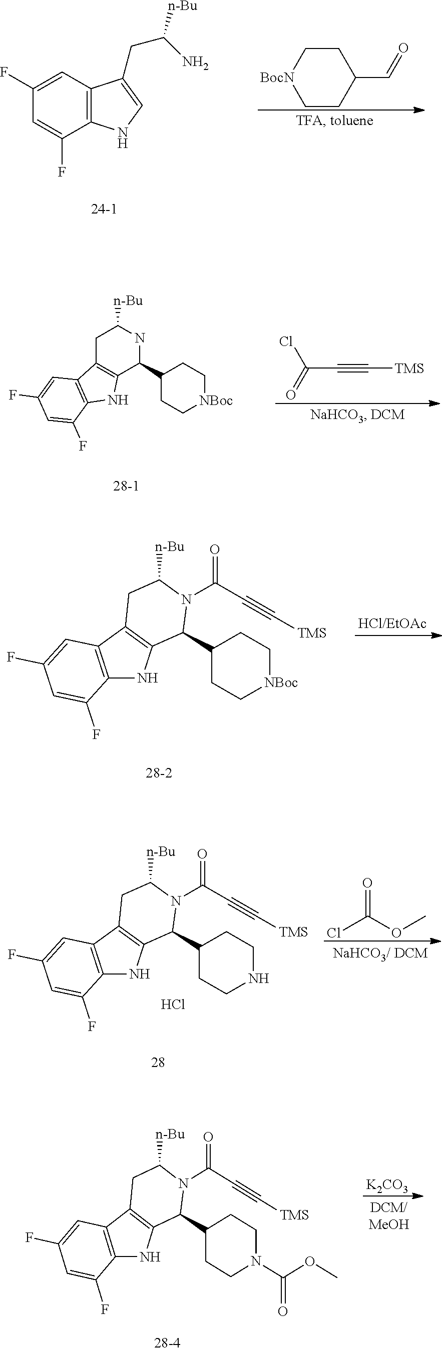

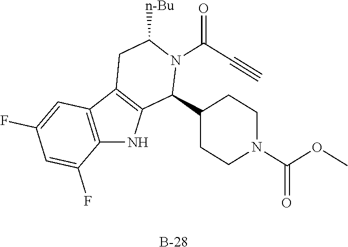

To a solution of 24-1 (200 mg, 792.70 μmol, 1 eq) in toluene (20 mL) were added tert-butyl 4-formylpiperidine-1-carboxylate (169.06 mg, 792.70 μmol, 1 eq) and TFA (9.04 mg, 79.27 μmol, 5.87 uL, 0.1 eq). The mixture was stirred at 120° C. for 2 hr to give a yellow solution. TLC (PE/EtOAc=2/1) showed the reaction was completed. The mixture was added TEA (3 mL) and concentrated under vacuum. The residue was purified by column chromatography (SiO₂, Petroleum ether/Ethyl acetate=100/0 to 75/25 to 68/32) to give 28-1. ¹H NMR (400 MHz, CDCl₃) δ ppm 7.85 (brs, 1H), 6.93 (dd, J=2.0 Hz, J=8.8 Hz, 1H), 6.70 (t, J=4.8 Hz, 1H), 4.21 (brs, 1H), 3.71 (d, J=6.8 Hz, 1H), 3.09-3.04 (m, 1H), 2.78 (dd, J=4.8 Hz, J=15.6 Hz, 1H), 2.66 (brs, 2H), 2.34-2.24 (m, 1H), 1.90-1.58 (m, 3H), 1.56-1.51 (m, 3H), 1.50 (s, 9H), 1.48-1.35 (m, 5H), 0.95 (t, J=6.8 Hz, 3H).

To a solution of 28-1 (45 mg, 100.55 μmol, 1 eq) in DCM (4 mL)/H₂O (2 mL) were added NaHCO₃ (67.57 mg, 804.36 μmol, 31.28 uL, 8 eq) and 3-trimethylsilylprop-2-ynoyl chloride (0.1 M, 2.01 mL, 2 eq) at 0° C. The mixture was stirred at 0° C. for 2 hr to give a yellow solution. TLC (PE/EtOAc=2/1) showed the reaction was completed. The reaction mixture was quenched with H₂O (10 mL) and extracted with DCM (10 mL*3). The organic layers were dried over Na₂SO₄ and concentrated to give the crude product. The residue was purified by column chromatography (SiO₂, Petroleum ether/Ethyl acetate=100/0 to 74/26) to give 28-2. ¹H NMR (400 MHz, CDCl₃) δ ppm 8.61 (brs, 1H), 6.97 (dd, J=1.6 Hz, J=8.8 Hz, 1H), 6.75 (t, J=2.4 Hz, 1H), 5.24-5.10 (m, 1H), 4.66 (brs, 1H), 2.95-2.90 (m, 2H), 2.65-2.55 (m, 2H), 1.90-1.55 (m, 6H), 1.50 (s, 9H), 1.48-1.24 (m, 6H), 0.86-0.82 (m, 3H), 0.28 (s, 9H).

To a stirred solution of 28-2 (21 mg, 36.73 μmol, 1 eq) in HCl/EtOAc (4 M, 10 mL, 1089.09 eq) at 0° C. for 2 hr to give a white solution. LCMS showed the reaction was completed. The reaction mixture was concentrated under vacuum. The 28-3 was used for next step without further purification

To a solution of 28-3 (18.66 mg, 39.56 μmol, 1 eq) in DCM (15 mL)/H₂O (5 mL) were added NaHCO₃ (99.71 mg, 1.19 mmol, 46.16 uL, 30 eq) and methyl carbonochloridate (37.39 mg, 395.63 μmol, 30.64 uL, 10 eq) at 0° C. The mixture was stirred at 0° C. for 0.5 hr to give a white solution. TLC (PE:EtOAc=3:1) showed the mixture was completed. The reaction mixture was diluted with H₂O (30 ml) and extracted with DCM (40 mL*2). The organic layers were dried over Na₂SO₄ and concentrated to give 28-4. It was used the next step without further purification. ¹H NMR (400 MHz, CDCl₃) δ ppm 8.34-8.06 (m, 1H), 6.95 (dd, J=1.6 Hz, J=7.2 Hz, 1H), 6.75 (t, J=9.2 Hz, 1H), 5.11 (d, J=3.6 Hz, 1H), 4.66 (brs, 1H), 4.15-3.55 (m, 3H), 2.94-2.60 (m, 5H), 1.90-1.84 (m, 1H), 1.48-1.20 (m, 10H), 0.90-0.82 (m, 3H), 0.26 (s, 9H).

To a solution of 28-4 (35 mg, 66.08 μmol, 1 eq) in DCM (10 mL)/MeOH (1 mL) were added K₂CO₃ (36.53 mg, 264.30 μmol, 4 eq) at 0° C. The mixture was stirred at 0° C. for 1 hr to give a yellow solution. TLC (PE:EtOAc=1:1) showed the mixture was completed. The mixture was diluted with DCM (5 mL) and added H₂O (5 mL). The organic layer was extracted with DCM (10 mL*3) and dried over Na₂SO₄ and concentrated to give the crude product (72 mg). The residue was purified by column chromatography (SiO₂, Petroleum ether/Ethyl acetate=100/0 to 59/41) to give B-28. LC-MS (m/z): 458.1 [M+H]⁺

¹H NMR (400 MHz, CDCl₃) δ ppm 8.70-8.20 (m, 1H), 6.95 (dd, J=2.8 Hz, J=8.8 Hz, 1H), 6.75 (t, J=5.2 Hz, 1H), 5.24-5.10 (m, 1H), 4.68 (brs, 1H), 4.28-3.70 (m, 2H), 3.65-3.58 (m, 3H), 3.24-3.10 (m, 1H), 2.78-2.68 (m, 1H), 2.67-2.50 (m, 3H), 2.06-1.62 (m, 1H), 1.48-1.20 (m, 10H), 0.90-0.82 (m, 3H).

### Procedure B-8: Compound B-29

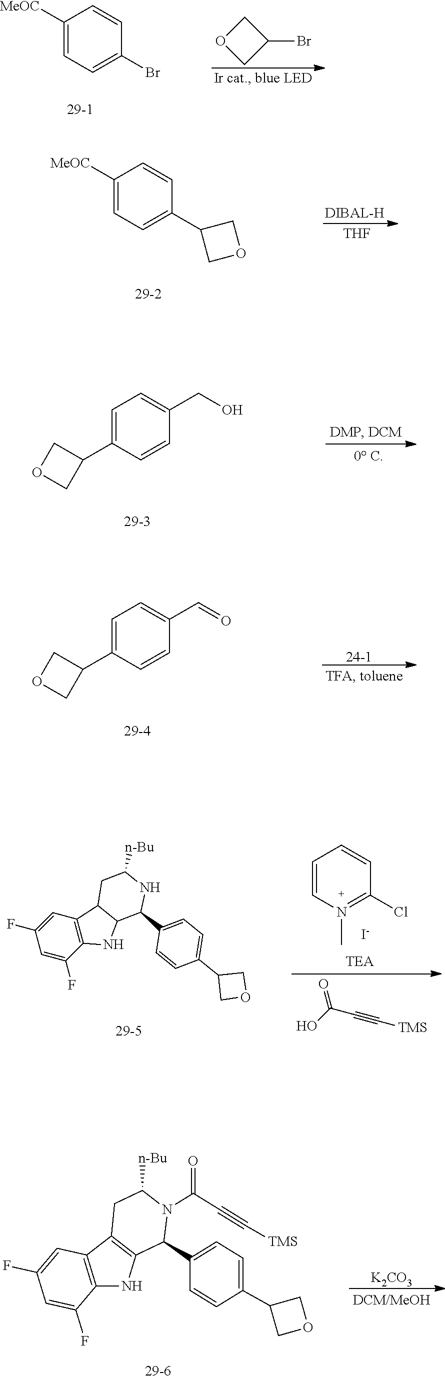

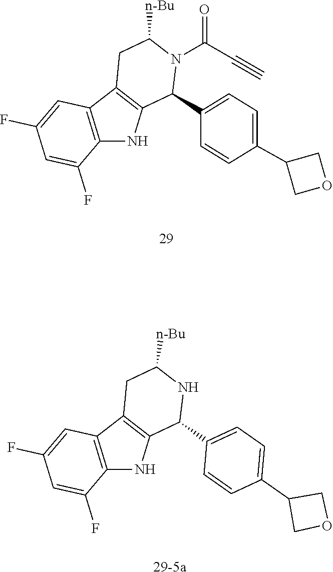

To a solution of 29-1 (1 g, 4.65 mmol, 1 eq) and 3-bromooxetane (1.5 g, 10.95 mmol, 2.35 eq) in DME (10 mL) was added bis[3,5-difluoro-2[5-(trifluoromethyl)-2-pyridyl]phenyl]iridium (1+); 4-tert-butyl-2-(4-tert-butyl-2-pyridyl)pyridine; hexafluorophosphate (52.17 mg, 46.50 μmol, 0.01 eq), Na₂CO₃ (985.74 mg, 9.30 mmol, 2 eq) and tris(trimethylsilyl)silane (1.16 g, 4.65 mmol, 1.43 mL, 1 eq) under N₂. To a separated vial was added 4,4′-di-tert-butyl-2,2′-bipyridine (6.24 mg, 23.25 μmol, 0.005 eq) and dichloronickel; 1,2-dimethoxyethane (5.11 mg, 23.25 μmol, 0.005 eq) in DME (2 mL), the precatalyst solution was stirred for 5 min under N₂, after which, it was syringed into the reaction vessel. The result mixture was stirred at 20° C. and irradiated with blue LED lamp for 6 h to give a brown mixture. TLC (EtOAc/PE=8:1) showed the reaction was completed. The mixture was filtered through celite. The filtrate was concentrated to give the crude product (2 g). The crude product was purified by flash column (SiO₂, EtOAc in PE from 0 to 8%) to give 29-2. ¹H NMR (400 MHz, CDCl₃) δ ppm 8.05 (d, J=8.4 Hz, 2H), 7.47 (d, J=8.4 Hz, 2H), 5.15-5.08 (m, 2H), 4.80-4.75 (m, 2H), 3.93 (s, 3H).

To a solution of methyl 29-2 (230 mg, 1.20 mmol, 1 eq) in THF (5 mL) was added LAH (136.25 mg, 3.59 mmol, 3 eq) at 0° C. The mixture was stirred at 0° C. for 2 h to give a yellow white suspension. TLC (PE/EtOAc=4:1) showed the reaction was completed. The reaction was quenched with saturated H₂O (30 mL) at 0° C. and dried over sodium sulfate, filtered and concentrated to give 29-3. ¹H NMR (400 MHz, CDCl₃): δ ppm 7.45-7.30 (m, 4H), 5.15-5.08 (m, 2H), 4.80-4.70 (m, 2H), 4.67 (s, 2H), 4.29-4.18 (m, 1H).

To a solution of 29-3 (210 mg, 1.28 mmol, 1 eq) in DCM (5 mL) was added DMP (813.67 mg, 1.92 mmol, 593.92 uL, 1.5 eq) at 0° C. The mixture was stirred at 0° C. for 1 h to give a white mixture. TLC (PE/EtOAc=3:1) showed the reaction was completed. The reaction was quenched with H₂O (50 mL) and extracted with DCM (50 mL×3). The combined organic layers were washed with brine (50 mL) and dried over sodium sulfate, filtered and concentrated to give the crude product (0.5 g). The crude product was purified by flash column (SiO₂, EtOAc in PE from 0 to 8%) to give 29-4. ¹H NMR (400 MHz, CDCl₃): δ ppm 10.06 (s, 1H), 7.91 (d, J=8.0 Hz, 2H), 7.58 (d, J=8.0 Hz, 2H), 5.17-5.09 (m, 2H), 4.82-4.74 (m, 2H), 4.36-4.27 (m, 1H).

To a solution of 24-1 (77.78 mg, 308.29 μmol, 1 eq) and 29-4 (50 mg, 308.29 μmol, 1 eq) in toluene (10 mL) was added TFA (52.73 mg, 462.44 μmol, 34.24 uL, 1.5 eq). The mixture was stirred at 120° C. for 2 h to give a yellow solution. LCMS and TLC (EtOAc/PE=1:3) showed the reaction was completed. The reaction was concentrated to give the residue. The residue was diluted with EtOAc (30 mL) and basic with saturated NaHCO₃ (40 mL). The separated aqueous phase was extracted with EtOAc (30 mL×3). The combined organic layers were washed with brine (30 mL) and dried over sodium sulfate, filtered and concentrated to give the crude product (0.14 g). The crude product was purified by prep-TLC (SiO₂, PE/EtOAc=1:1.5) to give 29-5 and 29-5a. ¹H NMR (400 MHz, CDCl₃): δ ppm 7.79 (s, 1H), 7.38 (d, J=8.0 Hz, 2H), 7.23 (d, J=8.0 Hz, 2H), 7.01 (dd, J=2.4 Hz, J=9.2 Hz, 1H), 6.72 (t, J=11.2 Hz, J=9.2 Hz, 1H), 5.22 (s, 1H), 5.10-5.04 (m, 2H), 4.80-4.74 (m, 2H), 4.29-4.20 (m, 1H), 3.13-3.04 (m, 1H), 2.91 (dd, J=4.0 Hz, J=14.2 Hz, 1H), 2.51-2.44 (m, 1H), 1.58-1.32 (m, 6H), 0.89 (t, J=7.2 Hz, 3H).

To a solution of 3-trimethylsilylprop-2-ynoic acid (9.33 mg, 65.58 μmol, 1 eq) in DCM (2 mL) was added 2-chloro-1-methyl-pyridin-1-ium; iodide (25.13 mg, 98.37 μmol, 1.5 eq). The mixture was stirred at 10° C. for 1 h. Then 29-5 (26 mg, 65.58 μmol, 1 eq) and TEA (9.95 mg, 98.37 μmol, 13.69 uL, 1.5 eq) in DCM (2 mL) was added to the mixture drop wise. The result mixture was stirred at 10° C. for another 1 h to give a yellow solution. TLC (PE/EtOAc=1:1) showed the reaction was completed. The reaction was diluted with H₂O (30 mL) and extracted with DCM (20 mL×3). The combined organic layers were washed with brine (20 mL) and dried over with sodium sulfate, filtered and concentrated to give the crude product. Combined with 30 mg of the crude product which was from 10 mg of 29-5, the crude product was purified by prep-TLC (SiO₂, PE/EtOAc=1:1) to give 29-6.

To a solution of 29-6 (15 mg, 28.81 μmol, 1 eq) in DCM (5 mL)/MeOH (1 mL) were added K₂CO₃ (7.96 mg, 57.62 μmol, 2 eq) at 0° C. The mixture was stirred at 0° C. for 1.5 hr to give a white solution. TLC (PE/EtOAc=2:1) showed the mixture was completed. The mixture was added H₂O (5 mL), extracted with DCM (8 mL*3). The combined organic layers were washed with brine (10 mL) and dried over Na₂SO₄, filtered and concentrated to give the crude product. The crude product was purified by prep-TLC (SiO₂, PE/EtOAc=1.5:1) to give B-29. LC-MS (M/Z): 449.1 [M+1-1]⁺

¹H NMR (400 MHz, CDCl₃): δ ppm 7.74 (s, 1H), 7.41-7.31 (m, 3H), 7.01-6.96 (m, 1H), 6.74-6.60 (m, 1H), 5.87 (s, 1H), 5.09-5.00 (m, 3H), 4.76-4.68 (m, 2H), 4.20-4.14 (m, 1H), 3.31-3.20 (m, 1H), 3.13 (s, 1H), 3.05-2.97 (m, 1H), 1.69-1.63 (m, 2H), 1.48-1.32 (m, 4H), 0.90-0.86 (m, 3H).

### Procedure B-8: Compound B-30

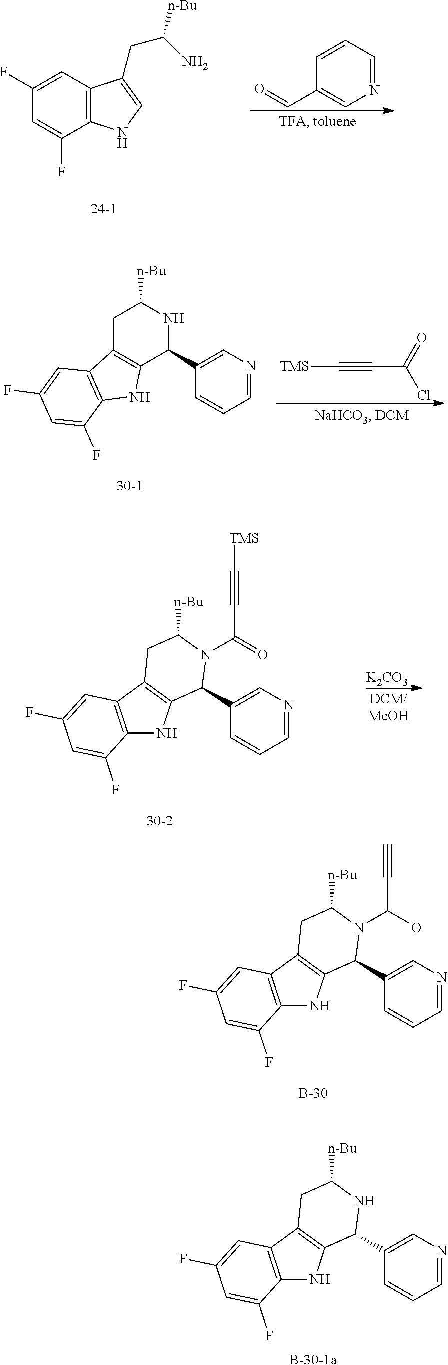

To a solution of 24-1 (200 mg, 792.70 μmol, 1 eq) in toluene (20 mL) were added pyridine-3-carbaldehyde (84.91 mg, 792.70 μmol, 74.48 uL, 1 eq) and TFA (135.58 mg, 1.19 mmol, 88.04 uL, 1.5 eq). The mixture was stirred at 120° C. for 1 hr to give a yellow suspension. TLC (PE:EtOAc=1:3) showed the reaction was completed. The reaction mixture was combined with ES11454-114 (200 mg R1) for work up. The mixture was added TEA (3 mL) and H₂O (10 ml), extracted with EtOAc (15*3 mL). The organic layers were dried over Na₂SO₄ and concentrated to give the crude product. The residue was purified by column chromatography (SiO₂, Petroleum ether/Ethyl acetate=100/0 to 20/80) to give 30-1a and 30-1. ¹H NMR (400 MHz, CDCl₃) δ ppm 8.58-8.50 (m, 2H), 7.93 (brs, 1H), 7.51 (dd, J=6.0 Hz, J=2.0 Hz, 1H), 7.25-7.23 (m, 1H), 7.0 (dd, J=6.0 Hz, J=8.8 Hz, 1H), 6.71 (t, J=2.0 Hz, 1H), 5.26 (s, 1H), 3.00-2.96 (m, 1H), 2.91-2.86 (m, 1H), 2.50-2.44 (m, 1H), 1.54-1.30 (m, 6H), 0.89 (t, J=6.8 Hz, 3H).

To a solution of 30-1 (41 mg, 120.09 μmol, 1 eq) in DCM (10 mL) was added NaHCO₃ (80.71 mg, 960.76 μmol, 37.37 uL, 8 eq) and 3-trimethylsilylprop-2-ynoyl chloride (0.1 M, 2.40 mL, 2 eq) at 0° C. The reaction mixture was allowed to stir at 10° C. for 2 hr to give a yellow suspension. TLC (PE/EtOAc=1:3) showed the mixture was completed. The reaction mixture was quenched with H₂O (20 mL) and extracted with DCM (20 mL*3). The organic layers were dried over Na₂SO₄ and concentrated to give the crude product. The residue was purified by column chromatography (SiO₂, Petroleum ether/Ethyl acetate=70/30 to 65/35) to give 30-2. ¹H NMR (400 MHz, CDCl₃) δ ppm 8.65 (s, 1H), 8.50 (s, 1H), 8.42-8.40 (m, 1H), 7.57-7.52 (m, 1H), 7.18 (t, J=3.2 Hz, 1H), 7.00 (dd, J=2.0 Hz, J=15.6 Hz, 1H), 6.74 (t, J=2.0 Hz, 1H), 5.94 (s, 1H), 5.01-4.93 (m, 1H), 3.29 (dd, J=4.8 Hz, J=2.0 Hz, 1H), 3.06-3.04 (d, J=15.6 Hz, 1H), 1.63-1.56 (m, 2H), 1.42-1.29 (m, 4H), 0.90 (t, J=7.2 Hz, 3H), 0.26 (s, 9H).

To a solution of 30-2 (34 mg, 73.02 μmol, 1 eq) in DCM (5 mL)/MeOH (0.5 mL) were added K₂CO₃ (40.37 mg, 292.09 μmol, 4 eq) at 0° C. The mixture was stirred at 0° C. for 2 hr to give a yellow suspension. TLC (eluting with: PE/EtOAc=1/1) showed the mixture was completed. The mixture was diluted with DCM (5 mL) and added H₂O (5 mL). The organic layer was extracted with DCM (20 mL*3) and dried over Na₂SO₄ and concentrated to give the crude product. The residue was purified by column chromatography (SiO₂, Petroleum ether/Ethyl acetate=100/0 to 48/52) to give B-30. LC-MS (m/z): 394.0 [M+H]⁺

¹H NMR (400 MHz, CDCl₃) δ ppm 8.64-8.63 (m, 1H), 8.47-8.43 (m, 1H), 8.14 (brs, 1H), 7.59-7.53 (m, 1H), 7.24-7.16 (m, 1H), 7.00 (d, J=8.4 Hz, 1H), 6.73 (t, J=9.2 Hz, 1H), 5.91 (s, 1H), 5.02 (brs, 1H), 3.31-3.26 (m, 1H), 3.16 (s, 1H), 3.04 (d, J=14.8 Hz, 1H), 1.59-1.55 (m, 2H), 1.43-1.28 (m, 4H), 0.89 (t, J=7.2 Hz, 3H).

### Procedure B-8: Compound B-32

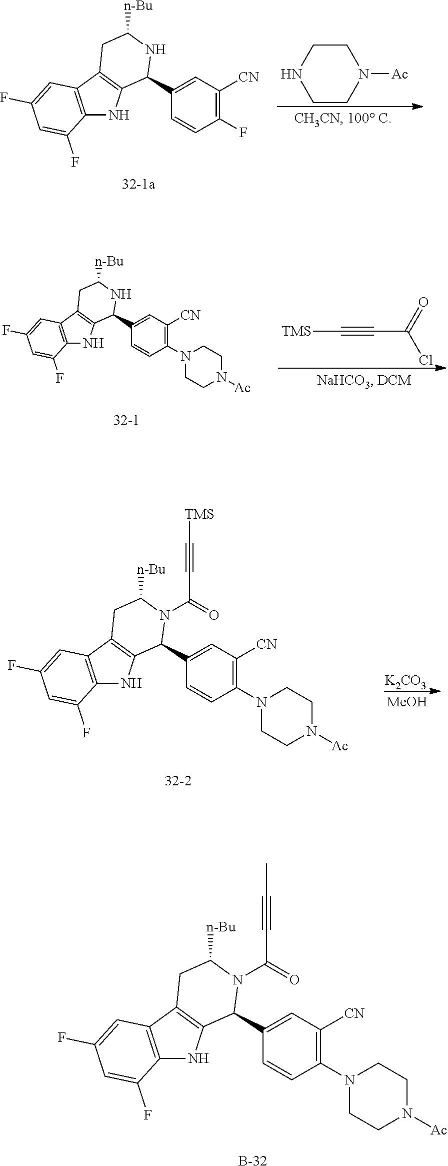

To a solution of 32-1a (160 mg, 417.31 μmol, 1 eq) in CH₃CN (5 mL) was added 1-piperazin-1-ylethanone (1.07 g, 8.35 mmol, 20 eq). The mixture was stirred at 100° C. for 12 hr to give a yellow solution. LCMS showed the desired mass was found, and 32-1a remained. The reaction was stirred at 100° C. for 20 hr. LCMS and TLC (eluting with: EtOAc/MeOH=10/1) showed the reaction was completed. The reaction mixture was quenched with EtOAc (30 mL*3). The organic layers were dried over Na₂SO₄ and concentrated to give the crude product. The crude product was purified by flash column (eluting with: PE/EtOAc=100% PE to 100% EtOAc) to give 32-1. ¹H NMR (400 MHz, CDCl₃) δ=8.05-7.95 (m, 1H), 7.40-7.30 (m, 2H), 7.00-6.88 (m, 2H), 6.66-6.60 (m, 1H), 5.11 (s, 1H), 3.78-3.70 (m, 2H), 3.60 (m, 6H), 3.20-3.00 (m, 4H), 2.86-2.70 (m, 2H), 2.07 (s, 3H), 1.44-1.20 (m, 6H), 0.83 (t, J=5.4 HZ, 3H).

To a solution of 32-1 (50.00 mg, 101.71 μmol, 1 eq) in DCM (5 mL)/H₂O (2 mL) were added NaHCO₃ (170.89 mg, 2.03 mmol, 79.12 uL, 20 eq) and 3-trimethylsilylprop-2-ynoyl chloride (0.14 M, 2.18 mL, 3 eq) at 0° C. The mixture was stirred at 0° C. for 0.5 hr to give a yellow solution. LCMS showed the reaction was completed. The reaction mixture was diluted with H₂O (20 mL) and extracted with DCM (30 mL*3). The organic layers were dried over Na₂SO₄ and concentrated to give 32-2 (65 mg, crude) as a yellow solid. Used for next step without further purification.

To a solution of 32-2 (65.00 mg, 105.56 μmol, 1 eq) in DCM (5 mL)/MeOH (1 mL) was added K₂CO₃ (29.18 mg, 211.11 μmol, 2 eq) at 0° C. The mixture was stirred at 0° C. for 0.5 hr to give a yellow solution. TLC (eluting with: EtOAc/PE=2/1) showed the reaction was completed. The reaction mixture was quenched with H₂O (20 mL) and extracted with DCM (20 mL*3). The organic layers were dried over Na₂SO₄ and concentrated to give the crude product. The crude product was purified by flash column (eluting with: PE/EtOAc=100% to 80%) to give B-32. LC-MS (m/z): 544.1[M+1-1]⁺

¹H NMR (400 MHz, CDCl₃) δ=7.75-8.60 (m, 1H), 7.45 (d, J=2.4 Hz, 1H), 7.0-7.27 (m, 1H), 7.40-7.32 (m, 1H), 6.98-6.88 (m, 1H), 6.80-6.72 (m, 1H), 6.70-6.60 (m, 1H), 5.78 (s, 1H), 4.95-4.82 (m, 1H), 3.70-3.50 (m, 4H), 3.20-2.90 (m, 6H), 2.09 (s, 3H), 1.51-1.16 (m, 6H), 0.80 (t, J=7.0 Hz, 3H).

### Procedure B-8: Compound B-34

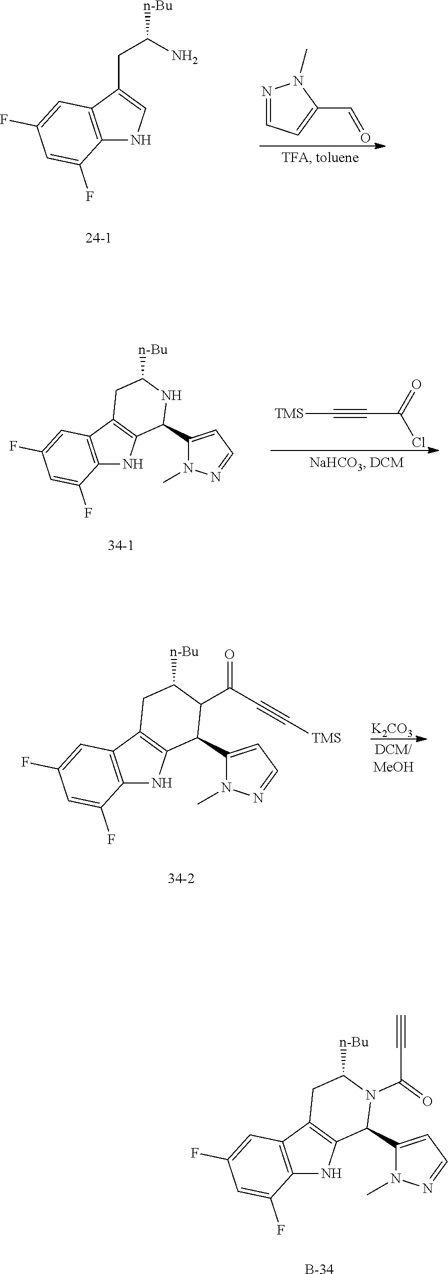

To a solution of 24-1 (200 mg, 792.70 μmol, 1 eq) in toluene (20 mL) were added 2-methylpyrazole-3-carbaldehyde (87.29 mg, 792.70 μmol, 1 eq) and TFA (135.58 mg, 1.19 mmol, 88.04 uL, 1.5 eq). The mixture was stirred at 120° C. for 2 hr to give a yellow solution. TLC (eluting with: EtOAc/PE=2/1) showed the reaction was completed. The reaction mixture was basified to pH=8 with Et₃N. The mixture was concentrate to give the crude product. The crude product was purified by flash column (eluting with: PE/EtOAc=0% to 80%) to give 34-1. ¹H NMR (400 MHz, CDCl₃) δ=7.89 (s, 1H), 7.27 (s, 1H), 6.92 (dd, J=2.0 Hz, J=8.8 Hz, 1H), 6.66-6.60 (m, 1H), 5.68 (s, 1H), 5.20 (s, 1H), 3.96 (s, 3H), 2.88-2.80 (m, 1H), 2.78-2.72 (m, 1H), 2.34-2.24 (m, 1H), 1.44-1.21 (m, 6H), 0.82 (t, J=7.2 Hz, 3H).

To a solution of 34-1 (110 mg, 319.40 μmol, 1 eq) in DCM (10 mL)/H₂O (3 mL) were added NaHCO₃ (268.31 mg, 3.19 mmol, 124.22 uL, 10 eq) and 3-trimethylsilylprop-2-ynoyl chloride (153.95 mg, 958.19 μmol, 3 eq) at 0° C. The mixture was stirred at 0° C. for 0.5 hr to give a yellow solution. LCMS showed the reaction was completed. The reaction mixture was quenched with H₂O (20 mL) and extracted with DCM (30 mL*3). The organic layers were dried over Na₂SO₄ and concentrated to give 34-2. Used for next step without further purification.

To a solution of 34-2 (160 mg, 341.43 μmol, 1 eq) in DCM (5 mL)/MeOH (1 mL) was added NaHCO₃ (28.68 mg, 341.43 μmol, 13.28 uL, 1 eq) at 0° C. The mixture was stirred at 0° C. for 0.5 hr to give a yellow solution. LCMS and TLC (eluting with: PE/EtOAc=1/1) showed the reaction was completed. The reaction mixture was quenched with H₂O (20 mL) and extracted with DCM (20 mL*3). The organic layers were dried over Na₂SO₄ and concentrated to give the crude product. The crude product was purified by flash column (eluting with: PE/EtOAc=0% to 50%) to give B-34. LC-MS (m/z): 397.0[M+H]⁺

¹H NMR (400 MHz, CDCl₃) δ=8.02-7.94 (m, 1H), 7.31-7.28 (m, 1H), 6.95-6.88 (m, 1H), 6.69-6.20 (m, 2H), 5.95-5.76 (m, 1H), 4.56-3.60 (m, 4H), 3.13 (s, 1H), 3.10-2.25 (m, 2H), 2.04-1.60 (m, 2H), 1.28-1.03 (m, 4H), 0.84-0.74 (m, 3H)

### Procedure B-8: Compound B-35

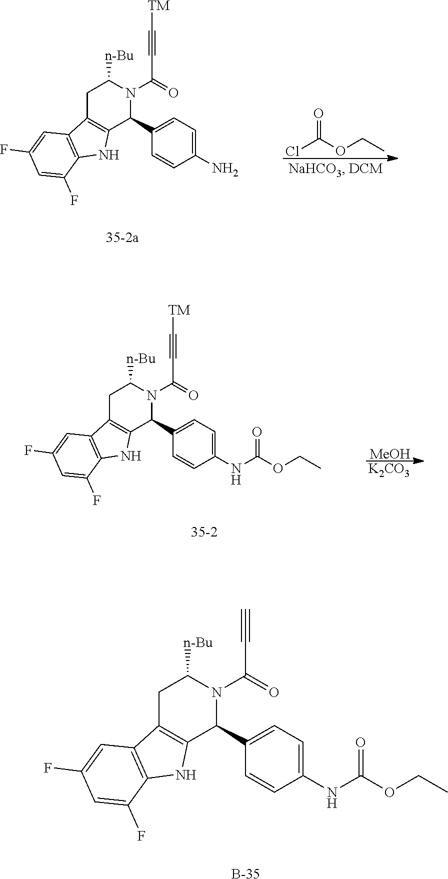

To a solution of 35-2a (50 mg, 104.25 μmol, 1 eq) in DCM (5 mL)/H₂O (1 mL) were added NaHCO₃ (131.36 mg, 1.56 mmol, 60.81 uL, 15 eq) at 0° C. The mixture was stirred at 0° C. for 0.5 hr to give a yellow solution. Then the mixture were added ethyl carbonochloridate (11.31 mg, 104.25 μmol, 9.92 uL, 1 eq) at 0° C. for 2 hr to give a yellow solution. TLC (PE/EtOAc=3/1) showed the reaction was completed. The reaction mixture was diluted with H₂O (30 ml) and extracted with DCM (40 mL*2). The organic layers were dried over Na₂SO₄ and concentrated to give the crude product. The residue was purified by column chromatography (SiO₂, Petroleum ether/Ethyl acetate=100/0 to 72/28) to give 35-2. ¹H NMR (400 MHz, CDCl₃) δ ppm 8.08 (s, 1H), 7.38-7.30 (m, 1H), 7.25-7.17 (m, 3H), 7.01-6.90 (dd, J=1.6 Hz, J=8.8 Hz, 1H), 6.67-6.60 (m, 1H), 6.23-6.15 (m, 1H), 5.91 (s, 1H), 4.90 (brs, 1H), 4.27-4.20 (m, 2H), 2.29-2.74 (m, 2H), 1.72-1.59 (m, 2H), 1.40-1.27 (m, 6H), 0.90-0.76 (m, 3H), 0.25 (s, 9H).

To a solution of 35-2 (48 mg, 87.00 μmol, 1 eq) in DCM (5 mL)/MeOH (0.5 mL) were added K₂CO₃ (48.10 mg, 348.02 μmol, 4 eq) at 0° C. The mixture was stirred at 0° C. for 2 hr to give a yellow solution. TLC (PE/EtOAc=2/1) showed the reaction was completed. The mixture was diluted with DCM (5 mL) and added H₂O (5 mL). The organic layer was extracted with DCM (10 mL*3) and dried over Na₂SO₄ and concentrated to give the crude product. The residue was purified by column chromatography (SiO₂, Petroleum ether/Ethyl acetate=100/0 to 67/33) to give B-35. LC-MS (m/z): 502.0 [M+Na]⁺

¹H NMR (400 MHz, CDCl₃) δ ppm 8.19-8.03 (m, 1H), 7.23-5.87 (m, 7H), 5.00-3.84 (m, 3H), 3.29-2.80 (m, 3H), 1.77-1.61 (m, 2H), 1.39-1.02 (m, 6H), 0.90-0.80 (m, 3H).

### Procedure B-8: Compound B-50

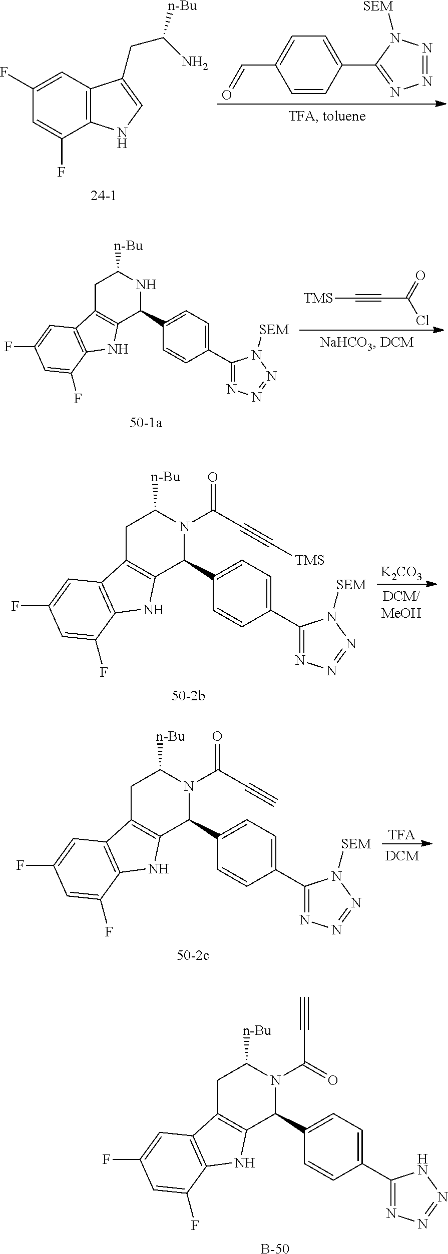

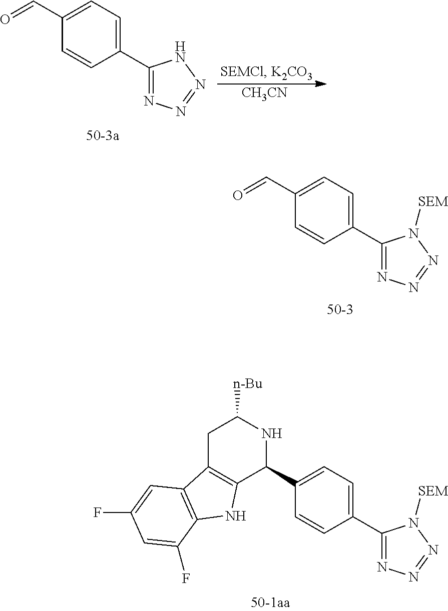

To a solution of 4-(1H-tetrazol-5-yl)benzaldehyde (430 mg, 2.47 mmol, 1 eq) in CH₃CN (10 mL) were added SEM-Cl (823.27 mg, 4.94 mmol, 873.95 uL, 2 eq) and K₂CO₃ (1.02 g, 7.41 mmol, 3 eq). The mixture was stirred at 85° C. for 12 hr to give a yellow suspension. TLC (eluting with: PE/EtOAc=3/1) showed the reaction was completed. The reaction mixture was concentrated to give the crude product. The crude product was purified by flash column (eluting with: PE/EtOAc=0% to 25%) to give 50-3 and 50-3. ¹H NMR (400 MHz, CDCl₃) δ=10.31 (s, 1H), 8.38 (d, J=8.4 Hz, 2H), 8.38 (d, J=6.8 Hz, 2H), 6.25-5.90 (m, 2H), 3.95-3.50 (m, 3H), 1.02-0.84 (m, 3H), 0.19 (s, 9H).

To a solution of 50-5 (150 mg, 594.53 μmol, 1 eq) in toluene (10 mL) were added 50-3 (180.98 mg, 594.53 μmol, 1 eq) and TFA (67.79 mg, 594.53 μmol, 44.02 uL, 1 eq). The mixture was stirred at 120° C. for 2 hr to give a yellow solution. TLC (eluting with: PE/EtOAc=1/1) showed the reaction was completed. The reaction mixture was basified to pH=8 with Et₃N. The mixture was purified by flash column (eluting with: PE/EtOAc=0% to 35%) to give 50-1a and 50-1aa. ¹H NMR (400 MHz, CDCl₃) δ=8.18-8.00 (m, 2H), 7.65-7.58 (m, 2H), 7.51 (s, 1H), 7.00-6.94 (m, 1H), 6.70-6.62 (m, 1H), 5.93-5.70 (m, 2H), 5.28 (s, 1H), 3.82-3.72 (m, 2H), 3.20-3.10 (m, 1H), 2.86-2.80 (m, 1H), 2.59-2.48 (m, 1H), 1.67-1.60 (m, 3H), 1.45-1.25 (m, 5H), 1.00-0.90 (m, 3H).

To a solution of 50-1a (60 mg, 111.38 um μmol, 1 eq) in DCM (5 mL) were added NaHCO₃ (28.07 mg, 334.13 μmol, 13.00 uL, 3 eq) and 3-trimethylsilylprop-2-ynoyl chloride (0.141 M, 947.90 uL, 1.2 eq) at 0° C. The mixture was stirred at 0° C. for 1 hr to give a yellow solution. LCMS and TLC (eluting with: PE/EtOAc=3/1) showed the reaction was completed. The mixture was quenched with H₂O (10 mL) and extracted with DCM (20 mL*3). The organic layers were dried over Na₂SO₄ and concentrated to the crude product. The crude product was purified by prep-TLC (eluting with: PE/EtOAc=3/1) to give 50-2b.

To a solution of 50-2b (40 mg, 60.34 μmol, 1 eq) in DCM (5 mL)/MeOH (2 mL) were added K₂CO₃ (25.02 mg, 181.02 μmol, 3 eq) at 0° C. The mixture was stirred at 0° C. for 0.5 hr to give a yellow solution. LCMS showed the reaction was completed. The reaction mixture was quenched with H₂O (15 mL) and extracted with DCM (20 mL*3). The organic layers were dried over Na₂SO₄ and concentrated to give 50-2c. Used for next step without further purification.

To a solution of 50-2c (20 mg, 33.86 μmol, 1 eq) in DCM (3 mL) was added TFA (308.00 mg, 2.70 mmol, 0.2 mL, 79.79 eq). The mixture was stirred at 20° C. for 2 hr to give a yellow solution. LCMS showed the reaction was completed. The reaction mixture was combined with ES5350-1459 for purification. The reaction mixture was concentrated to give the crude product. The crude product was purified by prep-HPLC (column: Welch Xtimate C18 150*25 mm*5 um; mobile phase: [water (0.05% HCl)-ACN]; B %: 55%-85%, 8.5 min) to give B-50. LC-MS (m/z): 461.1[M+H]⁺

¹H NMR (400 MHz, MeOD) δ=8.10-7.90 (m, 2H), 7.63-7.58 (m, 2H), 7.09-7.02 (m, 1H), 6.79-6.68 (m, 1H), 6.03 (s, 1H), 4.10-4.03 (m, 1H), 3.53-3.48 (m, 1H), 3.20-3.04 (m, 1H), 1.68-1.33 (m, 6H), 0.91 (t, J=7.2 Hz, 3H)

### Procedure B-8: Compound B-51

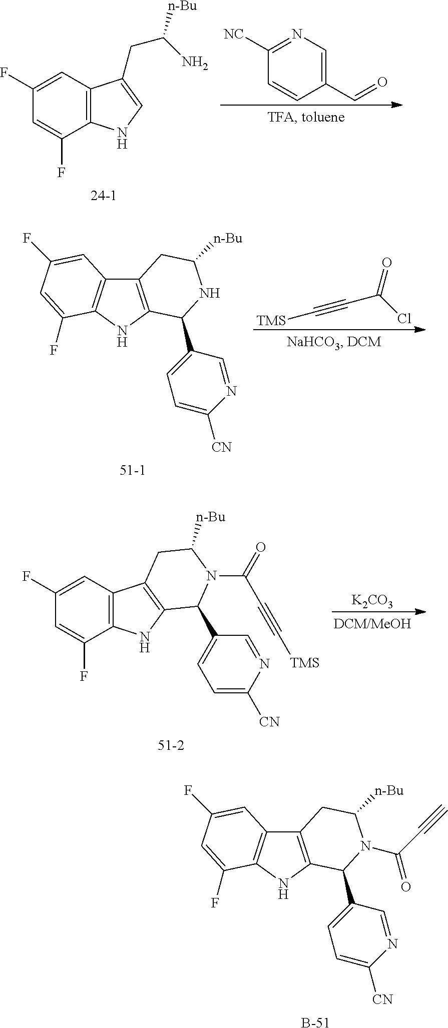

To a solution of 24-1 (200 mg, 792.70 μmol, 1 eq) in toluene (10 mL) were added 5-formylpyridine-2-carbonitrile (104.73 mg, 792.70 μmol, 1 eq) and TFA (90.39 mg, 792.70 μmol, 58.69 uL, 1 eq). The reaction mixture was stirred at 120° C. for 2 hr to give a yellow solution. TLC (eluting with: PE/EtOAc=3/1) showed the reaction was completed. The reaction mixture was basified to pH=8 with Et₃N. The reaction mixture was purified by flash column (eluting with: PE/EtOAc=0% to 70%) to give 51-1. ¹H NMR (400 MHz, CDCl₃) δ=8.72 (s, 1H), 7.92 (s, 1H), 7.67 (s, 2H), 7.02 (dd, J=2.0 Hz, J=8.8 Hz, 1H), 6.77 (t, J=1.6 Hz, 1H), 5.31 (s, 1H), 2.92-2.80 (m, 2H), 2.49-2.42 (m, 1 Hz), 1.52-1.26 (m, 6H), 0.91 (t, J=6.8 Hz, 3H).

To a solution of 51-1 (60 mg, 163.75 μmol, 1 eq) in DCM (5 mL) were added NaHCO₃ (68.78 mg, 818.76 μmol, 31.84 uL, 5 eq) and 3-trimethylsilylprop-2-ynoyl chloride (0.141 M, 2.32 mL, 2 eq). The mixture was stirred at 0° C. for 0.5 hr to give a yellow solution. LCMS and TLC (eluting with: PE/EtOAc=2/1) showed the reaction was completed. The reaction mixture was quenched with H₂O (15 mL) and extracted with DCM (20 mL*3). The organic layers were dried over Na₂SO₄ and concentrated to give the crude product. The crude product was purified by flash column (eluting with: PE/EtOAc=0% to 50%) to give 51-2. ¹H NMR (400 MHz, CDCl₃) δ=8.50 (s, 1H), 7.68 (s, 1H), 7.54-7.50 (m, 1H), 7.39 (d, J=8.0 Hz, 1H), 6.81 (d, J=6.4 Hz, 1H), 6.57 (t, J=2.0 Hz, 1H), 5.71 (s, 1H), 4.84 (brs, 1H), 3.09 (dd, J=4.4 Hz, J=15.6 Hz, 2H), 2.86 (d, J=14.8 Hz, 1H), 1.45-1.10 (m, 6H), 0.70 (t, J=6.8 Hz, 3H), 0.70 (s, 9H).

To a solution of 51-2 (47 mg, 95.80 μmol, 1 eq) in DCM (5 mL)/MeOH (2 mL) was added K₂CO₃ (26.48 mg, 191.59 μmol, 2 eq) at 0° C. The mixture was stirred at 0° C. for 0.5 hr to give a yellow solution. LCMS showed the reaction was completed. The reaction mixture was quenched with H₂O (20 mL) and extracted with DCM (20 mL*3). The organic layers were dried over Na₂SO₄ and concentrated to give the crude product. The crude product was purified by flash column (PE/EtOAc=0% to 50%) to give B-51. LC-MS (m/z): 419.1 [M+1-1]⁺

¹H NMR (400 MHz, CDCl₃) δ=8.69 (s, 1H), 8.18-8.14 (m, 1H), 7.64-7.60 (m, 1H), 7.51 (d, J=8.0 Hz, 1H), 6.94 (dd, J=2.0 Hz, J=8.8 Hz, 1H), 6.69 (t, J=2.0 Hz, 1H), 5.85 (s, 1H), 5.12-4.92 (m, 1H), 3.22 (dd, J=4.4 Hz, J=15.2 Hz, 1H), 3.13 (s, 1H), 2.98 (d, J=15.6 Hz, 1H), 1.56-1.50 (m, 2H), 1.30-1.20 (m, 4H), 0.81 (t, J=6.8 Hz, 3H).

### Procedure B-8: Compound B-53

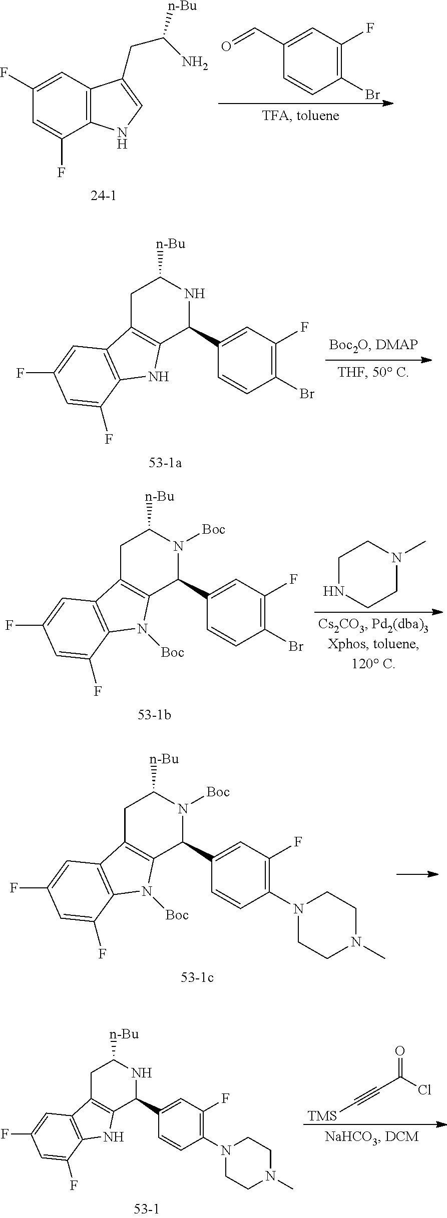

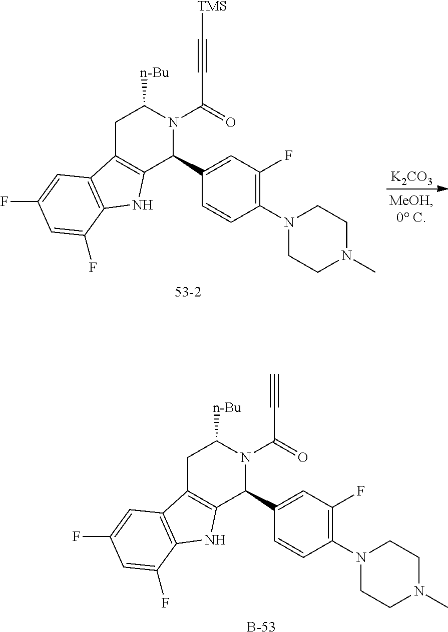

To a solution of 24-1 (700 mg, 2.77 mmol, 1 eq) in toluene (20 mL) were added 4-bromo-3-fluorobenzaldehyde (563.24 mg, 2.77 mmol, 1 eq) and TFA (474.53 mg, 4.16 mmol, 308.13 uL, 1.5 eq). The mixture was stirred at 120° C. for 2 hr top give a yellow suspension. TLC (eluting with: PE/EtOAc=2/1) showed the reaction was completed. The reaction mixture was concentrated to give the residue. The residue was dissolved in DCM (20 mL) and basified to pH=8 with Et₃N. The mixture was purified by flash column (eluting with: PE/EtOAc=100% PE to 30%) to give 53-1a. ¹H NMR (400 MHz, CDCl₃) δ=7.79 (s, 1H), 7.58-7.45 (m, 1H), 7.07-6.92 (m, 2H), 6.90-6.85 (m, 1H), 6.78-6.69 (m, 1H), 5.24 (s, 1H), 3.00-2.90 (m, 1H), 2.89 (dd, J=4.0 Hz, J=15.2 Hz, 1H), 2.50-2.42 (m, 1H), 1.52-1.47 (m, 2H), 1.38-1.25 (m, 4H), 0.93 (t, J=6.8 Hz, 3H).

To a solution of 53-1a (260 mg, 594.56 μmol, 1 eq) in THF (10 mL) were added Boc₂O (1.04 g, 4.76 mmol, 1.09 mL, 8 eq) and DMAP (3.63 mg, 29.73 μmol, 0.05 eq). The mixture was stirred at 40° C. for 40 hr to give a yellow solution. TLC (eluting with: PE/EtOAc=8/1) showed the reaction was completed. The reaction mixture was quenched with H₂O (30 mL) and extracted with EtOAc (30 mL*3). The organic layers were dried over Na₂SO₄ and concentrated to give the crude product. The crude product was purified by flash column (eluting with: PE/EtOAc=100% PE to 10%) to give 53-1b. ¹H NMR (400 MHz, CDCl₃) δ=7.40-7.35 (m, 1H), 7.00-6.92 (m, 1H), 6.87-6.80 (m, 2H), 6.79-6.68 (m, 1H), 6.65-6.63 (m, 1H), 3.45 (brs, 1H), 2.86-2.74 (m, 1H), 2.64 (dd, J=4.0 Hz, J=16.0 Hz, 1H), 2.05-1.87 (m, 1H), 1.52-1.47 (m, 18H), 1.25-0.94 (m, 6H), 0.76 (t, J=6.8 Hz, 3H).

To a solution of 53-1b (400 mg, 627.43 μmol, 1 eq) in toluene (10 mL) were added 1-methylpiperazine (314.22 mg, 3.14 mmol, 347.97 uL, 5 eq), Cs₂CO₃ (1.02 g, 3.14 mmol, 5 eq), XPhos (119.64 mg, 250.97 μmol, 0.4 eq) and Pd₂(dba)₃ (114.91 mg, 125.49 μmol, 0.2 eq) at N₂. The mixture was stirred at 120° C. for 12 hr to give a yellow suspension. LCMS and TLC (eluting with: EtOAc/MeOH=10/1) showed the reaction was completed. The reaction mixture was diluted with DCM (20 mL). The mixture was purified by prep-flash column (eluting with: EtOAc/MeOH=10/1) to give 53-1c. ¹H NMR (400 MHz, CDCl₃) δ=6.89-6.64 (m, 6H), 3.02-2.95 (m, 4H), 2.78-2.42 (m, 6H), 2.23 (s, 3H), 2.08-1.95 (m, 1H), 1.39 (s, 9H), 1.31 (s, 9H), 1.06-0.73 (m, 6H), 0.72 (t, J=6.8 Hz, 3H).

53-1c (180 mg, 274.07 μmol, 1 eq) was dissolved in HCl/dioxane (4 M, 10 mL, 145.95 eq) at 0° C. The mixture was allowed to stir at 20° C. for 12 hr to give a yellow solution. LCMS showed the reaction was completed. The reaction mixture was basified to pH=8 with Sat.NaHCO₃. The mixture was concentrated to give the crude product. The crude product was dissolved in DCM/EtOH (10/1, 60 mL). The mixture was stirred at 20° C. for 0.5 hr. The mixture was filtered and washed with DCM (30 mL*2). The filtrate was concentrated to give 53-1. ¹H NMR (400 MHz, CDCl₃) δ=8.18 (brs, 1H), 7.05-6.96 (m, 1H), 6.94-6.86 (m, 3H), 6.76-6.70 (m, 1H), 6.15 (s, 1H), 3.15-2.90 (m, 5H), 2.48-2.40 (m, 1H), 2.37 (s, 3H), 1.52-1.22 (m, 6H), 0.91 (t, J=6.8 Hz, 3H).

To a solution of 53-1 (54 mg, 118.28 μmol, 1 eq) in DCM (5 mL)/H2O (1 mL) were added NaHCO₃ (99.36 mg, 1.18 mmol, 46.00 uL, 10 eq) and 3-trimethylsilylprop-2-ynoyl chloride (0.2 M, 650.54 uL, 1.1 eq) at 0° C. The reaction mixture was stirred at 0° C. for 0.5 hr to give a yellow solution. LCMS and TLC (eluting with: EtOAc/MeOH=5/1) showed the reaction was completed. The reaction mixture was quenched with H₂O (10 mL) and extracted with DCM (30 mL*3). The organic layers were dried over Na₂SO₄ and concentrated to give the crude product. The crude product was purified by prep-TLC (eluting with: EtOAc/MeOH=5/1) to give 53-2.

To a solution of 53-2 (40 mg, 68.88 μmol, 1 eq) in DCM (5 mL)/MeOH (0.5 mL) was added K₂CO₃ (10.47 mg, 75.76 μmol, 1.1 eq) at 0° C. The reaction mixture was stirred at 0° C. for 0.5 hr to give a yellow solution. LCMS and TLC (eluting with: EtOAc/MeOH=5/1) showed the reaction was completed. The reaction mixture was quenched with DCM (30 mL*3). The organic layers were dried over Na₂SO₄ and concentrated to give the crude product. The crude product was purified by prep-TLC (eluting with: EtOAc/MeOH=5/1) to give B-53. LC-MS (m/z): 509.1 [M+H]⁺

¹H NMR (400 MHz, CDCl₃) δ=8.25-7.78 (m, 1H), 6.98-6.60 (m, 5H), 6.50-6.73 (m, 1H), 6.89-6.60 (m, 1H), 3.20-2.66 (m, 7H), 2.59-2.44 (m, 4H), 2.32 (s, 1H), 1.09-0.85 (m, 6H), 0.80-0.72 (m, 3H).

### Procedure B-8: Compound B-54

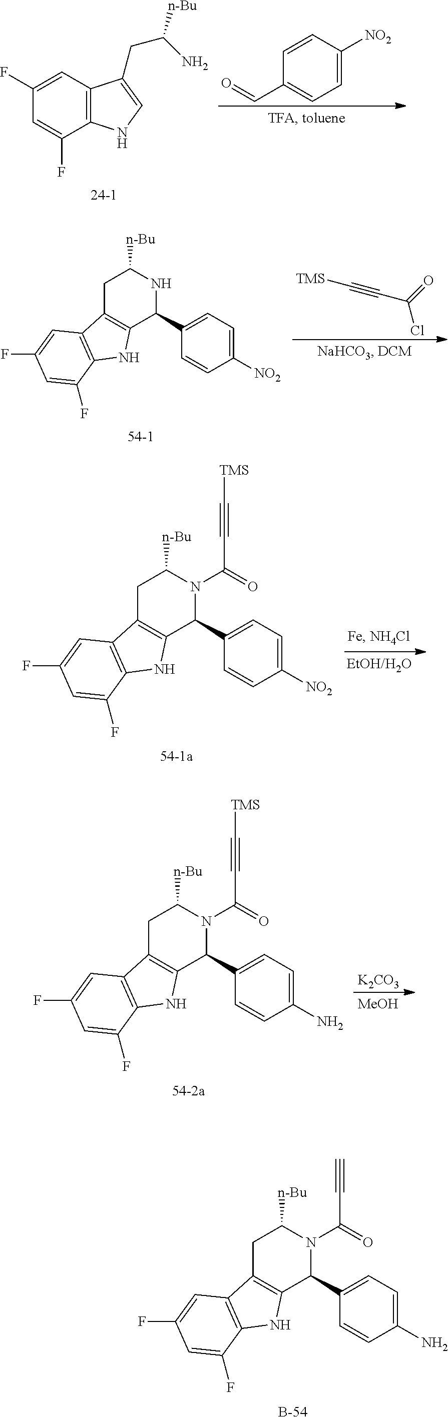

To a solution of 24-1 (200 mg, 792.70 μmol, 1 eq) in toluene (10 mL) were added 4-nitrobenzaldehyde (119.79 mg, 792.70 μmol, 1 eq) and TFA (90.39 mg, 792.70 μmol, 58.69 uL, 1 eq). The mixture was stirred at 100° C. for 12 hr to give a yellow solution. LCMS and TLC (eluting with: PE/EtOAc=2/1) showed the reaction mixture was completed. The reaction mixture was concentrated to give the residue. The residue was dissolved in DCM (10 mL) and basified to pH=8 with Et₃N. The mixture was purified by flash column (eluting with: PE/EtOAc=100% to 30%) to give 54-1. ¹H NMR (400 MHz, CDCl₃) δ ppm 8.20 (d, J=8.8 Hz, 2H), 7.83 (brs, 1H), 7.43 (d, J=8.8 Hz, 2H), 7.01 (dd, J=2.0 Hz, J=8.8 Hz, 1H), 6.77 (t, J=7.2 Hz, 1H), 5.30 (s, 1H), 2.95-2.84 (m, 2H), 2.48-2.2.43 (m, 1H), 1.57-1.47 (m, 3H), 1.45-1.28 (m, 3H), 0.89 (t, J=7.2 Hz, 3H).

To a solution of 54-1 (270 mg, 700.56 μmol, 1 eq) in DCM (2 mL) was added NaHCO₃ (470.81 mg, 5.60 mmol, 217.97 uL, 8 eq) and 3-trimethylsilylprop-2-ynoyl chloride (0.1 M, 14.01 mL, 2 eq) at 0° C. The reaction mixture was allowed to stir at 10° C. for 1.5 hr to give a yellow suspension. TLC (PE/EtOAc=3:1) showed the mixture was completed. The reaction mixture was quenched with H₂O (20 mL) and extracted with DCM (20 mL*3). The organic layers were dried over Na₂SO₄ and concentrated to give 54-1a.

To a solution of 54-1a (381 mg, 747.62 μmol, 1 eq) in EtOH (20 mL)/H₂O (5 mL) were added NH₄C₁ (799.82 mg, 14.95 mmol, 20 eq) and Fe (417.51 mg, 7.48 mmol, 10 eq). The mixture was stirred at 15° C. for 12 hr to give a yellow suspension. TLC (PE/EtOAc=3:1) showed the reaction mixture was completed. The reaction mixture was filtered on celite. The filtrate was extracted with EtOAc (30 mL*3). The organic layers were dried over Na₂SO₄ and concentrated to give the crude product as a yellow solid. The crude product was purified by flash column (eluting with: 0% EtOAc in PE to 23%) to give 54-2a.

To a solution of 54-2a (86 mg, 179.30 μmol, 1 eq) in DCM (5 mL)/MeOH (0.5 mL) were added K₂CO₃ (27.26 mg, 197.23 μmol, 1.1 eq) at 0° C. The mixture was stirred at 0° C. for 2 hr to give a yellow suspension. TLC (eluting with: PE/EtOAc=1/1) showed the mixture was completed. The mixture was diluted with DCM (5 mL) and added H₂O (5 mL). The organic layer was extracted with DCM (20 mL*3) and dried over Na₂SO₄ and concentrated to give the crude product. The crude product was purified by flash column (eluting with: 0% EtOAc in PE to 35% EtOAc in PE) to give B-54. LC-MS (m/z): 408.1 [M+H]⁺

¹H NMR (400 MHz, CHLOROFORM-d) δ ppm 7.80-7.76 (m, 1H), 7.09-6.94 (m, 3H), 6.75-6.55 (m, 4H), 4.90-4.88 (m, 1H), 3.77-3.60 (m, 2H), 3.22-2.75 (m, 4H), 1.81-1.64 (m, 2H), 1.44-1.25 (m, 4H), 0.91-0.75 (m, 3H)

### Procedure B-8: Compound B-57

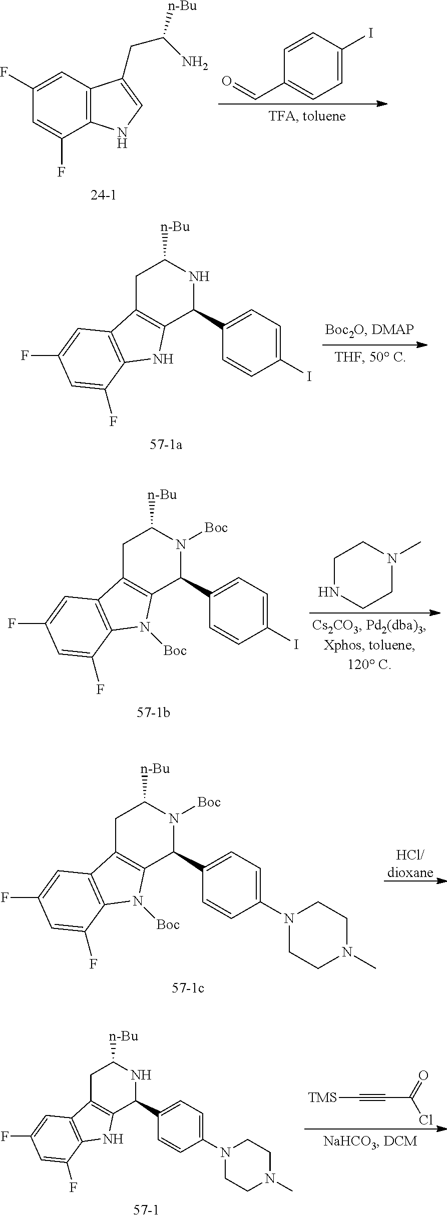

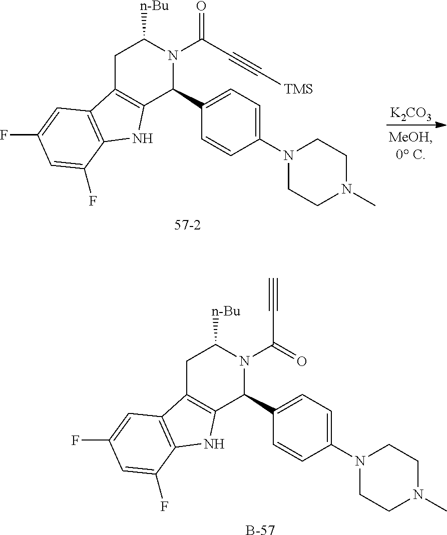

To a solution of 24-1 (500 mg, 1.98 mmol, 1 eq) in toluene (10 mL) were added 4-iodobenzaldehyde (459.80 mg, 1.98 mmol, 1 eq) and TFA (338.95 mg, 2.97 mmol, 220.10 uL, 1.5 eq). The reaction mixture was stirred at 120° C. for 12 hr to give a yellow solution. LCMS and TLC (eluting with: PE/EtOAc=2/1) showed the reaction was completed. The reaction mixture was basified to pH=8 with Et₃N. The mixture was purified by flash column (eluting with: PE/EtOAc=100% to 30%) to give 57-1a. ¹H NMR (400 MHz, CDCl₃) δ=7.90 (s, 1H), 7.67 (d, J=8.0 Hz, 2H), 7.05-6.94 (m, 3H), 6.75 (t, J=2.0 Hz, 1H), 5.15 (s, 1H), 3.05-2.96 (m, 2H), 2.48-2.42 (m, 1H), 1.51-1.30 (m, 6H), 0.91 (t, J=7.2 Hz, 3H).

To a solution of 57-1b (370 mg, 793.47 μmol, 1 eq) in THF (10 mL) were added Boc₂O (1.39 g, 6.35 mmol, 1.46 mL, 8 eq) and DMAP (9.69 mg, 79.35 μmol, 0.1 eq). The reaction mixture was stirred at 50° C. for 12 hr to give a yellow solution. LCMS and TLC (eluting with: PE/EtOAc=10/1) showed the reaction was completed. The reaction mixture was diluted with DCM (15 mL). The mixture was purified by flash column (eluting with: PE/EtOAc=100% to 10%) to give 57-1b. ¹H NMR (400 MHz, CDCl₃) δ=7.53 (d, J=12.4 Hz, 2H), 6.90-6.64 (m, 5H), 3.36 (brs, 1H), 2.85-2.74 (m, 1H), 2.59 (d, J=4.0 Hz, J=16.0 Hz, 1H), 2.07-1.98 (m, 1H), 1.40 (s, 9H), 1.31 (s, 9H), 1.15-0.97 (m, 6H), 0.85-0.69 (m, 3H).

To a solution of 57-1b (150 mg, 225.04 μmol, 1 eq) in toluene (10 mL) were added 1-methylpiperazine (27.05 mg, 270.05 μmol, 29.95 uL, 1.2 eq), Cs₂CO₃ (219.97 mg, 675.13 μmol, 3 eq), XPhos (21.46 mg, 45.01 μmol, 0.2 eq) and Pd₂(dba)₃ (20.61 mg, 22.50 μmol, 0.1 eq) under N₂. The mixture was stirred at 120° C. under N₂ for 12 hr to give a yellow solution. LCMS and TLC (eluting with: EtOAc/MeOH=10/1) showed the reaction was completed. The reaction mixture diluted with DCM (30 mL). The mixture was purified by flash column (eluting with: EtOAc/MeOH=100% to 15%) to give 57-1c. ¹H NMR (400 MHz, CDCl₃) δ=6.99-6.83 (m, 2H), 6.82-6.75 (m, 1H), 6.75-6.68 (m, 4H), 3.33 (brs, 1H), 3.25-3.05 (m, 4H), 2.85-2.77 (m, 1H), 2.50 (s, 3H), 2.35-2.02 (m, 1H), 1.48 (s, 9H), 1.25 (s, 9H), 1.15-0.94 (m, 6H), 0.71 (t, J=6.8 Hz, 3H).

57-1 (100 mg, 156.55 μmol, 1 eq) was dissolved in HCl/dioxane (4 M, 10 mL, 255.51 eq). The mixture was stirred at 10° C. for 1 hr. LCMS showed no desired mass was found, and 57-1 was remained. The mixture was stirred at 15° C. for 12 hr again to give a yellow solution. LCMS showed the reaction was completed. The reaction mixture was concentrated to give the crude product. The crude product was basified to 8 with Sat.NaHCO₃. The mixture was concentrated to give 57-2. Used for next step without further purification.

To a solution of 57-1 (68 mg, 155.05 μmol, 1 eq) in DCM (5 mL) were added NaHCO₃ (104.20 mg, 1.24 mmol, 48.24 uL, 8 eq) and 3-trimethylsilylprop-2-ynoyl chloride (49.83 mg, 310.11 μmol, 2 eq) at 0° C. The reaction mixture allowed to stir at 15° C. for 12 hr to give a yellow suspension. LCMS and TLC (eluting with: EtOAc/MeOH=5/1) showed the reaction was completed. The reaction mixture was filtered on celite. The filtrate was concentrated to give the crude product. The crude product was purified by prep-TLC (eluting with: EtOAc/MeOH=5/1) to give 57-2.

To a solution of 57-2 (20 mg, 35.54 μmol, 1 eq) in MeOH (5 mL) was added K₂CO₃ (5.89 mg, 42.65 lima 1.2 eq). The mixture was stirred at 0° C. for 1 hr to give a yellow solution. LCMS and TLC (eluting with: EtOAc/MeOH=5/1) showed the reaction was completed. The reaction mixture was concentrated to give the crude product with purging with N₂. The crude product was purified by prep-TLC (eluting with: EtOAc/MeOH=5/1) to give B-57. LC-MS (m/z): 491.2 [M+1-1]⁺

¹H NMR (400 MHz, CDCl₃) δ=7.96-7.67 (m, 1H), 7.15-7.08 (m, 2H), 6.95-6.50 (m, 5H), 4.88-3.68 (m, 1H), 3.35-3.15 (m, 5H), 3.13-2.87 (m, 4H), 2.69-2.50 (m, 1H), 2.48-2.38 (m, 4H), 1.42-1.30 (m, 6H), 0.75-0.68 (m, 3H).

### Procedure B-8: Compound B-58

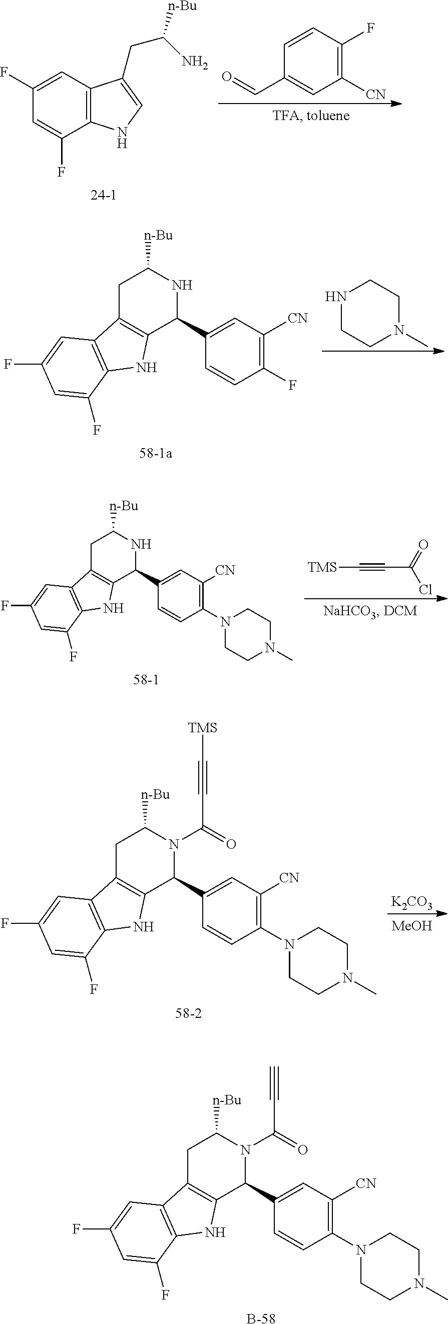

To a solution of 24-1 (300 mg, 1.19 mmol, 1 eq) in toluene (20 mL) were added 2-fluoro-5-formyl-benzonitrile (177.31 mg, 1.19 mmol, 1 eq) and TFA (203.37 mg, 1.78 mmol, 132.06 uL, 1.5 eq). The mixture was stirred at 120° C. for 12 hr to give a yellow solution. TLC (eluting with: PE/EtOAc=2/1) showed the reaction was completed. The reaction mixture was diluted with DCM (30 mL) and basified to pH=8 with. The mixture was flash column (eluting with: PE/EtOAc=100% PE to 30%) to give 58-1a. ¹H NMR (400 MHz, CDCl₃) δ=7.76 (brs, 1H), 7.35-7.30 (m, 2H), 7.03 (t, J=8.8 Hz, 1H), 6.85-6.70 (dd, J=2.4 Hz, J=8.8 Hz, 1H), 6.59 (t, J=1.6 Hz, 1H), 5.03 (s, 1H), 2.75-2.60 (m, 2H), 2.30-2.18 (m, 1H), 1.45-1.05 (m, 6H), 0.73 (t, J=6.8 Hz, 3H).

To a solution of 58-1a (130 mg, 339.06 μmol, 1 eq) in CH₃CN (5 mL) was added 1-methylpiperazine (101.88 mg, 1.02 mmol, 112.83 uL, 3 eq). The mixture was stirred at 100° C. for 12 hr to give yellow solution. LCMS showed no desired mass was found and 58-1a was remained. The reaction was stirred at 100° C. for 12 hr to give a yellow solution. LCMS showed the reaction was completed. The reaction mixture was concentrated to give concentrated to give 58-1. Used for next step without further purification. ¹H NMR (400 MHz, CDCl₃) δ=8.50 (brs, 1H), 7.27-7.20 (m, 1H), 6.95-6.80 (m, 2H), 6.64-6.60 (m, 1H), 3.65-3.51 (m, 4H), 3.35-3.25 (m, 2H), 3.18-3.08 (m, 5H), 1.48-1.20 (m, 6H), 0.82 (t, J=3.2 Hz, 3H).

To a solution of 58-1 (250.00 mg, 539.30 μmol, 1 eq) in DCM (5 mL)/H₂O (1 mL) were added NaHCO₃ (453.07 mg, 5.39 mmol, 209.75 uL, 10 eq) and 3-trimethylsilylprop-2-ynoyl chloride (0.176 M, 9.19 mL, 3 eq) at 0° C. The reaction mixture was stirred at 0° C. for 0.5 hr to give a yellow solution. LCMS and TLC (eluting with: EtOAc/MeOH=5/1) showed the reaction was completed. The reaction mixture was quenched with H₂O (10 mL) and extracted with DCM (30 mL*3). The organic layers were dried over Na₂SO₄ and concentrated to give the crude product. The crude product was purified by prep-TLC (eluting with: EtOAc/MeOH=5/1) to give 58-2.

To a solution of 58-2 (200 mg, 340.27 μmol, 1 eq) in DCM (10 mL)/MeOH (1 mL) were added K₂CO₃ (51.73 mg, 374.29 μmol, 1.1 eq) at 0° C. The mixture was stirred at 0° C. for 1 hr to give a yellow solution. LCMS and TLC (eluting with: DCM/MeOH=10/1) showed the reaction was completed. The reaction mixture was quenched with H₂O (10 mL) and extracted with DCM (20 mL*3). The organic layers were dried over Na₂SO₄ and concentrated to give the crude product. The crude product was purified by flash column (eluting with: EtOAc/MeOH=0% to 10%) to give 30 mg of desired product. But it was not impure. The 30 mg was purified by prep-TLC (eluting with: DCM/MeOH=10/1) to give B-58. LC-MS (m/z):

516.1 [M+H]⁺ⁱH NMR (400 MHz, CDCl₃) δ=7.76-7.32 (m, 2H), 6.98-6.80 (m, 3H), 6.70-6.65 (m, 1H), 4.91 (brs, 1H), 3.25-3.15 (m, 5H), 3.08 (s, 1H), 3.00-2.89 (m, 1H), 2.65-2.58 (m, 4H), 2.34 (s, 3H), 1.32-1.18 (m, 6H), 0.85-0.75 (m, 3H)

### Procedure B-8: Compound B-59

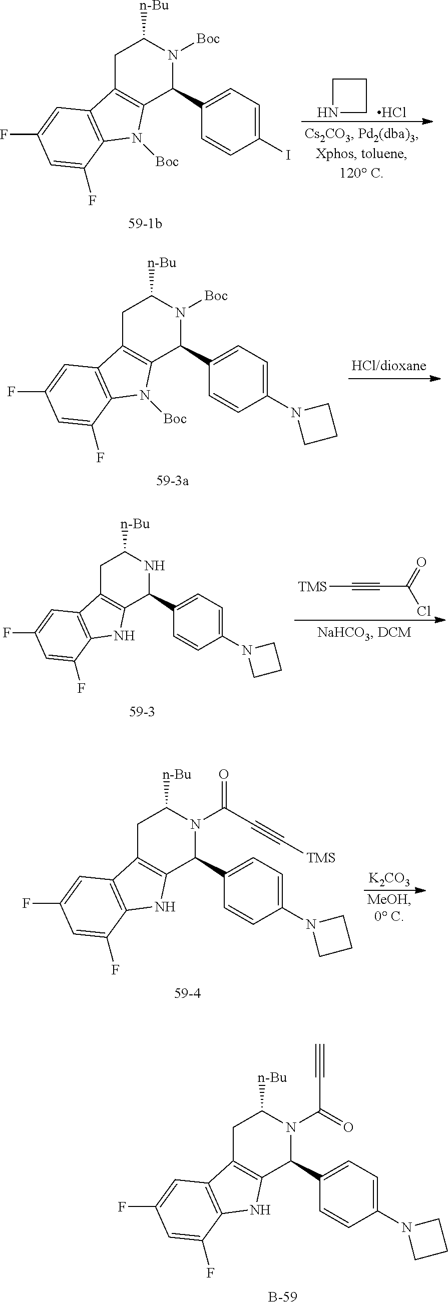

To a solution of 59-1b (250 mg, 375.07 μmol, 1 eq) in toluene (10 mL) were added azetidine (175.45 mg, 1.88 mmol, 207.39 uL, 5 eq, HCl), Cs₂CO₃ (977.65 mg, 3.00 mmol, 8 eq), XPhos (71.52 mg, 150.03 μmol, 0.4 eq) and Pd₂(dba)₃ (68.69 mg, 75.01 μmol, 0.2 eq) under N₂. The reaction mixture was stirred at 120° C. for 3 hr to give a yellow solution. TLC (eluting with: PE/EtOAc=8/1) showed the reaction was completed. The reaction mixture was concentrated to give the crude product. The crude product was purified by flash column (eluting with: PE/EtOAc=100% PE to 20%) to give 59-3a. ¹H NMR (400 MHz, CDCl₃) δ=7.58-7.34 (m, 1H), 6.90-6.78 (m, 3H), 6.70-6.68 (m, 2H), 6.27 (s, J=6.4 Hz, 2H), 3.90-3.70 (m, 4H), 2.29-2.04 (m, 2H), 1.37 (s, 9H), 1.26 (s, 9H), 0.98-0.80 (m, 7H), 0.79-0.70 (m, 3H).

To a solution of 59-3a (250 mg, 419.66 μmol, 1 eq) in DCM (10 mL) was dissolved TFA (3.35 g, 29.36 mmol, 2.17 mL, 69.96 eq). The reaction mixture was stirred at 15° C. for 12 hr to give a yellow solution. TLC (eluting with: EtOAc/PE=2/1) showed the reaction was completed. The mixture was concentrated to give the crude product. The crude product was basified to pH=8 with Sat.NaHCO₃ and extracted with DCM (30 mL*3). The organic layers were dried over Na₂SO₄ and concentrated to give 59-3. Used for next step without further purification.

To a solution of 59-4 (40 mg, 101.14 μmol, 1 eq) in DCM (5 mL) was added 2-chloro-1-methyl-pyridin-1-ium; iodide (38.76 mg, 151.71 μmol, 1.5 eq) at 20° C. The mixture was stirred at 10° C. for 0.5 hr.

Then 3-trimethylsilylprop-2-ynoic acid (14.39 mg, 101.14 μmol, 1 eq) and Et₃N (15.35 mg, 151.71 mol, 21.12 uL, 1.5 eq) in DCM (5 mL) was added dropwise at 20° C. The mixture was stirred at 10° C. for 1 hr to give a yellow solution. LCMS showed the reaction was completed. The reaction mixture was quenched with (20 mL) and extracted with DCM (20 mL*3). The organic layers were dried over Na₂SO₄ and concentrated to give 59-4. Used for next step without further purification. ¹H NMR (400 MHz, CDCl₃) δ=7.28-7.22 (m, 3H), 6.55-6.48 (m, 2H), 6.15-6.05 (m, 2H), 3.85-3.70 (m, 2H), 3.08-2.90 (m, 4H), 2.32-2.20 (m, 2H), 1.38-1.18 (m, 8H), 0.82-0.77 (m, 3H), 0.20-0.08 (m, 9H).

To a solution of 59-4 (40 mg, 76.97 μmol, 1 eq) in DCM (5 mL)/MeOH (0.5 mL) was added K₂CO₃ (11.70 mg, 84.66 μmol, 1.1 eq) at 0° C. The mixture was stirred at 0° C. for 1 hr to give a yellow solution. LCMS and TLC (eluting with: PE/EtOAc=2/1) showed the reaction was completed. The reaction mixture was quenched with H₂O (10 mL) and extracted with DCM (30 mL*3). The organic layers were dried over Na₂SO₄ and concentrated to give the crude product. The crude product was purified by flash column (eluting with: PE/EtOAc=100% PE to 30%) to give B-59. LC-MS (m/z): 448.1[M+H]⁺

¹H NMR (400 MHz, CDCl₃) δ=7.90-7.66 (m, 1H), 7.05-6.86 (m, 3H), 6.69-6.60 (m, 1H), 6.35-6.20 (m, 2H), 5.79 (s, 1H), 4.81 (brs, 2H), 3.85-3.72 (m, 4H), 3.18-2.60 (m, 3H), 2.36-2.20 (m, 2H), 1.63-1.58 (m, 1H), 1.48-1.02 (m, 6H), 0.80-0.70 (m, 3H).

### Procedure B-8: Compound B-60

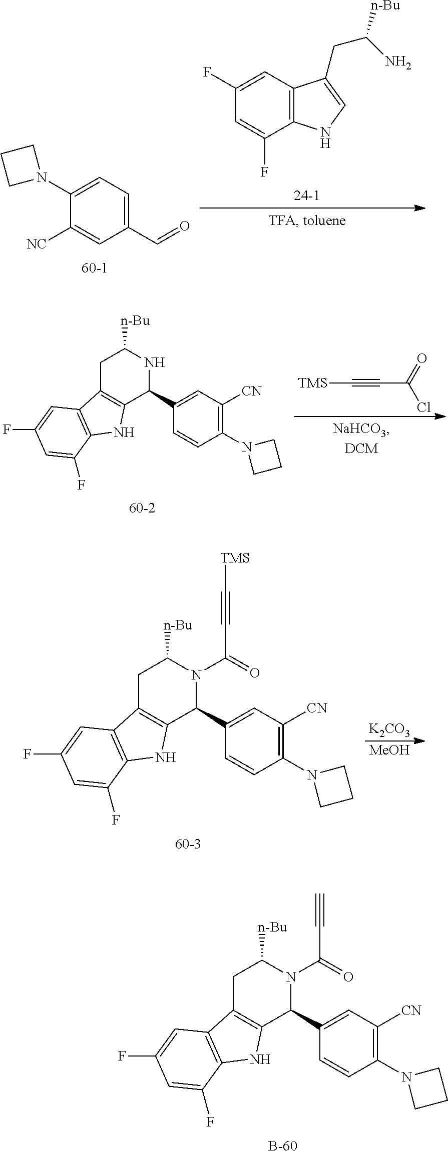

To a solution of 60-1 (200 mg, 1.07 mmol, 1 eq) in toluene (10 mL) were added 24-1 (270.99 mg, 1.07 mmol, 1 eq) and TFA (122.47 mg, 1.07 mmol, 79.52 uL, 1 eq). The mixture was stirred at 100° C. for 12 hr to give a yellow solution. TLC (PE:EtOAc=1:1) showed R1 and R2 was remained and have new spot found. The mixture was concentrated under vacuum and added TEA (2 mL). The residue was purified by flash column (SiO₂, Petroleum ether/Ethyl acetate=0% to 50%) to give 60-2. ¹H NMR (400 MHz, CDCl₃) δ ppm 7.73 (s, 1H), 7.23-7.20 (m, 1H), 7.17 (s, 1H), 7.02-6.96 (m, 1H), 6.79-6.67 (m, 1H), 6.38 (d, J=8.8 Hz, 1H), 5.09 (s, 1H), 4.21 (t, J=7.2 Hz, 4H), 3.02-2.92 (m, 1H), 2.89-2.80 (m, 1H), 2.47-2.34 (m, 3H), 1.54-1.47 (m, 2H), 1.38-1.29 (m, 4H), 0.91 (t, J=7.2 Hz, 3H).

To a solution of 60-2 (26 mg, 61.83 μmol, 1 eq) in DCM (5 mL)/H₂O (1 mL) were added NaHCO₃ (51.94 mg, 618.32 μmol, 24.05 uL, 10 eq) and 3-trimethylsilylprop-2-ynoyl chloride (0.2 M, 340.07 uL, 1.1 eq) at 0° C. The reaction mixture was stirred at 0° C. for 0.5 hr to give a yellow solution. LCMS and TLC (eluting with: PE/EtOAc=2/1) showed the reaction was completed. The reaction mixture was quenched with H₂O (10 mL) and extracted with DCM (30 ML*3). The organic layers were dried over Na₂SO₄ and concentrated to give the crude product. The crude product was purified by prep-TLC (eluting with: PE/EtOAc=2/1) to give 60-3.

To a solution of 60-3 (20 mg, 36.72 μmol, 1 eq) in DCM (5 mL)/MeOH (0.5 mL) was added K₂CO₃ (5.58 mg, 40.39 μmol, 1.1 eq) at 0° C. The reaction mixture was stirred at 0° C. for 0.5 hr to give a yellow solution. LCMS and TLC (eluting with: PE/EtOAc=2/1) showed the reaction was completed. The reaction mixture was quenched with H₂O (10 mL) and extracted with DCM (30 mL*3). The organic layers were dried over Na₂SO₄ and concentrated to give the crude product. The crude product was purified by prep-TLC (eluting with: PE/EtOAc=2/1) to give B-60. LC-MS (m/z): 473.1[M+H]⁺

¹H NMR (400 MHz, CDCl₃) δ=7.90-7.70 (m, 1H), 7.17-7.10 (m, 1H), 6.95-6.85 (m, 1H), 6.70-6.60 (m, 1H), 6.34-6.20 (m, 1H), 5.74-4.86 (m, 1H), 4.20-4.02 (m, 1H), 3.20-2.85 (m, 3H), 2.38-2.22 (m, 3H), 1.20-0.85 (m, 6H), 0.84-0.78 (m, 3H).

### Procedure B-8: Compound B-61

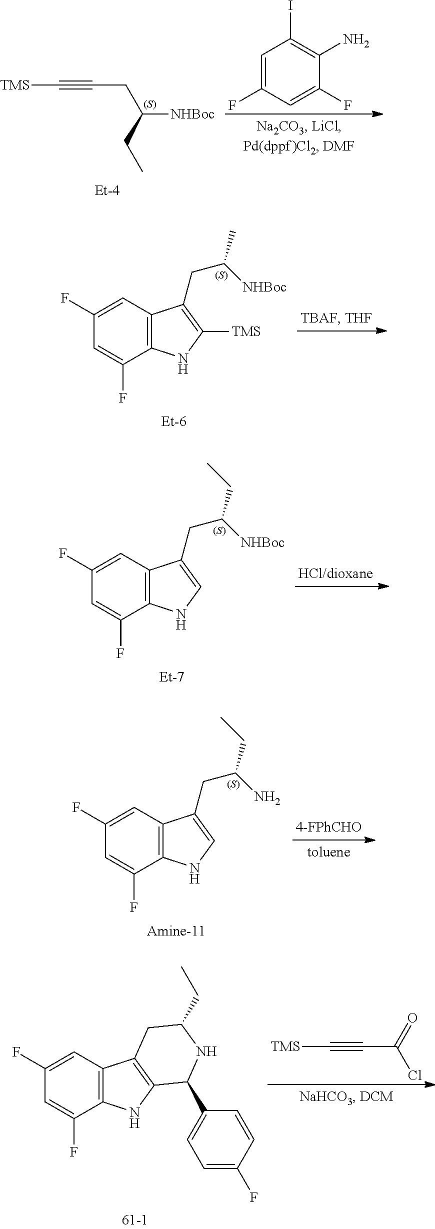

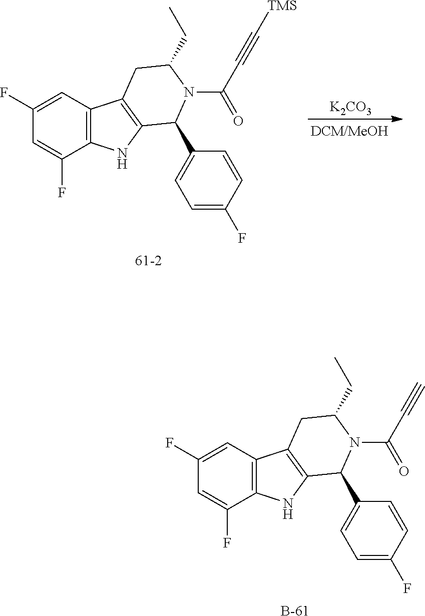

To a solution of 2,4-difluoro-6-iodoaniline (10 g, 39.22 mmol, 1 eq) and Et-4 (10.57 g, 39.22 mmol, 1 eq) in DMF (100 mL) were added Na₂CO₃ (8.31 g, 78.43 mmol, 2 eq), Pd(dppf)Cl₂.CH₂Cl₂ (2.8 g, 3.43 mmol, 8.74e-2 eq) and LiCl (1.66 g, 39.22 mmol, 803.13 uL, 1 eq) under N₂. The mixture was stirred at 100° C. under N₂ for 12 h to give a brown solution. TLC (PE/EtOAc=10:1) showed the reaction was completed. The reaction was diluted with EtOAc (60 mL) and filtered through celite. The filtrate was washed with brine (70 mL×3) and dried over sodium sulfate, filtered and concentrated to give the crude product. The crude product was purified by flash column (SiO₂, PE/EA from 100/0 to 95/5) to give Et-6. ¹H NMR (400 MHz, CDCl₃) δ ppm 8.04 (brs, 1H), 7.2-7.04 (m, 1H), 6.77-6.68 (m, 1H), 4.34 (brs, 1H), 3.84-3.70 (m, 1H), 305-2.94 (m, 1H), 2.86-2.74 (m, 1H), 1.66-1.57 (m, 1H), 1.42-1.39 (m, 1H), 1.43 (m, 9H), 0.91-0.95 (m, 3H), 0.42 (s, 9H).

To a solution of Et-6 (8.9 g, 22.44 mmol, 1 eq) in THF (50 mL) was added TBAF (1 M, 67.33 mL, 3 eq) at 0° C. The mixture was stirred at 30° C. for 12 h to give a brown solution. LCMS and TLC (PE/EtOAc=5:1) showed the reaction was completed. The reaction was diluted with H₂O (70 mL) and extracted with EtOAc (60 mL×3). The combined organic layers were washed with brine (60 mL) and dried over sodium sulfate, filtered and concentrated to give the crude product. The crude product was purified by flash column (SiO₂, EtOAc in PE from 0 to 20%) to give Et-7. ¹H NMR (400 MHz, CDCl₃) δ ppm 8.28 (brs, 1H), 7.12-7.03 (m, 2H), 6.77-6.65 (m, 1H), 4.34 (brs, 1H), 3.80 (brs, 1H), 2.94-2.73 (m, 2H), 1.70 (s, 1H), 1.64-1.50 (m, 1H), 1.47 (s, 9H), 0.96 (t, J=7.2 Hz, 3H).

A solution of Et-7 (1.6 g, 4.93 mmol, 1 eq) in HCl/dioxane (4 M, 44.31 mL, 35.93 eq) was stirred at 15° C. for 2 h to give a brown solution. LCMS and TLC (PE/EtOAc=1:1) showed the reaction was completed. The reaction was concentrated to give the crude product. The crude product was dissolved in H₂O (10 mL) and adjusted with saturated NaHCO₃ to pH=8, concentrated to give the residue. The residue was washed with DCM/EtOH (50 mL/5 mL) and filtered to give Amine-11. ¹H NMR (400 MHz, CDCl₃) δ ppm 8.25 (brs, 1H), 7.13 (s, 1H), 7.08 (dd, J=2.0 Hz, J=9.2 Hz, 1H), 6.76 (t, J=2.0 Hz, 1H), 3.05-2.94 (m, 1H), 2.89-2.83 (m, 1H), 2.60-2.54 (m, 1H), 1.65-1.50 (m, 2H), 1.45-1.32 (m, 2H), 1.02 (t, J=7.2 Hz, 3H).

To a solution of Amine-11 (50 mg, 222.97 μmol, 1 eq) in toluene (5 mL) was added 4-fluorobenzaldehyde (27.67 mg, 222.97 μmol, 23.45 uL, 1 eq) and TFA (25.42 mg, 222.97 μmol, 16.51 uL, 1 eq). The mixture was stirred at 120° C. for 2 hr to give a yellow solution. TLC (PE/EtOAc=1:1) was showed 4-fluorobenzaldehyde was remained and have new two spots had found. The mixture was added TEA (1 mL) and concentrated under vacuum. The crude product was purified by Prep-TLC (PE/EtOAc=5/4) to give 61-1. ¹H NMR (400 MHz, CDCl₃) δ ppm 7.76 (s, 1H), 7.22-7.18 (m, 2H), 7.07-6.97 (m, 3H), 6.76-6.67 (m, 1H), 5.22 (s, 1H), 3.40-3.30 (m, 1H), 3.01-2.83 (m, 2H), 2.50-2.40 (m, 1H), 1.58-1.52 (m, 2H), 0.97 (t, J=7.6 Hz, 3H).

To a solution of 3-trimethylsilylprop-2-ynoyl chloride (0.1 M, 1.97 mL, 1.71 eq) in DCM (1 mL) was added NaHCO₃ (77.31 mg, 920.25 μmol, 35.79 uL, 8 eq) and 61-1 (38 mg, 115.03 μmol, 1 eq) at 0° C. The reaction mixture was allowed to stir at 10° C. for 2 hr to give a yellow suspension. TLC (PE/EtOAc=1:1) showed the mixture was completed. The reaction mixture was quenched with H₂O (20 mL) and extracted with DCM (20 mL*3). The organic layers were dried over Na₂SO₄ and concentrated to give 61-2. Used for next step without further purification.

To a solution of 61-2 (88 mg, 193.59 μmol, 1 eq) in DCM (5 mL)/MeOH (0.5 mL) were added K₂CO₃ (53.51 mg, 387.19 μmol, 2 eq) at 0° C. The mixture was stirred at 0° C. for 3 hr to give a yellow suspension. TLC (eluting with: PE/EtOAc=3/1) showed the mixture was completed. The mixture was diluted with DCM (5 mL) and added H₂O (5 mL). The organic layer was extracted with DCM (20 mL*3) and dried over Na₂SO₄ and concentrated to give the crude product. The crude product was purified by flash column (eluting with: PE/EtOAc=100/0 to 80/20) to give B-61. LC-MS (m/z): 383.0 [M+H]⁺

¹H NMR (400 MHz, CDCl₃) δ ppm 8.07-7.83 (m, 1H), 7.35-7.28 (m, 1H), 7.15-6.86 (m, 4H), 6.79-6.67 (m, 1H), 6.63-5.58 (m, 1H), 4.94-3.70 (m, 1H), 3.30-3.24 (m, 1H), 3.14 (s, 1H), 3.06-2.76 (m, 1H), 2.45-1.60 (m, 2H), 1.01-0.77 (m, 3H).

### Procedure B-10: Compound B-66

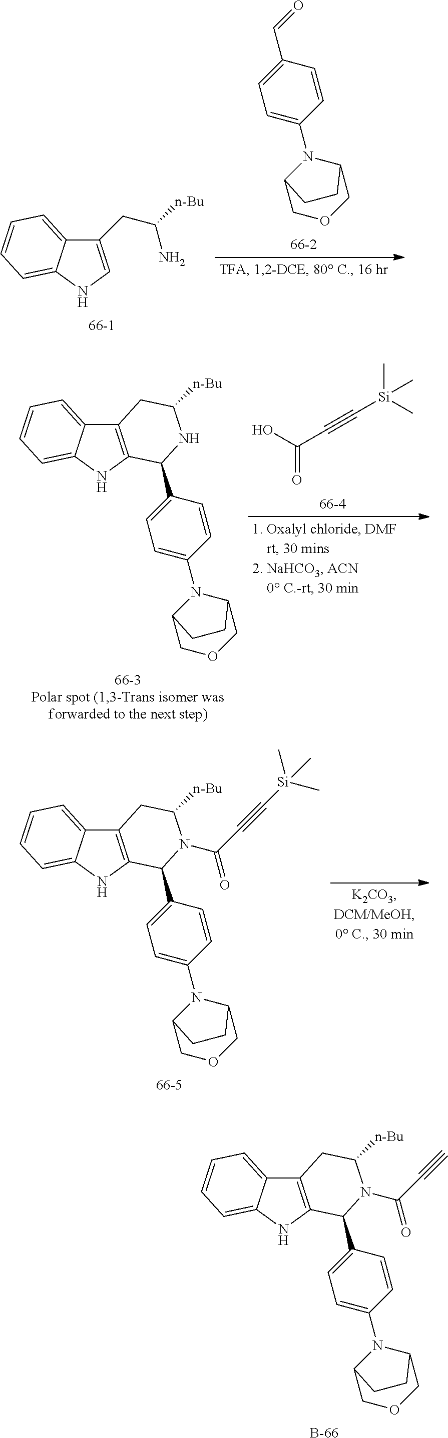

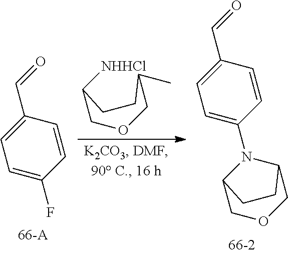

### Preparation of Compound 66-2. 4-(3-oxa-8-azabicyclo[3.2.1]octan-8-yl)benzaldehyde

To a stirred solution of (1R,5S)-3-oxa-8-azabicyclo[3.2.1]octane hydrochloride (1.57 g, 10.5 mmol, 1.3 equiv.) DMSO (20.0 mL) was added potassium carbonate (5.57 g, 40.3 mmol, 5.0 equiv) at room temperature followed by the addition of 4-fluorobenzaldehyde (1.0 g, 8.06 mmol, 1.0 equiv) and then the reaction mixture was heated to 90° C. and stirred for 16 h. TLC (40% EtOAc in hexane) showed that the reaction was complete after this time. The reaction mixture was then cooled to room temperature, diluted with EtOAc (150 mL) and washed with water (20.0 mL). Organic layer was dried over anhydrous sodium sulfate, filtered and concentrated under reduced pressure to get the crude. The crude was purified by silica gel column chromatography using 20-25% EtOAc in hexane as an eluent to afford 4-(3-oxa-8-azabicyclo[3.2.1]octan-8-yl)benzaldehyde 66-2 (0.51 g, 29.1% yield). LC-MS (ES) m/z: 218.1 [M+H]⁺

¹H NMR (400 MHz, DMSO-d6) δ ppm 1.91-1.99 (m, 4H), 3.46 (d, J=10.8 Hz, 2H), 3.61 (d, J=10.8 Hz, 2H), 4.37 (s, 2H), 6.93 (d, J=8.4 Hz, 2H), 7.67 (d, J=8.8 Hz, 2H), 9.66 (s, 1H).

### Preparation of compound 66-3. 8-(4-((1S,3 S)-3-butyl-2,3,4,9-tetrahydro-1H-pyrido[3,4-b]indol-1-yl)phenyl)-3-oxa-8-azabicyclo[3.2.1]octane

To a solution of (2S)-1-(1H-indol-3-yl)hexan-2-amine (0.5 g, 2.31 mmol, 1.0 equiv) in 1,2-dichloroethane (5.0 mL) taken in a sealed tube was added 4-{3-oxa-8-azabicyclo[3.2.1]octan-8-yl}benzaldehyde 66-2 (452 mg, 2.08 mmol, 0.9 equiv) at room temperature followed by the addition of TFA (0.36 mL, 4.62 mmol, 2.0 equiv.) and then the reaction was stirred at 80° C. for 10 h. TLC (5% MeOH/DCM) showed that the reaction was completed after 10 h. The reaction mixture was quenched with saturated NaHCO₃ solution and extracted into EtOAc (1×20 mL). Combined organic layer was dried over anhydrous Na₂SO₄, filtered and concentrated under reduced pressure to get the crude. Obtained crude product was purified by silica gel flash column chromatography MeOH/DCM 1-2% as eluent to give 8-{4-[(1S,3S)-3-butyl-1H,2H,3H,4H,9H-pyrido[3,4-b]indol-1-yl]phenyl}-3-oxa-8-azabicyclo[3.2.1]octane 66-3 (110 mg, 11.45%). LC-MS (ES) m/z: 416.2 [M+H]⁺

¹H NMR (400 MHz, DMSO-d6) δ ppm 0.81-0.91 (m, 4H), 1.16-1.89 (m, 11H), 2.25-2.31 (m, 1H), 2.65-2.78 (m, 2H), 3.06-3.48 (m, 3H), 3.55-4.06 (m, 2H), 4.16 (s, 2H), 5.01 (s, 1H), 6.75 (d, J=8.4 Hz, 1H), 6.90-7.00 (m, 4H), 7.21 (d, J=8.0 Hz, 1H), 7.38 (d, J=8.0 Hz, 1H), 10.64 (s, 1H).

### Preparation of compound 66-5. 1-((1 S,3 S)-1-(4-(3-oxa-8-azabicyclo[3.2.1]octan-8-yl)phenyl)-3-butyl-1,3,4,9-tetrahydro-2H-pyrido[3,4-b]indol-2-yl)-3-(trimethyl silyl)prop-2-yn-1-one

Step 1: To a stirred solution of 3-(trimethylsilyl)propiolic acid (0.103 g, 0.724 mmol, 1.0 equiv) in DMF (0.002 mL, 0.002 mmol, 0.04 equiv) was added oxalyl chloride (0.063 mL, 0.796 mmol, 1.1 equiv), was added at room temperature and stirred for 30 min. After completion, the reaction mass was concentrated under nitrogen atmosphere and taken to next step (0.130 g, crude).

Step 2: To a stirred solution of 8-{4-[(1 S,3 S)-3-butyl-1H,2H,3H,4H,9H-pyrido[3,4-b]indol-1-yl]phenyl}-3-oxa-8-azabicyclo[3.2.1]octane 66-3 (100 mg, 241 μmol, 1.0 equiv.) in ACN (5.0 mL) was added sodium bicarbonate (0.141 g, 1.68 mmol, 7.0 equiv.) at 0° C., stirred at 0° C. for 15 mins and then 3-(trimethylsilyl)propioloyl chloride (0.090 g, 0.722 mmol, 2.0 equiv.) in ACN (2.0 mL) was added at 0° C. and the reaction was stirred at room temperature for 30 mins. Reaction was monitored by TLC (70% EtOAc in hexane). After this time the reaction mixture was diluted with EtOAc (100 mL) and was washed with water (10 mL). Organic layer was dried over anhydrous sodium sulfate, filtered and concentrated under reduced pressure to afford 1-[(1S,3S)-3-butyl-1-(4-{3-oxa-8-azabicyclo[3.2.1]octan-8-yl}phenyl)-1H,2H,3H,4H,9H-pyrido[3,4-b]indol-2-yl]-3-(trimethylsilyl)prop-2-yn-1-one 66-5 (110 mg, crude). LC-MS (ES) m/z: 540.3 [M+H]⁺

### Preparation of compound B-66 1-((1 S,3 S)-1-(4-(3-oxa-8-azabicyclo[3.2.1]octan-8-yl)phenyl)-3-butyl-1,3,4,9-tetrahydro-2H-pyrido[3,4-b]indol-2-yl)prop-2-yn-1-one

To a solution of 1-[(1S,3 S)-3-butyl-1-(4-{3-oxa-8-azabicyclo[3.2.1]octan-8-yl}phenyl)-1H,2H,3H,4H,9H-pyrido[3,4-b]indol-2-yl]-3-(trimethylsilyl)prop-2-yn-1-one 66-5 (120 mg, 222 μmol, 1 eq.) in DCM (8.0 mL)/MeOH (2 ml) under nitrogen atmosphere was added dipotassium carbonate (215 mg, 7 eq., 1.56 mmol) at 0° C. and then the reaction mixture was stirred at 0° C. for 30 mins. TLC (50% EtOAc in hexane) showed that the reaction was completed in 30 mins. The reaction mixture was quenched with water at 0° C. and diluted with DCM (10 mL), stirred the reaction mixture at room temperature for 5 mins. The reaction mixture was then extracted into DCM (50 mL) and was washed with water (5 mL) and brine (5 mL). Organic layer was dried over anhydrous sodium sulfate, filtered and concentrated to get the crude. The crude was purified by preparative TLC using 45% EtOAc in hexane as an eluent (eluted twice) to afford the title compound 1-[(1 S,3 S)-3-butyl-1-(4-{3-oxa-8-azabicyclo[3.2.1]octan-8-yl}phenyl)-1H,2H,3H,4H,9H-pyrido[3,4-b]indol-2-yl]prop-2-yn-1-one B-66 (0.037 mg, Yield: 35.7 5). LC-MS (ES) m/z: 468.3 [M+H]⁺, HPLC: 96.82%, Chiral HPLC ee: 99.88%

¹HNMR_VT at 100° C. (400 MHz, DMSO-d6) ppm 0.75-0.78 (m, 3H), 1.15-1.35 (m, 6H), 1.54 (s, 1H), 1.81-1.82 (m, 2H), 1.89-1.91 (m, 2H), 2.95 (s, 4H), 3.40-3.43 (m, 2H), 3.66-3.68 (m, 2H), 4.04 (s, 1H), 4.27 (s, 1H), 6.74 (s, 1H), 6.95-6.98 (m, 2H), 7.02-7.05 (m, 2H), 7.09 (d, J=8.4 Hz, 2H), 7.27 (d, J=8.0 Hz, 1H), 7.43 (d, J=7.2 Hz, 1H), 10.85 (s, 1H).

### Procedure B-11—Synthesis of Compound B-69

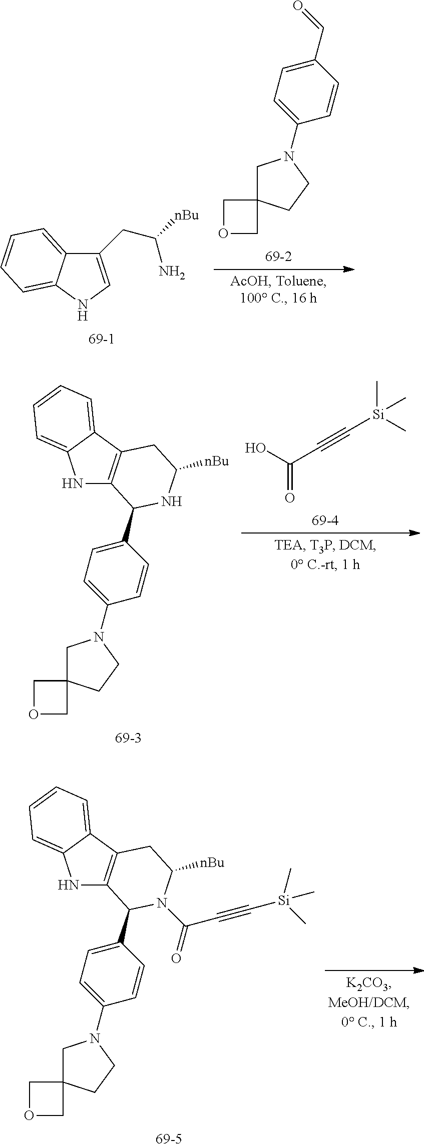

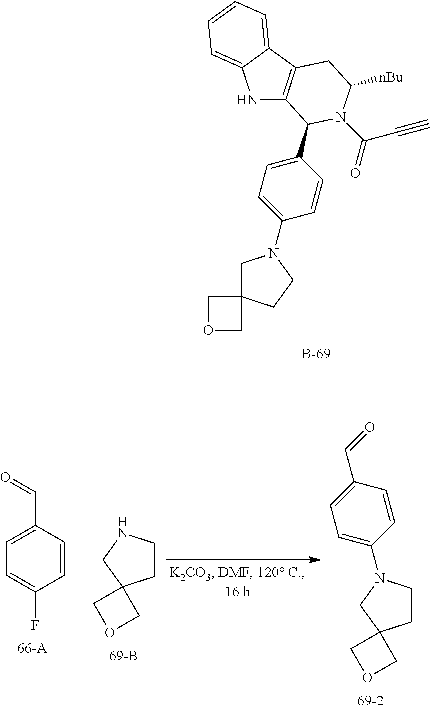

### Preparation of compound 69-2. 4-(2-oxa-6-azaspiro[3.4]octan-6-yl)benzaldehyde

To a stirred solution of 2-oxa-6-azaspiro[3.4]octane 69-B (1.19 g, 10.5 mmol, 1.3 equiv) in DMF (15.0 mL) was added potassium carbonate (2.26 g, 16.1 mmol, 2.0 equiv) at room temperature followed by the addition of 4-fluorobenzaldehyde 66-A (1.0 g, 8.06 mmol, 1.0 equiv) and then the reaction mixture was heated to 120° C. and stirred for 16 h. TLC (40% EtOAc in hexane) showed that the reaction was complete after this time. The reaction mixture was then cooled to room temperature and diluted with ice and then extracted with EtOAc (50 mL). Organic layer was dried over anhydrous sodium sulfate, filtered and concentrated under reduced pressure to get the crude. The crude was purified by silica gel column chromatography using 20-25% EtOAc in hexane as an eluent to afford 4-{2-oxa-6-azaspiro[3.4]octan-6-yl}benzaldehyde 69-2 (1.20 g, 68.3% yield). LC-MS (ES) m/z: 218.1 [M+H]⁺

¹H NMR (400 MHz, DMSO-d6): δ ppm 2.25-2.28 (m, 2H), 3.29-3.37 (m, 2H), 3.60 (s, 2H), 4.50-4.58 (m, 4H), 6.62 (d, J=8.4 Hz, 2H), 7.66 (d, J=8.8 Hz, 2H), 9.64 (s, 1H),

### Preparation of compound 69-3. 6-(4-((1S,3 S)-3-butyl-2,3,4,9-tetrahydro-1H-pyrido[3,4-b]indol-1-yl)phenyl)-2-oxa-6-azaspiro[3.4]octane

To a solution of (2S)-1-(1H-indol-3-yl)hexan-2-amine 69-1 (850 mg, 3.93 mmol, 1.0 equiv.) in Toluene (10.0 mL) taken in sealed tube was added 4-{2-oxa-6-azaspiro[3.4]octan-6-yl}benzaldehyde 69-2 (1.11 g, 1.3 equiv, 5.11 mmol) at room temperature followed by the addition of acetic acid (0.67 ml, 3 equiv, 7.86 mmol) and then the reaction was stirred at 100° C. for 16 hr. Reaction mixture was cooled to room temperature and evaporated under reduced pressure. obtained crude was quenched with saturated sodium bicarbonate solution then extracted with ethyl acetate (2×30 mL). Combined organic layer was washed with brine, dried over anhydrous sodium sulphate. Organic layer was filtered and concentrated under reduced pressure to get crude product. Crude was purified by flash column chromatography using ethyl acetate in hexane as eluent. Product was isolated at 20-22% ethyl acetate in hexane. Product fractions collected and concentrated under reduced pressure to get 6-{4-[(1S,3S)-3-butyl-1H,2H,3H,4H,9H-pyrido[3,4-b]indol-1-yl]phenyl}-2-oxa-6-azaspiro[3.4]octane 69-3. LC-MS (ES) m/z: 416.3 [M+H]

### Preparation of compound 69-5. 1-((1 S,3 S)-1-(4-(2-oxa-6-azaspiro[3.4]octan-6-yl)phenyl)-3-butyl-1,3,4,9-tetrahydro-2H-pyrido[3,4-b]indol-2-yl)-3-(trimethyl silyl)prop-2-yn-1-one

Procedure: To a stirred solution of 3-(trimethylsilyl)prop-2-ynoic acid 69-4 (0.103 g, 0.722 mmol, 1.2 equiv) in DCM (8.0 mL) at 0° C. was added triethylamine (0.254 mL, 1.80 mmol, 3.0 equiv) followed by Propanephosphonic acid anhydride (T3P) (50 wt. % in EA, 1.1 mL, 0.902 mmol, 1.5 equiv). After stirring for minutes 6-{4-[(1S,3 S)-3-butyl-1H,2H,3H,4H,9H-pyrido[3,4-b]indol-1-yl]phenyl}-2-oxa-6-azaspiro[3.4]octane 69-3 (250 mg, 0.602 μmol, 1.0 equiv.) was added. Then reaction was stirred at room temperature for 1 h. Reaction was monitored by TLC (25% EtOAc in hexane). After this time the reaction mixture was diluted with EtOAc (25 mL) and was washed with water (5 mL). Organic layer was dried over anhydrous sodium sulfate, filtered and concentrated under reduced pressure get crude 69-5, which was taken forward without any purification.

### Preparation of compound B-69. 1-((1S,3S)-1-(4-(2-oxa-6-azaspiro[3.4]octan-6-yl)phenyl)-3-butyl-1,3,4,9-tetrahydro-2H-pyrido[3,4-b]indol-2-yl)prop-2-yn-1-one

To a solution of crude 1-[(1 S,3 S)-3-butyl-1-(4-{2-oxa-6-azaspiro[3.4]octan-6-yl}phenyl)-1H,2H,3H,4H,9H-pyrido[3,4-b]indol-2-yl]-3-(trimethylsilyl)prop-2-yn-1-one 69-5 (200 mg, 371 μmol, 1 eq.) in DCM (7.0 mL)/MeOH (1 ml) under nitrogen atmosphere was added dipotassium carbonate (358 mg, 2.59 mmol, 7 eq.) at 0° C. and then the reaction mixture was stirred at 0° C. for 30 mins. TLC (25% EtOAc in hexane) showed that the reaction was completed in 1 h. The reaction mixture was quenched with water at 0° C. and diluted with DCM (10 mL), stirred the reaction mixture at room temperature for 5 mins. The reaction mixture was then extracted into DCM (50 mL) and was washed with water (5 mL) and brine (5 mL). Organic layer was dried over anhydrous sodium sulfate, filtered and concentrated to get the crude. The crude was purified by preparative TLC using 25% EtOAc in hexane as an eluent (eluted twice) to afford the title compound 1-[(1 S,3 S)-1-{4-[(adamantan-1-yl)amino]phenyl}-3-butyl-9-methyl-1H,2H,3H,4H,9H-pyrido[3,4-b]indol-2-yl]prop-2-yn-1-one B-69 (0.039 g, yield: 22.51%). LC-MS (ES)m/z: 468.2 [M+H]⁺ HPLC: 95.83% Chiral HPLC ee:99.98%

¹H NMR (400 MHz, DMSO-d6) δ ppm 0.77 (s, 3H), 1.0-1.53 (m, 6H), 2.0-2.30 (m, 3 HO, 2.65 (s, 1H), 3.18 (m, 2H), 3.15 (m, 2 HO, 3.88 (s, 1H), 4.32 (s, 1H), 4.48-4.58 (m, 4H), 4.79+(s, 1H), 5.86 (s, 1H), 6.46 (m, 2H), 6.95-7.08 (m, 4H), 7.28 (m, 1H), 7.42 (d, J=7.6 Hz, 1H), 10.58 (s, 1H).

### Procedure B-12 Preparation of B-70

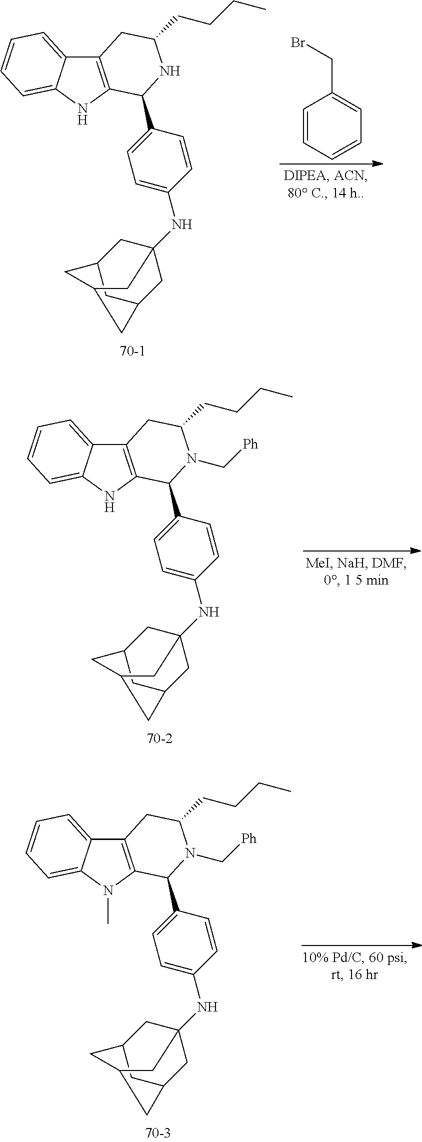 [SMILES: CCCC[C@H]1Cc2c([nH]c3ccccc23)[C@H](c2ccc(NC34CC5CC(CC(C5)C3)C4)cc2)N1 ; BrCc1ccccc1 ; CCCC[C@H]1Cc2c([nH]c3ccccc23)[C@H](c2ccc(NC34CC5CC(CC(C5)C3)C4)cc2)N1Cc1ccccc1 ; CCCC[C@H]1Cc2c(n(C)c3ccccc23)[C@H](c2ccc(NC34CC5CC(CC(C5)C3)C4)cc2)N1Cc1ccccc1]

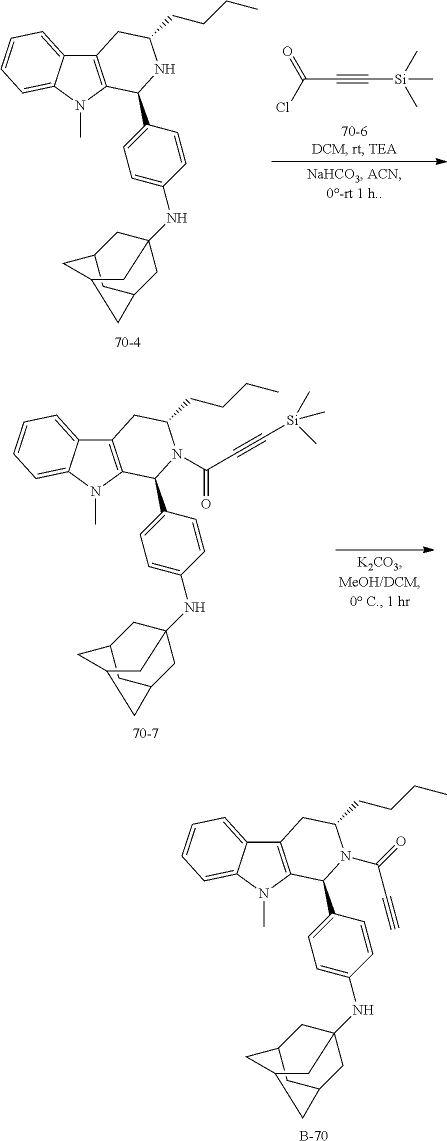

### Preparation of compound 70-2. (1R,3R,5 S)—N-(4-((1 S,3 S)-2-benzyl-3-butyl-2,3,4,9-tetrahydro-1H-pyrido[3,4-b]indol-1-yl)phenyl)adamantan-1-amine

To a solution of N-{4-((1S,3S)-3-butyl-1H,2H,3H,4H,9H-pyrido[3,4-b]indol-1-yl]phenyl}adamantan-1-amine 70-1 (400 mg, 882 μmol, 1 equiv) in acetonitrile (5.0 mL) was added DIPEA (462 μL, 2.65 mmol, 3 equiv) and (bromomethyl)benzene (209 μL, 1.76 mmol, 2 equiv) at room temperature. This reaction was stirred at 80° C. for 14 h. TLC (8% EtOAc in hexane) showed the reaction was completed. The reaction was cooled to room temperature and was concentrated under reduced pressure to get the crude product 70-2 (0.5 g, crude). This crude product was taken to next step. LCMS (ES) m/z=544.2 [M+H]⁺

### Preparation of compound 70-3. (1R,3R,5S)—N-(4-((1S,3S)-2-benzyl-3-butyl-9-methyl-2,3,4,9-tetrahydro-1H-pyrido[3,4-b]indol-1-yl)phenyl)adamantan-1-amine

To a stirred solution of tert-butyl (1 S,3 S)-1-{4-[(adamantan-1-yl)amino]phenyl}-3-butyl-1H,2H,3H,4H,9H-pyrido[3,4-b]indole-2-carboxylate 70-2 (0.500 g, 0.919 mmol, 1.0 equiv) in DMF (5 mL) at 0 C was added sodium hydride (60% in mineral Oil, 0.044 g, 1.08 mmol, 1.2 equiv). Then reaction mixture was stirred at same temperature for 15 minutes, then methyl iodide (0.090 mL, 1.35 mmol, 1.5 equiv) added. Then reaction mixture was allowed to stir at room temperature for 30 minutes. Then reaction mixture was quenched with ice water, extracted with ethyl acetate (2×15 mL). Combined organic layer was washed with brine (5 mL), dried over anhydrous sodium sulphate. Organic layer was filtered and concentrated under reduced pressure to get crude product 70-3.

### Preparation of compound 70-4. (1R,3R,5S)—N-(4-((1S,3S)-3-butyl-9-methyl-2,3,4,9-tetrahydro-1H-pyrido[3,4-b]indol-1-yl)phenyl)adamantan-1-amine

In a par hydrogenation vessel to a solution of N-{4-[(1S,3S)-2-benzyl-3-butyl-9-methyl-1H,2H,3H,4H,9H-pyrido[3,4-b]indol-1-yl]phenyl}adamantan-1-amine 70-3 (0.500 g, 0.896 mmol, 1.0 equiv) in methanol (20 mL) at rt, 10% palladium on carbon (50 mg) was added. Then reaction mixture was stirred under hydrogen at 60 psi for 16 h. Then reaction mixture was filtered through celite bed. Celite bed was washed with methanol. Organic layer was filtered and concentrated under reduced pressure. Obtained crude 70-4 was taken forward without further purification. LC-MS(ES)m/z: 468.4 [M+H]⁺

### Preparation of compound 70-7. 1-((1 S,3 S)-1-(4-(((1R,3R,5 S)-adamantan-1-yl)amino)phenyl)-3-butyl-9-methyl-1,3,4,9-tetrahydro-2H-pyrido[3,4-b]indol-2-yl)-3-(trimethyl silyl)prop-2-yn-1-one

To a stirred solution of N-{4-[(1 S,3 S)-3-butyl-9-methyl-1H,2H,3H,4H,9H-pyrido[3,4-b]indol-1-yl]phenyl}adamantan-1-amine 70-4 (0.210 g, 0.449 mmol, 1.0 equiv) in Acetonitrile (10.0 mL) at 0° C. was added sodium bicarbonate (0.302 g, 3.59 mmol, 8.0 equiv) in acetonitrile (2 mL) at 0° C. After stirring for 5 minutes 3-(trimethylsilyl)prop-2-ynoyl chloride 70-6 (0.072 g, 0.449 mmol, 1.2 equiv) was added at same temperature. Then reaction mixture was allowed to stirred at room temperature for 15 minutes. Then reaction mixture was diluted with water (5 mL), extracted with ethyl acetate (2×10 mL). Combined organic layer was washed with brine (5 mL), dried over anhydrous sodium sulphate. Organic layer was filtered and concentrated under reduced pressure to get crude product. Obtained crude product 70-7 was taken forward without further purification. LC-MS(ES)m/z: 592.3 [M+H]⁺

### Preparation of compound B-70. 1-((1S,3S)-1-(4-(((1R,3R,5S)-adamantan-1-yl)amino)phenyl)-3-butyl-9-methyl-1,3,4,9-tetrahydro-2H-pyrido[3,4-b]indol-2-yl)prop-2-yn-1-one

To a solution of 1-[(1S,3 S)-1-{4-[(adamantan-1-yl)amino]phenyl}-3-butyl-9-methyl-1H,2H,3H,4H,9H-pyrido[3,4-b]indol-2-yl]-3-(trimethylsilyl)prop-2-yn-1-one 70-7 (160 mg, 0.027 mmol, 1 eq.) in DCM (6.0 mL)/MeOH (1 ml) under nitrogen atmosphere was added dipotassium carbonate (261 mg, 7 eq., 1.89 mmol) at 0° C. and then the reaction mixture was stirred at 0° C. for 30 mins. TLC (30% EtOAc in hexane) showed that the reaction was completed in 30 mins. The reaction mixture was quenched with water at 0° C. and diluted with DCM (10 mL), stirred the reaction mixture at room temperature for 5 mins. The reaction mixture was then extracted into DCM (50 mL) and was washed with water (5 mL) and brine (5 mL). Organic layer was dried over anhydrous sodium sulfate, filtered and concentrated to get the crude. The crude was purified by preparative TLC using 30% EtOAc in hexane as an eluent to afford the title compound 1-[(1S,3S)-1-{4-[(adamantan-1-yl)amino]phenyl}-3-butyl-9-methyl-1H,2H,3H,4H,9H-pyrido[3,4-b]indol-2-yl]prop-2-yn-1-one (0.050 g, 35.59%). LC-MS(ES)m/z:520.4 [M+H]⁺ HPLC: 99.47%, Chiral HPLC: ee 97.08%

¹H NMR (400 MHz, DMSO-d6) δ ppm 0.74 (s, 3H), 1.15-1.40 (m, 6H), 1.61 (s, 7H), 1.73-1.82 (m, 6H), 2.02 (s, 3H), 3.47 (s, 3H), 4.34-4.75 (m, 2H), 6.68 (m, 2H), 6.9-7.01 (m, 2H), 7.04-7.3 (m, 3H), 7.48 (d, J=7.6 Hz, 1H).

### Procedure B-13—Preparation of B-71

Using procedures similar to the procedures described above, B-71 was prepared. LC-MS(ES)m/z:453.6 [M+H]⁺

¹H NMR (400 MHz, DMSO-d6) δ ppm 0.71-0.79 (m, 3H), 0.87 (d, J=6.0 Hz, 1H), 1.01-1.23 (m, 5H), 1.52-1.83 (m, 6H), 2.17 (s, 1H), 2.76-2.90 (m, 2H), 2.99-3.19 (m, 3H), 3.72 (s, 1H), 4.53 (d, J=5.2 Hz, 1H), 4.78 (bs, 1H), 5.24 (d, J=7.6 Hz, 1H), 6.37-6.52 (m, 3H), 6.89-7.07 (m, 4H), 7.22 (d, J=8.0 Hz, 1H), 7.43 (s, 1H), 10.79 (s, 1H).

Compounds as shown in Table B-1, can be or were, synthesized according to the procedures described above using the appropriate reagents and starting materials. Select data are shown in Table B-2.

TABLE B-2

| No. | MS [M + H]⁺ |
|---|---|
| B-1 | 558 |
| B-2 | 438.5 |
| B-3 | 457 |
| B-4 | 425 |
| B-5 | 454 |
| B-6 | 507 |
| B-7 | 534 |
| B-8 | 664 |
| B-9 | 664 |
| B-11 | 475.6 |
| B-13 | 504 |
| B-14 | 501 |
| B-15 | 477 |
| B-16 | 483.6 |
| B-17 | 506 |
| B-18 | 542 |
| B-19 | 494 |
| B-22 | 434 |
| B-23 | 400 |
| B-24 | 396.4 |
| B-25 | 521.6 |
| B-26 | 436 |
| B-27 | 540 |
| B-28 | 457.5 |
| B-29 | 448.5 |
| B-30 | 393.4 |
| B-31 | 393.4 |
| B-32 | 3.9 |
| B-33 | 396.4 |
| B-34 | 396.4 |
| B-35 | 479.5 |
| B-36 | 528 |
| B-37 | 504 |

### Procedure C-1: Synthesis of Compound C-5

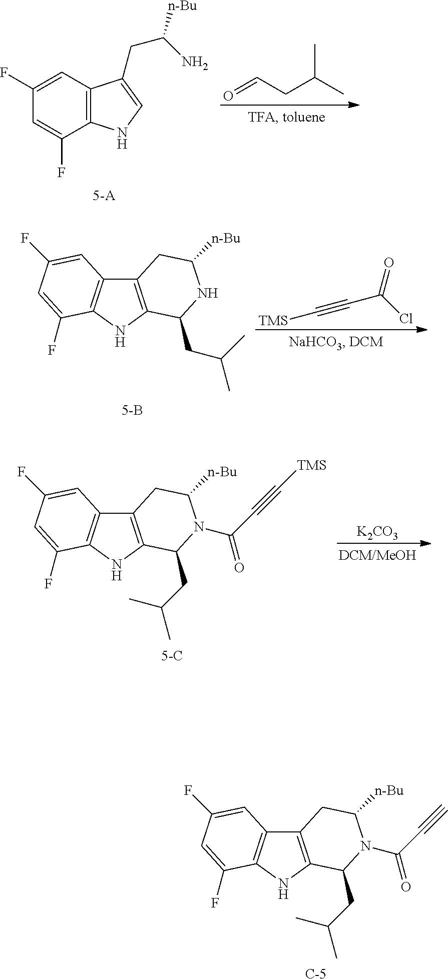

To a solution of 5-A (300 mg, 1.19 mmol, 1 eq) in DCM (10 mL) were added 3-methylbutanal (102.42 mg, 1.19 mmol, 130.47 uL, 1 eq) and TFA (135.58 mg, 1.19 mmol, 88.04 uL, 1 eq). The reaction was stirred at 50° C. for 12 hr to give a yellow solution. TLC (eluting with: PE/EtOAc=4/1) showed the reaction was completed. The reaction mixture was basified to pH=8 with Et₃N. The reaction mixture was purified by flash column (eluting with: PE/EtOAc=0% to 30%) to give 5-B. ¹H NMR (400 MHz, CDCl₃) δ=7.70 (s, 1H), 6.85 (dd, J=2.0 Hz, J=9.2 Hz, 1H), 6.60 (t, J=2.0 Hz, J=3.6 Hz, 1H), 4.04 (dd, J=4.4 Hz, J=10.6 Hz, 1H), 3.02-2.97 (m, 1H), 2.67 (d, J=11.2 Hz, 1H), 2.25-2.20 (m, 1H), 1.98-1.67 (m, 2H), 1.43-1.30 (m, 6H), 0.95 (d, J=3.2 Hz, 6H), 0.92 (t, J=7.2 Hz, 3H).

To a solution of 5-B (35 mg, 109.23 μmol, 1 eq) in DCM (10 mL) were added NaHCO₃ (55.06 mg, 655.39 μmol, 25.49 uL, 6 eq) and 3-trimethylsilylprop-2-ynoyl chloride (0.141 M, 774.69 uL, 1 eq) at 0° C. The mixture was stirred at 0° C. for 1 hr to give a yellow solution. LCMS and TLC (eluting with: PE/EtOAc=3/1) showed the reaction was completed. The reaction mixture was quenched with H₂O (10 mL) and extracted with DCM (20 mL*3). The organic layers were dried over Na₂SO₄ and concentrated to give the crude product. The crude product was purified by flash column (eluting with: PE/EtOAc=0% to 30%) to give 5-C.

To a solution of 5-C (22 mg, 49.48 μmol, 1 eq) in DCM (5 mL)/MeOH (2 mL) was added K₂CO₃ (13.68 mg, 98.96 μmol, 2 eq) at 0° C. The reaction was stirred at 0° C. for 1 hr to give a yellow solution. LCMS showed the reaction was completed. The reaction mixture was quenched with H₂O (10 mL) and extracted with DCM (20 mL*3). The organic layers were dried over Na₂SO₄ and concentrated to give the crude product. The crude product was purified by flash column (eluting with: PE/EtOAc=0% to 30%) to give C-5. LC-MS (m/z): 373.1[M+H]⁺. ¹H NMR (400 MHz, CDCl₃) δ=8.13-7.90 (m, 1H), 6.93-6.85 (m, 1H), 6.93-6.85 (m, 1H), 6.70-6.60 (m, 1H), 5.52-5.00 (m, 1H), 4.52-3.37 (m, 1H), 3.08-2.88 (m, 1H), 2.64-2.55 (m, 2H), 2.25-1.52 (m, 3H), 1.40-1.00 (m, 6H), 0.99-0.73 (m, 9H).

### Procedure C-4: Synthesis of Compound C-18

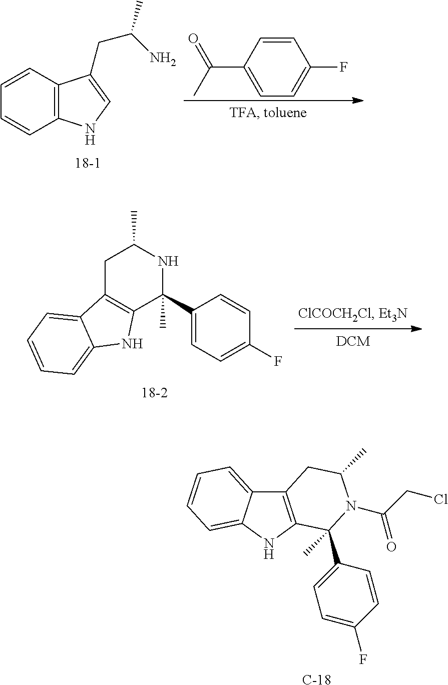

18-1 (200 mg, 1.15 mmol, 1 eq), 1-(4-fluorophenyl)ethanone (158.56 mg, 1.15 mmol, 139.09 uL, 1 eq) and TFA (196.31 mg, 1.72 mmol, 127.48 uL, 1.5 eq) were taken up into a microwave tube in toluene (3 mL). The sealed tube was heated at 140° C. for 2 h under microwave to give brown mixture. TLC showed starting material was remained, and one major new spot with lower polarity was detected. The reaction mixture was diluted with NaHCO₃ (aq. 30 mL) and extracted with ethyl acetate (20 mL×3). The combined organic phase was washed with brine (30 mL), dried with anhydrous Na₂SO₄, filtered and concentrated in vacuum to give a residue. The residue was purified by prep-TLC (SiO₂, Petroleum ether:Ethyl acetate=2:1) and dried by lyophilized to afford 18-2.

To a mixture of 18-2 (16 mg, 54.35 μmol, 1 eq) and TEA (55.00 mg, 543.54 μmol, 75.65 uL, 10 eq) in DCM (4 mL) was added 2-chloroacetyl chloride (9.21 mg, 81.53 μmol, 6.48 uL, 1.5 eq) at 0° C. The mixture was stirred at 0° C. for 1 h. LCMS showed the reaction was completed. The reaction mixture was concentrated in reduced pressure to give a residue. The residue was purified by prep-TLC (SiO₂, Petroleum ether: Ethyl acetate=2:1) and dried by lyophilized to afford C-18. LC-MS (m/z): 370.9 [M+H]⁺. ¹H NMR (400 MHz, CHLOROFORM-d) δ 1.40 (d, J=6.8 Hz, 3H), 2.16 (s, 3H), 2.89 (d, J=15.2 Hz, 1H), 3.34 (br dd, J=15.6, 5.6 Hz, 1H), 3.92 (br d, J=12.8 Hz, 1H), 4.16 (br d, J=12.4 Hz, 1H), 4.75 (br s, 1H), 6.83 (br t, J=8.4 Hz, 2H), 7.02-7.09 (m, 2H), 7.15 (br d, J=7.2 Hz, 1H), 7.20-7.24 (m, 2H), 7.38 (br s, 1H), 7.45 (br d, J=7.2 Hz, 1H).

### Procedure C-5: Synthesis of Compound C-4

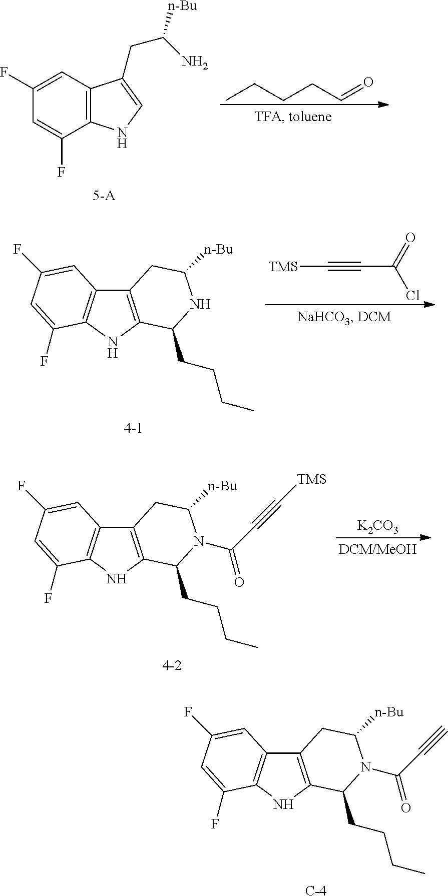

To a solution of 5-A in toluene (20 mL) were added pentanal (68.28 mg, 792.70 μmol, 84.29 uL, 1 eq) and TFA (135.58 mg, 1.19 mmol, 88.04 uL, 1.5 eq). The mixture was stirred at 120° C. for 2 hr to give a yellow solution. TLC (PE/EtOAc=1/1) showed new spots were found. The mixture was concentrated under vacuum and added TEA (2 mL). The residue was purified by column chromatography (SiO₂, Petroleum ether/Ethyl acetate=100/0 to 82/18) to give 5-1. ¹H NMR (400 MHz, CDCl₃) δ ppm 7.84 (brs, 1H), 6.93 (dd, J=2.0 Hz, J=9.2 Hz, 1H), 6.68 (t, J=3.6 Hz, 1H), 4.03 (t, J=6.8 Hz, 1H), 3.14-3.05 (m, 1H), 2.77 (dd, J=4.0 Hz, J=11.2 Hz, 1H), 2.37-2.30 (m, 1H), 1.81-1.70 (m, 2H), 1.60-1.51 (m, 2H), 1.51-1.31 (m, 8H), 1.00-0.93 (m, 6H).

To a solution of 5-1 in DCM (2 mL) were added NaHCO₃ (100.68 mg, 1.20 mmol, 46.61 uL, 8 eq) and 3-trimethylsilylprop-2-ynoyl chloride (0.1 M, 4.49 mL, 3 eq) at 0° C. The mixture was stirred at 0° C. for 1.5 hr to give a yellow solution. TLC (PE/EtOAc=1/1) showed the mixture was completed. The mixture was added H₂O (5 mL), extracted with DCM (8 mol*3). The combined organic layers were dried over NaSO₄, filtered and concentrated to give the crude product. The residue was purified by column chromatography (SiO₂, Petroleum ether/Ethyl acetate=100/0 to 85/15) to give 5-2. ¹H NMR (400 MHz, CDCl₃) δ ppm 8.17-8.02 (m, 1H), 7.00-6.92 (m, 1H), 6.77-6.70 (m, 1H), 5.43-5.36 (m, 1H), 5.17-3.92 (m, 1H), 3.00-2.88 (m, 2H), 2.06-1.88 (m, 2H), 1.62-1.20 (m, 6H), 0.96-0.84 (m, 6H), 0.27 (s, 9H).

To a solution of 5-2 (58 mg, 130.45 μmol, 1 eq) in DCM (5 mL)/MeOH (0.5 mL) were added K₂CO₃ (72.11 mg, 521.78 μmol, 4 eq) at 0° C. The mixture was stirred at 0° C. for 2 hr to give a yellow solution. TLC (PE/EtOAc=3/1) showed the mixture was completed. The mixture was added H₂O (5 mL), extracted with DCM (8 mL*3). The combined organic layers were dried over Na₂SO₄, filtered and concentrated to give the crude product. The residue was purified by column chromatography (SiO₂, Petroleum ether/Ethyl acetate=100/0 to 86/14) to give C-4. LC-MS (m/z): 373.0 [M−H]⁺

¹H NMR (400 MHz, CDCl₃) δ ppm 8.06-7.94 (m, 1H), 7.00-6.91 (m, 1H), 6.77-6.70 (m, 1H), 5.48-5.10 (m, 1H), 4.72-3.88 (m, 1H), 3.15 (s, 1H), 3.13-2.70 (m, 2H), 2.17-1.88 (m, 2H), 1.50-1.20 (m, 10H), 0.96-0.80 (m, 6H).

Compounds as shown in Table C-1, can be or were, synthesized according to the procedures described above using the appropriate reagents and starting materials. Select data are shown in Table C-2.

TABLE C-2

| No. | MS [M + H]⁺ |
|---|---|
| C-1 | 372 |
| C-2 | 372 |
| C-3 | 358.4 |
| C-4 | 372.5 |
| C-5 | 373.1 |
| C-8 | 323.4 |
| C-18 | 370.9 |

### BIOLOGICAL EXAMPLES

### Example 1: Cell Proliferation (Alamar Blue) Assay

Cell viability assay was performed to assess the potency of the compounds in human cancer cell lines 786-0 (renal cell carcinoma) and SJSA-1 (osteosarcoma). Additional cell lines, such as pancreatic cancer cell lines (Panc 02.13, BxPC-3, Panc 12, Panc 02.03, Panc 6.03, PSN-1, HPAC, and Capan-1), prostate cancer cell lines (PC-3, DU145, 22Rv1, NCI-H660, BPH1, LNCaP, BM-1604, and MDA PCa 2b), etc., can be tested in a similar method.

Cells (SJSA-1, 786-0 and A431) were seeded (5000 cells/100 μL/well) in 96-well tissue culture plate and incubated at 37° C./5% CO₂ for 16-24 hours. The cells were then treated with compounds (25 μL of 5×). The compound concentrations were 10-0.0005 μM prepared in 3-fold serial dilutions with final DMSO concentration of 1%. The plates were then incubated for 24 h at 37° C./5% CO₂ in a moist environment. Then Alamar Blue™ reagent (final concentration 1×-12.5 μL) was added to each well and incubated for 1.5 hours at 37° C./5% CO₂. The plates were read on fluorescence reader at 540 nm excitation and 590 nm emission wavelengths. The IC₅₀ values were subsequently determined using a sigmoidal dose-response curve (variable slope) in GraphPad Prism® 5 software. Table A-3, Table B-3 and Table C-3 show cell proliferation data for certain compounds as described herein.

TABLE A-3

| | IC₅₀ (μM) | | |
|---|---|---|---|
| No. | 786-O | SJSA-1 | A431 |
| A-1 | 0.03 | 0.145 | >10 |
| A-2 | 3.52 | 9.11 | 7.06 |
| A-3 | 0.017 | 0.017 | 8.36 |
| A-7 | 0.034 | 0.058 | 4.881 |

TABLE B-3

| | IC₅₀ (μM) | IC₅₀ (μM) | IC₅₀ (μM) | | IC₅₀ (μM) | IC₅₀ (μM) | IC₅₀ (μM) |
|---|---|---|---|---|---|---|---|
| No. | 786-O | SJSB-1 | A431 | No. | 786-O | SJSB-1 | A431 |
| B-1 | 0.001 | 0.003 | 2.7 | B-13 | 0.013 | 0.018 | 7.2 |
| B-2 | 0.006 | 0.014 | 3.2 | B-14 | 0.015 | 0.019 | 3 |
| B-3 | 0.009 | 0.018 | 4.7 | B-15 | 0.013 | 0.029 | >10 |
| B-4 | 0.008 | 0.015 | 5.9 | B-16 | 0.007 | 0.029 | 6.4 |
| B-5 | 0.002 | 0.002 | NT | B-17 | 0.006 | 0.008 | 3.6 |
| B-6 | 0.024 | 0.024 | 6.1 | B-18 | 0.005 | 0.006 | 7.8 |
| B-7 | 0.002 | 0.002 | 5.5 | B-19 | 0.367 | 0.294 | >10 |
| B-8 | 1.7 | 1.9 | NT | B-22 | 0.031 | 0.047 | 5.3 |
| B-9 | >10 | >10 | NT | B-23 | 0.507 | 0.431 | >10 |
| B-11 | 0.008 | 0.025 | 4.3 | B-24 | 0.123 | 0.167 | >10 |
| B-25 | 0.012 | 0.009 | 1.1 | B-57 | 0.059 | 0.063 | >10 |
| B-26 | 0.177 | 0.080 | 7.5 | B-58 | 0.135 | 0.150 | >10 |
| B-27 | 0.005 | 0.005 | 2 | B-59 | 0.011 | 0.016 | >10 |
| B-28 | 0.032 | 0.060 | 8.1 | B-60 | 0.310 | 0.706 | >10 |
| B-29 | 0.034 | 0.037 | >10 | B-61 | 0.062 | 0.068 | >10 |
| B-30 | 0.030 | 0.052 | >10 | B-62 | 0.029 | 0.021 | 6.4 |
| B-31 | 0.009 | 0.011 | 6.6 | B-63 | 0.061 | 0.059 | 5.8 |
| B-32 | 0.018 | 0.029 | 543.6 | B-64 | 0.008 | 0.012 | 4.449 |
| B-33 | 0.121 | 0.257 | >10 | B-65 | 0.015 | 0.016 | 3.53 |
| B-34 | 0.041 | 0.074 | 7.6 | B-66 | 0.012 | 0.028 | NT |
| B-35 | 0.010 | 0.019 | 6.6 | B-67 | 0.006 | 0.0025 | 2 |
| B-36 | 0.001 | 0.001 | 6.7 | B-68 | 0.045 | 0.0715 | >10 |
| B-37 | 0.001 | 0.004 | 4.6 | B-69 | 0.047 | 0.036 | >1 |
| B-43 | 3.9 | >10 | >10 | B-70 | 0.0165 | 0.066 | 4.757 |
| B-44 | 0.006 | 0.014 | | B-71 | 0.042 | 0.056 | >10 |
| B-45A | 0.0002 | 0.001 | 3.8 | B-72 | 0.222 | 0.223 | >10 |
| B-45B | 0.005 | 0.011 | 1.7 | B-73 | 0.14 | 0.22 | >10 |
| B-51 | 0.07 | 0.01 | 1.1 | B-74 | 0.05 | 0.04 | 4.4 |
| B-52 | 0.01 | 0.01 | 45 | B-75 | 0.03 | 0.06 | NT |
| B-53 | 0.029 | 0.051 | >10 | B-76 | 0.168 | 0.247 | >10 |
| B-54 | 0.046 | 0.069 | >10 | B-77 | 0.166 | 0.160 | 6.57 |
| B-55 | 0.040 | 0.053 | >10 | B-78 | 0.177 | 0.080 | 7.5 |
| B-66 | 0.030 | 0.104 | 4.354 | B-79 | 0.07 | 0.08 | 9.4 |
| B-80 | 0.043 | 0.093 | 6.8 | B-92 | 0.137 | 0.170 | >10 |
| B-81 | >10 | >10 | >10 | B-93 | 0.091 | 0.077 | >10 |
| B-82 | 0.20 | 0.16 | >10 | B-94 | 1.06 | 1.37 | >10 |
| B-83 | 0.12 | 0.12 | >10 | B-95 | 0.320 | 0.74 | >10 |
| B-84 | 0.086 | 0.084 | NT | B-96 | 0.080 | 0.293 | N/A |
| B-85 | 0.012 | 0.024 | >10 | B-97 | 0.071 | 0.0455 | 6.35 |
| B-86 | 0.227 | 0.239 | >10 | B-98 | 0.181 | NT | >10 |
| B-87 | >10 | >10 | >10 | B-99 | 0.207 | 0.504 | >10 |
| B-88 | 0.704 | 0.389 | >10 | B-100 | 0.0285 | 0.04 | 7.845 |
| B-89 | 0.008 | 0.002 | >10 | B-101 | 0.155 | NT | 7.97 |
| B-90 | 0.013 | 0.030 | >10 | NT indicates not tested. | | | |
| B-91 | 0.076 | 0.075 | N/A | | | | |

TABLE C-3

| | IC₅₀ (μM) | | |
|---|---|---|---|
| No. | 786-O | SJSA-1 | A431 |
| C-1 | 0.057 | 0.093 | >10 |
| C-2 | 0.023 | 0.027 | >10 |
| C-3 | 0.14 | 0.22 | >10 |
| C-4 | 0.048 | 0.045 | 4.4 |
| C-5 | 0.22 | 0.22 | >10 |
| C-6 | >10 | >10 | >10 |
| C-7 | 0.043 | 0.093 | 6.8 |
| C-11 | 0.098 | 0.22 | >10 |
| C-14 | 0.017 | 0.017 | >10 |
| C-18 | 0.4369 | 0.3317 | NT |
| C-19 | 2.318 | 1.89 | NT |
| C-20 | 4.491 | 3.98 | NT |
| C-21 | 0.07 | 0.08 | 9.4 |
| C-35 | 0.45 | NT | >10 |
| C-36 | 0.69 | NT | >10 |
| C-37 | 0.017 | 0.054 | >10 |
| C-38 | 0.437 | 0.332 | 2.947 |

NT indicates not tested.

### Example 2: GPX4 Inhibition Assay

Studies have shown that lipophilic antioxidants, such as ferrostatin, can rescue cells from GPX4 inhibition-induced ferroptosis. For instance, mesenchymal state GPX4-knockout cells can survive in the presence of ferrostatin, however, when the supply of ferrostatin is terminated, these cells undergo ferroptosis (see, e.g., Viswanathan et al., Nature 547:453-7, 2017). It has also been experimentally determined that that GPX4i can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, the ability of a molecule to induce ferroptotic cancer cell death, and that such ability is admonished by the addition of ferrostatin, is an indication that the molecule is an GPX4 inhibitor.

### Example 3: Method and Results of Western Blot Gel Mobility Shift of GPX4

A mobility shift of GPX4 Western blot assay was established to assess target engagement directly in cell-based assay after incubation with compounds and in tumors from mice treated with compounds. Mobility shift can be used as a pharmacodynamic marker for GPX4 irreversible inhibitors. For cell-based assay, cells that are sensitive to GPX4 inhibitors (e.g. MiaPaCa-2) were seeded in 10 cm (2-8×10⁶ cells) and grown overnight. Cell seeding number can be adjusted proportionally based on the surface area if smaller dishes are used. Next day, cells were treated with DMSO and various compounds at indicated concentrations for a period of time (e.g. 0.5, 1, 2, 4, 6, or up to 72 hours). Cells were then lysed in 0.3-0.5 mL of RIPA buffer (Sigma) supplemented with protease inhibitors (Roche) and phosphatase inhibitors (Sigma). Lysates were assayed for protein concentration using BCA kit (Pierce). Normalized amount of lysates (20-40 μg protein/lane) were run on 4-12% or 12% NuPage gel (Life Technologies) and the proteins were transferred to the PVDF or nitrocellulose membrane using iBlot® Transfer Stack (Life Technologies). The membranes were probed with primary antibodies shown in Table 4 at 4° C. overnight after blocking with 1×TBST containing 5% non-fat milk for one hour at room temperature. Similar antibodies from other vendors could also be used in Western blot analysis. After washing 5 times with 1×TBS containing 0.1% Tween20, the membranes were probed with 2^{nd} antibodies (e.g. Anti-mouse-HRP, Anti-rabbit-HRP, Anti-Goat-HRP, Anti-mouse IgG Dylight 800 conjugate or Anti-rabbit IgG DyLight 680 conjugate (1:10000; Cell signaling or similar IR 2^{nd} antibodies from different vendors) at room temperature for one hour. After washing 5 times, the membranes were scanned using ImageQuant-LAS-4010 (chemiluminiscence) (GE Healthcare) if HRP-conjugated secondary antibodies were used or Odyssey® Imaging System (Licor Biosciences) if infrared conjugated secondary antibodies were used.

TABLE 4
Primary antibodies used for Western blot analysis

| Antibody Name | Vendor | Cat No. | Species | MW | Dilution |
|---|---|---|---|---|---|
| β-Actin | Sigma | A5441 | Mouse | 43 kd | 1:10000 |
| (loading control) | | | | | |
| Vinculin | Sigma | V9131 | Mouse | 116 KD | 1:2000 |
| (loading control) | | | | | |
| GPX4 | Abcam | ab125066 | Rabbit | 22 kd | 1:1000 |
| GPX4 | Abcam | ab41787 | Rabbit | 22 kd | 1:1000 |

Select compounds were evaluated in cell-based Western blot analysis of GPX4. In DMSO treated sample, GPX4 ran as doublet—the major lower free or unbound GPX4 band and the minor upper band (likely glutathione-bound GPX4 (Cozza et al., Free Radical Biology and Medicine, Vol 112, pages 1-11, 2017)). The amount of upper band can be reduced if samples were boiled in excess amount of reducing agent DTT. GPX4 in SDS-PAGE reducing gel moved slower (appear as a larger molecular weight protein) when treated with covalent, irreversible inhibitors of GPX4 (e.g. RSL-3 and ML162) but not reversible inhibitors (e.g. ML210), presumably due to addition of the covalently linked small molecule to GPX4. Unlike glutathione-bound GPX4, the irreversible inhibitor bound GPX4 upper band can't be reduced by excess amount of DTT. Further, distance of the GPX4 mobility shift is correlated with the molecular weight of the irreversible GPX4 inhibitor shifted distance is bigger with larger irreversible inhibitors. Thus, this simple mobility shift of GPX4 Western blot can be used to conveniently assess direct target engagement in vitro, in cells and in tumors by irreversible inhibitors. Treatment of MiaPaCa-2 cells with certain compounds disclosed herein resulted in dose-dependent mobility shift of GPX4 from the lower unbound to upper bound bands. At concentrations greater than 50 nM (e.g., 100 nM), certain tested compounds of Formula B-I converted nearly all GPX4 to the upper bands.

### Example 4: Kinact/Ki Determination for GPX4 Inhibitors

Day 1—seed cells: Cells are seeded with 5×10⁵ Calu6 cells/well into 5×6-well plates.

Day 2—treat cells with Cmpd, prepare samples for gels: Cells are treated with 1, 0.75, 0.5, 0.25 and 0.1 μM inhibitor+2 μM Ferrostatin-1 for 0, 10, 20, 30, 45, 60 minutes. 10 μL of 1000×DMSO stock solutions are prepared for each compound dilution (1, 0.75, 0.5, 0.25, 0.1 mM). Complete cell culture media (EMEM+10% FBS) is prepared with 2 μM Ferrostatin-1 final conc. Drug solutions are prepared by adding 1000× inhibitors to Ferrostatin-1-supplemented media at 1× final concentration (1, 0.75, 0.5, 0.25, 0.1 μM) plus DMSO for use as a negative control.

Cell lysis buffer is prepared by diluting 5× cell lysis buffer (Cell Signaling Technology #9803) and 100× protease/phosphatase inhibitor cocktail (Cell Signaling Technology #5872) to 1× with DI water.

Cells are treated with drug solutions in 1-hour time course. One concentration of drug added to each 6-well plate at t=60, 45, 30, 20, 10, 0 minutes. Media is aspirated from cells in 1 well of each 6-well plate and add 1 mL of media w/drug+ferrostatin (t=60 min). Cells are returned to incubator between time points. Media is aspirated and drug added to cells at each subsequent time point. At t=10 min DMSO is added negative control to additional well.

At t=0 media is aspirated from cells, cells are washed with ice cold PBS and aspirated, 75 μL of 1× cell lysis buffer is added per well, bottom of plates scraped with cell scraper, and lysates transferred to 1.5 mL Eppendorf tubes at store at −20° C.

SDS-PAGE running buffer is prepared (2 L of 1×MES Bolt running buffer (ThermoFisher Scientific #B0002), and stored at 4 C overnight for use the next day).

Day 3—perform BCA assay and run gels: Lysates are thawed on ice, centrifuged at 18,000×g at 4 C for 10 minutes, and BCA assay is performed on supernatant following manufacturer protocol (ThermoFisher Scientific #23225). 3.6×LDS/BME sample buffer is prepared by mixing Bolt 4×LDS sample buffer (ThermoFisher Scientific #B0008) with 2-mercaptoethanol at a 10:1 ratio. In 96-well PCR plate 19 μL 3.6×LDS/BME sample buffer is added and 50 μL lysate samples. Lysates diluted to 1 mg/mL with 1×LDS/BME, plates heated at 95 C for 10 min in PCR machine, loaded 15 uL/well (15 ug total lysate) into 12% Bis-Tris Bolt gels, and gels run at 200V for 35 minutes (until dye front reaches bottom of gel) with cold 1×MES running buffer. After which time, gels are washed 5 minutes in water, 10 minutes in 20% Ethanol/water, and transferred to membrane with iBlot2 (ThermoFisher Scientific). Membrane was blocked 1 h at RT with Licor TBS blocking buffer (Licor #927-60001) and incubated with 1:1000 dilution of anti-GPX4 antibody (Abcam #ab125066) in Licor TBS blocking buffer at 4 C overnight with gentle rocking.

Day 4—develop blots, quantify gel shift: Membrane is washed with 1×TBST for 30 minutes (change wash buffer 3-4 times), incubated with Licor secondary antibody (Licor #926-68021) 1:40,000 in Licor TBS blocking buffer for 1 h at RT with gentle rocking, washed with 1×TBST for 30 minutes, scraped with Licor imager and bands quantied with Image studio.

### Example 5: Pharmacokinetics Studies

Institutional Animal Ethical Committee (IAEC) of Jubilant Biosys (IAEC/JDC/2019/188R (for Mice) and IAEC/JDC/2019/189R (for Rat) nominated by CPCSEA (Committee for the Purpose of Control and Supervision of Experiments on Animals) approved the mice and rat pharmacokinetic experiments. Male Balb/c mice (˜6-8 weeks old with body weight range of 22-25 g) and male SD rats (6-8 weeks old with body weight range of 200-250 g) were procured from Vivo Biotech, Hyderabad, India. Animals were quarantined in Jubilant Biosys Animal House for a period of 7 days with a 12:12 h light: dark cycles, and prior to the study the animals were stratified as per body weight.

Housing: The animals were group housed in standard polycarbonate cages, with stainless steel top grill where pelleted food and drinking water bottle are placed; corn cob was used as bedding material and changed at least twice a week or as required.

Diet ad libitum: Rodent feed manufactured by Altromin Spezialfutter GmbH & Co. KG., ImSeelenkamp20. D-32791 Lage, was provided.

Water ad libitum: Purified water was provided ad libitum to animals in polycarbonate bottles with stainless steel sipper tubes.

A) Procedure for Mice: Intravenous, oral and intraperitoneal pharmacokinetics study was done at doses of 5, 20 and 10 mg/kg respectively at dose volume of 10 mL/Kg for PO and IP while 5 mL/kg for IV route. Sparse sampling was done and at each time point three mice were used for blood sampling (˜100 μL) were collected from retro-orbital plexus at 0.083 (Only for IV), 0.25, 0.5, 1, 2, 4, 8, 10 (only for PO) and 24 h. Blood samples collected in tubes containing K₂.EDTA as anticoagulant and centrifuged for 5 min at 10,000 rpm in a refrigerated centrifuge (Biofuge, Heraeus, Germany) maintained at 4° C. for plasma separation.

Group I (IV) received test compound intravenously by tail vein at 5 mg/Kg in solution formulation prepared using 30% Kolliphore EL in WFI; dose volume: 5 mL/Kg; strength: 1 mg/mL.

Group II (PO) received test compound by per oral route using oral gavage needle at 20 mg/Kg in solution formulation prepared using 30% Kolliphore EL in WFI; dose volume: 10 mL/Kg; strength: 2 mg/mL.

Group III (IP) received test compound by intraperitoneal route at 10 mg/Kg in solution formulation prepared using 30% Kolliphore EL in WFI; dose volume: 10 mL/Kg; strength: 1 mg/mL.

B) Procedure for rat: Intravenous and oral pharmacokinetics study was done at a dose 2 and 10 mg/kg at dose volume of 2 and 10 mL/Kg. Serial blood sampling was done and at each time point (˜200 μL) were collected from retro-orbital plexus at 0.083 (Only for IV), 0.25, 0.5, 1, 2, 4, 8, 10 (only for PO) and 24 h. Blood samples were collected in tubes containing K₂.EDTA as anticoagulant and centrifuged for 5 min at 10,000 rpm in a refrigerated centrifuge (Biofuge, Heraeus, Germany) maintained at 4° C. for plasma separation.

Group I (IV) received test compound intravenously by tail vein at 2 mg/Kg in solution formulation prepared using 30% Kolliphore EL in WFI; dose volume: 2 mL/Kg; strength: 1 mg/mL.

Group II (PO) received test compound using oral gavage needle at 10 mg/Kg (solution formulation prepared using 30% Kolliphore EL in WFI; dose volume: 10 mL/Kg: strength: 1 mg/mL.

Blood concentration-time data of test compound was analyzed by non-compartmental method using Phoenix WinNonlin Version 8.1.

Table B-4 below shows certain pharmacokinetic parameters for compounds of Formula B-I.

TABLE B-4

| No. | B-3 | B-5 | B-25 | B-27 | B-31 | B-35 | B-36 | B-37 |
|---|---|---|---|---|---|---|---|---|
| Kinetic | 4 | 1 | 1 | <1 | 3 | <1 | <1 | <1 |
| solubility | | | | | | | | |
| (PBS, pH 7.4, | | | | | | | | |
| μM) | | | | | | | | |
| rat | — | 0.9 | 57 | — | 100 | 94 | 72/107/ | 74/137/ |
| hepatocytes_ | | | (151 min)/ | | (16 min)/ | (68 min)/ | 26/0.7 | 20/0.64 |
| % met @ 180 | | | 18/0.61 | | 174/0.94 | 41/0.78 | | |
| min (T1/2)/ | | | | | | | | |
| h Clᵢₙₜᵣ | | | | | | | | |
| (μL/min/10⁶ | | | | | | | | |
| cells)/extr. | | | | | | | | |
| Ratio | | | | | | | | |
| human | — | 0.82 | 42 | — | 97 | 73 | 3.5/2464/ | 38/270/ |
| hepatocytes_ | | | (245 min)/ | | (28 min)/ | (98 min)/ | 1.1/0.13 | 10/0.58 |
| % met @ 180 | | | 11/0.61 | | 99/0.93 | 28/0.8 | | |
| min (T1/2)/ | | | | | | | | |
| h Clᵢₙₜᵣ | | | | | | | | |
| (μL/min/10⁶ | | | | | | | | |
| cells)/extr. | | | | | | | | |
| Ratio | | | | | | | | |
| human blood | 90%_15 | 36%_15 | 80%_15 | 38%_15 | 9.7%_15 | 107%_15 | ~100%_15 | |
| stability_ | min | min | min | min | min | min | min | |
| % remaining_ | 55%_30 | 41%_30 | 38%_30 | 4%_30 | 0%_30 | 31%_30 | 96%_30 | |
| time | min | min | min | min | min | min | min | |
| | 36%_45 | 11%_45 | 28%_45 | 1%_45 | 0%_45 | 15%_45 | 82%_45 | |
| | min | min | min | min | min | min | min | |
| | 22%_60 | 9%_60 | 18%_60 | 0%_60 | 0%_60 | 8.4%_60 | 77%_60 | |
| | min | min | min | min | min | min | min | |
| | 1.5%_120 | 0%_120 | 0%_120 | 0%_120 | 0%_120 | 0%_120 | 51%_120 | |
| | min | min | min | min | min | min | min | |
| Rat IV-PK (2 | CL: 348 | | | | | | T_{1/2} 0.5 h | T_{1/2} 3.3 h |
| mg/kg) | ml/min/kg | | | | | | AUC (0-t) | AUC (0-t) |
| | | | | | | | 431 | 346 |
| | | | | | | | ng*h/mL | ng*h/mL |
| | | | | | | | CL 87 | CL 115 |
| | | | | | | | mL/min/kg | mL/min/kg |
| | | | | | | | Vd: 3.5 | Vd: 32 |
| | | | | | | | L/kg | L/kg |

Table B-5 below shows free drug percentage in the presence of protein binding for compound B-25.

TABLE B-5

| | Protein binding - free drug % |
|---|---|
| No. | (mouse) |
| B-25 | 9.4% |

Table B-6 below shows data from a whole blood stability assay along with percentage of unbound drug for certain compounds described herein.

TABLE B-6

| Summary of CD-1 Mouse Whole | | | |
|---|---|---|---|
| Blood Stability Assay | | | Mouse Plasma |
| (incubation at 37° C.) | | | Protein % Unbound |
| | Time Point | % | (Ultracentrifuge |
| No. | (min) | Remaining | method, 4 h at 37° C.) |
| B-22 | 0 | 100.0 | 3.7% (53% remaining) |
| | 30 | 70.3 | |
| | 60 | 55.0 | |
| B-23 | 0 | 100.0 | Not stable |
| | 30 | 4.0 | |
| | 60 | 0.5 | |
| B-26 | 0 | 100.0 | 6.1% (22% remaining) |
| | 30 | 44.7 | |
| | 60 | 26.7 | |
| B-27 | 0 | 100.0 | 7.8% (42% remaining) |
| | 30 | 78.0 | |
| | 60 | 72.7 | |
| B-28 | 0 | 100.0 | |
| | 30 | 9.6 | |
| | 60 | 2.1 | |
| B-29 | 0 | 100.0 | Not stable |
| | 30 | 18.0 | |
| | 60 | 3.9 | |
| B-30 | 0 | 100.0 | Not stable |
| | 30 | 1.9 | |
| | 60 | 0.5 | |
| B-31 | 0 | 100.0 | Not stable |
| | 30 | 2.1 | |
| | 60 | 0.2 | |
| B-34 | 0 | 100.0 | Not stable |
| | 30 | 9.4 | |
| | 60 | 1.1 | |
| B-50 | 0 | 100 | |
| | 30 | 22.3 | |
| | 60 | 10.5 | |
| B-51 | 0 | 100 | (4.8% remaining) |
| | 30 | 0 | |
| | 60 | 0 | |
| B-52 | 0 | 100 | (1.6% remaining) |
| | 30 | 0.6 | |
| | 60 | 0 | |

Table B-7 below shows solubility of certain compounds of Formula B-I at different pHs and after addition of sulfobutyl ether cyclodextrin (SBECD).

TABLE B-7

| No. | B-30 | B-31 | B-32 | B-33 | B-34 | B-35 |
|---|---|---|---|---|---|---|
| Kinetic solubility | <1 | 3 | <1 | N/T | 11 | <1 |
| (PBS, pH 7.4, μM) | | | | | | |
| Eq. solubility pH 3.0 | 0.70 | 0.015 | <0.001 | N/T | N/T | N/T |
| | mg/mL | mg/mL | mg/mL | | | |
| Eq. solubility pH 3.0 | 0.49 | 0.004 | <0.001 | <0.001 | <0.001 | N/T |
| PBS buffer | mg/mL | mg/mL | mg/mL | mg/mL | mg/mL | |
| Eq. solubility 20% | 0.24 | 0.047 | 0.009-0.018 | >1.09 | >1.01 | 0.01 |
| SBECD | mg/mL | mg/mL | mg/mL | mg/mL | mg/mL | mg/mL |

Table B-8 shows percent bound and unbound drug in mouse plasma and percentage drug remaining after 4 hours in mouse plasma for certain compounds of Formula B-I. Warfarin is a control.

TABLE B-8

| | | | | | % Remaining |
|---|---|---|---|---|---|
| | Concentration | Species/ | % Unbound | % | (t = 4 hr at |
| No. | (μM) | Matrix | (n = 3) | Bound | 37 C.) |
| B-53 | 2 | CD-1 Mouse | 8.3 | 91.7 | 22.0 |
| | | Plasma | | | |
| B-54 | 2 | CD-1 Mouse | 9.7 | 90.3 | 31.6 |
| | | Plasma | | | |
| B-55 | 2 | CD-1 Mouse | NA | NA | 21.5 |
| | | Plasma | | | |
| B-56 | 2 | CD-1 Mouse | 11.0 | 89.0 | 56.7 |
| | | Plasma | | | |
| Warfarin | 2 | CD-1 Mouse | 3.5 (SD 0.3) | 96.5 | 101.2 |
| | | Plasma | | | |

Table C-4 below shows a summary of CD-1 Mouse Whole Blood Stability Assay along with percentage of unbound drug for compound C-38

| Summary of CD-1 Mouse Whole | | | |
|---|---|---|---|
| Blood Stability Assay | | | Mouse Plasma |
| (incubation at 37° C.) | | | Protein % Unbound |
| | Time Point | % | (Ultracentrifuge |
| No. | (min) | Remaining | method, 4 h at 37° C.) |
| C-38 | 0 | 100.0 | 10.5% (82% |
| | 30 | 74.4 | remaining) |
| | 60 | 61.0 | |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A compound of formula A-I: 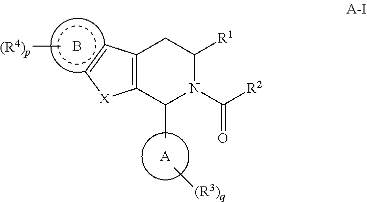
or a tautomer, stereoisomer, mixture of stereoisomers, isotopically enriched analog, or pharmaceutically acceptable salt thereof, wherein:
ring A is C₄-C₁₀cycloalkyl, heterocyclyl, aryl, or heteroaryl;
ring B is a 6-membered heteroaryl containing one or two N-atoms;
X is NR⁵, O or S;
p is 0, 1, 2 or 3;
q is 0, 1, 2 or 3;
R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OH, —C₁-C₆alkyl-OR⁸, or —Si(R¹⁵)₃;
R² is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently optionally substituted with one to three R¹⁰;
each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is optionally independently optionally substituted with one to three R¹⁰;
R⁵ is hydrogen or C₁-C₆alkyl;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of e is optionally independently substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl or C₃-C₁₀cycloalkyl;
each R¹³ is independently C₁-C₆alkyl or C₃-C₁₀cycloalkyl; and
each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, or heteroarylC₂-C₆alkenyl-.

2. The compound of claim 1, or a tautomer, stereoisomer, mixture of stereoisomers, isotopically enriched analog, or pharmaceutically acceptable salt thereof, represented by formula A-IA:

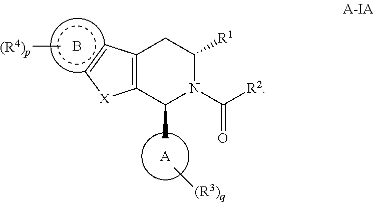

3. The compound of claim 1, or a tautomer, stereoisomer, mixture of stereoisomers, isotopically enriched analog, or pharmaceutically acceptable salt thereof, represented by formula A-II:

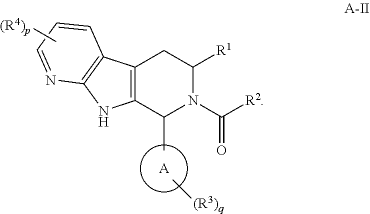

4. The compound of claim 1, or a tautomer, stereoisomer, mixture of stereoisomers, isotopically enriched analog, or pharmaceutically acceptable salt thereof, represented by formula A-III:

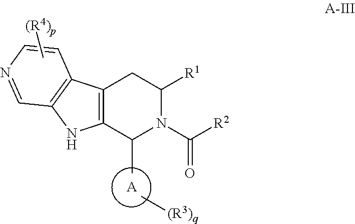

5. The compound of claim 1, or a tautomer, stereoisomer, mixture of stereoisomers, isotopically enriched analog, or pharmaceutically acceptable salt thereof, represented by formula A-IV:

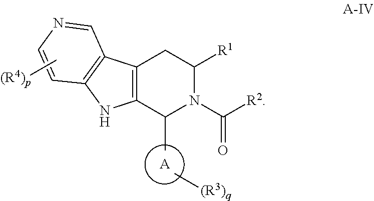

6. The compound of claim 1, or a tautomer, stereoisomer, mixture of stereoisomers, isotopically enriched analog, or pharmaceutically acceptable salt thereof, represented by formula A-V:

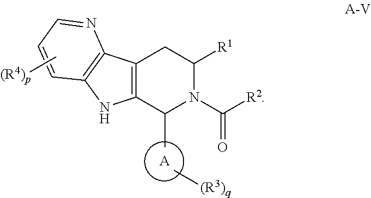

7. The compound of claim 1, or a tautomer, stereoisomer, mixture of stereoisomers, isotopically enriched analog, or pharmaceutically acceptable salt thereof, represented by formula A-VI:

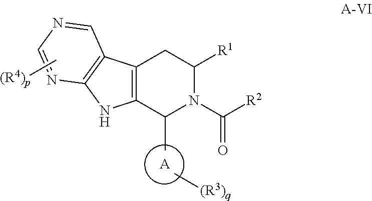

8. The compound of claim 1, or a tautomer, stereoisomer, mixture of stereoisomers, isotopically enriched analog, or pharmaceutically acceptable salt thereof, represented by formula A-VII:

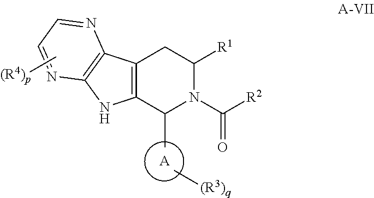

9. The compound of claim 1, or a tautomer, stereoisomer, mixture of stereoisomers, isotopically enriched analog, or pharmaceutically acceptable salt thereof, represented by formula A-VIII:

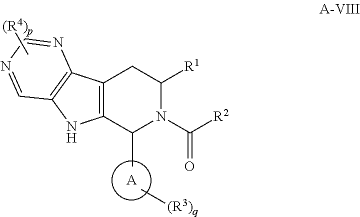

10. The compound of any preceding claim, wherein R² is C₁-C₂alkylhalo.

11. The compound of any preceding claim, wherein R² is CH₂Cl or CD₂Cl.

12. The compound of any preceding claim, wherein R² is —C≡CH.

13. The compound of claim 1, or a tautomer, stereoisomer, mixture of stereoisomers, isotopically enriched analog, or pharmaceutically acceptable salt thereof, represented by formula A-IX: 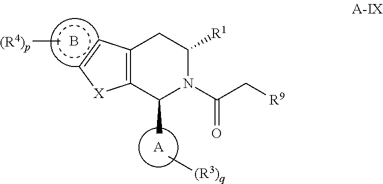
wherein R⁹ is halo.

14. The compound of claim 1, or a tautomer, stereoisomer, mixture of stereoisomers, isotopically enriched analog, or pharmaceutically acceptable salt thereof, represented by formula A-X:

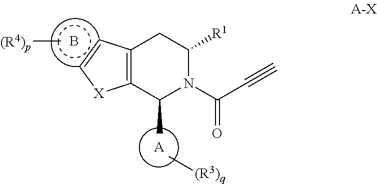

15. The compound of any preceding claim, wherein R¹ is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸.

16. The compound of any preceding claim, wherein R¹ is C₁-C₆alkyl, C₂-C₆alkenyl, C₂-C₆alkynyl, C₁-C₆haloalkyl, C₃-C₁₀cycloalkyl, —NH₂, —NHR⁸, —N(R⁸)₂, —OH, —OR⁸, —C₁-C₆alkyl-OH or —C₁-C₆alkyl-OR⁸.

17. The compound of any preceding claim, wherein R¹ is —C(O)OR⁶ or —C(O)N(R⁷)₂.

18. The compound of any preceding claim, wherein R¹ is C₁-C₆alkyl.

19. The compound of claim 1, or a tautomer, stereoisomer, mixture of stereoisomers, isotopically enriched analog, or pharmaceutically acceptable salt thereof, represented by formula A-XII:

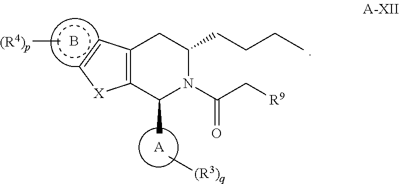

20. The compound of claim 1, or a tautomer, stereoisomer, mixture of stereoisomers, isotopically enriched analog, or pharmaceutically acceptable salt thereof, represented by formula A-XIII:

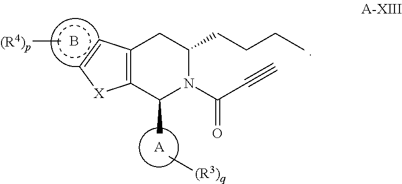

21. The compound of any preceding claim, wherein X is —NH—.

22. The compound of any preceding claim, wherein each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰.

23. The compound of any preceding claim, wherein each R⁴ is independently halo, —CN, —OH, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

24. The compound of any preceding claim, wherein ring A is C₄-C₁₀cycloalkyl.

25. The compound of any preceding claim, wherein ring A is heterocyclyl.

26. The compound of any preceding claim, wherein ring A is aryl.

27. The compound of any preceding claim, wherein ring A is heteroaryl.

28. The compound of any preceding claim, wherein each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently optionally substituted with one to three R¹⁰.

29. The compound of any preceding claim, wherein each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl is independently optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein
each R¹² is independently hydrogen, C₁-C₆alkyl or C₃-C₁₀cycloalkyl; and
each R¹³ is independently C₁-C₆alkyl or C₃-C₁₀cycloalkyl.

30. The compound of any preceding claim, wherein at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

31. The compound of any preceding claim, wherein at least one R³ is halo.

32. The compound of any preceding claim, wherein at least one R³ is —NHR⁸.

33. The compound of any preceding claim, wherein at least one R³ is —C(O)OR⁶ or —C(O)R⁶.

34. The compound of any preceding claim, wherein p is 0.

35. The compound of any preceding claim, wherein p is 1, 2 or 3.

36. The compound of any preceding claim, wherein p is 1.

37. The compound of any preceding claim, wherein p is 2.

38. The compound of any preceding claim, wherein q is 1.

39. The compound of any preceding claim, wherein q is 2.

40. A compound of formula C-I: 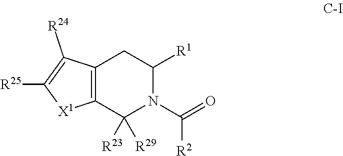
or a tautomer, stereoisomer, mixture of stereoisomers, isotopically enriched analog, or pharmaceutically acceptable salt thereof, wherein:
X is —NH—, —N(C₁-C₆alkyl)-, —O—, —S—, —N═CR¹⁶—, —CR¹⁶═CR¹⁶—, or —CR¹⁶═N—;
R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OH, —C₁-C₆alkyl-OR⁸, or —Si(R¹⁵)₃;
R² is —C₁-C₂haloalkyl, —C₂-C₃alkenyl, —C₂-C₃haloalkenyl, C₂alkynyl, or —CH₂OS(O)₂-phenyl, wherein the C₁-C₂alkylhalo and —C₂-C₃alkenylhalo are optionally substituted with one or two —CH₃, and the C₂alkynyl and phenyl are optionally substituted with one —CH₃;
R²³ is C₁-C₉alkyl, C₂-C₉alkenyl, C₂-C₉alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl, wherein each C₁-C₉alkyl, C₂-C₉alkenyl, C₂-C₉alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl of R²³ is independently optionally substituted with one to three R¹⁷;
R²⁹ is hydrogen or C₁-C₆alkyl; provided that when R²³ is C₃-C₁₀cycloalkyl, heterocyclyl, aryl, heteroaryl, then R²⁹ is C₁-C₆alkyl;
R²⁴ and R²⁵ are each independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R²⁴ and R²⁵ is independently optionally substituted with one to three R¹⁰; or
when X is —NH—, —N(C₁-C₆alkyl)-, —O—, or —S—; then R²⁴ and R²⁵ together with the atoms to which they are attached, can form a 6-membered aryl or 6-membered heteroaryl, wherein each aryl or heteroaryl is optionally substituted with one to three R¹⁴;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently further substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently further substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently further substituted with one to three R¹¹;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is optionally independently substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl or C₃-C₁₀cycloalkyl;
each R¹³ is independently C₁-C₆alkyl or C₃-C₁₀cycloalkyl;
each R¹⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁴ is independently optionally substituted with one to three R¹⁰;
each R¹⁵ is independently C₁-C₆alkyl, C₂-C₆alkenyl, aryl, heteroaryl, arylC₁-C₆alkyl-, arylC₂-C₆alkenyl-, heteroarylC₁-C₆alkyl-, and heteroarylC₂-C₆alkenyl-;
each R¹⁶ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁶ is independently optionally substituted with one to three R¹⁰; and
each R¹⁷ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R¹⁷ is independently optionally substituted with one to three R¹⁰.

41. The compound of claim 40, or a tautomer, stereoisomer, mixture of stereoisomers, isotopically enriched analog, or pharmaceutically acceptable salt thereof, represented by formula C-II:

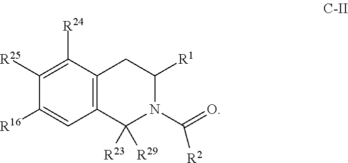

42. The compound of claim 40, or a tautomer, stereoisomer, mixture of stereoisomers, isotopically enriched analog, or pharmaceutically acceptable salt thereof, represented by formula C-III: 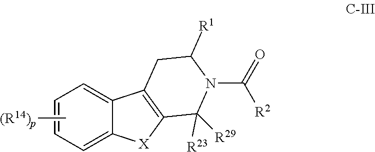
wherein
p is 0, 1, 2 or 3; and
q is 0, 1, 2 or 3.

43. The compound of claim 40, or a tautomer, stereoisomer, mixture of stereoisomers, isotopically enriched analog, or pharmaceutically acceptable salt thereof, represented by formula C-IV:

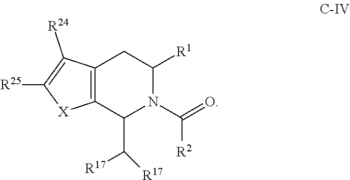

44. The compound of claim 40, or a tautomer, stereoisomer, mixture of stereoisomers, isotopically enriched analog, or pharmaceutically acceptable salt thereof, represented by formula C-VI: 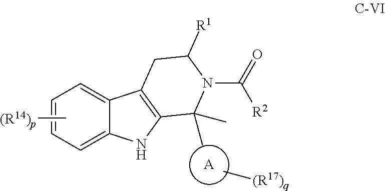
wherein
p is 0, 1, 2 or 3; and
q is 0, 1, 2 or 3.

45. The compound of one of claims 40-44, wherein R¹ is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸.

46. The compound of any one of claims 40-45, wherein R¹ is C₁-C₆alkyl, C₂-C₆alkenyl, C₂-C₆alkynyl, C₁-C₆haloalkyl, C₃-C₁₀cycloalkyl, —NH₂, —NHR⁸, —N(R⁸)₂, —OH, —OR⁸, —C₁-C₆alkyl-OH or —C₁-C₆alkyl-OR⁸.

47. The compound of any one of claims 40-46, wherein R¹ is —C(O)OR⁶ or —C(O)N(R⁷)₂.

48. The compound of any one of claims 40-47, wherein R² is —C₁-C₂alkylhalo.

49. The compound of any one of claims 40-48, wherein R² is-CH₂Cl or -CD₂Cl.

50. The compound of any one of claims 40-49, wherein R² is —C≡CH.

51. The compound of any one of claims 40-50, wherein each R²⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R²⁴ is independently optionally substituted with one to three R¹⁰.

52. The compound of any one of claims 40-51, wherein each R²⁴ is independently halo, —CN, —OH, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R²⁴ is independently optionally substituted with one to three R¹⁰.

53. The compound of any one of claims 40-52, wherein each R¹⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R¹⁴ is independently optionally substituted with one to three R¹⁰.

54. The compound of any one of claims 40-53, wherein each R¹⁴ is independently halo, —CN, —OH, —OR⁸, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R¹⁴ is independently optionally substituted with one to three R¹⁰.

55. The compound of any one of claims 40-54, wherein X is —NH—, —N(C₁-C₆alkyl)-, —O—, or —S—.

56. The compound of any one of claims 40-55, wherein X is —NH—.

57. The compound of any one of claims 40-56, wherein X is —N═CR¹⁶—, —CR¹⁶═CR¹⁶—, or —CR¹⁶═N—.

58. The compound of any one of claims 40-57, wherein X is —CR¹⁶═CR¹⁶—.

59. The compound of any one of claims 40-58, wherein X is —CH═CH—.

60. The compound of any one of claims 40-59, wherein R²³ or ring A is C₄-C₁₀cycloalkyl.

61. The compound of any one of claims 40-60, wherein R²³ or ring A is heterocyclyl.

62. The compound of any one of claims 40-61, wherein R²³ or ring A is aryl.

63. The compound of any one of claims 40-62, wherein R²³ or ring A is heteroaryl.

64. The compound of any one of claims 40-63, wherein p is 0.

65. The compound of any one of claims 40-64, wherein p is 1, 2 or 3.

66. The compound of any one of claims 40-65, wherein p is 1.

67. The compound of any one of claims 40-66, wherein p is 2.

68. The compound of any one of claims 40-67, wherein q is 1.

69. The compound of any one of claims 40-68, wherein at least one R¹⁷ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SFS, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

70. The compound of any one of claims 40-69, wherein at least one R¹⁷ is halo.

71. The compound of any one of claims 40-70, wherein at least one R¹⁷ is —NHR⁸.

72. The compound of any one of claims 40-71, wherein at least one R¹⁷ is —C(O)OR⁶ or —C(O)R⁶.

73. The compound of any one of claims 40-72, wherein each R¹⁷ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R²³ is independently optionally substituted with one to three R¹⁰.

74. The compound of any one of claims 40-73, wherein each R¹⁷ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl is independently optionally substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein
each R¹² is independently hydrogen, C₁-C₆alkyl or C₃-C₁₀cycloalkyl; and
each R¹³ is independently C₁-C₆alkyl or C₃-C₁₀cycloalkyl.

75. A compound selected from the group consisting of compounds listed in Table A-1, or a tautomer, stereoisomer, mixture of stereoisomers, isotopically enriched analog, or pharmaceutically acceptable salt thereof.

76. A compound selected from the group consisting of compounds listed in Table B-1, or a tautomer, stereoisomer, mixture of stereoisomers, isotopically enriched analog, or pharmaceutically acceptable salt thereof.

77. A compound selected from the group consisting of compounds listed in Table C-1, or a tautomer, stereoisomer, mixture of stereoisomers, isotopically enriched analog, or pharmaceutically acceptable salt thereof.

78. A pharmaceutical composition comprising a compound, or a tautomer, stereoisomer, mixture of stereoisomers, isotopically enriched analog, or pharmaceutically acceptable salt thereof, of any one of claims 1 to 77, and a pharmaceutically acceptable carrier.

79. A method of inhibiting GPX4 in a cell, comprising contacting a cell with an effective amount of a compound, or a tautomer, stereoisomer, mixture of stereoisomers, isotopically enriched analog, or pharmaceutically acceptable salt thereof, of any one of claims 1 to 78.

80. The method of claim 79, wherein the cell is a cancer cell.

81. A method of treating cancer in a subject, comprising administering to a subject having cancer a therapeutically effective amount of a compound, or a tautomer, stereoisomer, mixture of stereoisomers, isotopically enriched analog, or pharmaceutically acceptable salt thereof, of any one of claims 1 to 77.

82. The method of claim 81, wherein the cancer is adrenocortical cancer, anal cancer, biliary cancer, bladder cancer, bone cancer, brain cancer, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, or a soft tissue carcinoma.

83. The method of claim 82, wherein the cancer is osteosarcoma, glioma, astrocytoma, neuroblastoma, cancer of the small intestine, bronchial cancer, small cell lung cancer, non-small cell lung cancer, basal cell carcinoma, or melanoma.

84. The method of claim 83, wherein the cancer is a hematologic cancer.

85. The method of claim 84, wherein the hematologic cancer is acute lymphoblastic leukemia (ALL), acute myeloid leukemia (AML), lymphoma (e.g., Hodgkin's lymphoma, Non-Hodgkin's lymphoma, Burkitt's lymphoma), chronic lymphocytic leukemia (CLL), chronic myelogenous leukemia (CML), Hairy Cell chronic myelogenous leukemia (CML), or multiple myeloma.

86. The method of any one of claims 81 to 85, further comprising administering a therapeutically effective amount of a second therapeutic agent.

87. The method of claim 86, wherein the second therapeutic agent is an platinating agent, alkylating agent, anti-cancer antibiotic, antimetabolite, topoisomerase I inhibitor, topoisomerase II inhibitor, or antimicrotubule agent.
